(19) United States
(12) Patent Application Publication
(10) Pub. No.: US 2019/0100766 A1
(43) Pub. Date: Apr. 4, 2019

(54) METHOD FOR INCREASING NITROGEN-USE EFFICIENCY IN PLANTS

(71) Applicants: Nanjing Agricultural University, Nanjing (CN); Nanjing Meipu Biotechnology Limited Company, Nanjing (CN)

(72) Inventors: Xiaorong Fan, Nanjing (CN); Lin Cao, Nanjing (CN)

(21) Appl. No.: 16/065,232

(22) PCT Filed: Dec. 23, 2016
(86) PCT No.: PCT/CN2016/111749

§ 371 Date: Jun. 22, 2018

(30) Foreign Application Priority Data
Dec. 24, 2015 (CN) PCT/CN2015/098633

(51) Int. Cl. C12N 15/82; C07K 14/415

(52) U.S. Cl. CPC C12N 15/8273; C12N 15/8222; C07K 14/415

(57) ABSTRACT

Disclosed herein is a method of improving yield, growth and/or nitrogen use efficiency in plants comprising altering the expression profile of NRT. Also provided methods of making such plants, including nucleic acid constructs and genetically altered plants with the above traits.

### CROSS-REFERENCE TO RELATED APPLICATIONS

This application is a national stage application of PCT/CN2016/111749 filed Dec. 23, 2016 which claims a benefit of priority from PCT/CN2015/098633 filed Dec. 24, 2015, the entire disclosures of which are herein incorporated by reference.

### FIELD OF THE INVENTION

The invention relates to a method of improving yield, growth and/or nitrogen use efficiency in plants comprising altering the expression profile of a NRT2 nucleic acid. The invention also relates to methods of making such plants, including nucleic acid constructs and genetically altered plants with the above traits.

### INTRODUCTION

Nitrogen (N) nutrition affects all levels of plant function from metabolism to resource allocation, growth, and development (Crawford, 1995; Scheible et al., 1997; Stitt, 1999; Scheible et al., 2004). The most abundant source for N acquisition by plant roots is nitrate (N03−), which is present in naturally aerobic soils due to intensive nitrification from applied organic and fertilizer N. N03− serves as a nutrient and as a signal that induces changes in growth and gene expression (Crawford and Glass et al., 1998; Wang et al., 2000; Zhang and Forde et al., 2000; Coruzzi and Bush et al., 2001; Coruzzi and Zhou et al., 2001; Crawford and Forde et al., 2002; Kronzucker et al., 2000; Kirk & Kronzucker et al., 2005). In contrast, ammonium (NH4+) is the main form of available N in flooded rice-paddy soils due to the anaerobic soil conditions (Sasakawa and Yamamoto, 1978). To varying extents, all crop plants need to be able to manage uptake, transport and metabolism of both nitrate and ammonium according to the soil conditions and other factors, such as growth stage.

The use of nitrogen by plants involves several steps, including uptake, assimilation, translocation and, when the plant is ageing, recycling and remobilization. Two different NO3− uptake systems in plants, the high- and low-affinity NO3− uptake systems designated as HATS and LATS are regulated by NO3− supply and enable plants to cope, respectively, with low or high NO3− concentrations in soils (Fan et al., 2005).

The constitutive HATS (cHATS) and nitrate-inducible HATS (iHATS) operate to take up nitrate at low nitrate concentration in external medium with saturation in a range of 0.2-0.5 mM. In contrast, LATS functions in nitrate acquisition at higher external nitrate concentration. The uptake by LATS and HATS is mediated by nitrate transporters belonging to the families of NRT1 and NRT2, respectively. Uptake by roots is regulated by negative feedback, linking the expression and activity of nitrate uptake to the N status of the plant.

Both electrophysiological and molecular studies have shown that nitrate uptake through both HATS and LATS is an active process mediated by proton/nitrate co-transporters (Zhou et al., 2000; Miller et al., 2007 In the Arabidopsis genome, there are at least 53 and 7 members belonging to NRT1 and NRT2 families, respectively (Miller et al., 2007; Tsay et al., 2007). Several Arabidopsis NRT1 and NRT2 family members have been characterized for their functions in nitrate uptake and long distance transport. AtNRT1.1 (CHL1) is described as a transceptor playing multiple roles as a dual affinity nitrate transporter and a sensor of external nitrate supply concentration (Liu and Tsay, 2003; Gojon et al., 2011), and auxin transport at low nitrate concentrations. In contrast, AtNRT1.2 (NTL1) is a constitutively expressed low affinity nitrate transporter (Huang et al., 1999). AtNRT1.4 is a leaf petiole expressed nitrate transporter and plays a critical role in regulating leaf nitrate homeostasis and leaf development (Chiu et al., 2004). AtNRT1.5 is expressed in the root pericycle cells close to the xylem and is responsible for loading of nitrate into the xylem for root-to-shoot nitrate transport (Lin et al., 2008). AtNRT1.6 is expressed only in reproductive tissues and is involved in delivering nitrate from maternal tissue to the early developing embryo (Almagro et al., 2008). AtNRT1.7 functions in phloem loading of nitrate to allow transport out of older leaves and into younger leaves, indicating that source-to-sink remobilization of nitrate is mediated by the phloem (Fan et al., 2009). AtNRT1.8 is expressed predominantly in xylem parenchyma cells within the vasculature and plays the role in retrieval of nitrate from the xylem sap (Li et al., 2010). AtNRT1.9 facilitates loading of nitrate into the root phloem, enhancing downward transport in roots, and its knockout increases root to shoot xylem transport of nitrate (Wang and Tsay, 2011). Among the 7 NRT2 family members in Arabidopsis, both AtNRT2.1 and AtNRT2.2 have been characterized as contributors to iHATS. In the rice genome, five NRT2 genes have been identified (Araki and Hasegawa, 2006; Cai et al., 2008; Feng et al., 2011). OsNRT2.1 and OsNRT2.2 share an identical coding region sequence with different 5′- and 3′-untranscribed regions (UTRs) and have high similarity to the NRT2 genes of other monocotyledons, while OsNRT2.3 and OsNRT2.4 are more closely related to Arabidopsis NRT2 genes.

Some high-affinity NO3− transporters belonging to the NRT2 family have been shown to require a partner protein, NAR2, for their function (Xu et al., 2012). Quesada, Galvan & Fernandez (1994) identified the CrNar2 gene, which encodes a small protein of approximately 200 amino acid residues and which has no known transport activity, but is required for complementation of NO3− transport in Chlamydomonas reinhardtii mutants defective in uptake. In Arabidopsis, Okamoto et al. (2006) showed that both constitutive and NO3−-inducible HATS, but not LATS, depended on the expression of the NAR2-type gene, for example Arabidopsis AtNRT3.1. Orsel et al. (2006) used yeast split-ubiquitin and oocyte expression systems to show that AtNAR2.1 (AtNRT3.1) and AtNRT2.1 interacted to produce a functional HATS. Yong, Kotur & Glass (2010) showed that the NRT2.1 and NAR2.1 polypeptides interact directly at the plasma membrane to constitute an oligomer that may act as the functional unit for high-affinity NO3− influx in Arabidopsis roots. In rice, the OsNRT2.1, OsNRT2.2, and OsNRT2.3a gene products were similarly shown to require the protein encoded by OsNAR2.1 for NO3− uptake (Feng et al., 2011; Yan et al., 2011; Liu et al., 2014) and their interaction at the protein level was demonstrated using a yeast two hybrid assay and by western blotting (Yan et al., 2011; Liu et al., 2014). Rice seedling growth was improved slightly by increased OsNRT2.1 expression, but N uptake remained unaffected (Katayama et al., 2009) probably due to the absence of the interaction with OsNAR2.1, which is required for functional NO3− transport (Feng et al., 2011; Yan et al., 2011).

Plants adapt to changing environmental conditions by modifying their growth. Plant growth and development is a complex process involves the integration of many environmental and endogenous signals that, together with the intrinsic genetic program, determine plant form. Factors that are involved in this process include several growth regulators collectively called the plant hormones or phytohormones. Abiotic stress can negatively impact on plant growth leading to significant losses in agriculture. Even moderate stress can have significant impact on plant growth and thus yield of agriculturally important crop plants. Therefore, finding a way to improve growth, in particular under stress conditions, is of great economic interest.

There is a need to provide not only crop plants that have higher yields in stress and non-stress conditions, but that are more nutrient efficient to ensure sustainable crop production. Such crops are necessary for global food security and to reduce the costs and negative environmental effects of mineral fertiliser input, such as of air and water quality and losses of biodiversity. The present invention is aimed at addressing this need.

### SUMMARY OF THE INVENTION

We have altered the relative expression of the OsNRT2.1 gene, which encodes a high-affinity NO3− transporter, using the NO3−-inducible promoter of the OsNAR2.1 gene to drive OsNRT2.1 expression in transgenic rice plants. Transgenic lines expressing pOsNAR2.1:OsNRT2.1 constructs exhibited an increase in grain yield of 30.7% and 28.1% in T0 and T1 plants respectively compared to wild-type (WT) plants. The agricultural NUE (ANUE) of the pOsNAR2.1:OsNRT2.1 lines increased to 128% of that of WT plants. The dry matter transfer (DMT) into grain increased by 46% in the pOsNAR2.1:OsNRT2.1 lines relative to the WT. The expression of OsNRT2.1 in shoot and grain showed that OsNAR2.1 promoters increased the level of OsNRT2.1 expression to about 180% compared to the WT. Interestingly we also found that the OsNAR2.1 expression was increased in root, leaf sheaths and inter nodes of the pOsNAR2.1:OsNRT2.1 lines. Accordingly, driving expression of OsNRT2.1 from the OsNAR2.1 promoter not only increased NRT2.1 expression but altered its expression profile. We therefore show that altering the expression profile of NRT2.1 can improve yield and NUE in a crop plant.

In one aspect, the invention relates to a method for increasing growth, yield, biomass, agricultural nitrogen use efficiency (ANUE), N recovery efficiency (NRE) and/or total N content of a plant, the method comprising altering the expression profile of a NRT2 nucleic acid in a plant, wherein the NRT2 nucleic acid is selected from NRT2.1, NRT2.2 and/or NRT2.3a as defined in SEQ ID NOs: 1, 3 and 5 respectively, or a functional homologue or variant thereof.

In another aspect the invention relates to a nucleic acid construct comprising a nucleic acid sequence as defined in any one of SEQ ID Nos: 1, 3 or 5, or a functional variant or homolog thereof operably linked to a regulatory sequence, wherein said regulatory sequence is a nitrate-inducible promoter, and wherein preferably the nitrate-inducible promoter is a NAR2.1 promoter comprising a sequence as defined in SEQ ID No: 7 or a functional homologue or variant thereof.

In another aspect, the invention relates to a vector comprising a nucleic acid construct as described herein.

In a further aspect, the invention relates to a host cell comprising a nucleic acid construct as described herein.

In yet another aspect, the invention relates to a transgenic plant expressing the nucleic acid construct as described herein.

In another aspect, the invention relates to a transgenic plant expressing a nucleic acid sequence comprising a sequence as defined in any one of SEQ ID Nos 1, 3 or 5, or a functional variant or homolog thereof operably linked to a nitrate-inducible promoter, wherein the nitrate-inducible promoter comprises a nucleic acid sequence as defined in SEQ ID NO: 7 or a homologue or variant thereof.

In a further aspect the invention relates to a method for making a transgenic plant having increased growth, biomass, yield, agricultural nitrogen use efficiency (ANUE), N recovery efficiency (NRE) and/or total N content, the method comprising introducing and expressing in a plant or plant cell a nucleic acid construct as described herein.

The invention also relates to the use of the nucleic acid construct as described herein to increase growth, biomass, yield, agricultural nitrogen use efficiency (ANUE), N recovery efficiency (NRE) and/or total N content of a plant of a plant.

In a further aspect the invention relates to a method of producing a mutant plant that has increased growth, biomass, yield, agricultural nitrogen use efficiency (ANUE), N recovery efficiency (NRE) and/or total N content of a plant, the method comprising introducing a mutation into the plant genome, wherein said mutation is introduced by mutagenesis or targeted genome editing, and wherein said mutation introduces at least one or more additional copy of
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or
a NRT 2.1, NRT 2.2 or NRT 2.3a gene sequence operably linked to a NAR2.1 promoter sequence.

In a further aspect, the invention relates to a genetically altered plant, wherein said plant carries a mutation in its genome and wherein said mutation introduces one or more additional copy of a
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or
a NRT 2.1, NRT 2.2 or NRT 2.3a gene sequence operably linked to a NAR2.1 promoter sequence; into the plant genome.

In a yet further aspect the invention relates to a method of altering the expression ratio of NRT 2.1, NRT 2.2 and/or NRT 2.3a to NAR2.1 in a plant, the method comprising introducing and expressing the nucleic acid construct as described herein in a plant.

In an alternative embodiment, the invention relates to a method of altering the expression ratio of NRT 2.1, NRT 2.2 and/or NRT 2.3a to NAR2.1 in a plant, the method comprising introducing at least one mutation into the genome of a plant, wherein said mutation introduces one or more additional copy of
an NRT 2.1, NRT 2.2 or NRT 2.3a gene sequence such that at least one sequence is operably linked to an endogenous nitrate-inducible promoter, preferably an endogenous NAR2.1 promoter sequence;
an NAR 2.1 promoter sequence, such that said promoter sequence is operably linked to at least one endogenous NRT 2.1, NRT 2.2 or NRT 2.3a gene sequence and/or
an NRT 2.1, NRT 2.2 or NRT 2.3a gene sequence and a NAR 2.1 promoter sequence and wherein said mutation is introduced using mutagenesis or targeted genome editing.

In another aspect, the invention relates to a genetically altered plant characterised by a lower expression ratio of NRT2.1:NAR2.1, NRT2.2: NAR 2.1 and/or NRT2.3a:NAR2.1 compared to said ratio in a control plant.

In a final aspect there is provided a plant obtained or obtainable by the method as defined in any method of the invention.

The invention is further described in the following non-limiting figures.

### FIGURES

FIG. 1 Characterization of transgenic lines.

(a) Gross morphology of pUbi:OsNRT2.1 transgenic lines (OE1, OE2, and OE3) and the WT. (b) Gross morphology of pOsNAR2.1:OsNRT2.1 transgenic lines (O6, O7, and O8) and the WT. (c, d) Real-time quantitative RT-PCR analysis of endogenous OsNRT2.1 expression in various transgenic lines and wild-type (WT) plants. (c) pUbi:OsNRT2.1 transgenic lines (OE1, OE2, and OE3) and the WT, (d) pOsNAR2.1:OsNRT2.1 transgenic lines (O6, O7, and O8) and the WT. RNA was extracted from Leaf blade I, culm, and root. (e, f) Grain yield and dry weight per plant for transgenic and WT plants grown in the field. Dry weight mean values are for all aboveground biomass, including grain yield. (e) pUbi:OsNRT2.1 transgenic lines and WT, (f) pOsNAR2.1:OsNRT2.1 transgenic lines and WT. Statistical analysis was performed on data derived from the T3 generation. Error bars: SE (n=3). Significant differences between transgenic lines and WT are indicated by different letters (P<0.05, one-way ANOVA).

FIG. 2 N content in various parts of WT and transgenic plants at two growth stages. (a) Sixty days after transplant, anthesis stage. (b) Ninety days after transplant, maturity stage. Error bars: SE (n=3). Statistical analysis was performed on data derived from the T3 generation. Significant differences between transgenic lines and WT are indicated by different letters (P<0.05, one way ANOVA).

FIG. 3 Expression pattern of OsNRT2.1 and OsNAR2.1.

Relative expression of (a) OsNRT2.1 and (b) OsNAR2.1 in various organs at 14 days after pollination. pUbi:OsNRT2.1 represents the average of OE1, OE2, and OE3. pOsNAR2.1:OsNRT2.1 represents the average of O6, O7, and O8. Statistical analysis was performed on data derived from the T4 generation. We defined developing seed of WT expression was set equal to 1. Error bars: SE (n=3). Significant differences between transgenic lines and WT are indicated by different letters (P<0.05, one-way ANOVA).

FIG. 4 Growth status of the WT and transgenic lines during the experimental growth period.

(a) Changes in OsNRT2.1 expression over the experimental growth period. (b) Changes in OsNAR2.1 expression over the experimental growth period. RNA was extracted from culms. (c) Dry weight. Dry weight mean values are for all aboveground biomass, including grain yield. (d) Growth rate. Samples were collected at 15-day intervals after seedlings were transplanted to the field. Statistical analysis was performed on data derived from the T3 generation. Error bars: SE (n=3). D in x-axis means the day after transplanting. The asterisk at the end of time course indicates their statistical significant differences among plants and # indicates their statistical significant differences during the growth stages (P<0.05, ANCOVA).

FIG. 5 Ratios of OsNRT2.1 to OsNAR2.1 expression in culms of WT and transgenic lines over the course of the study.

The ratios of OsNRT2.1 and OsNAR2.1 expression during different periods at 15-day intervals after seedlings were transplanted to the field in the culms of pUbi:OsNRT2.1 lines (OE1, OE2, OE3), pOsNAR2.1:OsNRT2.1 lines (O6, O7, and O8) and WT were presented.

FIG. 6 Comparison of grain yield, dry weight, and agronomic nitrogen-use efficiency (ANUE) between the WT and transgenic lines in the T1-T3 generations.

Dry weight mean values are for all aboveground biomass, including grain yield. For each mean, n=3. Significant differences between transgenic lines and WT are indicated by different letters (P<0.05, one-way ANOVA).

FIG. 7 Comparison of agronomic traits between WT and transgenic lines.

Statistical analysis was performed on data derived from the T3 generation. Significant differences between transgenic lines and WT are indicated by different letters (P<0.05, one-way ANOVA, n=3).

FIG. 8 Comparison of dry matter accumulation and N content between WT and transgenic lines.

Statistical analysis was performed on data derived from the T3 generation. For each mean, n=3. Significant differences between transgenic lines and WT are indicated by different letters (P<0.05, one-way ANOVA).

FIG. 9 Comparison of N use efficiency, dry matter transport efficiency and N transport efficiency between WT and transgenic rice lines.

Statistical analysis was performed on data derived from the T3 generation. Methods of calculations in FIG. 13. For each mean, n=3. Significant differences between transgenic lines and WT are indicated by different letters (P<0.05, one-way ANOVA).

FIG. 10 Primers used to amplify the OsNRT2.1 open reading frame.

FIG. 11 Primers used to amplify the OsNAR2.1 and Ubiquitin promoters

FIG. 12 Primers used to detect OsActin, OsNAR2.1, and OsNRT2.1 gene expression.

FIG. 13 Methods of NUE calculations.

FIG. 14 Real-time quantitative RT-PCR analysis of endogenous OsNRT2.1 and OsNAR2.1 expression in various transgenic lines and wild-type (WT) plants.

FIG. 15 Diagram of (a) pUbi:OsNRT2.1 and (b) pOsNAR2.1:OsNRT2.1 constructs. LB, left border; RB, right border; 35S, cauliflower mosaic virus 35S promoter; Ubi1-1, ubiquitin promoter; pOsNAR2.1, OsNAR2.1 promoter; NOS, nopaline synthase terminator.

FIG. 16 Characterization of T0 generation transgenic lines.

(a, b) Real-time quantitative RT-PCR analysis of endogenous OsNRT2.1 expression in various transgenic lines and the WT. (a) pUbi:OsNRT2.1 transgenic lines and the WT. (b) pOsNAR2.1:OsNRT2.1 transgenic lines and the WT. RNA was extracted from culms. Error bars: SE (n=3). (c, d) Yield per plant from transgenic and WT plants grown in the field. (c) pUbi:OsNRT2.1 transgenic lines and the WT. (d) pOsNAR2.1:OsNRT2.1 transgenic lines and WT. (e, f) Dry weight per plant of transgenic lines and WT plants grown in the field. (e) pUbi:OsNRT2.1 transgenic lines and WT. (f) pOsNAR2.1:OsNRT2.1 transgenic lines and WT. Error bars: SE (n=3). Significant differences between transgenic lines and WT are indicated by different letters (P<0.05, one way ANOVA).

FIG. 17 Grain yield and dry weight of WT and T1 generation transgenic plants.

(a) pUbi:OsNRT2.1 transgenic lines and WT, (b) pOsNAR2.1:OsNRT2.1 transgenic lines and WT. Error bars: SE (n=3).

FIG. 18 Southern blot analysis of transgene copy number.

Genomic DNA isolated from T1 generation pUbi:OsNRT2.1 and pOsNAR2.1:OsNRT2.1 transgenic plants was digested with the HindIII and EcoRI restriction enzymes. A hygromycin gene probe was used for hybridization. M, marker; P, positive control.

FIG. 19 Grain yield, dry weight and ANUE of WT and T4 generation transgenic plants under low and normal N supplies.

Grain yield and dry weight under nitrogen fertilizer was applied at a rate of (a) 180 kg N/ha and (b) 300 kg N/ha. (c) ANUE under 180 kg N/ha and 300 kg N/ha supplies. Error bars: SE (n=3). Significant differences between transgenic lines and WT are indicated by different letters (P<0.05, one way ANOVA).

FIG. 20 The diagram of RNA sampling in T4 generation transgenic lines and WT plants. RNA was extracted from 14 days after pollination.

FIG. 21 Ratios of OsNRT2.1 to OsNAR2.1 expression in different organs of WT and transgenic lines.

The ratios of OsNRT2.1 and OsNAR2.1 expression in different organs of pUbi:OsNRT2.1 lines (OE1, OE2, OE3), pOsNAR2.1:OsNRT2.1 lines (O6, O7, and O8) and WT were presented at 14 days after pollination.

FIG. 22 RNA sampling in T3 generation transgenic lines and WT plants.

RNA was extracted from leaf blade I and culm. (a) Indicating the plants at fifteen, thirty, and forty-five days after transplanting. (b) Indicating the plants at sixty, seventy-five, and ninety days after transplanting.

FIG. 23 Changes in genes expression in leaf blade I throughout the experimental growth period.

(a) Changes in OsNRT2.1 expression. (b) Changes in OsNAR2.1 expression. After seedlings were transplanted, RNA was extracted from leaf blade I and collected at 15-day intervals. Statistical analysis was performed on data derived from the T3 generation. Error bars: SE (n=3).

FIG. 24 Ratios of OsNRT2.1 and OsNAR2.1 expression in the leaf blade I of WT and transgenic plants during different periods.

The ratio of OsNRT2.1 and OsNAR2.1 expression during different period in the leaf blade I of pUbi:OsNRT2.1 lines (OE1, OE2, OE3), pOsNAR2.1:OsNRT2.1 lines (O6, O7, and O8) and WT were presented.

FIG. 25 A field experiment picture of WT and T3 generation transgenic plants. The picture was taken on 1 Oct. 2014 at Nanjing.

FIG. 26 Alignment of NAR and NRT2 homologues.

FIG. 27 pOsNAR2.1:OsNRT2.1 and WT morphology Gross morphology of pOsNAR2.1:OsNRT2.1 lines (O6 and O7) and the WT grown with (a) Control, (b) 10% PEG, (c) 100 mM NaCl and (d) Cold. Rice seedlings of WT and transgenic plants were grown in IRRI solution for 2 weeks, and were then grown with different different stress conditions for 9 days. bar=10 m

FIG. 28 Comparison of growth of wild-type (WT) and pOsNAR2.1:OsNRT2.1 transgenic plants under different stress conditions.

(a) Fresh weight and (b) Root/shoot Ratio. Error bars: SE (n=4 plants). Significant differences between WT and transgenic lines are indicated by different letters (P<0.05, one-way ANOVA), wherein values associated with different letters are statistically different from each other.

FIG. 29 Comparison of fresh weight increased compared with wild-type (WT) of control.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated by reference.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place.

The aspects of the invention involve recombination DNA technology and exclude embodiments that are solely based on generating plants by traditional breeding methods.

For the purposes of the invention, a “mutant” plant is a plant that has been genetically altered compared to the naturally occurring wild type (WT) plant. In one embodiment, a mutant plant is a plant that has been altered compared to the naturally occurring wild type (WT) plant using a mutagenesis method, such as the mutagenesis methods described herein. In one embodiment, the mutagenesis method is targeted genome modification or genome editing. In one embodiment, the plant genome has been altered compared to wild type sequences using a mutagenesis method. In one example, mutations can be used to insert a NRT2.1, NRT 2.2 and/or NRT2.3a gene sequence to enhance levels of expression of a NRT2.1 NRT 2.2 and/or NRT2.3a (and/or NAR2.1) nucleic acid compared to a wild-type plant. In this example, the NRT2.1, NRT 2.2 and/or NRT2.3a gene sequence is operably linked to an endogenous NAR2.1 promoter. Such plants have an altered phenotype as described herein, such as an increased growth, yield, biomass, agricultural nitrogen use efficiency (ANUE), N recovery efficiency (NRE) and/or total N content compared to wild type plants. Therefore, in this example, growth, yield, biomass, agricultural nitrogen use efficiency (ANUE), N recovery efficiency (NRE) and/or total N content is conferred by the presence of an altered plant genome, for example, a mutated endogenous NAR2.1 promoter sequence. In a preferred embodiment, the endogenous promoter sequence is specifically targeted using targeted genome modification and the presence of a mutated NAR2.1 promoter sequence is not conferred by the presence of transgenes expressed in the plant.

According to all aspects of the invention, including the method above and including the plants, methods and uses as described below, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

A “plant promoter” comprises regulatory elements which mediate the expression of a coding sequence segment in plant cells. The promoters upstream of the nucleotide sequences useful in the nucleic acid constructs described herein can also be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoter is increased by modification of their sequence, or that they are replaced completely by more active promoters, even promoters from heterologous organisms. For expression in plants, the NRT2.1, NRT2.2 and/or 2.3a nucleic acid molecule is, as described above, preferably linked operably to or comprises a suitable promoter which expresses the gene at the right point in time and with the required spatial expression pattern. In one embodiment, the regulatory sequence is a tissue specific promoter. Tissue specific promoters are transcriptional control elements that are only active in particular cells or tissues at specific times during plant development. Alternatively, the promoter is a nitrate-inducible promoter. Examples of nitrate-inducible promoters comprise the promoters for NRT2.1, NRT 2.3a and promoters of nitrate reductase genes, such as NIA and NIR. In a preferred embodiment, the tissue specific promoter comprises SEQ ID No. 7 or a functional variant or homolog thereof.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

The term “operably linked” as used herein refers to a functional linkage between the promoter sequence and the gene of interest, such that the promoter sequence is able to initiate transcription of the gene of interest.

We have transformed the open reading frame (ORF) of the OsNRT2.1 gene into rice with expression driven by the OsNAR2.1 promoter to alter the expression profile of OsNRT2.1 in rice plants and to investigate the biological function of this altered expression profile in vivo.

Transgenic lines expressing the OsNRT2.1 gene under the control of the OsNAR2.1 promoter exhibited greatly increased growth, yield, and biomass compared with transgenic lines expressing OsNRT2.1 under the control of a ubiquitin promoter. We analysed OsNRT2.1 and OsNAR2.1 expression patterns during whole plant growth and show that modification of the ratio of OsNRT2.1 to OsNAR2.1 expression in stems altered rice growth and agricultural N-use efficiency (ANUE).

By comparison, transgenic lines expressing pUbi:OsNRT2.1 increased total biomass including yields of approximately 21% compared with wild-type (WT) plants. The agricultural NUE (ANUE) of the pUbi:OsNRT2.1 lines decreased to 83% of that of WT plants, and the dry matter transfer (DMT) into grain decreased by 68% in the pUbi:OsNRT2.1 lines. The expression of OsNRT2.1 in shoot and grain showed that Ubi enhanced OsNRT2.1 expression by 7.5-fold averagely. Interestingly we also found that the OsNAR2.1 was expressed higher in all the organs of pUbi:OsNRT2.1 lines.

Therefore, in one aspect of the invention, there is provided a method for increasing growth, yield, biomass, agricultural nitrogen use efficiency (ANUE), N recovery efficiency (NRE), and/or total N content of a plant preferably under stress or non-stress conditions, the method comprising altering the expression profile of a NRT2 nucleic acid in a plant. In an alternative aspect, there is a provided a method for improving stress tolerance and/or mitigating the effects of stress on a plant, the method comprising altering the expression profile of a NRT2 nucleic acid in a plant. In one embodiment, this means altering the levels of a NRT2 nucleic acid in a plant and/or altering the protein levels of a NRT2 protein in a plant.

In one embodiment the stress tolerance is tolerance to abiotic stress, preferably wherein the abiotic stress is cold, drought and/or high salt conditions. In another embodiment of the invention, the stress is abiotic stress, preferably wherein the abiotic stress is cold, drought and/or high salt conditions.

In one embodiment, the NRT2 nucleic acid is selected from NRT2.1, NRT2.2 and/or NRT2.3a as defined in SEQ ID NOs: 1, 3 and 5 respectively, or a functional homologue or variant thereof and encodes a NRT2.1 NRT2.2 and NRT2.3a protein as defined in SEQ ID NOs: 2, 4 and 6 respectively or a functional variant thereof.

In one embodiment, the method comprises introducing and expressing into a plant a nucleic acid construct comprising or consisting of a NRT 2.1, NRT 2.2 and/or NRT 2.3a nucleic acid sequence operably linked to a regulatory sequence, wherein said regulatory sequence is a nitrate-inducible promoter and wherein preferably expression of the nucleic acid construct alters the expression profile of the NRT2 nucleic acid. In one embodiment, the nitrate-inducible promoter directs expression of said nucleic acid in the roots and culms of a plant. In a further embodiment, the nitrate-inducible promoter is not a NRT2.1 promoter. Preferably, the NO3−-inducible promoter is a NAR2.1 promoter as defined in SEQ ID No: 7 or a functional homologue or variant thereof. Preferably, the NRT 2.1, NRT 2.2 or NRT 2.3a nucleic acid sequence is selected from SEQ ID NO: 1, 3 or 5 or a functional homologue or variant thereof. In one embodiment, the NRT 2.1, NRT 2.2 or NRT 2.3a nucleic acid and regulatory sequence are from the same plant family, genus or species. In an alternative embodiment, the NRT 2.1, NRT 2.2 or NRT 2.3a nucleic acid and regulatory sequence are from a different plant family, genus or species.

In an alternative embodiment, the method comprises introducing a mutation into the plant genome, wherein said mutation is the insertion of at least one or more additional copy of
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or;
a NRT 2.1, NRT 2.2 or NRT 2.3a gene sequence operably linked to a NAR2.1 promoter sequence;

wherein such mutation is introduced using targeted genome editing, and wherein, preferably said mutation results in an altered expression profile of a NRT2 nucleic acid.

In a preferred embodiment, the NRT2.1, 2.2 or 2.3a gene sequence is selected from SEQ ID No: 1, 3 or 5 or a functional homologue or variant thereof. In another preferred embodiment, the NAR2.1 promoter sequence is SEQ ID NO: 7 or a functional homologue or variant thereof.

In one embodiment, the expression profile of a NRT nucleic acid is altered compared to a control plant. In a further embodiment, altering the expression profile comprises increasing the levels of a NRT nucleic acid in the roots and culms, particularly internodes and/or leaf sheaths of a plant. In a further preferred embodiment, altering the expression profile comprises altering the relative expression ratios of NRT to NAR in a plant. In one embodiment, the ratio of NRT2.1, NRT2.2 or NRT2.3a to NAR2.1 in a plant is reduced compared to the ratio in a control plant. In a further embodiment, the ratio is altered in the stem or culm of a plant.

In another embodiment, the NRT2.1:NAR2.1, NRT2.2:NAR 2.1 or NRT2.3a:NAR2.1 ratio is below at least 7:1, preferably below 6:1, preferably below 5:1, more preferably below 4:1 and even more preferably 3.6:1 in plant organs compared with a ratio of at least 7:1, preferably below 6:1, preferably below 5:1, more preferably below 4:1 and even more preferably 3.9:1 in control plants and wherein the ratio is lower than that in control plants.

In another embodiment, the NRT2.1:NAR2.1, NRT2.2:NAR 2.1 or NRT2.3a:NAR2.1 ratio is below at least 7:1, preferably below 6:1, more preferably below 5:1, and even more preferably 4.7:1 in plant culms compared with a ratio of at least below 10:1, preferably below 9:1, more preferably below 8:1 and even more preferably 7.2:1 in control plants, and wherein the ratio is lower than that in control plants.

In one embodiment, the method for increasing growth, yield, biomass, agricultural nitrogen use efficiency (ANUE), N recovery efficiency (NRE), stress tolerance and/or total N content in a plant and/or mitigating the effects of stress on a plant, can further include steps comprising one or more of: assessing the phenotype of the transgenic plant, measuring NUE and/or NO3− uptake, comparing NUE and/or NO3− uptake to that of a control plant, measuring total N content, measuring yield and/or and comparing yield and/or biomass to that of a control plant.

In a further embodiment of the above described methods, the method increases growth, yield, biomass, agricultural nitrogen use efficiency (ANUE) and/or N recovery efficiency (NRE) under low N input (e.g. 180 kg N/ha or lower). Accordingly, in one embodiment, the method increases growth, yield, agricultural nitrogen use efficiency (ANUE), biomass and/or N recovery efficiency (NRE) under nitrogen stress conditions. In another embodiment, the method increases growth, yield, agricultural nitrogen use efficiency (ANUE) and/or N recovery efficiency (NRE) under normal (e.g. 300 kg/Nha) or high N input.

According to the various aspects of the invention, the observed phenotypes, e.g. increased growth, yield, agricultural nitrogen use efficiency (ANUE), N recovery efficiency (NRE) and/or total N content in the transgenic plant is increased by about 5%-50% or more compared to a control plant, for example by at least 5%, 10%, 15%, 20%, 25%, 30%, 35%, 40%, 45% or 50%. Preferably, growth is measured by measuring hypocotyl or stem length. In one embodiment, total N content is measured using the Kjeldahl method.

The terms “increase”, “improve” or “enhance” as used according to the various aspects of the invention are interchangeably. Growth, yield, biomass, agricultural nitrogen use efficiency (ANUE) and/or N recovery efficiency (NRE) is increased by at least 5%-50% or more compared to a control plant, for example by at least 5%, 10%, 15%, 20%, 25%, 30%, 35%, 40%, 45% or 50%.

The term “yield” includes one or more of the following non-limitative list of features: early flowering time, biomass (vegetative biomass (root and/or shoot biomass) and/or seed/grain biomass), seed/grain yield (including grain number per panicle) panicle length, seed setting rate, seed/grain viability and germination efficiency, seed/grain size, starch content of grain, early vigour, greenness index, increased growth rate, delayed senescence of green tissue. The term “yield” in general means a measurable produce of economic value, typically related to a specified crop, to an area, and to a period of time. Individual plant parts directly contribute to yield based on their number, size and/or weight. The actual yield is the yield per square meter for a crop and year, which is determined by dividing total production (includes both harvested and appraised production) by planted square metres.

Thus, according to the invention, yield comprises one or more of and can be measured by assessing one or more of: increased seed yield per plant, increased seed filling rate, increased number of filled seeds, increased harvest index, increased viability/germination efficiency, increased number or size of seeds/capsules/pods/grain, increased growth or increased branching, for example inflorescences with more branches, increased biomass or grain fill. Preferably, increased yield comprises an increased number of grain/seed/capsules/pods, increased biomass, increased growth, increased number of floral organs and/or floral increased branching. Yield is increased relative to a control plant. For example, the yield is increased by 2%, 3%, 4%, 5%-50% or more compared to a control plant, for example by at least 10%, 15%, 20%, 25%, 30%, 35%, 40%, 45% or 50%.

The term “nitrogen use efficiency” or NUE can be defined as being yield of crop (e.g. yield of grain). Alternatively, NUE can be defined as agricultural NUE that means grain yield/N. The overall N use efficiency of plants comprises both uptake and utilization efficiencies and can be calculated as UpE. In one embodiment, NUE is increased by 5%-50% or more compared to a control plant, for example by at least 5%, 10%, 15%, 20%, 25%, 30%, 35%, 40%, 45% or 50%. In another embodiment, nitrogen uptake is increased by 5%-50% or more compared to a control plant, for example by at least 5%, 10%, 15%, 20%, 25%, 30%, 35%, 40%, 45% or 50%.

The term “nitrogen recovery efficiency” (NRE) can be defined as the N recovered by the plant per unit N applied. In one embodiment, NRE is increased by 5%-50% or more compared to a control plant, for example by at least 5%, 10%, 15%, 20%, 25%, 30%, 35%, 40%, 45% or 50%. In one embodiment, total N content is increased in the culm and/or grain of the plant.

In a further aspect of the invention, there is provided a method of increasing dry matter at anthesis (DMA), dry matter at maturity (DMM), total N accumulation at anthesis (TNAA), total N accumulation at maturity (TNAM), dry matter translocation (DMT), post-anthesis N uptake (PANU) and/or N translocation (NT), the method comprising altering the expression profile of a NRT2 nucleic acid in a plant, as defined above. In a further aspect of the invention, there is provided a method of decreasing the contribution of pre-anthesis N to grain N accumulation (CPNGN), the method comprising altering the expression profile of a NRT2 nucleic acid in a plant as described herein. According to the various aspects described herein, the observed phenotype is increased or decreased compared to a control plant, as already defined herein. In one embodiment, the increase or decrease in the observed phenotype is 5%-90% or more compared to a control plant, for example by at least 5%, 10%, 15%, 20%, 25%, 30%, 35%, 40%, 45% 50%, 55%, 60%, 65%, 70%, 75%, 80%, 85% or 90%.

The method may further comprise screening plants for those that have an altered expression profile of a NRT2 nucleic acid as described herein and/or which have any phenotype described herein, such as increased growth, yield, biomass and/or nitrogen use efficiency, and selecting a plant with that phenotype, such as increased growth, yield, biomass and/or nitrogen use efficiency. In another embodiment, further steps include measuring increased growth, yield, biomass and/or nitrogen use efficiency in said plant progeny or part thereof and comparing said phenotype to that of a control plant. In one embodiment, the progeny plant is stably transformed with the nucleic acid construct described herein and comprises the exogenous polynucleotide which is heritably maintained in the plant cell. The method may include steps to verify that the construct is stably integrated. The method may also comprise the additional step of collecting seeds from the selected progeny plant.

The invention also extends to a plant obtained or obtainable by a method as described herein, for example a method for increasing growth, yield, biomass, agricultural nitrogen use efficiency (ANUE), N recovery efficiency (NRE), stress tolerance and/or total N content and/or mitigating the effects of stress on a plant.

In another aspect of the invention there is provided a nucleic acid construct comprising a NRT 2.1, NRT 2.2 and/or NRT 2.3a nucleic acid sequence operably linked to a regulatory sequence, wherein said regulatory sequence is a NO3− inducible promoter. In one embodiment, the NO3−-inducible promoter is a NAR2.1 promoter as defined in SEQ ID No: 7 or a functional homologue or variant thereof. Preferably, the NRT 2.1, NRT 2.2 and/or NRT 2.3a nucleic acid sequence is selected any from any one of SEQ ID NO: 1, 3 or 5, or a functional homologue or variant thereof.

In a preferred embodiment, there is provided a nucleic acid construct comprising OsNRT2.1 operably linked to a regulatory sequence, wherein said regulatory sequence is the OsNAR2.1 promoter and wherein the nucleic acid construct comprises or consists of SEQ ID NO: 1 or a functional variant or homolog thereof and encodes a NRT2.1 protein as defined in SEQ ID NO: 2 or a functional variant thereof. In one embodiment, the NRT2 nucleic acid and regulatory sequence are from the same plant family, genus or species. In an alternative embodiment, the NRT2 nucleic acid and regulatory sequence are from a different plant family, genus or species.

In another aspect, the invention relates to an isolated host cell transformed with a nucleic acid construct or vector as described above. The host cell may be a bacterial cell, such as Agrobacterium tumefaciens, or an isolated plant cell. The invention also relates to a culture medium or kit comprising a culture medium and an isolated host cell as described below.

The nucleic acid construct or vector described above can be used to generate transgenic plants using transformation methods known in the art and described herein.

Thus, in a further aspect, the invention relates to a transgenic plant expressing the nucleic acid construct as described herein.

The invention also relates to a genetically altered plant expressing a nucleic acid sequence comprising a sequence selected from any one of SEQ ID NO: 1, 3 or 5 or a functional variant or homolog thereof operably linked to a nitrate-inducible promoter. In one embodiment, the nitrate-inducible promoter is a NAR2.1 promoter. In another embodiment, the NAR2.1 promoter sequence comprises SEQ ID NO. 7 or a functional variant or homolog thereof. The plant is characterised in that it shows increased growth, yield, biomass, agricultural nitrogen use efficiency (ANUE), N recovery efficiency (NRE) and/or total N content compared to a control or wild-type plant. In a further aspect the invention relates to genetically altered plant expressing an exogenous nucleic acid sequence comprising a sequence selected from SEQ ID NO 1, 3 or 5 or a functional variant or homolog wherein said exogenous sequence is expressed in the root, leaf sheaths, inter nodes and/or grain of the plant.

According to the methods described herein, plants express a polynucleotide “exogenous” to an individual plant that is a polynucleotide which is introduced into the plant by any means other than by a sexual cross. Examples of means by which this can be accomplished are described below. In one embodiment of the method, an exogenous nucleic acid is expressed in the transgenic plant which is a nucleic acid construct comprising a NAR2.1 promoter gene sequence and a NRT2.1, NRT2.2 and/or NRT2.3a gene sequence that is not endogenous to said plant but is from another plant species. For example, the pOsNAR2.1:OsNRT2.1 construct can be expressed in another plant that is not rice. In one embodiment of the method, an endogenous nucleic acid construct is expressed in the transgenic plant. For example, the pOsNAR2.1:OsNRT2.1 construct can be expressed in rice.

In another aspect, the invention relates to a method for making a transgenic plant having increased growth, yield, biomass, agricultural nitrogen use efficiency (ANUE), N recovery efficiency (NRE), stress tolerance and/or total N content and/or mitigating the effects of stress on a plant, the method comprising introducing and expressing in a plant or plant cell a nucleic acid construct as described herein. In a preferred embodiment, the method increases grain yield in a plant.

In one embodiment, the observed phenotypes, e.g. increased growth, yield, agricultural nitrogen use efficiency (ANUE), N recovery efficiency (NRE), stress tolerance and/or total N content is increased by about 5%-50% or more compared to a control plant, for example by at least 5%, 10%, 15%, 20%, 25%, 30%, 35%, 40%, 45% or 50%. Preferably, growth is measured by measuring hypocotyl or stem length. In one embodiment, total N content is measured using the Kjeldahl method.

The method may further comprise regenerating a transgenic plant from the plant or plant cell wherein the transgenic plant comprises in its genome a nucleic acid sequence selected from SEQ ID NO: 1, 3 or 5, or a functional variant or homolog thereof operably linked to a regulatory sequence and obtaining a progeny plant derived from the transgenic plant, wherein said progeny exhibits increased growth, yield, biomass, agricultural nitrogen use efficiency (ANUE), N recovery efficiency (NRE), stress tolerance and/or total N content and/or the effects of stress on a plant are mitigated. In a preferred embodiment, the regulatory sequence is a NAR2.1 promoter, as defined above.

Transformation methods for generating a transgenic plant of the invention are known in the art. Thus, according to the various aspects of the invention, a nucleic acid construct as defined herein is introduced into a plant and expressed as a transgene. The nucleic acid construct is introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker. Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect, the invention relates to a method for increasing growth, yield, biomass, agricultural nitrogen use efficiency (ANUE), N recovery efficiency (NRE), stress tolerance and/or total N content in a plant and/or mitigating the effects of stress on a plant, the method comprising introducing and expressing a nucleic acid construct as defined above in a plant.

The method for increasing growth, yield, biomass, agricultural nitrogen use efficiency (ANUE), N recovery efficiency (NRE), stress tolerance and/or total N content in a plant and/or mitigating the effects of stress on a plant, the method comprising introducing and expressing a nucleic acid construct as described above can include further steps comprising one or more of: assessing the phenotype of the transgenic plant, measuring NUE and/or NO3− uptake, comparing NUE and/or NO3− uptake to that of a control plant, measuring total N content, measuring yield and/or and comparing yield and/or biomass to that of a control plant.

In another embodiment, the invention relates to the use of a nucleic acid construct as described herein in increasing growth, yield, biomass, agricultural nitrogen use efficiency (ANUE), N recovery efficiency (NRE) and/or total N content in a plant.

In a further aspect of the invention, there is provided a method of increasing dry matter at anthesis (DMA), dry matter at maturity (DMM), total N accumulation at anthesis (TNAA), total N accumulation at maturity (TNAM), dry matter translocation (DMT), post-anthesis N uptake (PANU) and/or N translocation (NT), the method comprising introducing and expressing a nucleic acid construct as described herein. In a further aspect of the invention, there is provided a method of decreasing the contribution of pre-anthesis N to grain N accumulation (CPNGN), the method comprising introducing and expressing a nucleic acid construct as described herein. According to the various aspects described herein, the observed phenotype is increased or decreased compared to a control plant, as already defined herein. In one embodiment, the increase or decrease in the observed phenotype is 5%-90% or more compared to a control plant, for example by at least 5%, 10%, 15%, 20%, 25%, 30%, 35%, 40%, 45% 50%, 55%, 60%, 65%, 70%, 75%, 80%, 85% or 90%.

In another aspect, the invention relates to a genetically altered or mutant plant with an altered expression profile of a NRT2 nucleic acid and/or altered protein levels of a NRT2 protein, wherein said NRT2 nucleic acid or protein is selected from NRT2.1, 2.2 and/or NRT2.3a, and wherein said increase results from a mutation in the plant genome, wherein said mutation is introduced by mutagenesis or targeted genome editing. In this embodiment, the expression profile is altered relative to the profile in a control or wild-type plant, as defined elsewhere herein. In one embodiment, targeted genome editing is used to modify (i.e. insert) at least one or more additional copy of
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, NRT2.2 or NRT2.3a gene sequence and/or;
a NRT 2.1, NRT 2.2 or NRT 2.3a gene sequence operably linked to a NAR2.1 promoter sequence into the plant genome.

In a further embodiment, the levels of NRT2.1, NRT2.2 and/or NRT2.3a expression are altered (or increased) in the roots and/or culms of a plant. In a further embodiment, the mutation also results in an increase in expression or protein levels of NAR2.1 NRT2.2 and/or NRT2.3a in the plant.

In a preferred embodiment, the NRT2.1, NRT 2.2 and/or NRT2.3a gene sequence is selected from any of SEQ ID No: 1, 3 or 5, or a functional homologue or variant thereof. In another preferred embodiment, the NAR2.1 promoter sequence is SEQ ID NO: 7 or a functional homologue or variant thereof.

In another aspect of the invention there is provided a method for producing a mutant plant that has increased growth, yield, biomass, agricultural nitrogen use efficiency (ANUE), N recovery efficiency (NRE), stress tolerance and/or total N content and/or mitigating the effects of stress on a plant, the method comprising introducing a mutation into the plant genome, wherein said mutation is the insertion of at least one or more additional copy of
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or;
a NRT 2.1, NRT 2.2 or NRT 2.3a gene sequence operably linked to a NAR2.1 promoter sequence into the plant genome.

In a preferred embodiment, the mutation is introduced by mutagenesis or targeted genome editing. In a further embodiment, the mutation also results in increased expression of NRT2.1

In an alternative aspect of the invention there is provided a method for increasing growth, yield, agricultural nitrogen use efficiency (ANUE), N recovery efficiency (NRE) and/or total N content in a plant, the method comprising producing a mutant plant, wherein said plant carries a mutation in the plant genome as defined above. In a preferred embodiment, the mutation is inserted using targeted genome editing.

In one embodiment, the method for increasing growth, yield, biomass, agricultural nitrogen use efficiency (ANUE), N recovery efficiency (NRE) and/or total N content in a mutant plant as defined above can include further steps comprising one or more of: assessing the phenotype of the mutant plant, measuring NUE and/or NO3− uptake, comparing NUE and/or NO3− uptake to that of a control plant, measuring total N content, measuring yield and/or and comparing yield and/or biomass to that of a control plant.

In a preferred embodiment of the above described plant and methods, the nucleic acid sequence (i.e. gene sequence) of NRT2.1, NRT 2.2 and/or NRT2.3a is selected from any one of SEQ ID NOs: 1, 3 or 5 and encodes a NRT2.1, NRT 2.2 and/or NRT2.3a protein as defined in SEQ ID NO: 2, 4 or 6 respectively or a functional variant or homolog thereof of either SEQ ID NO: 1, 3 or 5. In a further embodiment, the nucleic acid sequence of the NAR2.1 promoter is 7 or a functional variant or homolog thereof.

In the above embodiments an ‘endogenous’ nucleic acid may refer to the native or natural sequence in the plant genome. In one embodiment, the endogenous OsNRT 2.1 sequence comprises a sequence as defined in SEQ ID NO: 1, the endogenous OsNRT2.2 sequence comprises a sequence as defined in SEQ ID NO: 2, the endogenous OsNRT2.3a sequence comprises a sequence as defined in SEQ ID NO: 3 and the endogenous pOsNAR2.1 sequence comprises a sequence as defined in SEQ ID NO: 7. Also included in the scope of this invention are functional variants and homologs of the above identified sequences.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of Fokl to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the Fokl nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus.

Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Cas9 expression plasmids for use in the methods of the invention can be constructed as described in the art.

Thus, aspects of the invention involve targeted mutagenesis methods, specifically genome editing, and in a preferred embodiment exclude embodiments that are solely based on generating plants by traditional breeding methods.

As discussed above, the inventors have also surprisingly shown that expressing a NRT2 nucleic acid, preferably a NRT2.1, NRT2.2 and/or NRT2.3a nucleic acid under the control of a nitrate-inducible promoter, preferably a NAR2.1 promoter not only alters the expression profile of the NRT2 nucleic acid, but also alters the expression ratio of NRT2.1, 2.2 and/or 2.3a: NAR2.1 in the plant.

Accordingly, in one aspect, there is provided a method of altering the expression ratio of NRT 2.1, NRT 2.2 and/or NRT 2.3a to NAR2.1 in a plant. In one embodiment, the method comprising introducing and expressing a nucleic acid construct as described herein to alter the expression ratio. In an alternative embodiment, the method comprises introducing a mutation into a plant to produce a genetically altered or mutant plant with an altered expression ratio, as also described above.

In one embodiment, the ratio of NRT 2.1, NRT 2.2 and/or NRT 2.3a to NAR2.1 in the plant is reduced compared to the ratio in a control plant. In a further embodiment, the ratio is altered in the stem or culm of a plant.

In another embodiment, the NRT2.1:NAR2.1, NRT2.2:NAR 2.1 or NRT2.3a:NAR2.1 ratio is below at least 7:1, preferably below 6:1, preferably below 5:1, more preferably below 4:1 and even more preferably 3.6:1 in plant organs compared with a ratio of at least 7:1, preferably below 6:1, preferably below 5:1, more preferably below 4:1 and even more preferably 3.9:1 in control plants and wherein the ratio is lower than that in control plants.

In another embodiment, the NRT2.1:NAR2.1, NRT2.2: NAR 2.1 and/or NRT2.3a:NAR2.1 ratio is below at least 7:1, preferably below 6:1, more preferably below 5:1, and even more preferably 4.7:1 in plant culms compared with a ratio of at least below 10:1, preferably below 9:1, more preferably below 8:1 and even more preferably 7.2:1 in control plants, and wherein the ratio is lower than that in control plants.

In another aspect of the invention there is provided a transgenic plant characterised by a lower expression ratio of NRT2.1:NAR2.1, NRT2.2: NAR 2.1 and/or NRT2.3a:NAR2.1 compared to said ratio in a control plant. Again, in one embodiment, the plant has a lower ratio in the culm or stem of the plant. In another embodiment, the NRT2.1:NAR2.1, NRT2.2: NAR 2.1 or NRT2.3a:NAR2.1 ratio is below at least 7:1, preferably below 6:1, preferably below 5:1 and even more preferably 4.7:1 in plants expressing the nucleic acid construct of the invention and the ratio is below at least 10:1, preferably below 9:1, more preferably below 8:1 and even more preferably 7.2:1 in control plants, and wherein the ratio in the culm of the transgenic plant is lower than that in control plants.

In one embodiment, the transgenic plant expresses the nucleic acid construct as described herein. In an alternative embodiment, the transgenic plant expresses a nucleic acid sequence comprising a sequence selected from any one of SEQ ID NO: 1, 3 or 5 or a functional variant or homolog thereof operably linked to a nitrate-inducible promoter. In one embodiment, the nitrate-inducible promoter is a NAR2.1 promoter. In another embodiment, the NAR2.1 promoter sequence comprises SEQ ID NO. 7 or a functional variant or homolog thereof.

In another aspect, there is also provided a genetically altered or mutant plant a lower expression ratio of NRT2.1:NAR2.1, NRT2.2: NAR 2.1 and/or NRT2.3a:NAR2.1 compared to said ratio in a control plant, wherein said altered ratio results from a mutation in the plant genome and wherein said mutation modifies (i.e. inserts) at least one or more additional copy of
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or;
a NRT 2.1, NRT 2.2 or NRT 2.3a gene sequence operably linked to a NAR2.1 promoter sequence.

In a preferred embodiment, the mutation is introduced by mutagenesis or targeted genome editing.

A plant is defined elsewhere, but in one embodiment is rice.

In another aspect of the invention there is provided a screening method for detecting a plant variety that has an increased growth, yield, biomass, agricultural nitrogen use efficiency (ANUE), N recovery efficiency (NRE) and/or total N content, the method comprising determining the expression ratio of NRT2.1:NAR2.1, NRT2.2: NAR 2.1 and/or NRT2.3a:NAR2.1 in at least one plant, and selecting said plant or plants with the lowest ratio. In a preferred embodiment, the selected plants are further propagated by a variety of means, such as those described above. In a further preferred embodiment, the ratio is determined in the culm of the plant. In one embodiment, the plant expresses the nucleic acid construct as described herein. In an alternative embodiment, the plant is a genetically altered plant as described herein.

In a further aspect of the invention, there is provided a method for altering growth, yield, biomass, and/or nitrogen use efficiency, N recovery efficiency (NRE) and/or total N content of a plant, the method comprising altering, the expression ratio of NRT2.1:NAR2.1, NRT2.2: NAR 2.1 and/or NRT2.3a:NAR2.1 in a plant. In one embodiment, the ratio of NRT2.1:NAR2.1, NRT2.2: NAR 2.1 and/or NRT2.3a:NAR2.1 expression is altered by expressing a nucleic acid construct as defined herein in a plant. In an alternative embodiment, the expression ratio of NRT2.1:NAR2.1, NRT2.2: NAR 2.1 and/or NRT2.3a: NAR2.1 is altered by introducing at least one mutation, as defined above, into the plant genome. In one embodiment, the method reduces the expression ratio of ratio of NRT2.1:NAR2.1, NRT2.2: NAR 2.1 and/or NRT2.3a:NAR2.1.

In a preferred embodiment of the above described methods, the nucleic acid sequence of NRT2.1 is selected from SEQ ID NO: 1 or a functional variant or homolog thereof, NRT 2.2 is selected from SEQ ID No: 3 or a functional variant or homolog thereof and NRT2.3a is selected from SEQ ID NO: 5 or a functional variant or homolog thereof and encodes a NRT2.1, 2.2 and 2.3a protein of SEQ ID NO: 2, 4 and 6 respectively, or a functional variant or homolog thereof. In another embodiment, the nucleic acid sequence of NAR2.1 comprises a sequence as defined in SEQ ID NO: 8 and encodes a NAR2.1 protein as defined in SEQ ID NO: 9 or a functional variant or homolog of either SEQ ID NO: 8 or 9.

In a final aspect, the invention relates to a method of co-expressing a NAR2.1 and NRT 2.1, NRT 2.2 and/or NRT 2.3a nucleic acid, the method comprising introducing and expressing the construct as defined herein in a plant. In an alternative embodiment, the invention relates to a method of co-expressing a NAR2.1 and NRT 2.1, NRT 2.2 and/or NRT 2.3a nucleic acid, the method comprising introducing a mutation, as defined herein, into the plant genome. In one embodiment of the method NAR2.1 and NRT 2.1, NRT 2.2 and/or NRT 2.3a are co-expressed in the root, leaf sheath, internodes and/or grain of the plant. In a further embodiment, NAR2.1 and NRT 2.1, NRT 2.2 and/or NRT 2.3a are not co-expressed in the leaf blades. In a further embodiment, the plant is rice, and the method relates to the co-expression of OsNAR2.1 and OsNRT2.1.

The term “functional variant of a nucleic acid sequence” as used herein with reference to any of SEQ ID Nos: 1, 3, 5 or 8 or SEQ ID NO: 7 refers to a variant gene sequence or part of the gene sequence which retains the biological function of the full non-variant sequence, for example confers increased biomass, growth, yield and/or nitrogen use efficiency (NUE) when expressed in a transgenic plant. A functional variant also comprises a variant of the gene of interest which has sequence alterations that do not affect function, for example in non-conserved residues. Also encompassed is a variant that is substantially identical, i.e. has only some sequence variations, for example in non-conserved residues, compared to the wild type sequences as shown herein and is biologically active.

Thus, it is understood, as those skilled in the art will appreciate, that the aspects of the invention, including the methods and uses, encompasses not only a nucleic acid sequence or amino acid sequence comprising or consisting a sequence selected from SEQ ID NO. 1 to 9 but also functional variants or parts of these SEQ ID NOs that do not affect the biological activity and function of the resulting protein. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

A skilled person will understand that the invention is not limited to aspects using OsNRT2.1, OsNRT2.2, OsNRT2.3a and/or the OsNAR2.1 promoter (pOsNAR2.1). Thus, in one embodiment of the aspects of the invention, the nucleic acid sequence encodes a homologue of OsNRT2.1, OsNRT2.2, OsNRT2.3a and/or pOsNAR2.1

The term homologue as used herein also designates an OsNRT2.1, OsNRT2.2, OsNRT2.3a or pOsNAR2.1 orthologue from other plant species. A homologue of OsNRT2.1, OsNRT2.2 or OsNRT2.3a polypeptide or a OsNRT2.1, OsNRT2.2, OsNRT2.3a or pOsNAR2.1 nucleic acid sequence respectively has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NOs: 2, 4 or 6 or to the nucleic acid sequences as shown by SEQ ID NOs: and 1, 3, 5, 7, 8, 9 or 10. In one embodiment, overall sequence identity is at least 37%. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%.

Functional variants of OsNRT2.1 or pOsNAR2.1 homologs are also within the scope of the invention.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution.

Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Examples of homologues are shown in FIG. 26 and in SEQ ID Nos 13 to 60.

Suitable homologues can be identified by sequence comparisons and identifications of conserved domains. There are predictors in the art that can be used to identify such sequences. The function of the homologue can be identified as described herein and a skilled person would thus be able to confirm the function, for example when overexpressed in a plant.

Thus, the OsNRT2.1, OsNRT2.2, OsNRT2.3a and/or pOsNAR2.1 nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologues. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

According to the invention, preferred OsNRT2.1, OsNRT2.2, OsNRT2.3a and/or pOsNAR2.1 homologues are selected from maize, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar, forage or turf grass.

According to the various aspects of the invention, the stress is preferably cold conditions, water shortage, for example drought conditions, or salinity (high salt). In another embodiment the method of the invention is for improving a plants tolerance to cold, drought conditions or salinity.

A plant according to the various aspects of the invention, including the transgenic plants, methods and uses described herein may be a monocot or a dicot plant.

A dicot plant may be selected from the families including, but not limited to Asteraceae, Brassicaceae (e.g. Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine, bell pepper, chili or citrus species.

A monocot plant may, for example, be selected from the families Arecaceae, Amaryffidaceae or Poaceae. For example, the plant may be a cereal crop, such as maize, wheat, rice, barley, oat, sorghum, rye, millet, buckwheat, or a grass crop such as Lolium species or Festuca species, or a crop such as sugar cane, onion, leek, yam or banana.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

Preferably, the plant is a crop plant. By crop plant is meant any plant which is grown on a commercial scale for human or animal consumption or use. In a preferred embodiment, the plant is a cereal.

Most preferred plants are maize, rice, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar. In a most preferred embodiment, the plant is rice.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. The invention also relates to food products and food supplements comprising the plant of the invention or parts thereof.

A control plant as used herein according to all of the aspects of the invention is a plant which has not been modified according to the methods of the invention. Accordingly, in one embodiment, the control plant does not have an altered expression profile of a NRT2 nucleic acid. In an alternative embodiment, the control plant does not express the nucleic acid construct described herein, nor has the plant been genetically modified, as described above. In one embodiment, the control plant is a wild type plant. The control plant is typically of the same plant species, preferably having the same genetic background as the modified plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Stress” is herein described as an unfavourable condition or substance that affects or blocks a plant's metabolism, growth or development. “Abiotic” stress is defined as stress resulting from nonliving factors, such as drought, extreme temperatures, salinity (e.g. 100 mM NaCl) and pollutants, for example heavy metals. The effect of stress on a plant and/or the tolerance of a plant to stress can be assessed by comparing the growth rate and/or yield of the plant in stress and non-stress conditions.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### EXAMPLES

### Example 1

1.1. Materials and Methods

Construction of Vectors and Rice Transformation

We amplified the OsNRT2.1/OsNRT2.2 open reading frame (ORF) sequence, which is identical for both genes, from cDNA isolated from Oryza sativa L. ssp. Japonica cv. Nipponbare using the primers listed in FIG. 10. We amplified the OsNAR2.1 and ubiquitin promoters from the pOsNAR2.1(1698 bp):GUS (Feng et al., 2011) and pUbi:OsPIN2 (Chen et al., 2012) constructs, respectively, using the primers listed in FIG. 11. The PCR products were cloned into the pMD19-T vector (TaKaRa) and confirmed by restriction enzyme digestion and DNA sequencing. The pUbi:OsNRT2.1 and pOsNAR2.1:OsNRT2.1 vectors were constructed as shown in FIG. 15. These constructs were introduced into Agrobacterium tumefaciens strain EHA105 by electroporation and then transformed into rice as described previously (Tang et al., 2012).

1.1.1. Southern Blot Analysis

Transgene copy number was determined by Southern blot analysis following procedures described previously (Jia et al., 2011). Briefly, genomic DNA was extracted from leaves of wild type (WT) and digested with HindIII and EcoRI restriction enzymes. The digested DNA was separated on a 1% (w/v) agarose gel, transferred to a Hybond-N+ nylon membrane, and hybridized with hygromycin-resistance gene.

1.1.2. Biomass, Total Nitrogen (N) Measurement, and Calculation of N Use Efficiency (NUE)

WT and transgenic rice plants were harvested at 9:00 AM and heated at 105° C. for 30 min. Panicles, leaves, and culms were then dried at 75° C. for 3 days. Dry weights were recorded as biomass values. Samples collected at 15-day intervals from WT and transgenic lines grown in soil in pots were used to calculate whole plant biomass values.

Total N content was measured using the Kjeldahl method (Li et al., 2006). The total dry weight (biomass) was estimated as the sum of weights of all plant parts. Total N accumulation was estimated as the sum of the N contents of all plant parts. Agronomic NUE (ANUE, g/g) was calculated as (grain yield−grain yield of zero-N plot)/N supply; N recovery efficiency (NRE, %) was calculated as (total N accumulation at maturity for N-treated plot−total N accumulation at maturity of zero-N plot)/N supply; physiological NUE (PNUE, g/g) was calculated as (grain yield −grain yield of zero-N plot)/total N accumulation at maturity; and the N harvest index (NHI, %) was calculated as (grain N accumulation at maturity/total N accumulation at maturity. Dry matter and N translocation and translocation efficiency method for the calculation of the reference in Ntanos et al. (2002) and Zhang et al. (2009). Dry matter translocation (DMT, g/m2) was calculated as dry matter at anthesis−(dry matter at maturity−grain yield); DMT efficiency (DMTE, %) was calculated as (DMT/dry matter at anthesis)×100%; the contribution of pre-anthesis assimilates to grain yield (CPAY, %) was calculated as (DMT/grain yield)×100%; the harvest index (HI, %) was calculated as (grain yield/dry matter at maturity)×100%; post-anthesis N uptake (PANU, g/m2) was calculated as total N accumulation at maturity−total N accumulation at anthesis; N translocation (NT, g/m2) was calculated as total N accumulation at anthesis−(total N accumulation at maturity−grain N accumulation at maturity); N translocation efficiency (NTE, %) was calculated as (NT/total N accumulation at anthesis)×100%; the contribution of pre-anthesis N to grain N accumulation (CPNGN, %) was calculated as (NT/grain N accumulation at maturity)×100% (FIG. 13).

1.1.3. Growth Conditions

T0, T2, T3 and T4 generation plants were grown in plots at the Nanjing Agricultural University in

Nanjing, Jiangsu (FIG. 25). T1 generation plants were grown in Sanya, Hainan. Jiangsu is in a subtropical monsoon climate zone. Chemical properties of the soils in the plots at the Nanjing Agricultural University included organic matter, 11.56 g/kg; total N content, 0.91 g/kg; available P content, 18.91 mg/kg; exchangeable K, 185.67 mg/kg; and pH 6.5. Basal applications of 30 kg P/ha as Ca(H2PO4)2 and 60 kg/K ha (KCl) were made to all plots 3 days before transplanting. N fertilizer accounted for 40%, 30% and 40% of the total N fertilizer was applied prior to transplanting, at tillering, just before the heading stage, respectively.

1.1.4. Stress Conditions

Rice ‘Wuyunjing 7’ seeds and transgenic plants were surface sterilized with 10% (v/v) hydrogen peroxide for 30 min and then rinsed thoroughly with deionized water. The sterilized seeds were germinated on plastic supporting netting (mesh of 1 mm-2) mounted in plastic containers for 2 week. Uniform seedlings were selected and then transferred to a tank containing 8 L of International Rice Research Institute (IRRI) nutrient solution (1.25 mM NH4NO3, 0.3 mM KH2PO4, 0.35 mM K2SO4, 1 mM CaCl2.2H2O, 1 mM MgSO4.7H2O, 0.5 mM Na2SiO3, 20 μM NaFeEDTA, 20 μM H3BO3, 9 μM MnCl2.4H2O, 0.32 μM CuSO4.5H2O, 0.77 μM ZnSO4.7H2O, and 0.39 μM Na2MoO4.2H2O, pH 5.0). All the plants were grown in a growth room with a 16-h-light (30° C.)/8-h-dark (22° C.) photoperiod, and the relative humidity was controlled at approximately 70%. The solution was refreshed every 2 day. Treatment was carried out after two weeks of growth, and the seedlings were then grown with different stress conditions for 9 days. The cold treatment were grown outdoor, the maximum temperature and the minimum temperature were 18° C. and 2° C. 10% polyethylene glycol (PEG) was used to simulate drought stress.

1.1.5. The Field Experiments for Yield Harvest

T0-T4 generation seedlings were planted in the same experiment site in Nanjing, except T1 in Sanya. Seeds generation transgenic lines and WT were surface sterilized with 10% (v:v) hydrogen peroxide (H₂O₂) for 30 min and rinsed thoroughly with deionized water. The transgenic seeds were soaked in water containing 25 mg/L hygromycin and the WT seeds were soaked in water. After 3 days, the sterilized seeds were sown evenly in wet soil. The similar seedlings were transplanted to field plots after germination three weeks.

T1-T3 plants were planted in plots fertilized at a rate of 300 kg N/ha as urea and in plots without N fertilization. Plots were 2×2.5 m in size with the seedlings planted in a 10×10 array. Plants at the edges of all four sides of each plot were removed at maturity to avoid the influence of edge effects. Four points, each containing four seedlings, totally 16 seedlings, were selected randomly within the remaining centre 8×8 array of plants and samples were collected (Ookawa et al., 2010; Pan et al., 2013; Khuram et al., 2013; Srikanth et al., 2015). Yield and biomass values determined from these four points in each plot were used to calculate the yield per hectare and biomass of each line and 3 random plots for each line were designed in the experiment (FIG. 25).

T3 generation plants were sampled at 15-day intervals for determination of grain yield, biomass, and N content. The growth rate was the dry weight of the weight increase in the unit time after seedlings were transplanted to the plots.

T4 generation plants were planted in a plots fertilized at a rate of 0.180 and 300 kg N/ha as urea. Same random field plots with 3 replicates were designed as T1-T3 plants for yield and biomass values determined from these four points were used to calculate the yield and biomass per plant and ANUE of each line.

1.1.6. Expression Ratio Analysis

We did two experiments to address this ratio pattern. The first experiment to determine the same tissue as in rice culm, the expression of OsNRT2.1 and OsNAR2.1 at each 15 day after transplanting into field. We sampled the culm of rice plants in the field and put samples into liquid N2 and ground sample to extract RNA. Total RNAs were prepared from the various tissues of WT and transgenic plants using TRIzol reagent (Vazyme Biotech Co., Ltd, http://www.vazyme.com). Real time PCR was carried as described before (Li et al., 2014). All primers used for qRT-PCR are listed in FIG. 12. First, we compared OsNRT2.1 and OsNAR2,1 expression with OsActin gene to get the expression data. Then, using the expression data of OsNAR2.1 as the X-axis and expression data of OsNRT2.1 as Y-axis in the same sample as the expression of OsNAR2.1, draw the points and making linage with the points as well as calculated the formula of Y and X relationship. The slope will be the ratio of OsNRT2.1 and OsNAR2.1. The same method was used in the second experiment to investigate the expression pattern of OsNRT2.1 and OsNAR2.1 in different organs in the same stage as the grain filling stage.

1.1.7. mRNA Sampling and qRT-PCR Assay

In order to investigate the expression pattern in plant organs we sampled mRNA for seeds, palea and lemma, leaf blade I, leaf blade II, leaf blade III, leaf sheath I, leaf sheath II, leaf sheath III, inter node I, inter node II, inter node III and newly developed root (3 cm from root tips) at the grain filling stage (described in FIG. 20). Tracking rice in the whole growth period of gene expression in T3 generation, we sampled mRNA from culms including leaf sheath and inter node I (described in FIG. 22) at 15 d, 30d, 45d, 60d, 75d, 90d after transplanting.

Total RNAs were prepared from the various tissues of WT and transgenic plants using TRIzol reagent (Vazyme Biotech Co., Ltd, http://www.vazyme.com). Real time PCR was carried as described before (Li et al., 2014). All primers used for qRT-PCR are listed in FIG. 12.

1.1.8. Statistical Analysis

Data were analyzed by Tukey test of one way analysis of variance (ANOVA), except that analysis of covariate (ANCOVA) was used in the biomass and growth rate during growth stages (FIG. 4ab). Different letters on the histograms or after mean values indicate statistically significant differences at P<0.05 between the transgenic plants and WT (one way ANOVA). The asterisk at the end of time course indicates their statistical significant differences among plants and # indicates their statistical significant differences during the growth stages at P<0.05 (ANCOVA). All statistical evaluations were conducted using the IBM SPSS Statistics ver. 20 software. (SPSS Inc., Chicago, Ill.)

1.2. Results

1.2.1. Generation of Transgenic Rice Plants Expressing pUbi:OsNRT2.1 and pOsNAR2.1:OsNRT2.1 Constructs and Field Analysis of Traits

The ubiquitin promoter (pUbi) has been used as a strong promoter in a variety of applications in gene transfer studies and was shown to drive gene expression most actively in rapidly dividing cells (Cornejo et al., 1993). Overexpression of just the OsNRT2.1 gene in rice was previously shown to not increase NO3− uptake (Katayama et al., 2009).

We introduced pUbi:OsNRT2.1 (FIG. 15a) and pOsNAR2.1:OsNRT2.1 (FIG. 15b) expression constructs into Wuyunjing 7 (WYJ7), a rice cultivar that produces high yields in Jiangsu province, using Agrobacterium tumefaciens-mediated transformation. We generated 23 lines exhibiting increased OsNRT2.1 expression, including 12 pUbi:OsNRT2.1 lines and 11 pOsNAR2.1:OsNRT2.1 lines (FIG. 16).

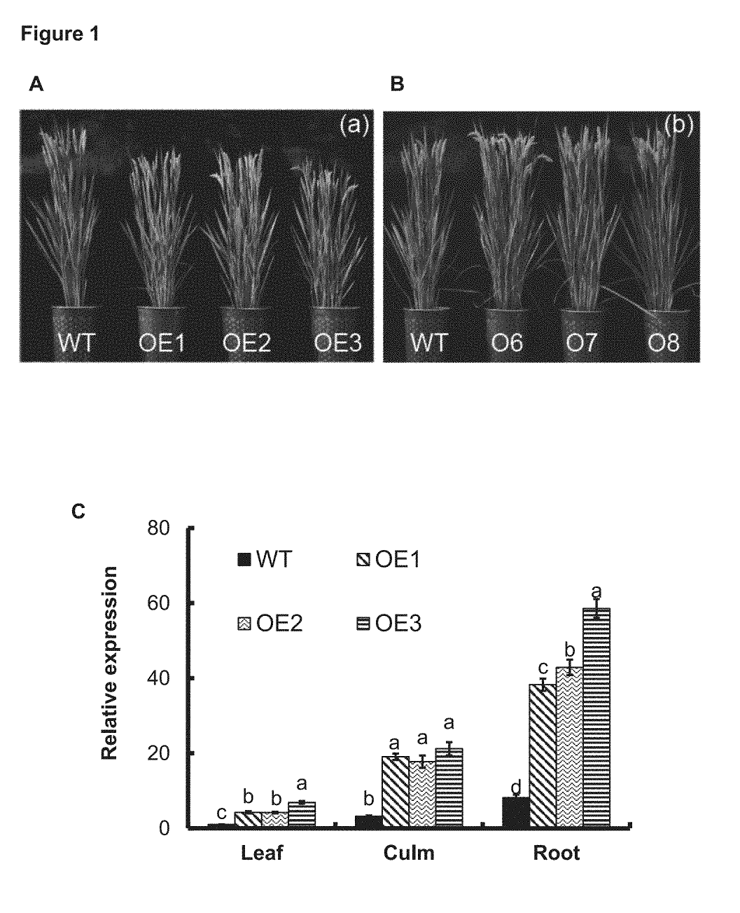

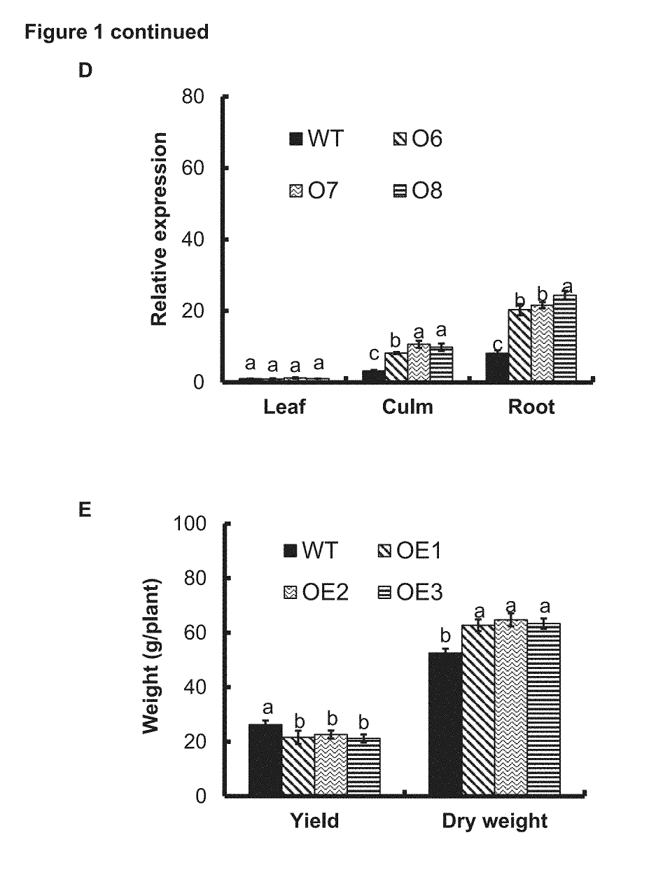

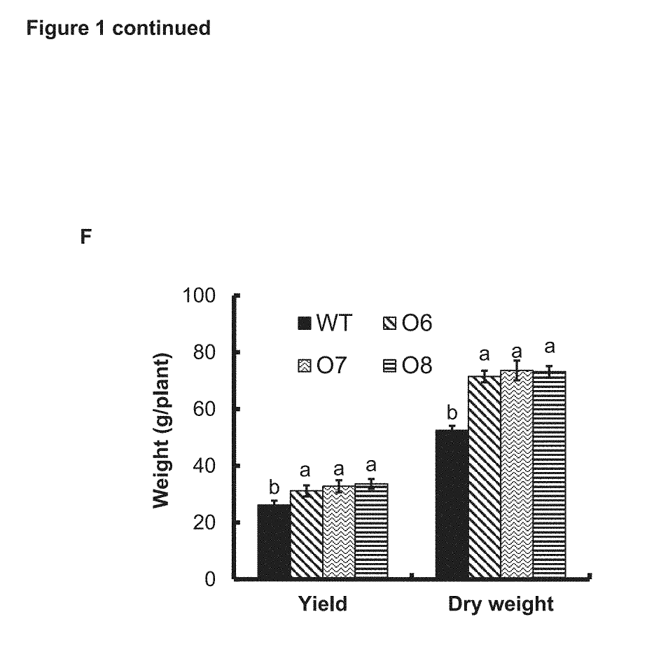

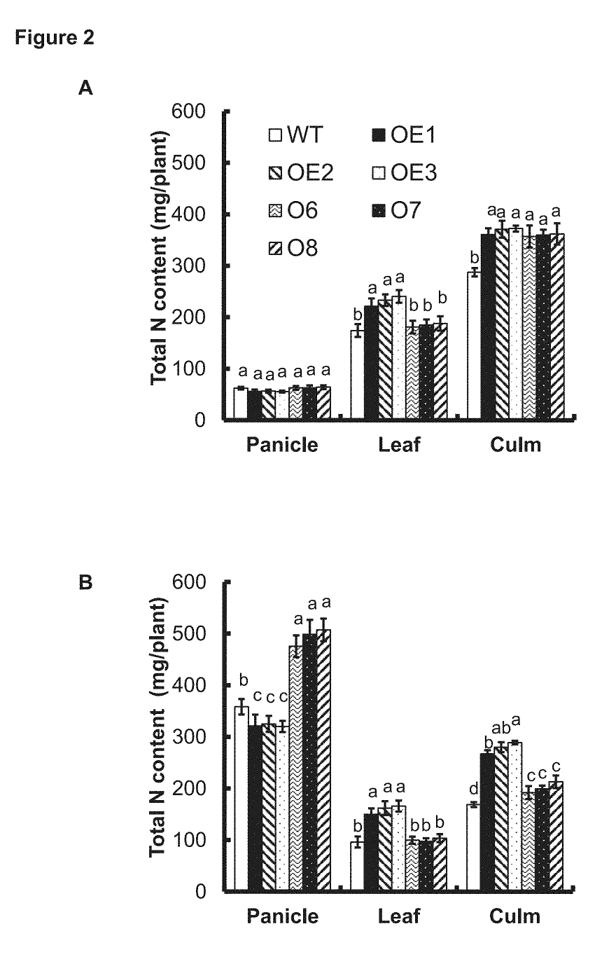

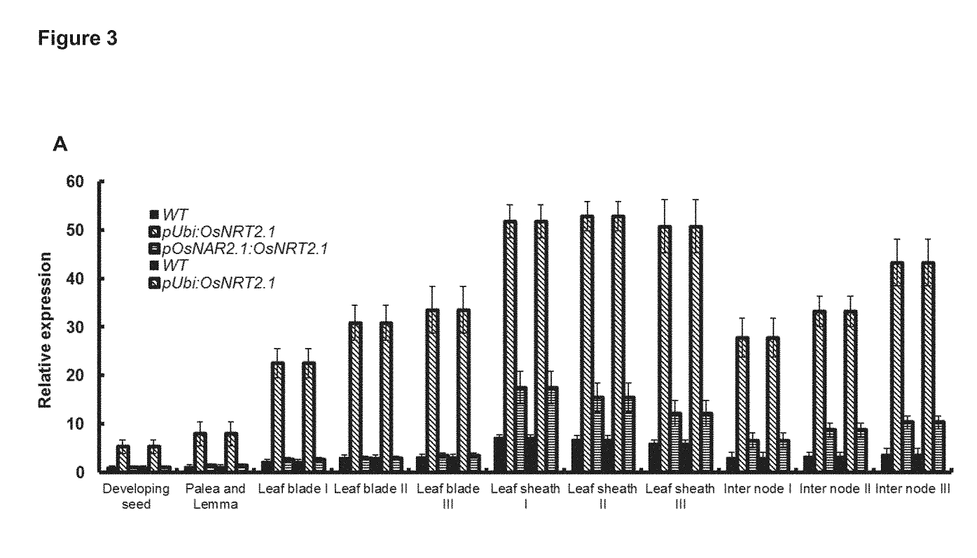

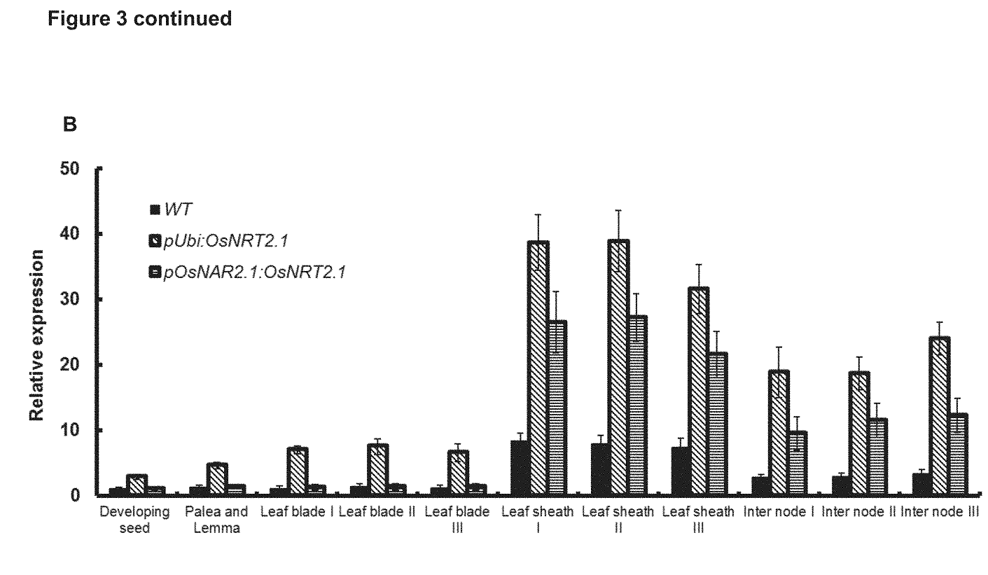

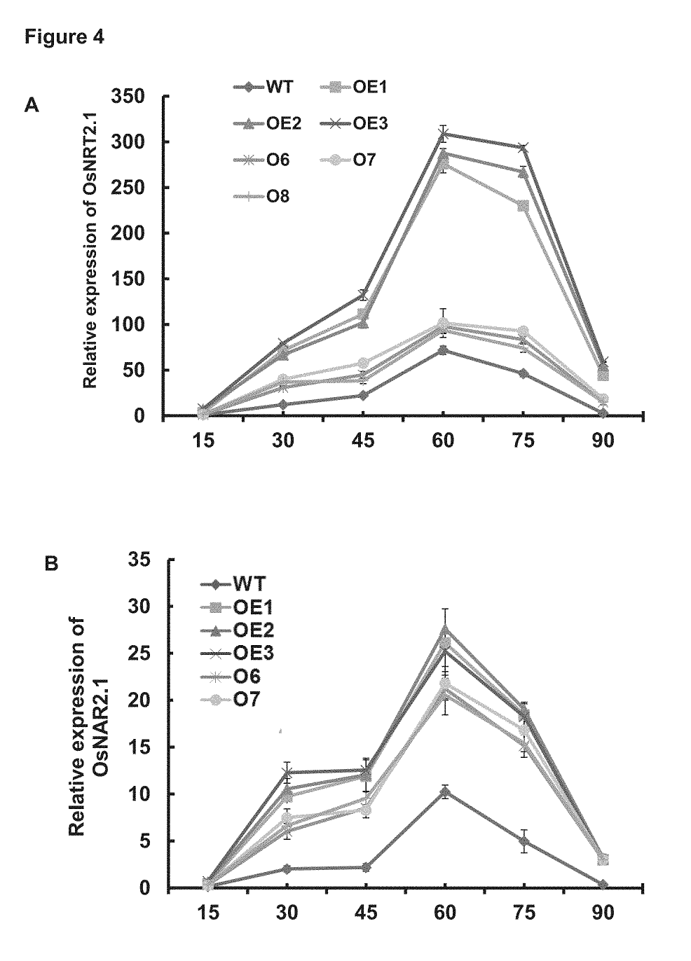

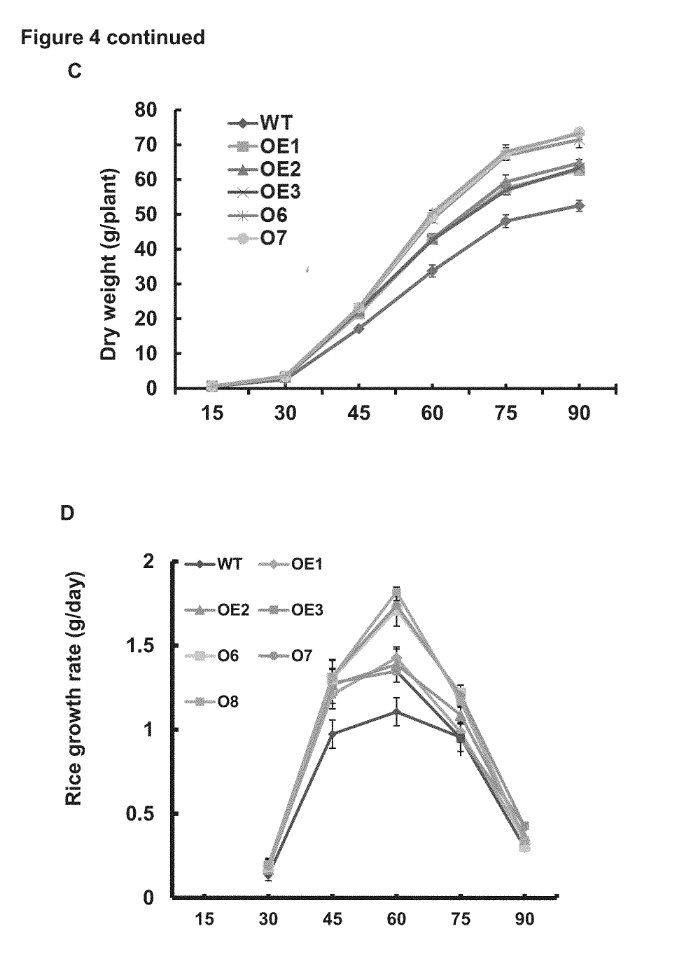

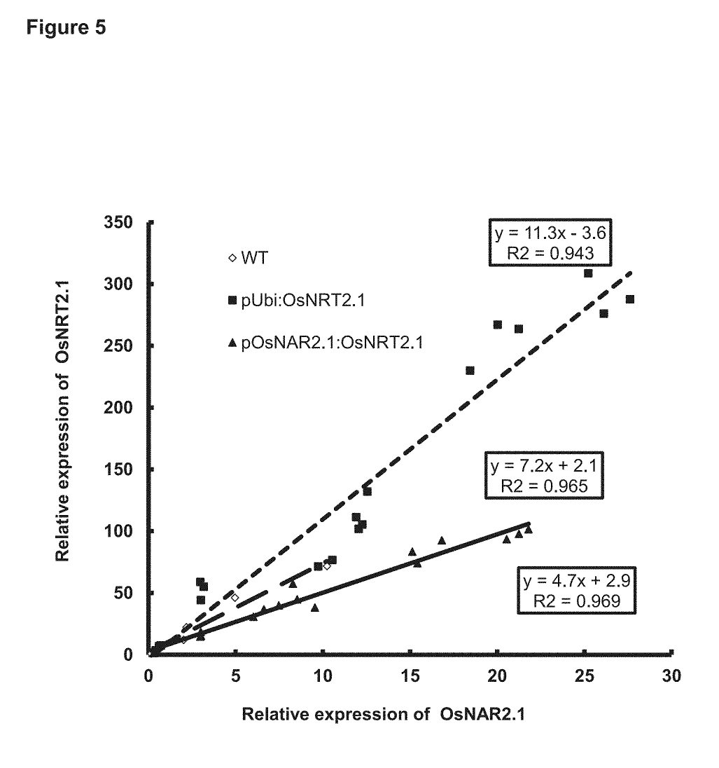

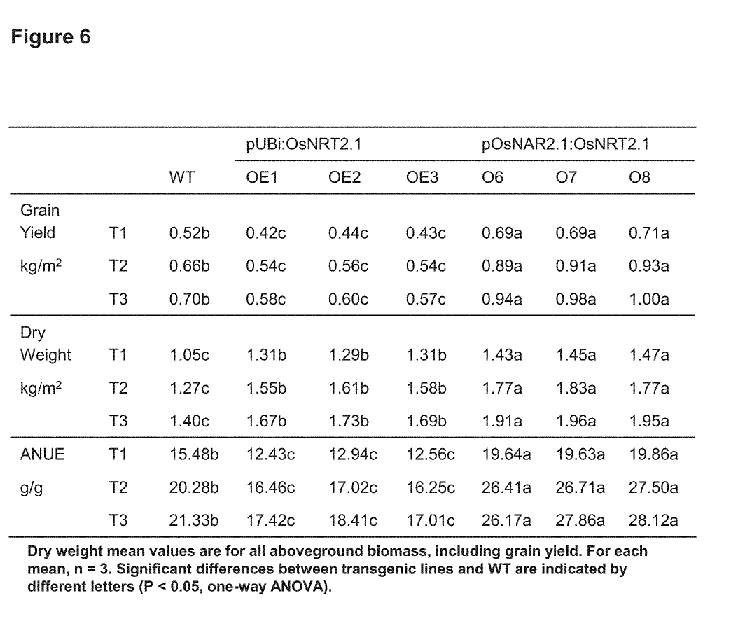

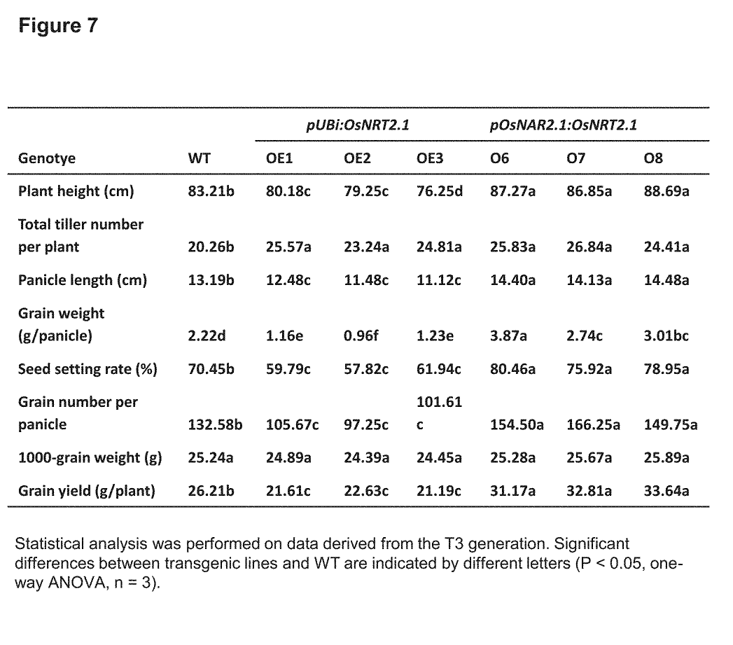

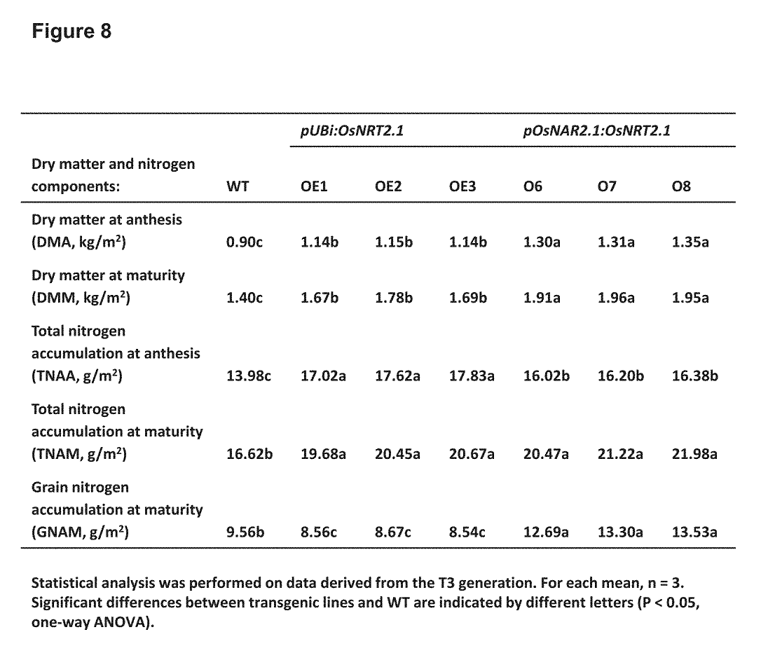

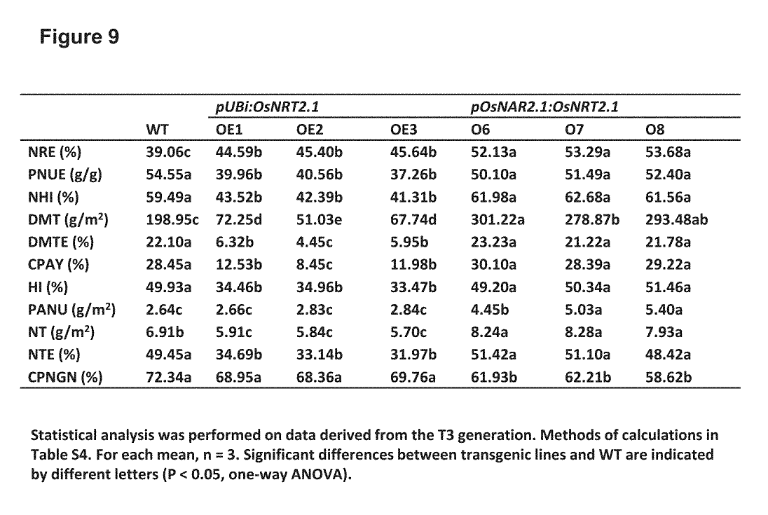

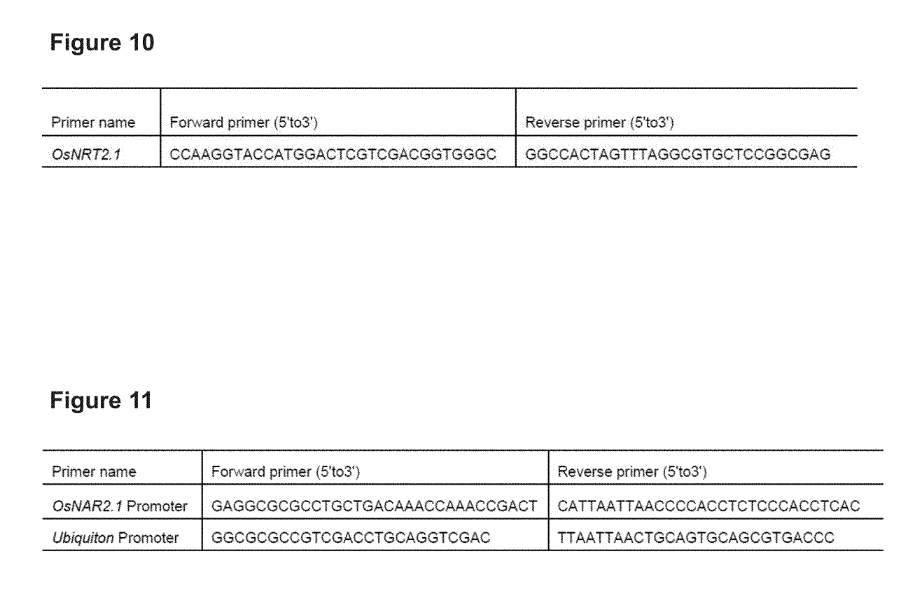

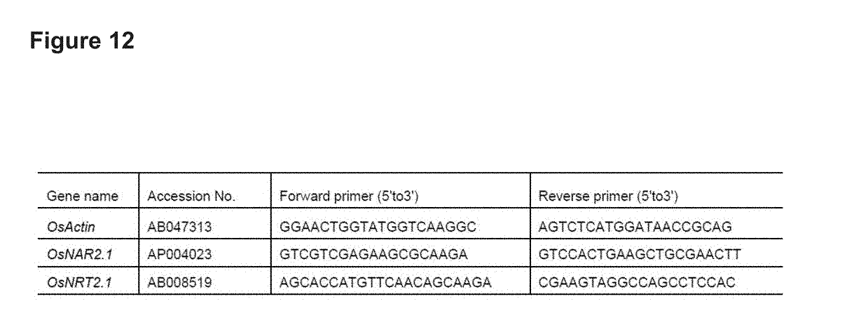

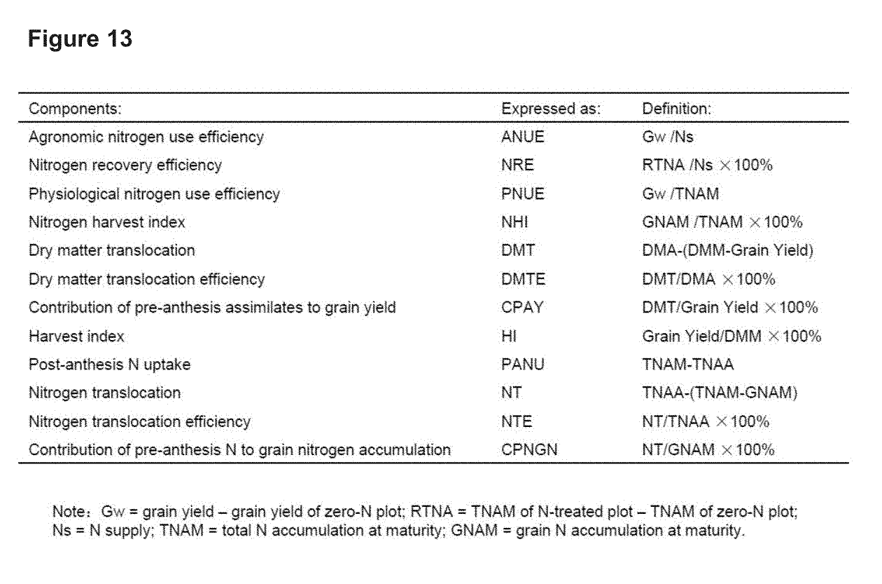

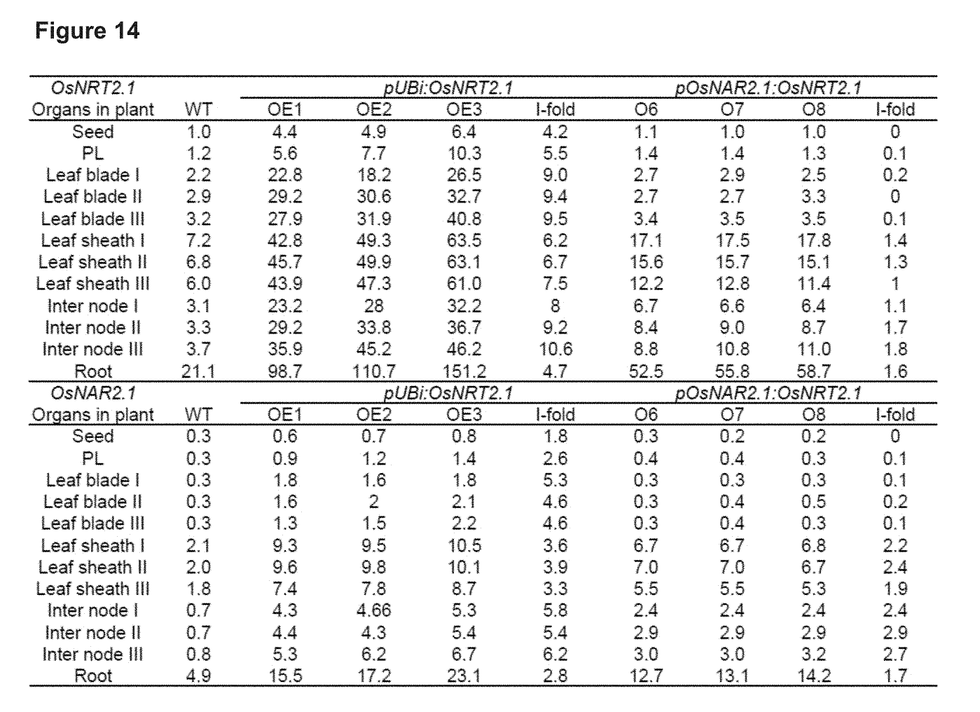

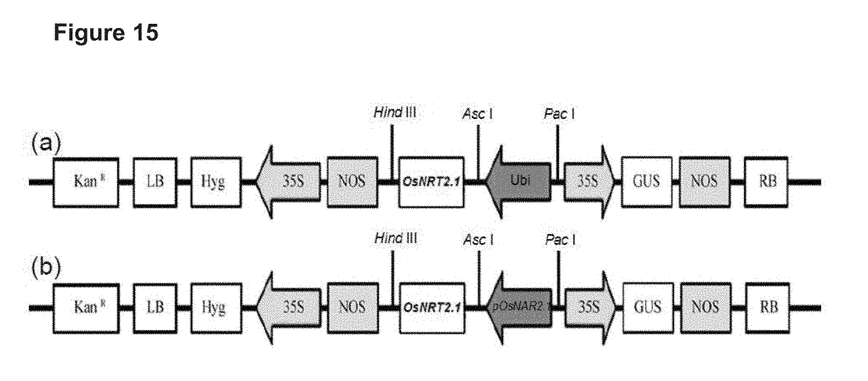

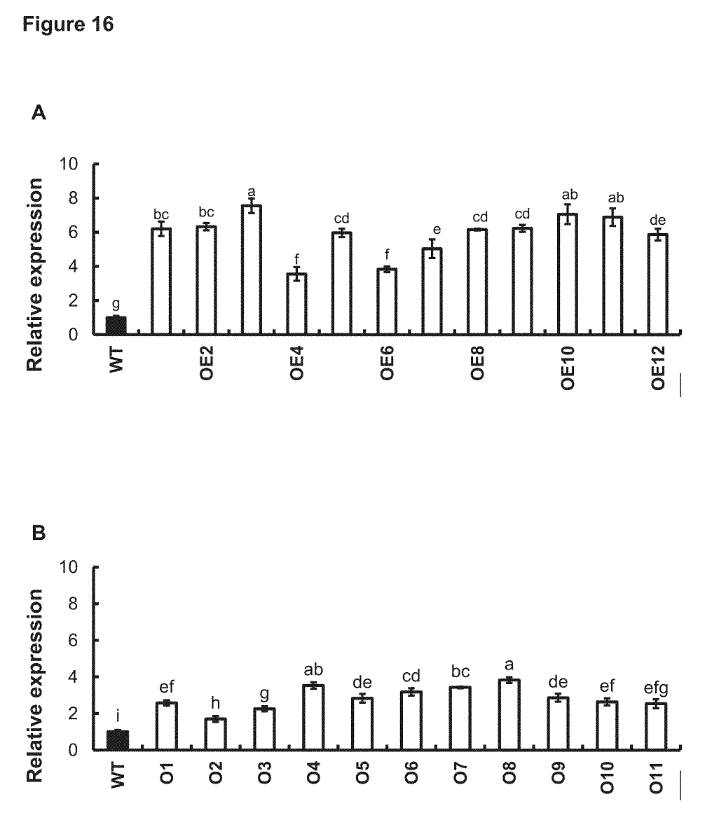

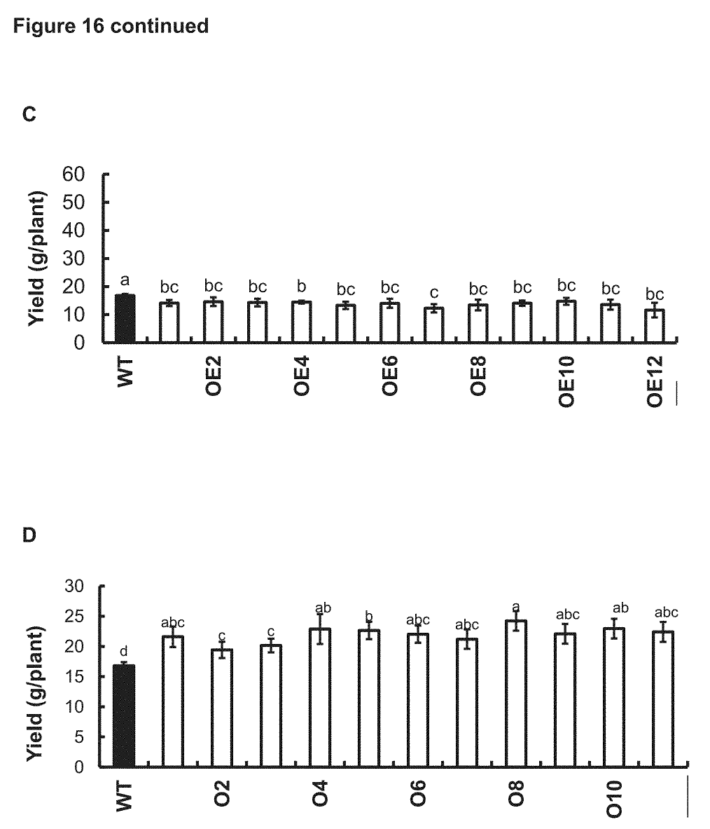

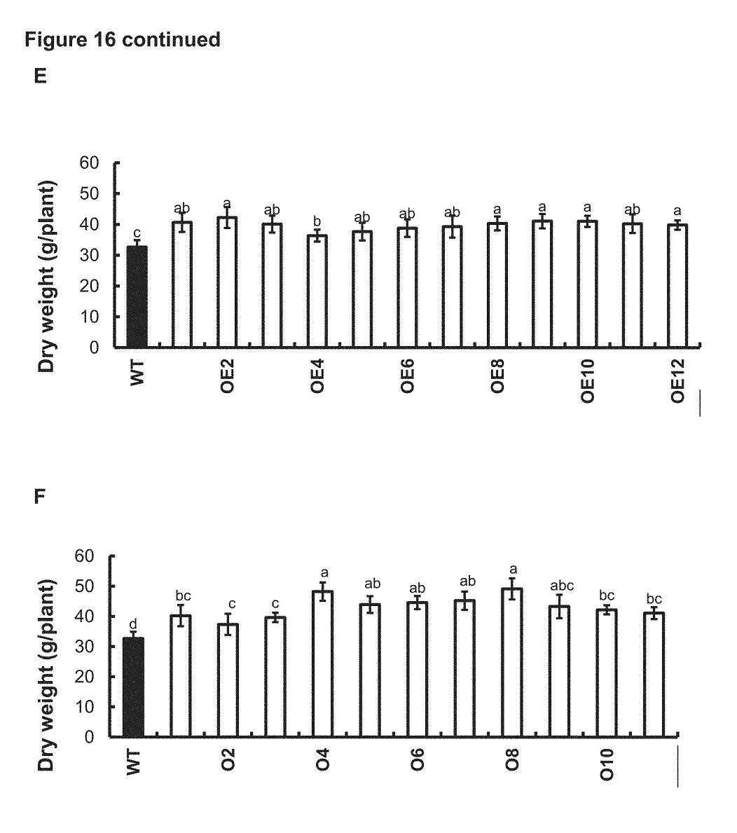

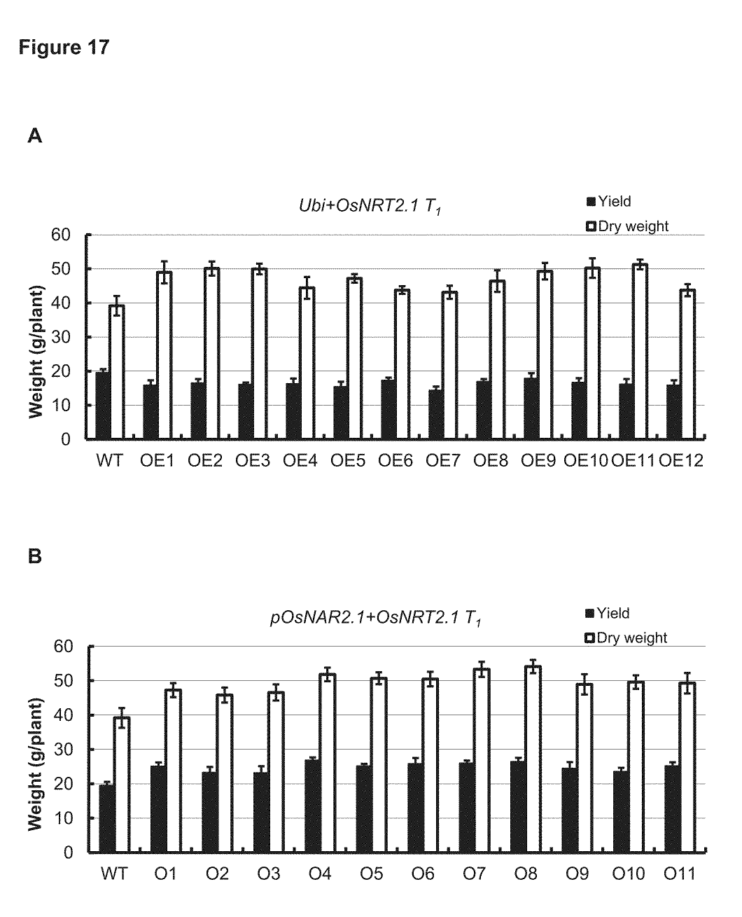

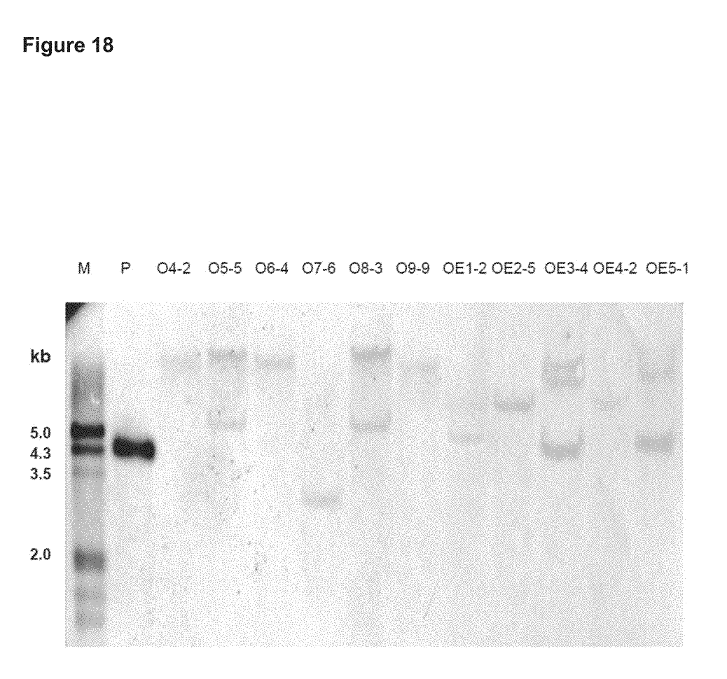

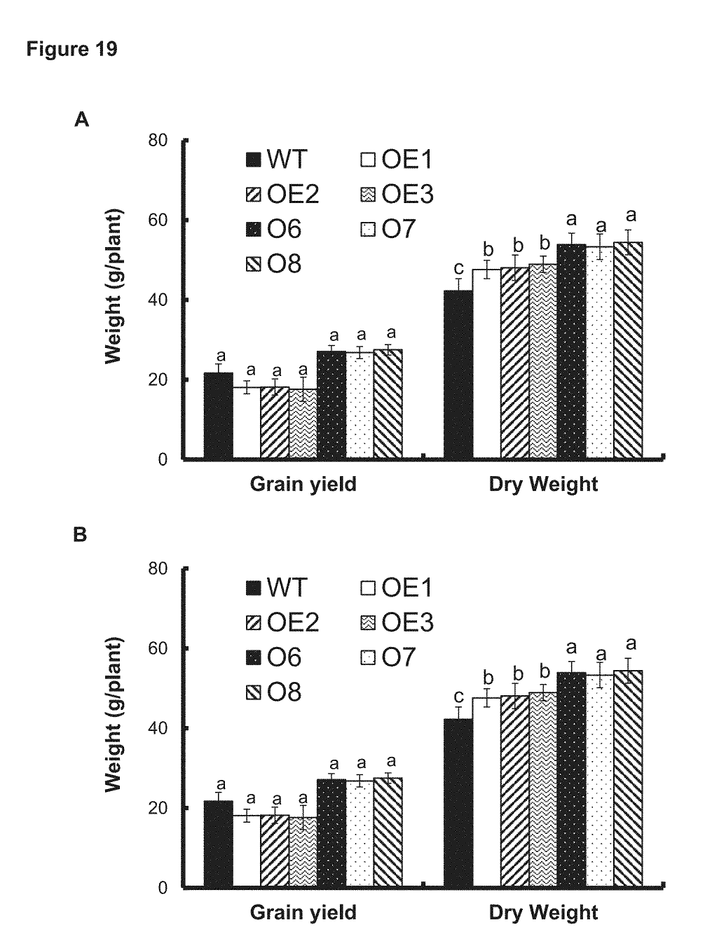

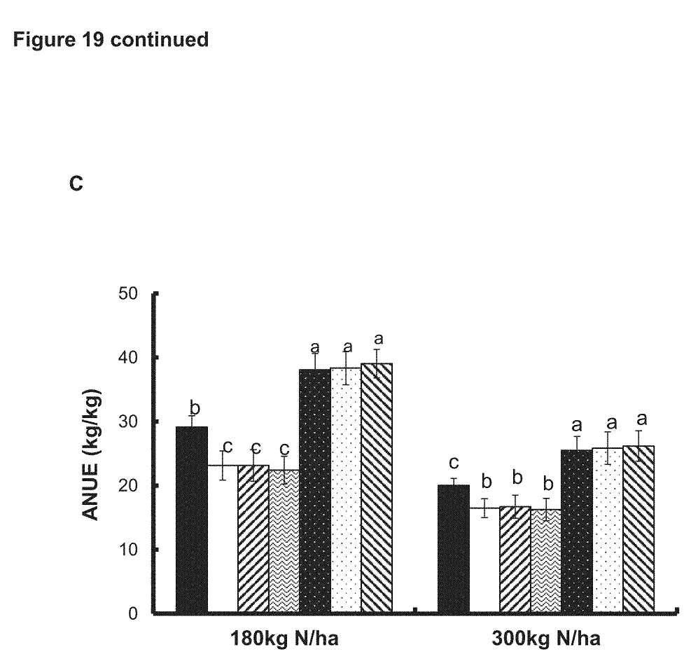

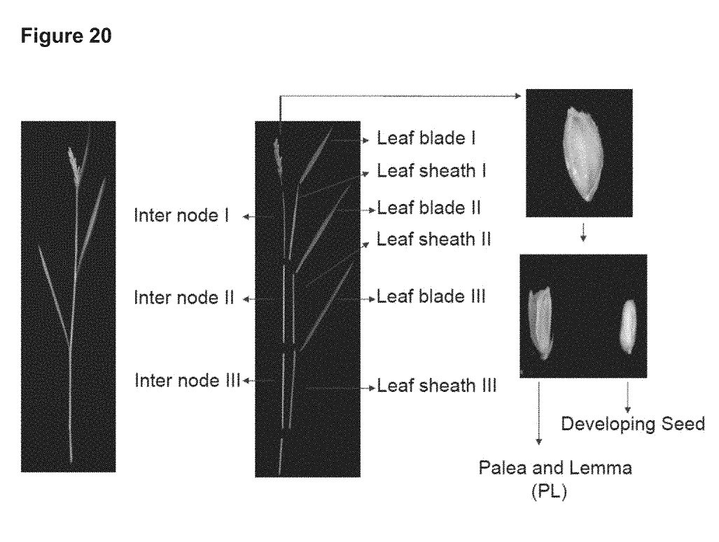

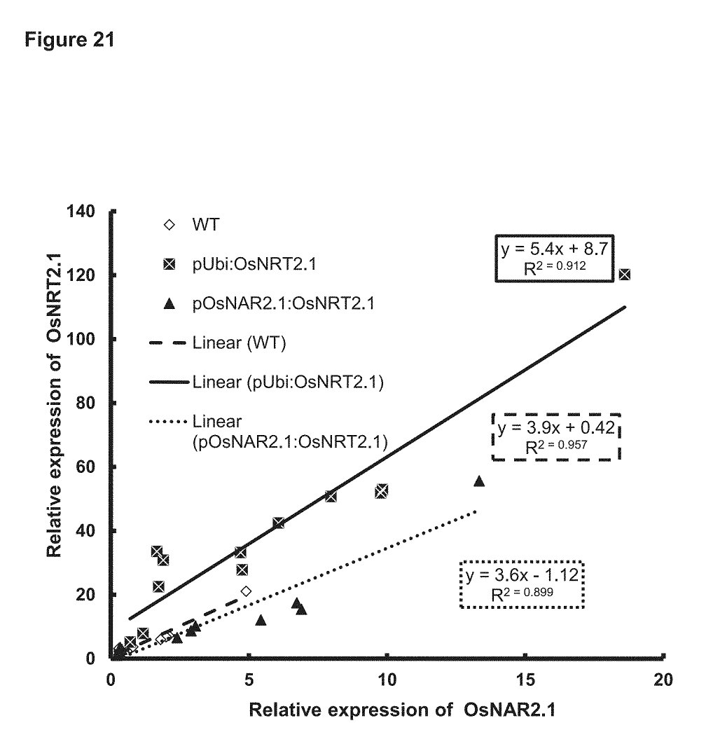

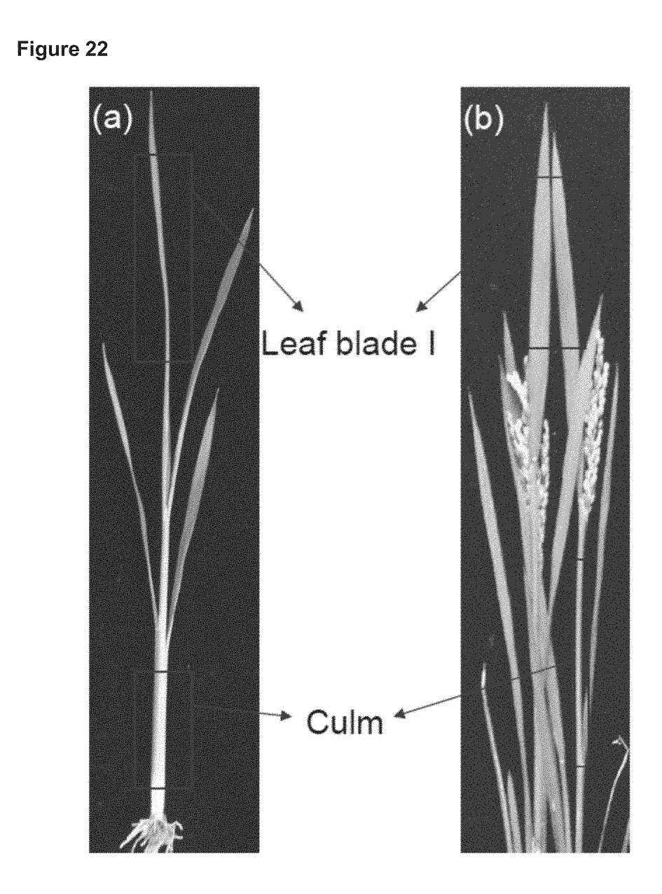

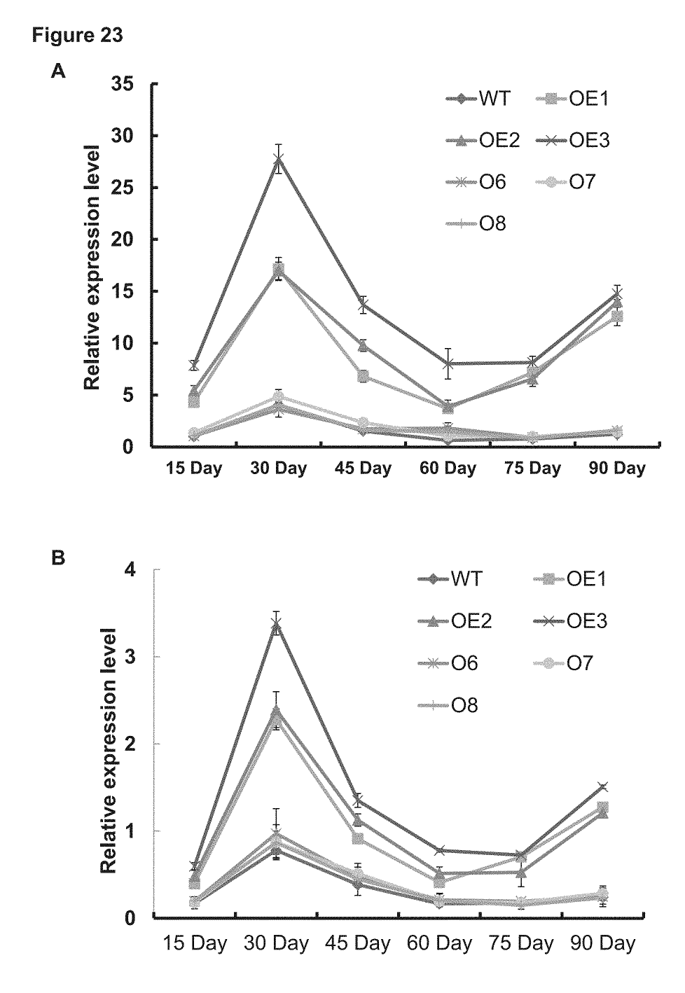

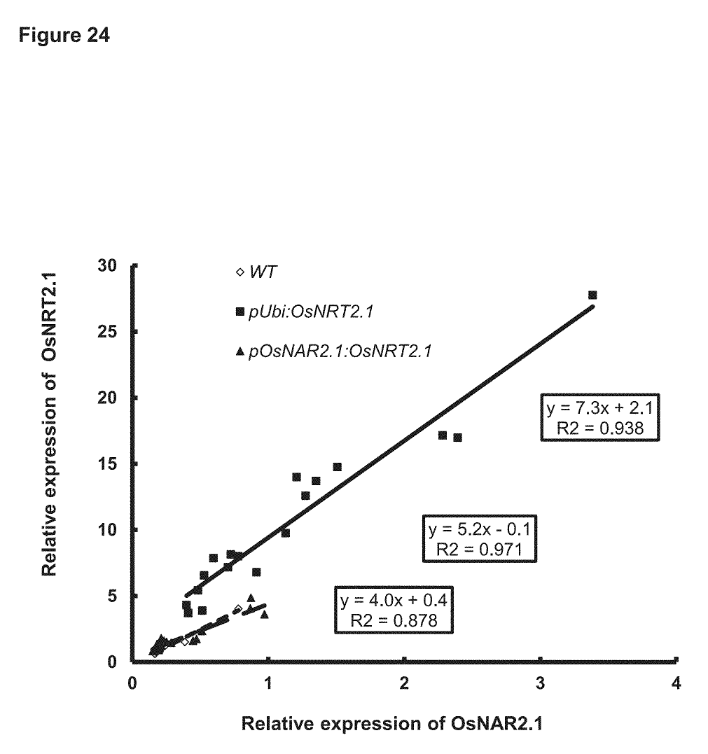

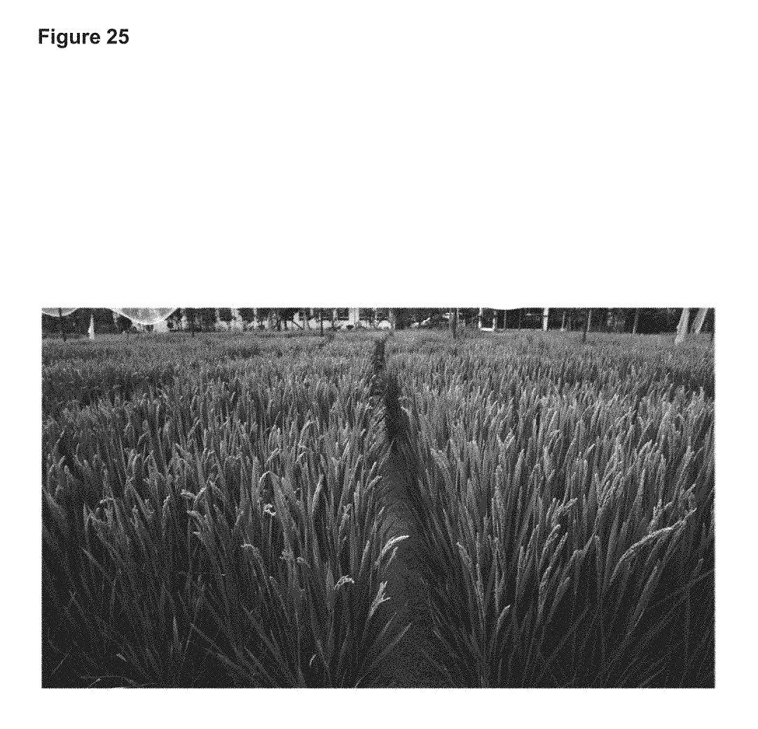

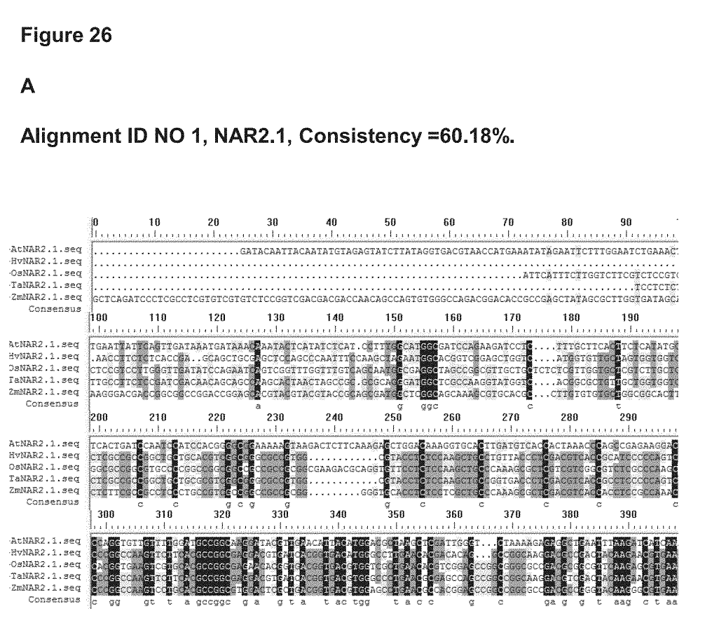

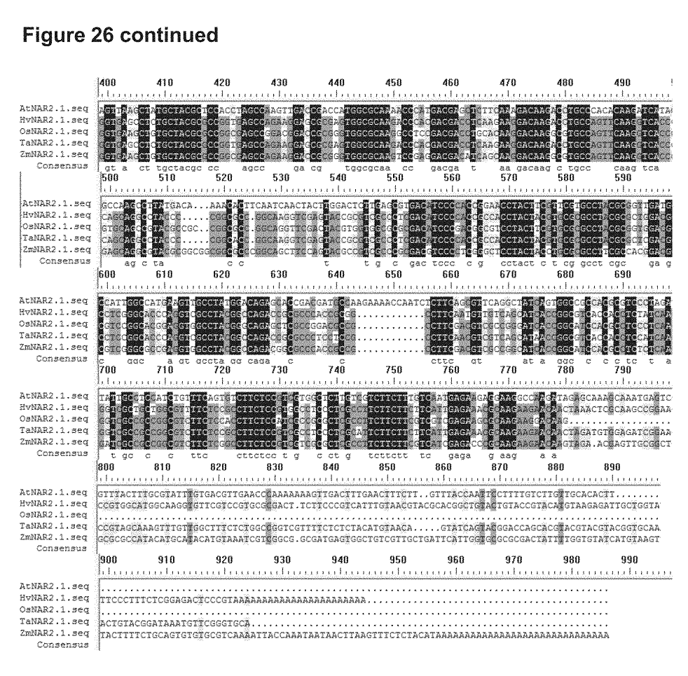

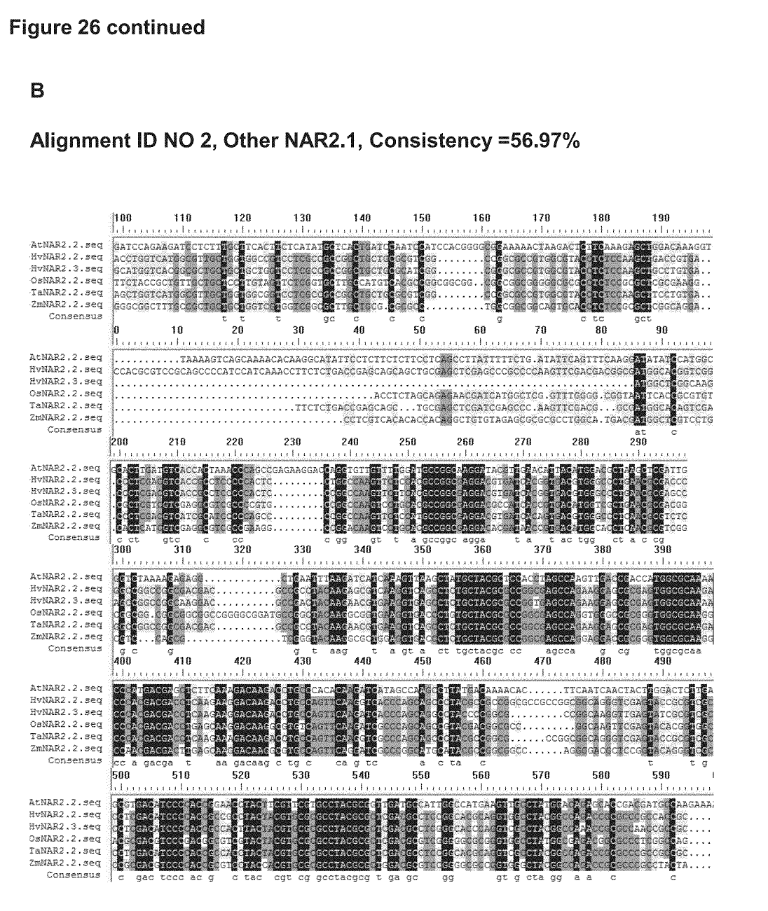

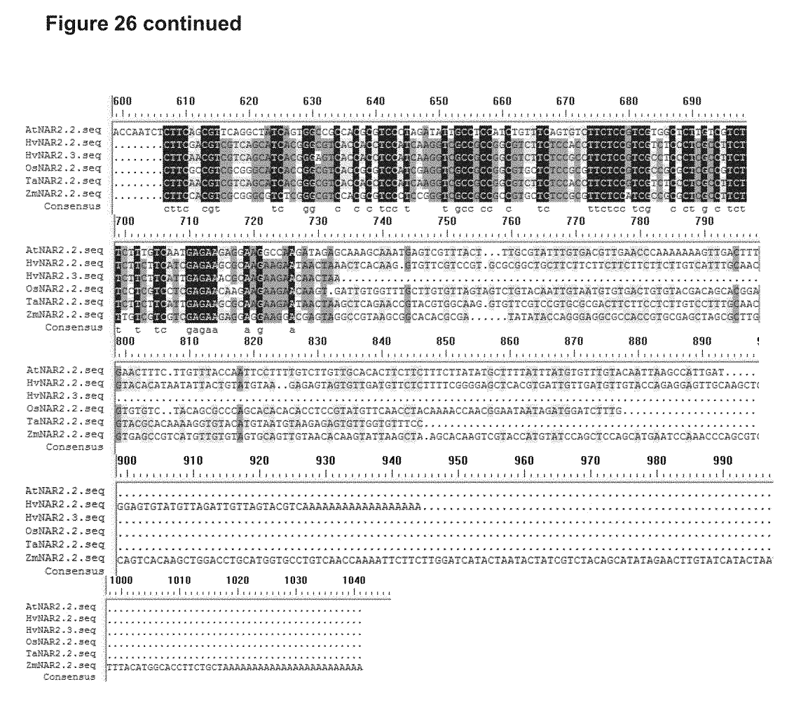

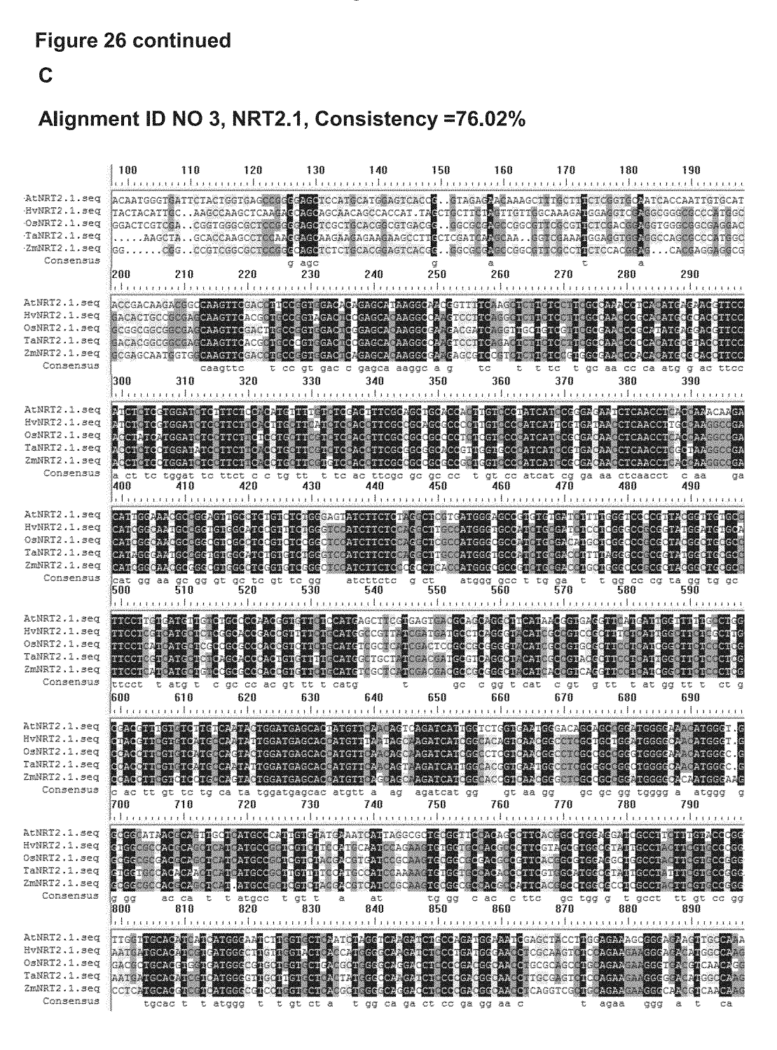

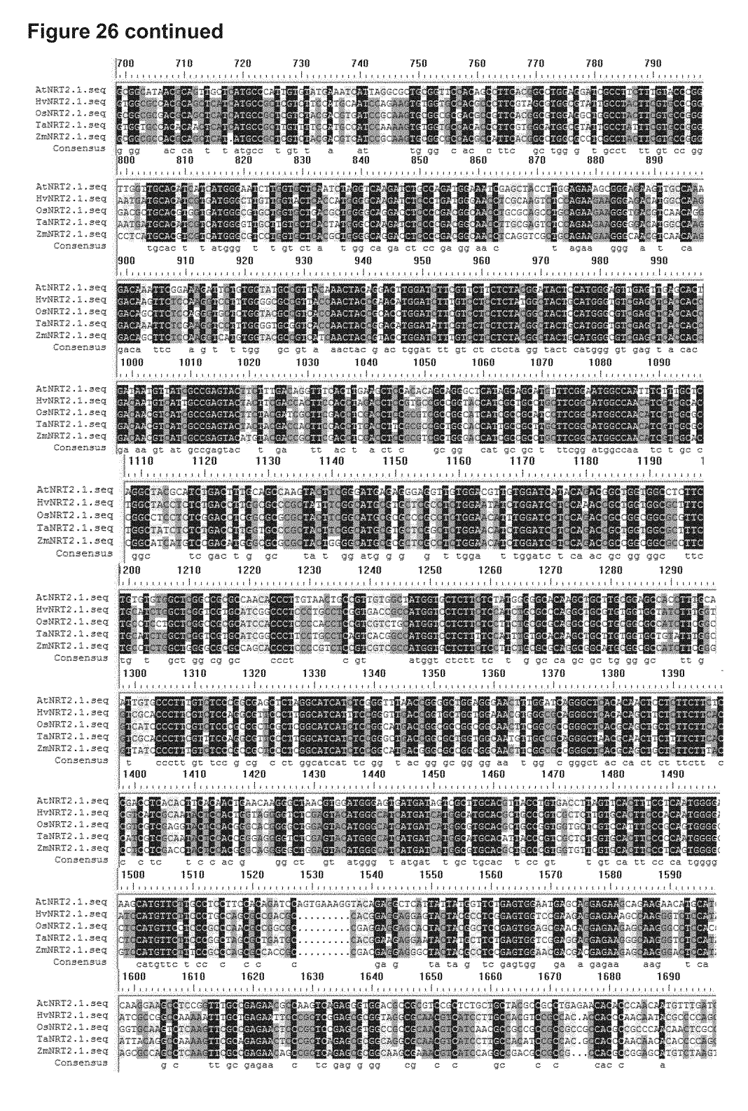

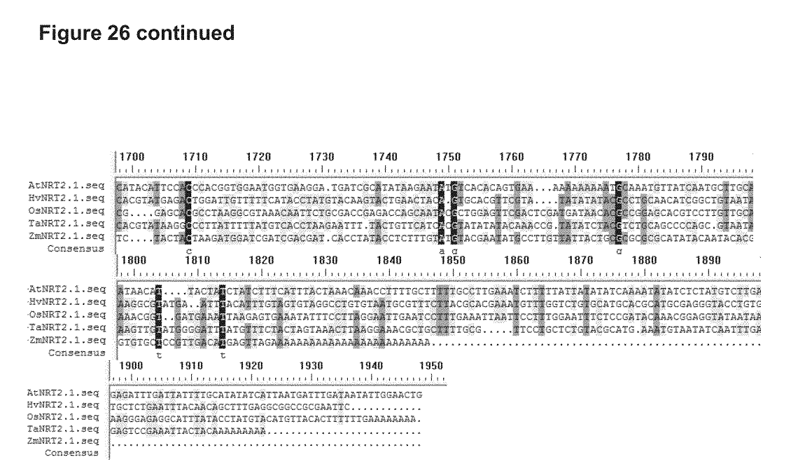

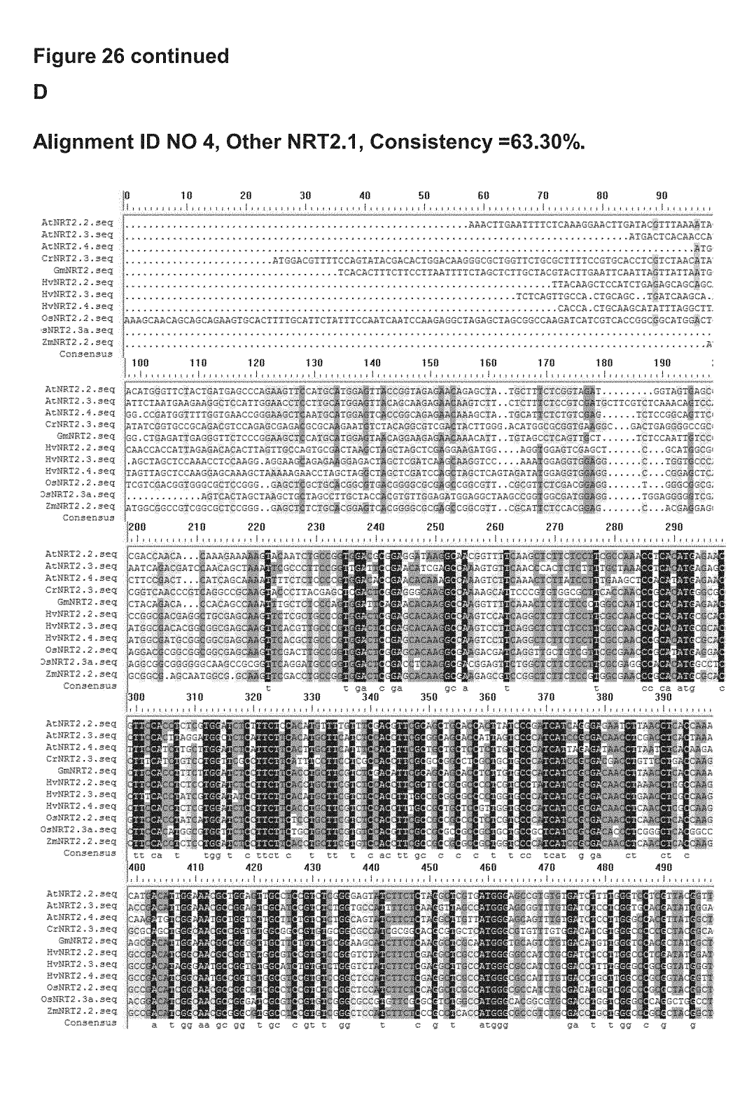

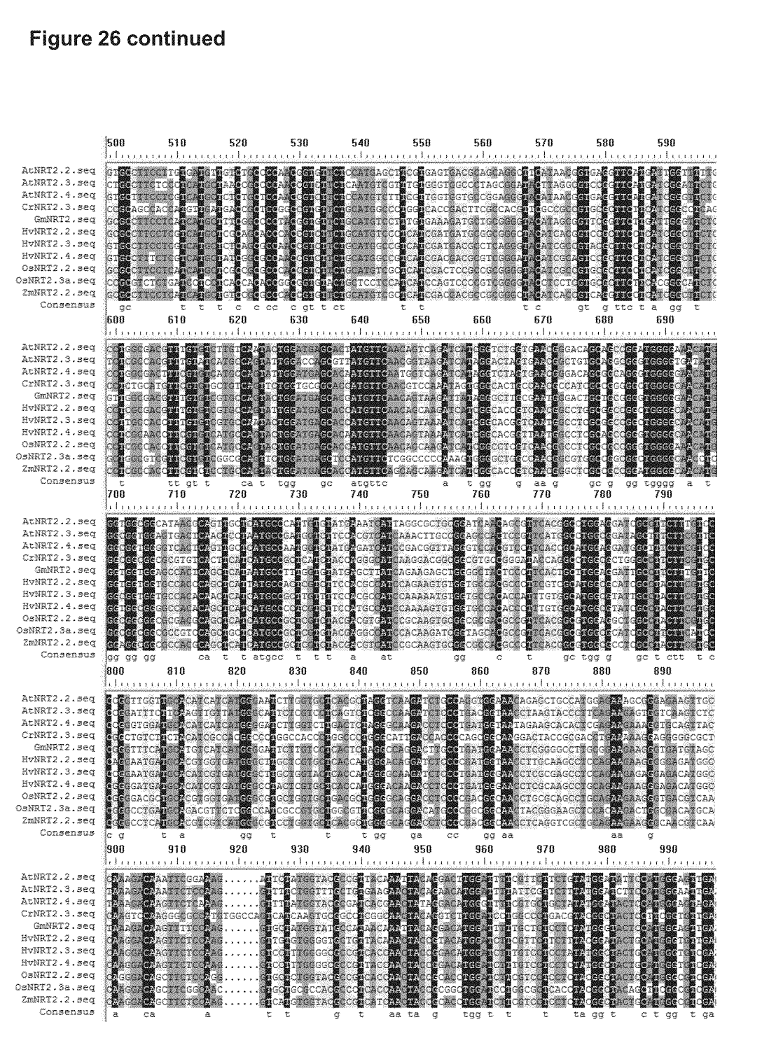

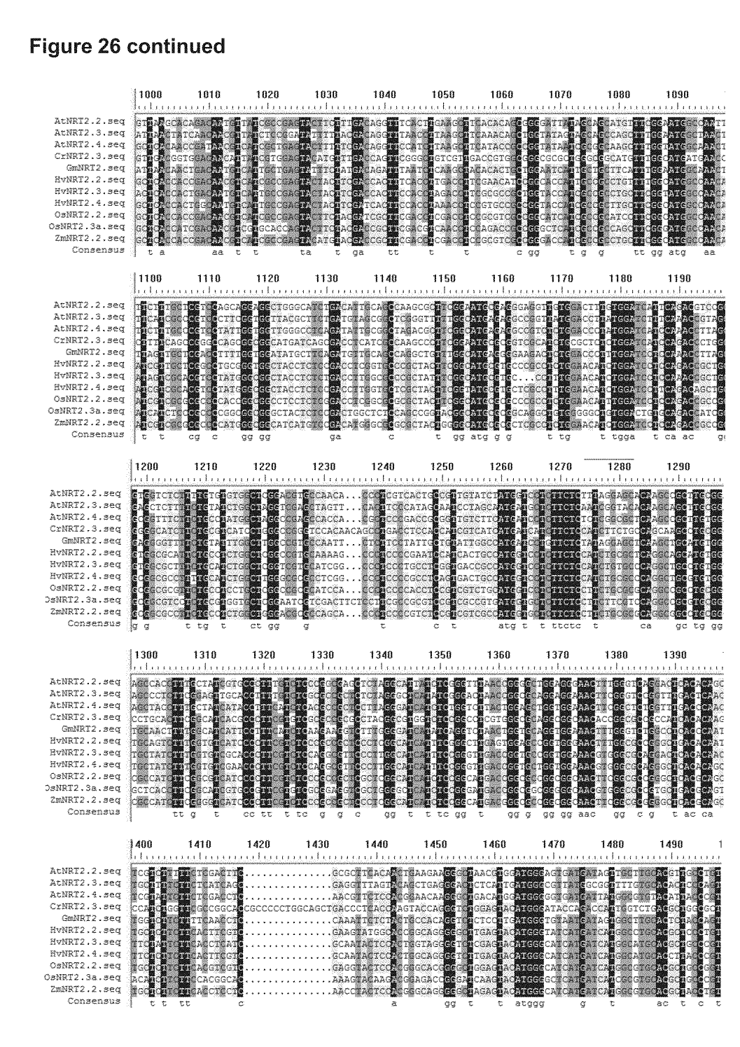

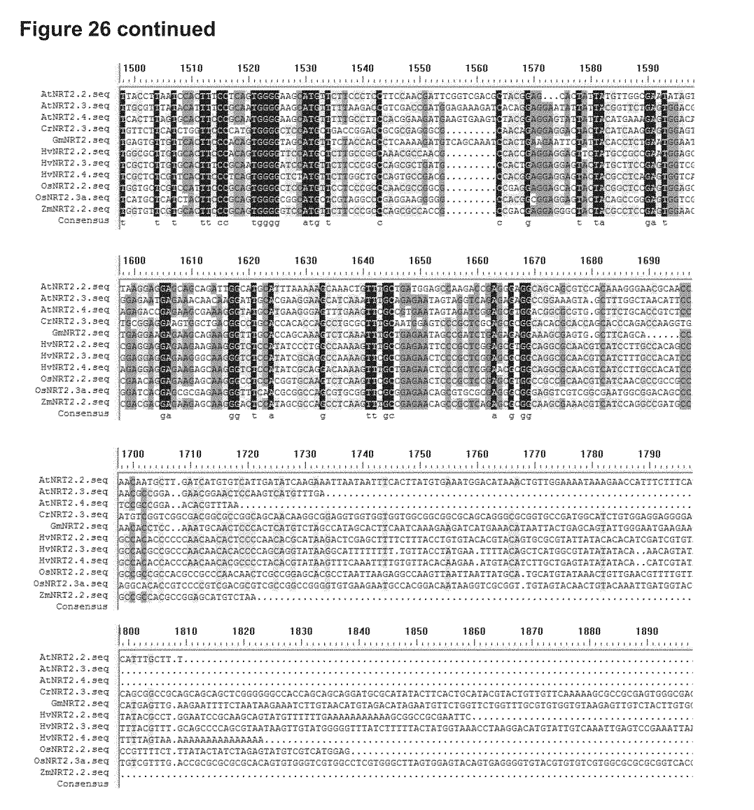

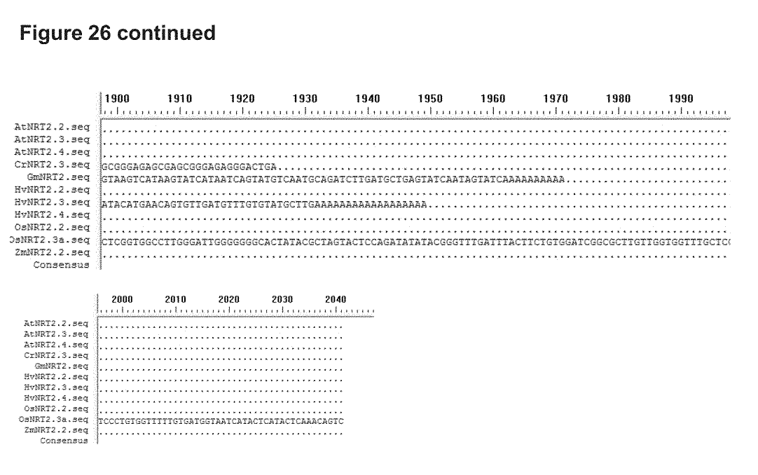

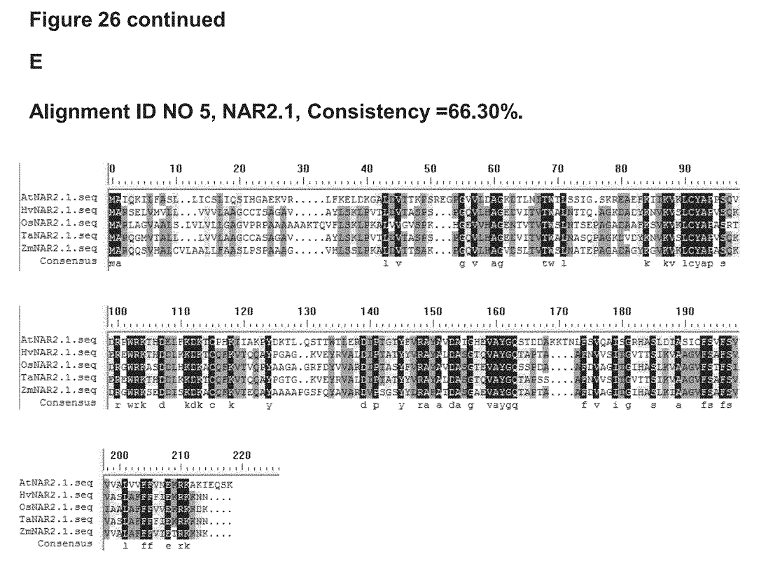

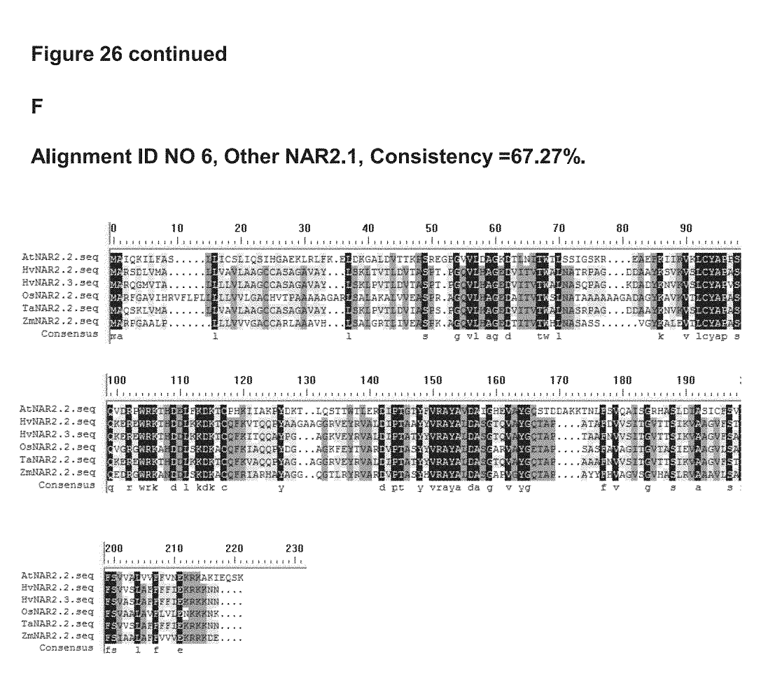

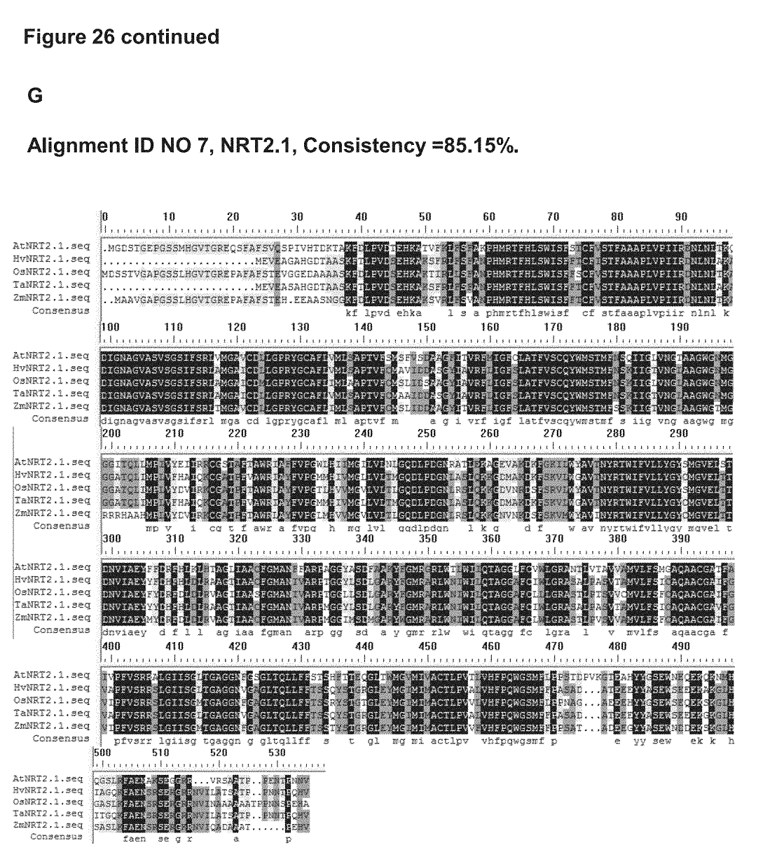

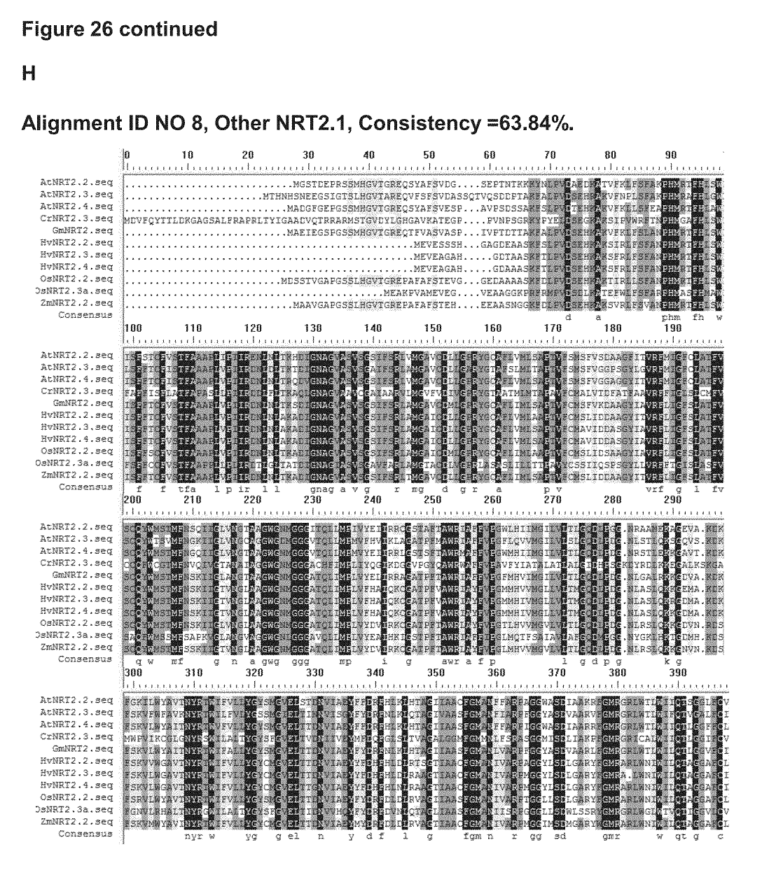

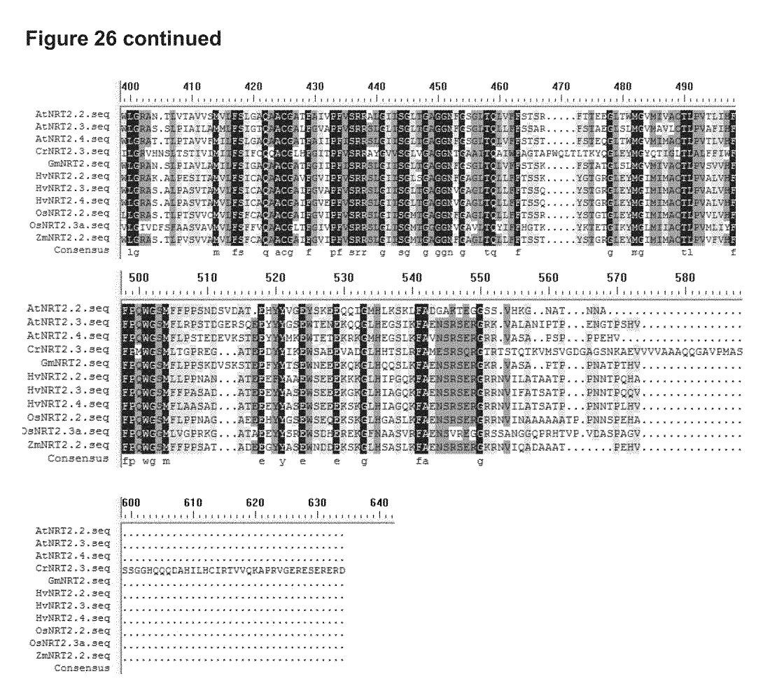

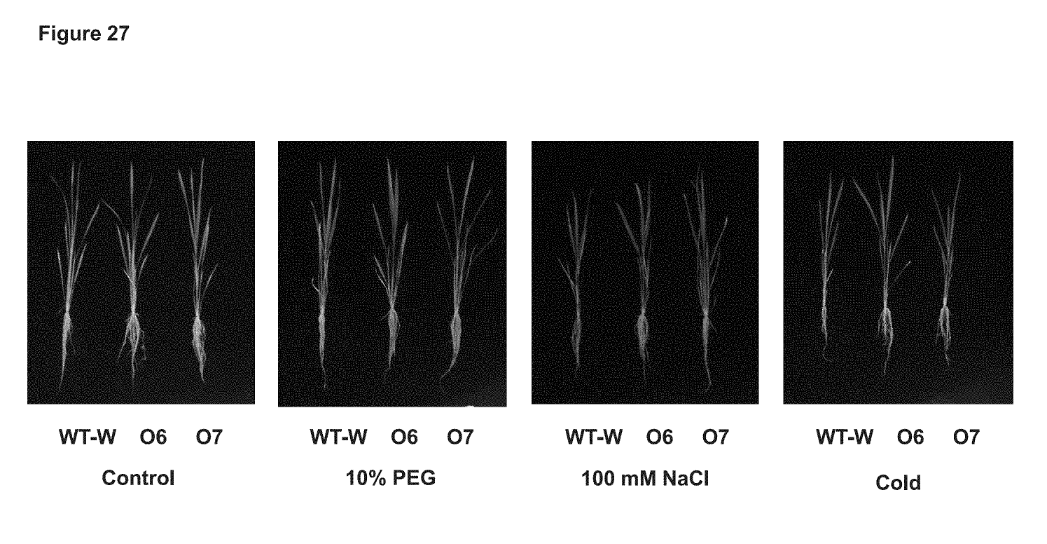

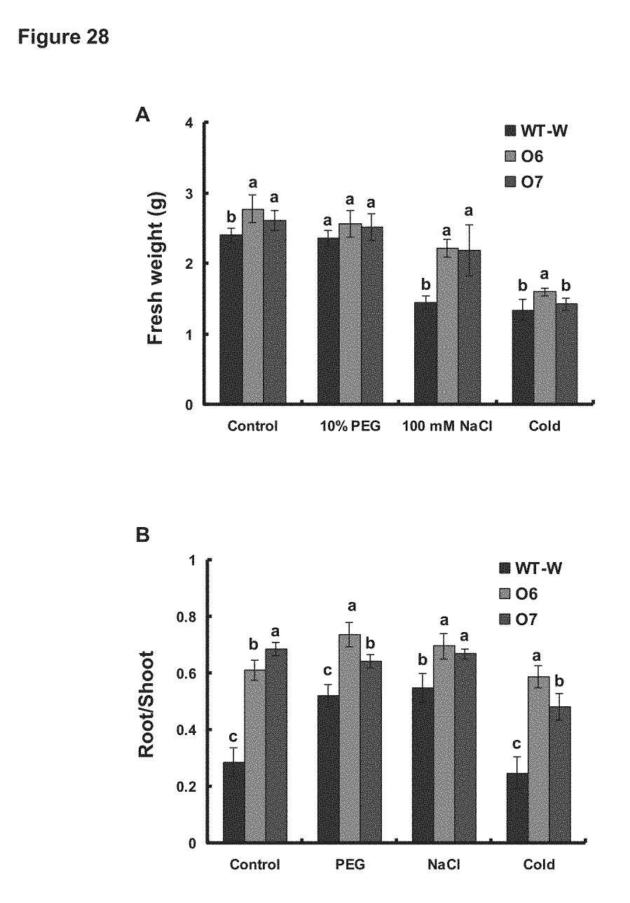

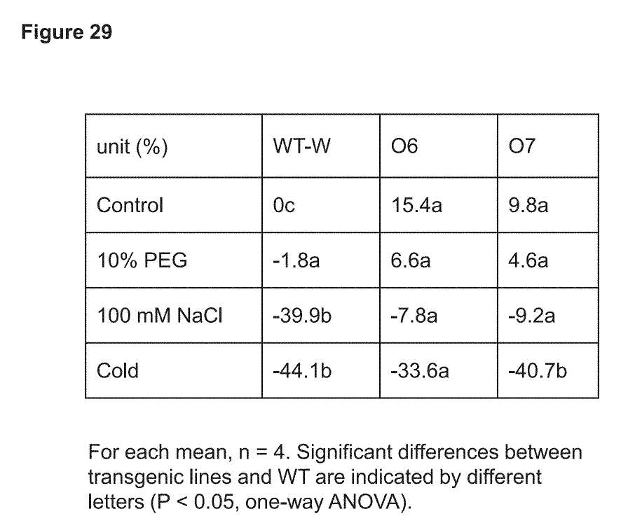

We analyzed grain yield and biomass of transgenic lines in the T0 and T1 generations. Relative to the wild-type (WT) plants, the biomass, including the grain yield, of the 12 pUbi:OsNRT2.1 lines increased by approximately 21.8% (FIG. 16e) and 20.9% (FIG. 17a) in T0 and T1 plants, respectively, but the grain yield decreased approximately 18.4% (FIG. 16c) and 16.6% (FIG. 17a) in T0 and T1 plants, respectively. Relative to the WT, the biomass, including the grain yield, of the 11 pOsNAR2.1:OsNRT2.1 lines increased by average values of 32.2% (FIG. 16f) and 27.1% (FIG. 17b) in T0 and T1 plants, respectively, and the grain yield increased by average values of 30.7% (FIG. 16d) and 28.1% (FIG. 17b) in T0 and T1 plants, respectively. Based on Southern blot analysis of T1 plants (FIG. 18) and RNA expression data for the T0 generation (FIG. 16a, b), we selected three independent pUbi:OsNRT2.1 T1 lines OE1-2, OE2-5, and OE3-4 (renamed as OE1, OE2, and OE3 (FIG. 1a)) and three independent pOsNAR2.1:OsNRT2.1 T1 lines O6-4, O7-6, and O8-3 (renamed as O6, O7, and O8 (FIG. 1b)).

Agricultural traits of these 6 lines were investigated in the field in the T1 through T4 generations, with particular focus on the T3 generation. OsNRT2.1 expression in roots was enhanced 4- to 7-fold in the OE1, OE2, and OE3 lines but only 2.5- to 3-fold in the O6, O7, and O8 lines relative to the WT. In culms, OsNRT2.1 expression was increased approximately six fold in the OE lines and approximately three fold in the O lines. In leaf blades, however, only the OE lines exhibited increased OsNRT2.1 expression (4 to 7-fold) compared with the WT, and no change in expression was observed in the O lines (FIG. 1c, d). The field data showed that both the OE and O lines exhibited increased growth and biomass but only the O lines produced higher yields than the WT (FIG. 1e, f).

Based on the agricultural traits of the T1-T4 generation plants in the field, the total aboveground biomass including grain yield increased by 21% for the pUbi:OsNRT2.1 lines and by 38% for the pOsNAR2.1:OsNRT2.1 lines, while the biomass without grain yield increased by 190% for the pUbi:OsNRT2.1 lines and by 160% for the pOsNAR2.1:OsNRT2.1 lines. The grain yields of the pUbi:OsNRT2.1 lines decreased over the three successive generations (FIG. 6), but the yields of the pOsNAR2.1:OsNRT2.1 lines increased significantly from the T1 to T3 generation (FIG. 6). The yields of the O lines were enhanced by approximately 33% in T1 plants grown at Ledong and by 34-42% in the T2 and T3 generations grown at Nanjing relative to the WT, while the OE lines exhibited lower yields than the WT by approximately 17% in all three generations (FIG. 6). We also analyzed the yield and the biomass of WT and T4 generation transgenic plants at Nanjing under low (180 kg N/ha) and normal N (300 kg N/ha) supplies. At the level of 180 kg N/ha, compared with WT, the yield of OE lines was reduced by 17%, and the biomass increased by 14%, while the yield and biomass of O lines was increased by 25% and 27% (FIG. 19a). At the level of 300 kg N/ha, the yield of OE lines was reduced by 16%, and the biomass increased by 12%, as for O lines the yield and biomass was increased by 21%, and 22% compared with WT (FIG. 19b).

The total tiller number per plant in the T3 generation at the harvest stage increased 27.1% on average for both pOsNAR2.1:OsNRT2.1 and pUbi:OsNRT2.1 transgenic plants relative to the WT with no difference between the transgenic lines (FIG. 7); however, the grain number per panicle differed significantly between the OE and O lines (FIG. 7). The grain number per panicle increased approximately 15% in the O lines, respectively; the panicle length increased in the O lines approximately 12%; and the Seed setting rate increased in the O lines by 14% relative to the WT (FIG. 7). The grain yields of the O lines increased by 24.2% relative to the WT (FIG. 7).

1.2.2. NUE of Transgenic Lines

Because biomass and yields increased in the pOsNAR2.1:OsNRT2.1 transgenic plants, we also analyzed ANUE in T1-T4 generations of transgenic plants, N recovery efficiency (NRE), physiological N use efficiency (PNUE), and N harvest index (NHI) traits at the harvest stage in T3 generation transgenic lines to determine whether N-use was altered in these plants, as modified the calculation method of the reference in Zhang et al. (2009). The ANUE of the O lines were enhanced by approximately 33% in T1 plants grown at Ledong and by 34-42% in the T2 and T3 generations grown at Nanjing relative to the WT, while the OE lines exhibited lower ANUE than the WT by approximately 17% in all three generations (FIG. 6). In T4 plants at Nanjing, at the level of 180 kg N/ha, compared with WT, the ANUE of OE lines was reduced by 22%, and the ANUE of O lines was increased by 33%, at the level of 300 kg N/ha, the ANUE of OE lines was reduced by 17%, and the ANUE of O lines was increased by 28% (FIG. 19c). In the OE lines, the NRE increased to approximately 115% of the WT; and the PNUE and NHI were reduced to approximately 71% of WT values. In the O lines, the ANUE increased to approximately 128% of the WT; the NRE increased to approximately 136% of the WT; and the PNUE and NHI were not significantly different from WT values (FIG. 9).

We sampled shoot tissues at the anthesis stage (60 days after transplanting) and the mature stage (90 days after transplanting) to determine the total N content. At the anthesis stage, total N was concentrated mainly in the culm with no difference between the OE and O lines, but with an increase of approximately 27% relative to WT. In leaves, the total N content was the same in the O and WT lines, but was approximately 33% higher in the OE lines. The total N content in the grain was the same in all lines (FIG. 2a). At the mature stage, total N was concentrated mainly in the grain, with the N content decreased by approximately 10% in the OE lines and increased by approximately 38% in the O lines relative to the WT (FIG. 2b).

1.2.3. Translocation of Dry Matter and N in Transgenic Lines

We investigated dry matter and N translocation in rice plants by determining dry matter at anthesis (DMA), dry matter at maturity (DMM), total N accumulation at anthesis (TNAA), and total N accumulation at maturity (TNAM). For the OE lines, the DMA, the DMM, the TNAA, and the TNAM increased by approximately 27%, 21%, 25%, and 21%, respectively, relative to the WT. For the O lines, the DMA, the SDMM, the TNAA, and the TNAM increased by approximately 46%, 38%, 15%, and 27%, respectively, relative to the WT (FIG. 8).

We also investigated the dry matter translocation (DMT), the DMT efficiency (DMTE), the contribution of pre-anthesis assimilates to grain yield (CPAY), and the harvest index (HI), based on the calculation method of the reference in Ntanos et al. (2002). For the OE lines, the DMT, DMTE, CPAY, and HI decreased by approximately 68%, 75%, 61%, and 31%, respectively, relative to the WT. For the O lines, the DMT increased by approximately 46%, while the DMTE, CPAY, and HI did not differ between the O lines and the WT (FIG. 9).

We investigated post-anthesis N uptake (PANU), N translocation (NT), NT efficiency (NTE), and the contribution of pre-anthesis N to grain N accumulation (CPNGN), as modified the calculation method of the reference in Ntanos et al. (2002) and Zhang et al. (2009). The PANU and CPNGN did not differ between the OE lines and the WT, but the NT and the NTE decreased by approximately 16% and 32%, respectively, in the OE lines relative to the WT. The NTE did not differ between the O lines and the WT, while the PANU and NT increased by approximately 87% and 18%, respectively, and the CPNGN decreased by approximately 16% in the O lines relative to the WT (FIG. 9).

1.2.4. Expression Patterns of OsNRT2.1 and OsNAR2.1 in Different Organs of WT and Transgenic Lines

Rice was previously shown to have a two-component NO3− uptake system consisting of OsNRT2.1 and OsNAR2.1, similar to the system in Arabidopsis (Feng et al., 2011; Yan et al., 2011; Liu et al., 2014). We analyzed the OsNRT2.1 and OsNAR2.1 expression patterns in WT and transgenic lines during the filling stage. The detail about RNA samples were described in FIG. 20 and methods. The OsNRT2.1 expression pattern in WT showed that OsNRT2.1 gene expressed most in root, secondly in leaf sheaths, thirdly in leaf blades and inter nodes, and least in grain including seed, palea and lemma (FIG. 14, FIG. 3a). As for OsNAR2.1, it was expressed also most in root, secondly in leaf sheaths, thirdly in inter nodes and least in grain and leaf blades (FIG. 14, FIG. 3b). The co-expression pattern of OsNRT2.1 and OsNAR2.1 happened in root, leaf sheaths, inter nodes and grain but not in leaf blades (FIG. 14, FIG. 21).

Compared with WT, the OsNRT2.1 expression increased by about 7.5 fold averagely in all organs of OE lines including root. The increase pattern of OsNRT2.1 in OE lines showed a similar trend as the native expression of OsNRT2.1 in WT which was most in root, secondly in leaf sheaths, thirdly in leaf blades and inter nodes, and least in grain (FIG. 14, FIG. 3a). It was very interesting that we found in OE lines that the OsNAR2.1 was also increased with the highest expression firstly in roots, secondly in leaf sheaths, thirdly in inter nodes, fourthly in leaf blades and least in grain (FIG. 14, FIG. 3b). The co-expression increase pattern of OsNRT2.1 and OsNAR2.1 occurred in all organs of OE lines (FIG. 14, FIG. 21).

Compared with WT, the OsNRT2.1 expression was not changed in grain and leaf blades in O lines and increased in leaf sheaths, inter nodes and root significantly with the same pattern as WT, which was most in root, secondly in leaf sheaths, thirdly in inter nodes, fourthly in leaf blades and least in grain (FIG. 14, FIG. 3a). For OsNAR2.1 expression in O lines, it was also not increased in grain and leaf blades but only significantly increased in leaf sheaths, inter nodes and root with the same pattern as WT, which was most in root, secondly in leaf sheaths, thirdly in inter nodes and least in grain and leaf blades (FIG. 14, FIG. 3b). The co-expression increase pattern of OsNRT2.1 and OsNAR2.1 occurred in leaf sheaths, inter nodes and root of O lines (FIG. 14, FIG. 21).

1.2.5. Expression Patterns of OsNRT2.1 and OsNAR2.1 in Different Growth Stages of WT and Transgenic Lines

In this study, we found that the OsNRT2.1 and OsNAR2.1 mRNA levels in the culms including the leaf sheath and inter node (FIG. 22) were significantly higher in all of the transgenic plants than in the WT plants (FIG. 4a, b). OsNRT2.1 expression was 3-20-fold higher in the OE lines than in WT, but was only 31-45% higher in the O lines than in WT (FIG. 4a). OsNAR2.1 expression was two to nine-fold higher in the OE lines than in WT and was one- to eight-fold higher in the O lines than in the WT (FIG. 4b). Throughout the experimental growth period, OsNRT2.1 expression was significantly higher in the culms of the OE lines than the O lines, but no significant difference in OsNAR2.1 expression was observed between the OE and O transgenic lines.

During the entire experimental growth period, no significant differences in OsNRT2.1 and OsNAR2.1 expression were found between the leaf blade I of the O lines and WT plants, but the expression levels of both OsNRT2.1 and OsNAR2.1 were upregulated significantly in the OE plants relative to the WT (FIG. 23).

1.2.6. Growth Rate in Transgenic Lines

N transport and the growth of rice biomass are closely related and OsNRT2.1 overexpression was previously shown to affect rice growth (Katayama et al., 2009). In this study, the OE and O lines began to show significantly higher biomass than WT plants at 45 days after transplanting and had accumulated 21% and 38% more biomass at 90 days (FIG. 4c). The growth rates of the OE and O lines reached peak values at 60 days and were higher than those of the WT plants (FIG. 4d). The growth rates of the OE and O lines were approximately 25% and 58% greater, respectively, than the WT. The growth rates of the transgenic and WT plants were identical after 75 days during the grain filling stage (FIG. 4d).

1.2.7. The Co-Expression of OsNRT2.1 and OsNAR2.1 in WT and Transgenic Plants

The expression pattern of OsNRT2.1 and OsNAR2.1 in different organs showed that there existed a strong co-expression pattern of these two genes in rice plants (FIG. 21). The co-expression pattern of OsNRT2.1 and OsNAR2.1 was altered very much in OE lines compared with O and WT lines (FIG. 21). The expression ratio of OsNRT2.1 to OsNAR2.1 5.4:1 in the OE organs was 3.6:1 in the O lines compared with 3.9:1 in WT organs (FIG. 21). Furthermore we specially investigated the ratio of OsNRT2.1 to OsNAR2.1 expression in root as 6.3:1 in the OE lines, 4.1:1 in the O lines, and 4.2:1 in WT plants, with no significant differences between the 0 lines and WT plants (FIG. 14).

The culm is important for N storage and translocation in rice shoots. In rice shoot, OsNRT2.1 and OsNAR2.1 expression was expressed most in leaf sheaths of culm (FIG. 3). Our expression data also confirmed that OsNRT2.1 and OsNAR2.1 expression in the culm could play a key role in NO3− remobilization. To further study the possible relationship between OsNRT2.1 and OsNAR2.1 expression and rice growth, we compared the ratio of OsNRT2.1 and OsNAR2.1 expression in rice plants. The expression ratio was approximately 11.3:1 in the OE lines and approximately 4.7:1 in the O lines compared with approximately 7.2:1 in WT plants (FIG. 5). We also investigated the ratio of OsNRT2.1 to OsNAR2.1 expression in leaf blade I. The expression ratio was 7.3:1 in the OE lines, 4:1 in the O lines, and 5.2:1 in WT plants, with no significant differences between the O lines and WT plants (FIG. 24). The ratio of OsNRT2.1 to OsNAR2.1 expression correlated with the grain yield.

1.2.8. The Response of pOsNAR2.1:OsNRT2.1 Lines to Stress

The effect of different stress conditions on the growth of rice seedlings of WT and transgenic plants (2 pOsNAR2.1:OsNRT2.1 cell lines, O6 and O7) was assessed (FIG. 27). Both O6 and 07 seedlings grew significantly more than WT seedlings, as determined by fresh weight (FIG. 28A, 29). This effect was maintained in high salt (100 mM NaCl) conditions for both O6 and O7, and cold conditions for O6.

The root system plays an important role in plant growth and resistance to stress and the root/shoot ratio reflects the root and shoot biomass accumulation relationship for a plant. In control conditions, the transgenic plants had significantly larger root systems relative to the control plants (FIG. 28B). This effect was maintained in stress conditions (FIG. 28B), indicating that the transgenic plants will produce a better crop yield compared to the control plants in both stress and non-stress conditions.

1.3. Discussion

N nutrition affects all levels of plant function, from metabolism to resource allocation, growth, and development (Crawford, 1995; Scheible et al., 1997; Stitt, 1999; Scheible et al., 2004). As one form of available N nutrient to plants, NO3− is taken up in the roots by active transport processes and stored in vacuoles in rice shoots (Fan et al., 2007; Li et al., 2008). In rice, OsNAR2.1 acts as a partner protein with OsNRT2.1 in the uptake and transport of NO3− (Yan et al., 2011; Tang et al., 2012; Liu et al., 2014). OsNAR2.1 gene expression was shown to be upregulated by NO3− and downregulated by NH4+ (Zhuo et al., 1999; Nazoa et al., 2003; Feng et al., 2011).

Rooke et al. (2000) reported that the maize Ubi-1 promoter had strong activity in young, metabolically active tissues and in pollen grains. Furthermore, Cornejo et al. (1993) performed histochemical localization of Ubi-GUS activity and showed that the Ubi promoter was most active in rapidly dividing cells; however, Chen et al. (2012) reported that the Ubi promoter drove strong OsPIN2 expression in all tissues. Chen et al. (2015) reported that ectopic expression of the WOX11 gene driven by the promoter of the OsHAK16 gene, which encodes a potassium (K) transporter that is induced by low K levels, led to an extensive root system, adventitious roots, and increased tiller numbers in rice. In contrast, WOX11 overexpression driven by the Ubi promoter induced ectopic crown roots in rice and failed to present any similar super growth phenotype in field (Zhao et al., 2009) as described by Chen et al. (2015). These results suggested that the use of a specific inducible promoter driving gene function could be a good strategy for plant breeding.

In this study, OsNRT2.1 expression was upregulated significantly in both the aboveground and underground parts of pUbi:OsNRT2.1 transgenic plants relative to WT (FIG. 1c), while OsNRT2.1 expression in pOsNAR2.1:OsNRT2.1 transgenic plants was increased significantly only in the roots and culms and not enhanced significantly in the leaves (FIG. 1d). Specific induction of expression by the OsNAR2.1 promoter in rice roots and culms based on GUS fusion data has been reported previously (Feng et al., 2012); therefore, we investigated the effects of tissue-specific induction of OsNRT2.1 expression in roots and culms on plant growth and NUE.

1.3.1. Effect of pOsNAR2.1:OsNRT2.1 Expression on NUE in Transgenic Rice

N redistribution during the reproductive stage was shown to vary significantly among cultivars and under various N management strategies (Souza et al., 1998). Mae and Ohira (1981) reported that a major proportion of N was redistributed from vegetative organs to panicles during grain filling, 64% of which was derived from leaf blades and 36% from culms. The NTE values of WT, pUbi:OsNRT2.1, and pOsNAR2.1:OsNRT2.1 plants were averagely 49.5%, 33.4%, and 50.3%, indicating that N transfer from the shoots into grain was significantly less in pUbi:OsNRT2.1 transgenic plants than in WT or pOsNAR2.1:OsNRT2.1 plants (FIG. 9). This lower level of N transfer from vegetative organs to grain during grain filling in pUbi:OsNRT2.1 plants affected spike formation and final grain yield compared with the WT and pOsNAR2.1:OsNRT2.1 plants (FIG. 6). The DMTE values for WT, pUbi:OsNRT2.1, and pOsNAR2.1:OsNRT2.1 plants were 22.1%, 5.5%, and 22.1%, averagely, (FIG. 9) demonstrating that markedly less dry matter was transferred into grain yield in the pUbi:OsNRT2.1 lines. These data confirmed that the transport of N and biomass during the transition from the flowering to harvest stages affected the final yield and NUE of rice (Zhang et al., 2009) and also indicated that the Ubi promoter decreased N and biomass translocation, while the OsNAR2.1 promoter did not.

In both types of OsNRT2.1 overexpression line, NT was reduced during the reproductive stage and NUE was reduced before flowering. The CPAY average values of the WT, pUbi:OsNRT2.1, and pOsNAR2.1:OsNRT2.1 plants were 28.5%, 11%, and 34.9%, respectively. The CPAY of the pOsNAR2.1:OsNRT2.1 plants was higher than WT plants that had higher CPAY than the pUbi:OsNRT2.1 plants (FIG. 9). The HI was much lower for the pUbi:OsNRT2.1 plants than for the WT or pOsNAR2.1:OsNRT2.1 plants (FIG. 9) indicating that the Ubi promoter affected NO3− uptake and N-use before the flowering stage and that levels of OsNRT2.1 overexpression in rice that were excessive did not benefit N-use during either the vegetative or reproductive stages.

1.3.2. The Co-Expression Pattern of OsNRT2.1 and OsNAR2.1 is an Important Factor Controlling N Transport in Rice

How to assess the effect of NO3− transporter expression on rice NUE is a key question for rice breeding. The NO3− transporter, OsNRT1.1B, was shown to improve the NUE of rice by approximately 30% (Hu et al., 2015), while our data showed that the higher expression level of the NO3− transporter was not relative to the higher yield and NUE of rice (Tables 1 and 4, & FIG. 4). After determining the expression levels of OsNRT2.1 and its partner gene, OsNAR2.1, we calculated the co-expression ratio of these genes in rice plants.

The co-expression pattern of OsNRT2.1 and OsNAR2.1 happened in WT and transgenic plants (FIG. 3, FIG. 4, and FIG. 14). However, the co-expression pattern of OsNRT2.1 and OsNAR2.1 was changed in OE lines compared with O and WT lines (FIG. 21), which suggested that a different promoter driving OsNRT2.1 had a different co-expression pattern with OsNAR2.1. But it is still not clear why increasing OsNRT2.1 expression would induce OsNAR2.1 expression and what mechanism exists behind the co-expression pattern of OsNRT2.1 and OsNAR2.1 in gene regulation.

However, the ratio changes of OsNRT2.1 to OsNAR2.1 expression may be a clue for explanation of rice growth and nitrogen use difference in WT and transgenic lines. The ratio changes of OsNRT2.1 to OsNAR2.1 expression in different organs was increased significantly in pUbi:OsNRT2.1 lines compared with WT and pOsNAR2.1:OsNRT2.1 lines (FIG. 21). Also during the growth stages, the ratio of OsNRT2.1 to OsNAR2.1 expression in culm (including the internode and leaf sheath) was increased in pUbi:OsNRT2.1 lines compared with WT and the pOsNAR2.1:OsNRT2.1 lines (FIG. 5). These data indicated that the interaction between OsNRT2.1 and OsNAR2.1 in pUbi:OsNRT2.1 plants differed from WT and that in the pOsNAR2.1:OsNRT2.1 lines. Furthermore, in culms pOsNAR2.1:OsNRT2.1 lines showed a lower expression ratio of these two genes, in which more OsNAR2.1 protein may be available to interact with OsNRT2.1 protein. Therefore, the efficiency of OsNRT2.1 function in rice plants should differ between the two types of transgenic plants resulting in different rice yield and NUE phenotypes. On the other hand, the high expression of OsNRT2.1 and OsNAR2.1 in all the organs of pUbi:OsNRT2.1 may result in other disadvantages for the plants, such as a high cost for mRNA synthesis. Alternatively, such high expression levels may disturb nitrogen transport in the leaf blades. All possibilities remain to be confirmed by further analysis.

In this study, we showed that rice yield and NUE could be improved by increase OsNRT2.1 expression, especially in combination with a lower expression ratio with its partner gene OsNAR2.1, which encodes a high-affinity NO3− transporter.

### REFERENCES

Almagro A, Lin S H, Tsay Y F (2008) Characterization of the Arabidopsis nitrate transporter NRT1.6 reveals a role of nitrate in early embryo development. Plant Cell 20: 3289-3299 Cermak, T., Doyle E L, Christian M, Wang L, Zhang Y, Schmidt C, Bailer J A, Somia N V,
Bogdanove A J, Voytas D F (2011) Efficient design and assembly of custom TALEN and other TAL effector-based constructs for DNA targeting. Nucleic Acids Res. 2011 Sep. 1; 39(17):7879.
Chen, G., Feng, H., Hu, Q., Qu, H., Chen, A., Yu, L. and Xu, G. (2015) Improving rice tolerance to potassium deficiency by enhancing OsHAK16p:WOX11-controlled root development. Plant Biotechnol. J. 13, 833-848.
Chen, Y., Fan, X., Song, W., Zhang, Y. and Xu, G. (2012) Over-expression of OsPIN2 leads to increased tiller numbers, angle and shorter plant height through suppression of OsLAZY1. Plant Biotechnol. J. 10, 139-149.
Chiu C C, Lin C S, Hsia A P, Su R C, Lin H L, Tsay Y F (2004) Mutation of a nitrate transporter, AtNRT1:4, results in a reduced petiole nitrate content and altered leaf development. Plant Cell Physiol 45: 1139-1148
Cornejo, M. J., Luth, D., Blankenship, K. M., Anderson, O. D., Blechl, A. E. (1993) Activity of a maize ubiquitin promoter in transgenic rice. Plant Mol. Biol. 23, 567-581.
Coruzzi, G., Bush, D. R. (2001) Nitrogen and carbon nutrient and metabolite signaling in plants. Plant Physiol. 125, 61-64.
Coruzzi, G. M., Zhou, L. (2001) Carbon and nitrogen sensing and signaling in plants: emerging ‘matrix effects’. Curr. Opin. Plant Biol, 4, 247-253.
Crawford, N. M. (1995) Nitrate: nutrient and signal for plant growth. Plant Cell. 7, 859-868.
Crawford, N. M., Forde, B. G. (2002) Molecular and developmental biology of inorganic nitrogen nutrition. In The Arabidopsis Book. Edited by Meyerowitz E M. Rockville, Md., American Society of Plant Biologists.
Crawford, N. M., Glass, A. D. M. (1998) Molecular and physiological aspects of nitrate uptake in plants. Trends Plant Sci. 3, 389-395.
Fan, X., Jia, L., Li, Y., Smith, S. J., Miller, A. J., Shen, Q. (2007) Comparing nitrate storage and remobilization in two rice cultivars that differ in their nitrogen use efficiency, J. Exp. Bot. 58, 1729-1740.
Fan, X., Shen, Q., Ma, Z., Zhu, H., Yin, X., Miller, A. J. (2005) A comparison of nitrate transport in four different rice (Oryza sativa L.) cultivars, Sci China C Life Sci. 48, 897-911.
Fan S C, Lin C S, Hsu P K, Lin S H, Tsay Y F (2009) The Arabidopsis nitrate transporter NRT1.7, expressed in phloem, is responsible for source-tosink remobilization of nitrate. Plant Cell 21: 2750-2761
Feng, H., Yan, M., Fan, X., Li, B., Shen, Q., Miller, A. J., Xu, G. (2011) Spatial expression and regulation of rice high-affinity nitrate transporters by nitrogen and carbon status. J. Exp. Bot. 62, 2319-2332.
Feng, Q., Zhang, Y., Hao, P., et al. (2002) Sequence and analysis of rice chromosome 4. Nature. 420, 316-320.
Gojon A, Krouk G, Perrine-Walker F, Laugier E (2011) Nitrate transceptor(s) in plants. J Exp Bot 62: 2299-2308
Hu, B., Wang, W., Ou, S., Tang, J., Li, H., Che, R., Zhang, Z., Chai, X., Wang, H., Wang, Y., Liang, C., Liu, L., Piao, Z., Deng, Q., Deng, K., Xu, C., Liang, Y., Zhang, L., Li, L., Chu, C. (2015) Variation in NRT1.1B contributes to nitrate-use divergence between rice subspecies. Nat. Genet. 47, 834-838.
Huang N C, Liu K H, Lo H J, Tsay Y F (1999) Cloning and functional characterization of an Arabidopsis nitrate transporter gene that encodes a constitutive component of low-affinity uptake. Plant Cell 11: 1381-1392
Jia, H., Ren, H., Gu, M., Zhao, J., Sun, S., Zhang, X., Chen, J., Wu, P., Xu, G. (2011) The phosphate transporter gene OsPht1; 8 is involved in phosphate homeostasis in rice. Plant Physiol. 156, 1164-1175.
Katayama, H., Mori, M., Kawamura, Y., Tanaka, T., Mori, M., Hasegawa, H. (2009) Production and characterization of transgenic rice plants carrying a high-affinity nitrate transporter gene (OsNRT2.1). Breeding Sci. 59, 237-243.
Khuram, M., Asif, I., Muhammad, H., Faisal, Z., Siddiqui, M. H., Mohsin, A. U., Bakht, H. F. S. G., Hanif, M. (2013) Impact of nitrogen and phosphorus on the growth, yield and quality of maize (Zea mays L.) fodder in Pakistan. Philipp J Crop Sci. 38, 43-46.
Kirk, G. J. D., Kronzucker, H. J. (2005) The potential for nitrification and nitrate uptake in the rhizosphere of wetland plants: a modelling study. Ann Bot. 96, 639-646.
Kronzucker, H. J., Glass, A. D. M., Siddiqi, M. Y., Kirk, G. J. D. (2000) Comparative kinetic analysis of ammonium and nitrate acquisition by tropical lowland rice: implications for rice cultivation and yield potential. New Phytol. 145, 471-476.
Li, B., Xin, W., Sun, S., Shen, Q. and Xu, G. (2006) Physiological and molecular responses of nitrogen-starved rice plants to re-supply of different nitrogen sources. Plant Soil. 287, 145-159.
Li, Y. L., Fan, X. R., Shen, Q. R. (2008) The relationship between rhizosphere nitrification and nitrogen-use efficiency in rice plants. Plant Cell Environ. 31, 73-85.
Li J Y, Fu Y L, Pike S M, Bao J, Tian W, Zhang Y, Chen C Z, Zhang Y, Li H M, Huang J, et al (2010) The Arabidopsis nitrate transporter NRT1.8 functions in nitrate removal from the xylem sap and mediates cadmium tolerance. Plant Cell 22: 1633-1646
Li, Y., Gu, M., Zhang, X., Zhang, J., Fan, H., Li, P., Li, Z., Xu, G. (2014) Engineering a sensitive visual tracking reporter system for real-time monitoring phosphorus deficiency in tobacco. Plant Biotechnol. J. 12, 674-684.
Lin S H, Kuo H F, Canivenc G, Lin C S, Lepetit M, Hsu P K, Tillard P, Lin H L, Wang Y Y, Tsai C B, et al (2008) Mutation of the Arabidopsis NRT1.5 nitrate transporter causes defective root-to-shoot nitrate transport. Plant Cell 20: 2514-2528
Liu K H, Tsay Y F (2003) Switching between the two action modes of the dual-affinity nitrate transporter CHL1 by phosphorylation. EMBO J 22: 1005-1013
Miller A J, Fan X, Orsel M, Smith S J, Wells D M (2007) Nitrate transport and signalling. J Exp Bot 58: 2297-2306
Liu, X., Huang, D., Tao, J., Miller, A. J., Fan, X., Xu, G. (2014) Identification and functional assay of the interaction motifs in the partner protein OsNAR2.1 of the two-component system for high-affinity nitrate transport. New Phytol. 204, 74-80.
Mae, T., Ohira, K. (1981) The remobilization of nitrogen related to leaf growth and senescence in rice plants (Oryza sativa L.). Plant Cell Physiol. 22, 1067-1074.
Miller A J, Fan X, Orsel M, Smith S J, Wells D M (2007) Nitrate transport and signalling. J Exp Bot 58: 2297-2306
Nazoa, P., Vidmar, J. J., Tranbarger, T. J., Mouline, K., Damiani, I., Tillard, P., Zhuo, D., Glass, A. D., Touraine, B. (2003) Regulation of the nitrate transporter gene AtNRT2.1 in Arabidopsis thaliana: responses to nitrate, amino acids, and developmental stage. Plant Mol. Biol. 52, 689-703.
Ntanos, D. A., Koutroubas, S. D. (2002) Dry matter and N accumulation and translocation for Indica and Japonica rice under Mediterranean conditions. Field Crop. Res. 74, 93-101.
Okamoto, M., Kumar, A., Li, W., Wang, Y., Siddiqi, M. Y., Crawford, N. M., Glass, A. D. (2006) High-affinity nitrate transport in roots of Arabidopsis depends on expression of the NAR2-like gene AtNRT3.1. Plant Physiol. 140, 1036-1046.
Ookawa, T., Hobo, T., Yano, M., Murata, K., Ando, T., Miura, H., Asano, K., Ochiai, Y., Ikeda, M., Nishitani, R., Ebitani, T., Ozaki, H., Angeles, E. R., Hirasawa, T., Matsuoka, M. (2010) New approach for rice improvement using a pleiotropic QTL gene for lodging resistance and yield. Nat Commun. 1, 132.
Orsel, M., Chopin, F., Leleu, O., Smith, S. J., Krapp, A., Daniel-Vedele, F., Miller, A. J. (2006) Characterization of a two-component high-affinity nitrate uptake system in Arabidopsis. Physiology and protein-protein interaction. Plant Physiol. 142, 1304-1317.
Pan, S., Rasul, F., Li, W., Tian, H., Mo, Z., Duan, M., Tang, X. (2013) Roles of plant growth regulators on yield, grain qualities and antioxidant enzyme activities in super hybrid rice (Oryza sativa L.). Rice. 6, 9.
Quesada, A., Galvan, A., Fernandez, E. (1994) Identification of nitrate transporter genes in Chlamydomonas reinhardtii. Plant J. 5, 407-419.
Rooke, L., Byrne, D., Salgueiro, S. (2000) Marker gene expression driven by the maize ubiquitin promoter in transgenic wheat. Ann Appl Biol. 136, 167-172.
Sasakawa, H., Yamamoto, Y. (1978) Comparison of the uptake of nitrate and ammonium by rice seedlings. Plant Physiol. 62, 665-669.
Sasaki, T., Matsumoto, T., Yamamoto, K., et al. (2002) The genome sequence and structure of rice chromosome 1. Nature. 420, 312-316.
Scheible, W. R., Gonzalez-Fontes, A., Lauerer, M., Muller-Rober, B., Caboche, M., Stitt, M. (1997) Nitrate acts as a signal to induce organic acid metabolism and repress starch metabolism in tobacco. Plant Cell. 9, 783-798.
Scheible, W. R., Morcuende, R., Czechowski, T., Fritz, C., Osuna, D., Palacios-Rojas, N., Schindelasch, D., Thimm, O., Udvardi, M. K. and Stitt, M. (2004) Genome-wide reprogramming of primary and secondary metabolism, protein synthesis, cellular growth processes, and the regulatory infrastructure of Arabidopsis in response to nitrogen. Plant Physiol. 136, 2483-2499.
Souza, S. R., Stark, E. M. L. M. and Fernandes, M. S. (1998) Nitrogen remobilization during the reproductive period in two Brazilian rice varieties. J. Plant Nutr. 21, 2049-2063.
Srikanth, B., Subhakara, R. I., Surekha, K., Subrahmanyam, D., Voleti, S. R., Neeraja, C. N. (2015) Enhanced expression of OsSPL14 gene and its association with yield components in rice (Oryza sativa) under low nitrogen conditions. Gene. doi: 10.1016/j.gene.2015.10.062.
Stitt, M. (1999) Nitrate regulation of metabolism and growth. Curr. Opin. Plant Biol. 2, 178-186. Tang, Z., Fan, X., Li, Q., Feng, H., Miller, A. J., Shen, Q., Xu, G. (2012) Knockdown of a rice stelar nitrate transporter alters long-distance translocation but not root influx. Plant Physiol. 160, 2052-2063.
Tsay Y F, Chiu C C, Tsai C B, Ho C H, Hsu P K (2007) Nitrate transporters and peptide transporters. FEBS Lett 581: 2290-2300
Wang, R., Guegler, K., LaBrie, S. T., Crawford, N. M. (2000) Genomic analysis of a nutrient response in Arabidopsis reveals diverse expression patterns and novel metabolic and potential regulatory genes induced by nitrate. Plant Cell. 12, 1491-1509.
Wang Y Y, Tsay Y F (2011) Arabidopsis nitrate transporter NRT1.9 is important in phloem nitrate transport. Plant Cell 23: 1945-1957
Xu, G., Fan, X., Miller, A. J. (2012) Plant nitrogen assimilation and use efficiency. Annu. Rev. Plant Biol. 63, 153-182.
Yan, M., Fan, X., Feng, H., Miller, A. J., Sheng, Q., Xu, G. (2011) Rice OsNAR2.1 interacts with OsNRT2.1, OsNRT2.2 and OsNRT2.3a nitrate transporters to provide uptake over high and low concentration ranges. Plant Cell Environ. 34, 1360-1372.
Yong, Z., Kotur, Z., Glass, A. D. (2010) Characterization of an intact two-component high-affinity nitrate transporter from Arabidopsis roots. Plant J. 63, 739-748.
Zhang, H., Forde, B. G. (2000) Regulation of Arabidopsis root development by nitrate availability. J. Exp. Bot. 51, 51-59.
Zhang, Y. L., Fan, J. B., Wang D. S., Shen Q. R. (2009) Genotypic Differences in Grain Yield and Physiological Nitrogen Use Efficiency Among Rice Cultivars. Pedosphere. 19, 681-691.
Zhao, Y., Hu, Y., Dai, M., Huang, L., Zhou, D. X. (2009) The WUSCHEL-related homeobox gene WOX11 is required to activate shoot-borne crown root development in rice. Plant Cell. 21, 736-748.
Zhuo, D., Okamoto, M., Vidmar, J. J., Glass, A. D. (1999) Regulation of a putative high-affinity nitrate transporter (Nrt2;1At) in roots of Arabidopsis thaliana. Plant J. 17, 563-568.
Zhou J J, Fernandez E, Galván A, Miller A J (2000) A high affinity nitrate transport system from Chlamydomonas requires two gene products. FEBS Lett 466: 225-227

### SEQUENCE INFORMATION

Oryza Sativa

| SEQ ID NO: 1: OsNRT2.1 AB008519 mRNA, complete cds | |
|---|---|
| 1 | aattgcatcc gagctcacct agcttctctc ttgcaaccag cgattcgatc gattccatct |
| 61 | ccaagaagca gcggctagca gcagctagta gttgccatgg actcgtcgac ggtgggcgct |
| 121 | ccggggagct cgctgcacgg cgtgacgggg cgcgagccgg cgttcgcgtt ctcgacggag |
| 181 | gtgggcggcg aggacgcggc ggcggcgagc aagttcgact tgccggtgga ctcggagcac |
| 241 | aaggcgaaga cgatcaggtt gctgtcgttc gcgaacccgc atatgaggac gttccaccta |
| 301 | tcatggatct ccttcttctc ctgcttcgtc tccaccttcg ccgccgcccc tctcgtcccc |
| 361 | atcatccgcg acaacctcaa cctcaccaag gccgacatcg gcaacgccgg cgtcgcctcc |
| 421 | gtctccggct ccatcttctc caggctcgcc atgggcgcca tctgcgacat gctcggcccg |
| 481 | cgctacggct gcgccttcct catcatgctc gccgcgccca ccgtcttctg catgtcgctc |
| 541 | atcgactccg ccgcggggta catcgccgtg cgcttcctca tcggcttctc cctcgccacc |
| 601 | ttcgtgtcat gccagtactg gatgagcacc atgttcaaca gcaagatcat cggcctcgtc |
| 661 | aacggcctcg ccgccgggtg gggaaacatg ggcggcggcg cgacgcagct catcatgccg |
| 721 | ctcgtctacg acgtgatccg caagtgcggc gcgacgccgt tcacggcgtg gaggctggcc |
| 781 | tacttcgtgc cggggacgct gcacgtggtg atgggcgtgc tggtgctgac gctggggcag |
| 841 | gacctccccg acggcaacct gcgcagcctg cagaagaagg gtgacgtcaa cagggacagc |
| 901 | ttctccaggg tgctctggta cgccgtcacc aactaccgca cctggatctt cgtcctcctc |
| 961 | tacggctact ccatgggcgt cgagctcacc accgacaacg tcatcgccga gtacttctac |
| 1021 | gatcgcttcg acctcgacct ccgcgtcgcc ggcatcatcg ccgcatcctt cggcatggcc |
| 1081 | aacatcgtcg cgcgccccac cggcggcctc ctctcggacc tcggcgcgcg ctacttcggc |
| 1141 | atgcgcgccc gcctctggaa catttggatc ctccagaccg ccggcggcgc gttctgcctc |
| 1201 | ctgctcggcc gcgcatccac cctccccacc tccgtcgtct gcatggtcct cttctccttc |
| 1261 | tgcgcgcagg ccgcctgcgg cgccatcttc ggcgtcatcc ccttcgtctc ccgccgctcg |
| 1321 | ctcggcatca tctccggcat gaccggcgcc ggcggcaact tcggcgccgg gctcacgcag |
| 1381 | ctgctcttct tcacgtcgtc gaggtactcc acgggcacgg ggctggagta catgggcatc |
| 1441 | atgatcatgg cgtgcacgct gccggtggtg ctcgtccatt tcccgcagtg gggctccatg |
| 1501 | ttcctcccgc ccaacgccgg cgccgaggag gagcactact acggctccga gtggagcgaa |
| 1561 | caggagaaga gcaagggcct ccacggtgca agtctcaagt tcgccgagaa ctcccgctcc |
| 1621 | gagcgtggcc gccgcaacgt catcaacgcc gccgccgccg ccgccacgcc gcccaacaac |
| 1681 | tcgccggagc acgcctaagg cgtaaacaat tctgcgaccg agaccagcaa tacgctggag |
| 1741 | ttcgactcga tgataacacg ccggagcacg tccttgttgc aaacggtgat gaaattaaga |
| 1801 | gtgaaatatt tccttaggaa ttgaatcctt tgaaattaat tcctttggaa tttctccgat |
| 1861 | acaaacggag gtataataag ggagaggcat ttatacctat gtacatgtta cacttttttg |
| 1921 | aaaaaaaa |
| SEQ ID NO: 2: OsNRT2.1 translation | |
| MDSSTVGAPGSSLHGVTGREPAFAFSTEVGGEDAAAASKFDLPV | |
| DSEHKAKTIRLLSFANPHMRTFHLSWISFFSCFVSTFAAAPLVPIIRDNLNLTKADIG | |
| NAGVASVSGSIFSRLAMGAICDMLGPRYGCAFLIMLAAPTVFCMSLIDSAAGYIAVRF | |
| LIGFSLATFVSCQYWMSTMFNSKIIGLVNGLAAGWGNMGGGATQLIMPLVYDVIRKCG | |
| ATPFTAWRLAYFVPGTLHVVMGVLVLTLGQDLPDGNLRSLQKKGDVNRDSFSRVLWYA | |
| VTNYRTWIFVLLYGYSMGVELTTDNVIAEYFYDRFDLDLRVAGIIAASFGMANIVARP | |
| TGGLLSDLGARYFGMRARLWNIWILQTAGGAFCLLLGRASTLPTSVVCMVLFSFCAQA | |
| ACGAIFGVIPFVSRRSLGIISGMTGAGGNFGAGLTQLLFFTSSRYSTGTGLEYMGIMI | |
| MACTLPVVLVHFPQWGSMFLPPNAGAEEEHYYGSEWSEQEKSKGLHGASLKFAENSRS | |
| ERGRRNVINAAAAAATPPNNSPEHA | |
| SEQ ID NO: 3: OSNRT2.2 AK109733 MRNA, COMPLETE CDS | |
| 1 | aaagcaacag cagcagaagt gcacttttgc attctatttc caatcaatcc aagaggctag |
| 61 | agctagcggc caagatcatc gtcaccggcg gcatggactc gtcgacggtg ggcgctccgg |
| 121 | ggagctcgct gcacggcgtg acggggcgcg agccggcgtt cgcgttctcg acggaggtgg |
| 181 | gcggcgagga cgcggcggcg gcgagcaagt tcgacttgcc ggtggactcg gagcacaagg |
| 241 | cgaagacgat caggttgctg tcgttcgcga acccgcatat gaggacgttc cacctatcat |
| 301 | ggatctcctt cttctcctgc ttcgtctcca ccttcgccgc cgcccctctc gtccccatca |
| 361 | tccgcgacaa cctcaacctc accaaggccg acatcggcaa cgccggcgtc gcctccgtct |
| 421 | ccggctccat cttctccagg ctcgccatgg gcgccatctg cgacatgctc ggcccgcgct |
| 481 | acggctgcgc cttcctcatc atgctcgccg cgcccaccgt cttctgcatg tcgctcatcg |
| 541 | actccgccgc ggggtacatc gccgtgcgct tcctcatcgg cttctccctc gccaccttcg |
| 601 | tgtcatgcca gtactggatg agcaccatgt tcaacagcaa gatcatcggc ctcgtcaacg |
| 661 | gcctcgccgc cgggtgggga aacatgggcg gcggcgcgac gcagctcatc atgccgctcg |
| 721 | tctacgacgt gatccgcaag tgcggcgcga cgccgttcac ggcgtggagg ctggcctact |
| 781 | tcgtgccggg gacgctgcac gtggtgatgg gcgtgctggt gctgacgctg gggcaggacc |
| 841 | tccccgacgg caacctgcgc agcctgcaga agaagggtga cgtcaacagg gacagcttct |
| 901 | ccagggtgct ctggtacgcc gtcaccaact accgcacctg gatcttcgtc ctcctctacg |
| 961 | gctactccat gggcgtcgag ctcaccaccg acaacgtcat cgccgagtac ttctacgatc |
| 1021 | gcttcgacct cgacctccgc gtcgccggca tcatcgccgc atccttcggc atggccaaca |
| 1081 | tcgtcgcgcg ccccaccggc ggcctcctct cggacctcgg cgcgcgctac ttcggcatgc |
| 1141 | gcgcccgcct ctggaacatt tggatcctcc agaccgccgg cggcgcgttc tgcctcctgc |
| 1201 | tcggccgcgc atccaccctc cccacctccg tcgtctgcat ggtcctcttc tccttctgcg |
| 1261 | cgcaggccgc ctgcggcgcc atcttcggcg tcatcccctt cgtctcccgc cgctcgctcg |
| 1321 | gcatcatctc cggcatgacc ggcgccggcg gcaacttcgg cgccgggctc acgcagctgc |
| 1381 | tcttcttcac gtcgtcgagg tactccacgg gcacggggct ggagtacatg ggcatcatga |
| 1441 | tcatggcgtg cacgctgccg gtggtgctcg tccatttccc gcagtggggc tccatgttcc |
| 1501 | tcccgcccaa cgccggcgcc gaggaggagc actactacgg ctccgagtgg agcgaacagg |
| 1561 | agaagagcaa gggcctccac ggtgcaagtc tcaagttcgc cgagaactcc cgctccgagc |
| 1621 | gtggccgccg caacgtcatc aacgccgccg ccgccgccgc cacgccgccc aacaactcgc |
| 1681 | cggagcacgc ctaattaaga ggccaagtta attaattatg catgcatgta taaactgttg |
| 1741 | aacgttttgt taccgttttc tttatactat ctagagtatg tcgtcatgga g |
| SEQ ID NO: 4: OsNRT2.2 translation | |
| MDSSTVGAPGSSLHGVTGREPAFAFSTEVGGEDAAAASKFDLPV | |
| DSEHKAKTIRLLSFANPHMRTFHLSWISFFSCFVSTFAAAPLVPIIRDNLNLTKADIG | |
| NAGVASVSGSIFSRLAMGAICDMLGPRYGCAFLIMLAAPTVFCMSLIDSAAGYIAVRF | |
| LIGFSLATFVSCQYWMSTMFNSKIIGLVNGLAAGWGNMGGGATQLIMPLVYDVIRKCG | |
| ATPFTAWRLAYFVPGTLHVVMGVLVLTLGQDLPDGNLRSLQKKGDVNRDSFSRVLWYA | |
| VTNYRTWIFVLLYGYSMGVELTTDNVIAEYFYDRFDLDLRVAGIIAASFGMANIVARP | |
| TGGLLSDLGARYFGMRARLWNIWILQTAGGAFCLLLGRASTLPTSVVCMVLFSFCAQA | |
| ACGAIFGVIPFVSRRSLGIISGMTGAGGNFGAGLTQLLFFTSSRYSTGTGLEYMGIMI | |
| MACTLPVVLVHFPQWGSMFLPPNAGAEEEHYYGSEWSEQEKSKGLHGASLKFAENSRS | |
| ERGRRNVINAAAAAATPPNNSPEHA | |
| SEQ ID NO: 5: OsNRT2.3a AK109776 mRNA, complete cds | |
| 1 | agtcactagc taagctgcta gccttgctac cacgtgttgg agatggaggc taagccggtg |
| 61 | gcgatggagg tggagggggt cgaggcggcg gggggcaagc cgcggttcag gatgccggtg |
| 121 | gactccgacc tcaaggcgac ggagttctgg ctcttctcct tcgcgaggcc acacatggcc |
| 181 | tccttccaca tggcgtggtt ctccttcttc tgctgcttcg tgtccacgtt cgccgcgccg |
| 241 | ccgctgctgc cgctcatccg cgacaccctc gggctcacgg ccacggacat cggcaacgcc |
| 301 | gggatcgcgt ccgtgtcggg cgccgtgttc gcgcgtctgg ccatgggcac ggcgtgcgac |
| 361 | ctggtcgggc ccaggctggc ctccgcgtct ctgatcctcc tcaccacacc ggcggtgtac |
| 421 | tgctcctcca tcatccagtc cccgtcgggg tacctcctcg tgcgcttctt cacgggcatc |
| 481 | tcgctggcgt cgttcgtgtc ggcgcagttc tggatgagct ccatgttctc ggcccccaaa |
| 541 | gtggggctgg ccaacggcgt ggccggcggc tggggcaacc tcggcggcgg cgccgtccag |
| 601 | ctgctcatgc cgctcgtgta cgaggccatc cacaagatcg gtagcacgcc gttcacggcg |
| 661 | tggcgcatcg ccttcttcat cccgggcctg atgcagacgt tctcggccat cgccgtgctg |
| 721 | gcgttcgggc aggacatgcc cggcggcaac tacgggaagc tccacaagac tggcgacatg |
| 781 | cacaaggaca gcttcggcaa cgtgctgcgc cacgccctca ccaactaccg cggctggatc |
| 841 | ctggcgctca cctacggcta cagcttcggc gtcgagctca ccatcgacaa cgtcgtgcac |
| 901 | cagtacttct acgaccgctt cgacgtcaac ctccagaccg ccgggctcat cgccgccagc |
| 961 | ttcgggatgg ccaacatcat ctcccgcccc ggcggcgggc tactctccga ctggctctcc |
| 1021 | agccggtacg gcatgcgcgg caggctgtgg gggctgtgga ctgtgcagac catcggcggc |
| 1081 | gtcctctgcg tggtgctcgg aatcgtcgac ttctccttcg ccgcgtccgt cgccgtgatg |
| 1141 | gtgctcttct ccttcttcgt ccaggccgcg tgcgggctca ccttcggcat cgtgccgttc |
| 1201 | gtgtcgcgga ggtcgctggg gctcatctcc gggatgaccg gcggcggggg caacgtgggc |
| 1261 | gccgtgctga cgcagtacat cttcttccac ggcacaaagt acaagacgga gaccgggatc |
| 1321 | aagtacatgg ggctcatgat catcgcgtgc acgctgcccg tcatgctcat ctacttcccg |
| 1381 | cagtggggcg gcatgctcgt aggcccgagg aagggggcca cggcggagga gtactacagc |
| 1441 | cgggagtggt cggatcacga gcgcgagaag ggtttcaacg cggccagcgt gcggttcgcg |
| 1501 | gagaacagcg tgcgcgaggg cgggaggtcg tcggcgaatg gcggacagcc caggcacacc |
| 1561 | gtccccgtcg acgcgtcgcc ggccggggtg tgaagaatgc cacggacaat aaggtcgcgg |
| 1621 | ttgtagtaca actgtacaaa ttgatggtac gtgtcgtttg accgcgcgcg cgcacagtgt |
| 1681 | gggtcgtggc ctcgtgggct tagtggagta cagtgagggg tgtacgtgtg tcgtggcgcg |
| 1741 | cgcggtcacc tcggtggcct tgggattggg ggggcactat acgctagtac tccagatata |
| 1801 | tacgggtttg atttacttct gtggatcggc gcttgttggt ggtttgctcc ctgtggtttt |
| 1861 | tgtgatggta atcatactca tactcaaaca gtc |
| SEQ ID NO: 6: OsNRT2.3a translation | |
| MEAKPVAMEVEGVEAAGGKPRFRMPVDSDLKATEFWLFSFARPH | |
| MASFHMAWFSFFCCFVSTFAAPPLLPLIRDTLGLTATDIGNAGIASVSGAVFARLAMG | |
| TACDLVGPRLASASLILLTTPAVYCSSIIQSPSGYLLVRFFTGISLASFVSAQFWMSS | |
| MFSAPKVGLANGVAGGWGNLGGGAVQLLMPLVYEAIHKIGSTPFTAWRIAFFIPGLMQ | |
| TFSAIAVLAFGQDMPGGNYGKLHKTGDMHKDSFGNVLRHALTNYRGWILALTYGYSFG | |
| VELTIDNVVHQYFYDRFDVNLQTAGLIAASFGMANIISRPGGGLLSDWLSSRYGMRGR | |
| LWGLWTVQTIGGVLCVVLGIVDFSFAASVAVMVLFSFFVQAACGLTFGIVPFVSRRSL | |
| GLISGMTGGGGNVGAVLTQYIFFHGTKYKTETGIKYMGLMIIACTLPVMLIYFPQWGG | |
| MLVGPRKGATAEEYYSREWSDHEREKGFNAASVRFAENSVREGGRSSANGGQPRHTVP | |
| VDASPAGV | |
| SEQ ID NO: 7: OSNAR2.1 PROMOTER | |
| GATTCCCCACCTCTCCCACCTCACTCCTACCTCACTCCTAGTCCTCTGCCGAAAGTACTTC | |
| CTCCGTTTCACAATGTAAGTCATTCTAATATTTTTCACATTCATATTGATGTTTGAATCTAGAT | |
| TGATATATATGTTTAGATTCGTTAGCATCGATATGAATATGGGAAATGCTAGAATGACTTATA | |
| TTGTGAAACAGAGTGAGTATCATGTAAAAGTTAGAAGGAAAAAAATAGAGCTGTTTGTGATG | |
| ATATGGGTGTGGTTGTGTTGTGTGAGCCGATGTCCATTGTACTGTACTCATTTTAAATGTAC | |
| GTACCGTTAACTTATATAGTTATATGCGTTTGATCATTTGTCAAAATTTAGTGAAACTTTAAA | |
| ATTTATTATACTTAAAGTATATTTAATGATAAATTTAAAATAAAATAAACTTTCAGCTGTTATG | |
| TTCAAAATCAACATCGTCAGATATTTTAAATTAAAGGTAGTACTTTTAAAAAAAGGATTTTTG | |
| CGGTGTGTCGTGGCGAAACTGCTACCAAGTTTCAATGATCATATGCCATTTCATAGGATAAT | |
| TACTCTCATCGTGGTAAGTAAGAATCGATTGCCTATTTTCGGCAGGCTGTTGTTTCAAAGCA | |
| TCGATCTGCTTGGACAACTTGAGCAAAGCTAGCTAGAACTGGGTCGATATAATTGCAGCAC | |
| TAGGCAATCAAGAGACGGAGCTGGCCACCAGCTAGCTGAGCTGAGCTGATATGATCAACA | |
| CAGTGCAGACTTGGTCGTGTTCGAGTTCGATCGACGGATGGCTGTCCTGCTCTTGCGCTC | |
| ATGCATGTCATCTCTTCGGAAGTAGGAGTACAGCAGTACTTGAGGAATATTATTAGAGAGT | |
| AAGTTGAACTGTTTTCAATAGTTCAGGGTGTAAACTAAGCTGAGGAATTGTTAGGAGGTTAA | |
| ATGCTGTGGCAAAATAGTTTGGAGGAGCGAAATGATTTTTTTTTCATATGAAAAACATCTAA | |
| ATTTATTTTTTGCCAAAACACTAGTATATCATCAAATTTTCATCCATTAAGAACGCCTTCTCA | |
| ATATTAATAATTCCAATGTGATATCTTAATGCTCAATGAACCTAAAATAGTTTGGATGAGTGA | |
| AATGGACTCTTTTTGAGTTTTTTTCCATATGAAAACATCTAAATTTATTTTTTTTTGCCAAAAC | |
| ACTGGTATATCATCAAATTTTCCTCCATTAAGAACGCCTTCTCAACGTTAATAACTCCAATGT | |
| TATTATCTTAATGCCAAATGAACCTACCATGAACGTCATGCTCACAATTTAATTAACAACAAC | |
| CGAGGCACTCAAGATCATTCGCGGTTGCCGCTTCTCACCGGTTGCCTGAACCCTTGGGAC | |
| CCCTCCAAAAGCTTAATTACCCCCAAAACCGCATGATCTCTCTCTTCTCTTCTCTTCTCACA | |
| CGTCGTCAAAGCCTCTGACTTTGGATATCCCCGACCCCACTAAACTTAATCAACTTGATCAT | |
| TACAACAATTAAGTTGCCTCTTGAATCCAACGAAGTAGCTGGTCAACTCTCCGAGCTCGTA | |
| GCCTCGCTCTCCCGCCTATAAATTCACCGATCGATCGATCGATCGATCTCAGCATCAGCAG | |
| CAGCAGCAGATTCATTTCTTGGTCTTCGTCTCCGTCTCCGTCCTTGGGTTGATATCCAGAAT | |
| CAGTCGGTTTGGTTTGTCAGCAATG | |
| SEQ ID NO: 8: OSNAR2.1 AP004023.2 MRNA, COMPLETE CDS | |
| 1 | ATTCATTTCT TGGTCTTCGT CTCCGTCTCC GTCCTTGGGT TGATATCCAG |
| 51 | AATCAGTCGG TTTGGTTTGT CAGCAATGGC GAGGCTAGCC GGCGTTGCTG |
| 101 | CTCTCTCGTT GGTGCTCGTC TTGCTCGGCG CCGGCGTGCC CCGGCCGGCG |
| 151 | GCCGCCGCCG CGGCGAAGAC GCAGGTGTTC CTCTCCAAGC TGCCCAAAGC |
| 201 | GCTCGTCGTC GGCGTCTCGC CCAAGCACGG TGAAGTCGTG CACGCCGGCG |
| 251 | AGAACACGGT GACGGTGACG TGGTCGCTGA ACACGTCGGA GCCGGCGGGC |
| 301 | GCCGACGCGG CGTTCAAGAG CGTGAAGGTG AAGCTGTGCT ACGCGCCGGC |
| 351 | GAGCCGGACG GACCGCGGGT GGCGCAAGGC CTCCGACGAC CTGCACAAGG |
| 401 | ACAAGGCGTG CCAGTTCAAG GTCACCGTGC AGCCGTACGC CGCCGGCGCC |
| 451 | GGCAGGTTCG ACTACGTGGT GGCGCGCGAC ATCCCGACGG CGTCCTACTT |
| 501 | CGTGCGCGCC TACGCGGTGG ACGCGTCCGG CACGGAGGTG GCCTACGGGC |
| 551 | AGAGCTCGCC GGACGCCGCC TTCGACGTCG CCGGGATCAC CGGCATCCAC |
| 601 | GCCTCCCTCA AGGTCGCCGC CGGCGTCTTC TCCACCTTCT CCATCGCCGC |
| 651 | GCTCGCCTTC TTCTTCGTCG TCGAGAAGCG CAAGAAGGAC AAG |
| SEQ ID NO: 9: OsNAR2.1 translation | |
| MARLAGVAALSLVLVLLGAGVPRPAAAAAAKTQVFLSKLPKALVVGVSPKHGEVVHAGENTVT | |
| VTWSLNTSEPAGADAAFKSVKVKLCYAPASRTDRGWRKASDDLHKDKACQFKVTVQPYAAGA | |
| GRFDYVVARDIPTASYFVRAYAVDASGTEVAYGQSSPDAAFDVAGITGIHASLKVAAGVFSTFSI | |
| AALAFFFVVEKRKKDK | |
| SEQ ID NO: 10: OsNAR2.2 AK109571 mRNA, complete cds | |
| 1 | acctctagca gagaacgatc atggctcggt ttggggcggt aattcaccgc gtgtttctac |
| 61 | cgctgttgct gctccttgta gttctcggtg cttgccatgt cacgccggcg gcggcggcgg |
| 121 | cgggggcgcg cctctccgcg ctcgcgaagg cgctcgtcgt cgaggcgtcg ccccgtgccg |
| 181 | gccaagtcct gcacgccggc gaggacgcca tcaccgtgac atggtcgctg aacgcgacgg |
| 241 | cggcggcggc ggcggccggg gcggatgccg gctacaaggc ggtgaaggtg accctgtgct |
| 301 | acgcgccggc gagccaggtg ggccgcgggt ggcgcaaggc ccacgacgac ctgagcaagg |
| 361 | acaaggcgtg tcagttcaag atcgcccagc agccgtacga cggcgccggc aagttcgagt |
| 421 | acacggtggc acgcgacgtc ccgacggcgt cgtactacgt gcgcgcctac gcgctcgacg |
| 481 | cgtcgggggc gcgggtggcc tatggcgaga cggcgccctc ggccagcttc gccgtcgcgg |
| 541 | gcatcaccgg cgtcaccgcg tccatcgagg tcgccgccgg cgtgctctcc gcgttctccg |
| 601 | tcgccgcgct cgccgtcttc ctcgtcctcg agaacaagaa gaagaacaag tgattgtggt |
| 661 | ttgcttgtgt tagtagtctg tacaattgta atgtgtgact gtgtacgaca gcacggagtg |
| 721 | tgtctacagc gcccagcaca cacacctccg tatgttcaac ctacaaaacc aacggaataa |
| 781 | tagatggatc tttg |

SEQ ID NO: 11: SEQ ID NO 11: OsNAR2.2 translation
MARFGAVIHRVFLPLLLLLVVLGACHVTPAAAAAGARLSALAKA
LVVEASPRAGQVLHAGEDAITVTWSLNATAAAAAAGADAGYKAVKVTLCYAPASQVGR
GWRKAHDDLSKDKACQFKIAQQPYDGAGKFEYTVARDVPTASYYVRAYALDASGARVA
YGETAPSASFAVAGITGVTASIEVAAGVLSAFSVAALAVFLVLENKKKNK
SEQ ID NO: 12: SEQ ID NO 12: OsNAR2.2 promoter
TTGGGCTTTTTCTTCTGTTGTGCTTGTGCTTGTGTTGGTCCGGAGGTTTTATGGGCTACTTG
GGCAAAATTGCATTGCCAACAGGCGAACTGCCATAGTAAAAAGAGAAAATTCATAAAATGT
CATCGATAAATATCCCAATTCAAACGAATTCGCTATCCATCTGTCGACAGATTTTCGTTTAAA
GATTTGTCGATTTTTGGGCCATATGATTTGCGTGGAGATTTGTGCTAACTAATACGATGGAT
AAAAGAGACAAGTCTGTGCGTAGCGGGGGATCGATTCCAAGAGCACTAGTTTTCCCTGTTA
AACGTACACCACAGAGCAATTAAGCCAAGTTGGTTCAAGACAAAATAAAGTTTATATTGAAG
TTACAGTGTTAGAAAACTAAAATTACAATGTAATTATACTATAGTTACATGTATGTAAATATG
GTGCAATTATAGTGTAACAATAATATAATTAAAATTACAGTGCAATTATACTATAATTACATAT
GTAACTATGGTGTAATTATAATGTAACAACAATGTAATTAAAATTACAGTGCAATTATACCAT
AATTACATATGTAACTATGGTGTAATTATAGTGTAACAACAATGTAATTAAAATTACAGTGTA
ACTATACCATAATTACATTTGTAACTATGGTGTAATTATAGTGTAACAACAATGTAACTTTAG
TAGATGGATTGGTAATATCTTTGTATGCACTAAAAAATATCTAGTAGATATCACACTATACAT
GAGCAGATGAGCTCTCAAGGATTAATAATTAATATGGATAAATAACCAACTAATTTTGATTAA
GAAACGAGAGTGTGAATTAACGTCGGAAGCCCATCCATACTATCGATTAGTAGATGCGGGA
CCTCAGCAGTGCTGTACACGTGTCATCCATCTATCGTTTATGGGTTTAATCCAAAGGTACAA
ATTTTGACAAATTTATGATGAAAAATCTGTCGACAGATGTGTAGACTCTCCCCAATTCAAAA
AATATCATCGATAAATCTAATCTCTTTAGAAATGTCATCGTACAAGTGCTTTTGTTCTAGAGT
TACCATCGTTGTTAAGTTTTCGGTTGCATCCATCTGTTAAGTGCTATAAAAAGATCATTTTAC
CCTATAAATTATTAAAGGTTGATTAAAAATTTTGCTCTTTTCGTTAGTTTACACTCAATTAGTT
TGCTCTTTACACTAAAATATTGAAAATTGACTGAAAGAGTAAAAGTTTTAATCATTTAATAATT
TGTAGGGGTAAAATGGTTTTTTATAGCACTCAACGAATGGCTACAAACGAAAATCTAACGAC
GATGATATTTTTAGAATAAAAGCGCTTGTACAATAATATTTTTAGGAAGCTGTGTTCGTTAAT
GGTATTTCTTGGATTGGAGGGCTCGTGATTTTTCTCCGGCAAACAGTGTACCGTGTTCTGA
CCACCACCAACAGACTGTCTGACTACTACCGTTAACGTGGTACTGCTACTAGTCTACTACT
ACTACTGTGCAGTGTACTACAGATACCACACAGCTGCATTAGCCTGCATTCGCCGCTTCAC
ATCGCCATGGCCCTCAAAAGGTCGACCGAGGTGCCCCATCGAGCCGACGAGACAGCCAA
ACGTACGTGCTCCGACAGTCAGACCCGCGTGAACCATCAAGCTCCGACTTTCGGAACCAC
CCATCGCTACCCTCCTCGACCCCACAAAGTTGAACTCCCCCGATCTCCCTCCCTCTCCACG
AGTCAACTTACTCGACGCTACCACGCCTATATATAAGCTACCGCTCCGCTCAAATGGCCTC
CACCTCTAGCAGAGAACGATCATG

Arabidopsis Thaliana

| SEQ ID NO: 13: AtNAR2.1 AJ311926.1 mRNA, complete cds | |
|---|---|
| 1 | gatacaatta caatatgtag agtatcttat aggtgacgta accatgaaat atagaattct |
| 61 | ttggaatctg aaactgaatt attcagttga taaatgataa acaaatactc atatctcatc |
| 121 | ctttggcatg gcgatccaga agatcctctt tgcttcactt ctcatatgct cactgatcca |
| 181 | atccatccac ggggcggaaa aagtaagact cttcaaagag ctggacaaag gtgcacttga |
| 241 | tgtcaccact aaacccagcc gagaaggacc aggtgttgtt ttggatgccg gcaaggatac |
| 301 | gttgaacatt acatggacgc taagctcgat tgggtctaaa agagaggctg aatttaagat |
| 361 | catcaaagtt aagctatgct acgctccacc tagccaagtt gaccgaccat ggcgcaaaac |
| 421 | ccatgacgag ctcttcaaag acaagacctg cccacacaag atcatagcca agccttatga |
| 481 | caaaacactt caatcaacta cttggactct tgagcgtgac atccccaccg gaacctactt |
| 541 | cgttcgtgcc tacgcggttg atgccattgg ccatgaagtt gcctatggac agagcaccga |
| 601 | cgatgccaag aaaaccaatc tcttcagcgt tcaggctatc agtggccgcc acgcgtccct |
| 661 | agatattgcc tccatctgtt tcagtgtctt ctccgtcgtg gctcttgtcg tcttctttgt |
| 721 | caatgagaag aggaaggcca agatagagca aagcaaatga gtcgtttact ttgcgtattt |
| 781 | gtgacgttga acccaaaaaa agttgacttt gaactttctt gtttaccaat tccttttgtc |
| 841 | ttgttgcaca ctt |
| SEQ ID NO: 14: AtNAR2.2 AJ310933.1 mRNA, complete cds | |
| 1 | taaaagtcag caaaacacaa ggcatattcc tcttctcttc ctcagcctta tttttctgat |
| 61 | attcagtttc aaggatatat ccatggcgat ccagaagatc ctctttgctt cacttctcat |
| 121 | atgctcactg atccaatcca tccacggggc ggaaaaacta agactcttca aagagctgga |
| 181 | caaaggtgca cttgatgtca ccactaaacc cagccgagaa ggaccaggtg ttgttttgga |
| 241 | tgccggcaag gatacgttga acattacatg gacgctaagc tcgattgggt ctaaaagaga |
| 301 | ggctgaattt aagatcatca aagttaagct atgctacgct ccacctagcc aagttgaccg |
| 361 | accatggcgc aaaacccatg acgagctctt caaagacaag acctgcccac acaagatcat |
| 421 | agccaagcct tatgacaaaa cacttcaatc aactacttgg actcttgagc gtgacatccc |
| 481 | caccggaacc tacttcgttc gtgcctacgc ggttgatgcc attggccatg aagttgccta |
| 541 | tggacagagc accgacgatg ccaagaaaac caatctcttc agcgttcagg ctatcagtgg |
| 601 | ccgccacgcg tccctagata ttgcctccat ctgtttcagt gtcttctccg tcgtggctct |
| 661 | tgtcgtcttc tttgtcaatg agaagaggaa ggccaagata gagcaaagca aatgagtcgt |
| 721 | ttactttgcg tatttgtgac gttgaaccca aaaaaagttg actttgaact ttcttgttta |
| 781 | ccaattcctt ttgtcttgtt gcacacttct tctttcttat atgcttttat ttatgtgttt |
| 841 | gtacaattaa gccattgat |
| SEQ ID NO: 15: ATNRT2.1 NM_100684.2 MRNA, COMPLETE CDS | |
| 1 | atcgatcaaa taaacttgaa tcaaatctca aacttgcaaa gaaacttgaa atattttata |
| 61 | acaatgggtg attctactgg tgagccgggg agctccatgc atggagtcac cggtagagaa |
| 121 | caaagctttg ctttctcggt gcaatcacca attgtgcata ccgacaagac ggccaagttc |
| 181 | gaccttccgg tggacacaga gcataaggca acggttttca agctcttctc cttcgccaaa |
| 241 | cctcacatga gaacgttcca tctctcgtgg atctctttct ccacatgttt tgtctcgact |
| 301 | ttcgcagctg caccacttgt ccctatcatc cgggagaatc tcaacctcac caaacaagac |
| 361 | attggaaacg ccggagttgc ctctgtctct gggagtatct tctctaggct cgtgatggga |
| 421 | gccgtgtgtg atcttttggg tccccgttac ggttgtgcct tccttgtgat gttgtctgcc |
| 481 | ccaacggtgt tctccatgag cttcgtgagt gacgcagcag gcttcataac ggtgaggttc |
| 541 | atgattggtt tttgcctggc gacgtttgtg tcttgtcaat actggatgag cactatgttc |
| 601 | aacagtcaga tcattggtct ggtgaatggg acagcagccg gatggggaaa catgggtggc |
| 661 | ggcataacgc agttgctcat gcccattgtg tatgaaatca ttaggcgctg cggttccaca |
| 721 | gccttcacgg cctggaggat cgccttcttt gtacccggtt ggttgcacat catcatggga |
| 781 | atcttggtgc tcaatctagg tcaagatctg ccagatggaa atcgagctac cttggagaaa |
| 841 | gcgggagaag ttgccaaaga caaattcgga aagattctgt ggtatgccgt tacaaactac |
| 901 | aggacttgga tcttcgttct tctctacgga tactccatgg gagttgagtt gagcactgat |
| 961 | aatgttatcg ccgagtactt ctttgacagg tttcacttga agctccacac agcagggctc |
| 1021 | atagcagcat gtttcggaat ggccaatttc tttgctcgtc cagcaggagg ctacgcatct |
| 1081 | gactttgcag ccaagtactt cgggatgaga gggaggttgt ggacgttgtg gatcatacag |
| 1141 | acggctggtg gcctcttctg tgtgtggctc ggccgcgcca acacccttgt aactgccgtt |
| 1201 | gtggctatgg tgctcttctc tatgggggca caagctgctt gcggagccac ctttgcaatt |
| 1261 | gtgccctttg tctcccggcg agctctaggc atcatctcgg gtttaaccgg ggctggaggg |
| 1321 | aactttggat cagggctcac acaactcctc ttcttctcga cctcacactt cacaactgaa |
| 1381 | caagggctaa cgtggatggg agtgatgata gtcgcttgca cgttacctgt gaccttagtt |
| 1441 | cactttcctc aatggggaag catgttcttg cctccttcca cagatccagt gaaaggtaca |
| 1501 | gaggctcatt attatggttc tgagtggaat gagcaggaga agcagaagaa catgcatcaa |
| 1561 | ggaagcctcc ggtttgccga gaacgccaag tcagagggtg gacgccgcgt ccgctctgct |
| 1621 | gctacgccgc ctgagaacac acccaacaat gtttgatcat acattccacc cacggtggaa |
| 1681 | tggtgaagga tgatcgcata taagaatatg tcacacagtg aaaaaaaaaa atgcaaatgt |
| 1741 | tatcaatgct tgcataacat tactatctat ctttcattta ctaaacaaac cttttgcttt |
| 1801 | ttgccttgaa atctttttat tatatatcaa aatatatctc tatgtcttga gatttgatta |
| 1861 | ttttgcatat atcattaatg atttgataat attggaactg |
| SEQ ID NO: 16: ATNRT2.2 NM_100685.1 MRNA, COMPLETE CDS | |
| 1 | aaacttgaat tttctcaaag gaacttgata cgtttaaaat acatgggttc tactgatgag |
| 61 | cccagaagtt ccatgcatgg agttaccggt agagaacaga gctatgcttt ctcggtagat |
| 121 | ggtagtgagc cgaccaacac aaagaaaaag tacaatctgc cggtggacgc ggaggataag |
| 181 | gcaacggttt tcaagctctt ctccttcgcc aaacctcaca tgagaacgtt ccacctctcg |
| 241 | tggatctctt tctccacatg ttttgtttcg acgttcgcag ctgcaccact tatcccgatc |
| 301 | atcagggaga atcttaacct caccaaacat gacattggaa acgctggagt tgcctccgtc |
| 361 | tcggggagta tcttctctag gctcgtgatg ggagccgtgt gtgatctttt gggtcctcgt |
| 421 | tacggttgtg ccttccttgt gatgttgtct gccccaacgg tgttctccat gagcttcgtg |
| 481 | agtgacgcag caggcttcat aacggtgagg ttcatgattg gtttttgcct ggcgacgttt |
| 541 | gtgtcttgtc aatactggat gagcactatg ttcaacagtc agatcatcgg tctggtgaac |
| 601 | gggacagcag ccggatgggg aaacatgggt ggcggcataa cgcagttgct catgcccatt |
| 661 | gtgtatgaaa tcattaggcg ctgcggatca acagcgttca cggcctggag gatcgccttc |
| 721 | tttgtccccg gttggttgca catcatcatg ggaatcttgg tgctcacgct aggtcaagat |
| 781 | ctgccaggtg gaaacagagc tgccatggag aaagcgggag aagttgccaa agacaaattc |
| 841 | ggaaagattc tatggtacgc cgttacaaat tacaggactt ggattttcgt tcttctgtat |
| 901 | ggatattcca tgggagttga gttaagcaca gacaatgtta tcgccgagta cttctttgac |
| 961 | aggtttcact tgaagcttca cacagcgggg attatagcag catgtttcgg aatggccaat |
| 1021 | ttctttgctc gtccagcagg aggctgggca tctgacattg cagccaagcg cttcggaatg |
| 1081 | cgagggaggt tgtggacttt gtggatcatt cagacgtccg gtggtctctt ttgtgtgtgg |
| 1141 | ctcggacgtg ccaacaccct cgtcactgcc gttgtatcta tggtcctctt ctctttagga |
| 1201 | gcacaagccg cttgcggagc cacctttgct atcgtgccct ttgtctcccg gcgagctcta |
| 1261 | ggcattatct cgggtttaac cggggctgga gggaactttg ggtcaggact cacacagctc |
| 1321 | gtctttttct cgacttcgcg cttcacaact gaagaagggc taacgtggat gggagtgatg |
| 1381 | atagttgctt gcacgttgcc tgttacctta atccactttc ctcagtgggg aagcatgttc |
| 1441 | ttccctcctt ccaacgattc ggtcgacgct acggagcact attatgttgg cgaatatagt |
| 1501 | aaggaggagc agcagattgg catgcattta aaaagcaaac tgtttgctga tggagccaag |
| 1561 | accgagggag gcagcagcgt ccacaaaggg aacgcaacca acaatgcttg atcatgtgtc |
| 1621 | attgatatca agaaattaat aatttcactt atgtgaaatg gacataaact gttggaaaat |
| 1681 | aaagaaccat ttctttcatc atttgcttt |
| SEQ ID NO: 17: AtNRT2.3 NM_125471.1 mRNA, complete cds | |
| 1 | atgactcaca accattctaa tgaagaaggc tccattggaa cctccttgca tggagttaca |
| 61 | gcaagagaac aagtcttctc tttctccgtc gatgcttcgt ctcaaacagt ccaatcagac |
| 121 | gatccaacag ctaaattcgc ccttccggtt gattccgaac atcgagccaa agtgttcaac |
| 181 | ccactctctt ttgctaaacc tcacatgaga gccttccact taggatggct ctcattcttc |
| 241 | acatgcttca tctccacctt cgcggcagca ccattagtcc ccatcatccg cgacaacctc |
| 301 | gacctcacta aaaccgacat tggaaacgcc ggagtcgcat ccgtctctgg tgccattttc |
| 361 | tcaaggttag ccatgggagc ggtttgtgat ctcctcggtg cacgatatgg gactgccttc |
| 421 | tccctcatgc taaccgcccc aaccgtcttc tcaatgtcgt ttgtgggtgg ccctagcgga |
| 481 | tacttaggcg tccggttcat gatcggattc tgtctcgcca cgtttgtatc atgccagtat |
| 541 | tggaccagcg ttatgttcaa cggtaagatc ataggactag tgaacggctg tgcaggcggg |
| 601 | tggggtgata tgggcggtgg agtgactcaa ctcctaatgc cgatggtctt ccacgtcatc |
| 661 | aaacttgccg gagccactcc gttcatggcc tggcggatag ctttcttcgt tcccggattt |
| 721 | cttcaagttg ttatgggcat tctcgtcctc agtctcggcc aagatctccc tgacggtaac |
| 781 | ctaagtaccc ttcagaagag tggtcaagtc tctaaagaca aattctccaa ggttttctgg |
| 841 | tttgctgtga agaactacag aacatggatt ttattcgttc tttatggatc ttccatggga |
| 901 | attgaattaa ctatcaacaa cgttatctcc ggatattttt acgacaggtt taaccttaag |
| 961 | cttcaaacag ctggtatagt agcagccagc tttggaatgg ctaacttcat cgcccgtccc |
| 1021 | ttcggtggtt acgcttctga tgtagcggct cgggtttttg gcatgagagg ccggttatgg |
| 1081 | accttatgga tctttcaaac cgtaggagct cttttctgta tctggctagg tcgagctagt |
| 1141 | tcacttccca tagcaatcct agcaatgatg ctcttctcaa tcggtacaca agcagcttgc |
| 1201 | ggagccctct tcggagttgc accttttgtc tcgcgccgct ctctagggct catatcggga |
| 1261 | ctaaccggcg caggaggaaa cttcgggtcc ggtttgactc aactgctttt cttctcatca |
| 1321 | gcgaggttta gtacagctga gggactctca ttgatgggcg ttatggcggt tttgtgcaca |
| 1381 | ctcccagttg cgtttataca ttttccgcaa tggggaagca tgtttttaag accgtcgacc |
| 1441 | gatggagaaa gatcacagga ggaatattat tacggttctg agtggacgga gaatgagaaa |
| 1501 | caacaaggat tgcacgaagg aagcatcaaa tttgcagaga atagtaggtc agagagaggc |
| 1561 | cggaaagtag ctttggctaa cattccaacg ccggagaacg gaactccaag tcatgtttga |
| SEQ ID NO: 18: AtNRT2.4 At5g60770 mRNA, complete cds | |
| 1 | atggccgatg gttttggtga accgggaagc tcaatgcatg gagtcaccgg cagagaacaa |
| 61 | agctatgcat tctctgtcga gtctccggca gttccttccg actcatcagc aaaattttct |
| 121 | ctccccgtgg acaccgaaca caaagccaaa gtcttcaaac tcttatcctt tgaagctcca |
| 181 | catatgagaa ctttccatct tgcttggatc tcattcttca cttgcttcat ttccactttc |
| 241 | gctgctgctc ctcttgtccc catcattaga gataacctta atctcacaag acaagatgtc |
| 301 | ggaaatgctg gtgttgcttc tgtctctggc agtatcttct ctaggcttgt tatgggagca |
| 361 | gtttgtgatc tccttgggcc acgttatggc tgtgctttcc tcgtcatgct ctctgctcca |
| 421 | accgtcttct ccatgtcttt cgttggtggt gccggagggt acataacggt gaggttcatg |
| 481 | atcgggttct gcctggcgac tttcgtgtca tgccagtatt ggatgagcac aatgttcaat |
| 541 | ggtcagatca taggtctagt gaacgggaca gcggcagggt gggggaacat gggcggtggg |
| 601 | gtcactcagt tgctcatgcc aatggtctat gagatcatcc gacggttagg gtccacgtcc |
| 661 | ttcaccgcat ggaggatggc tttcttcgtc cccgggtgga tgcacatcat catggggatc |
| 721 | ttggtcttga ctctagggca agacctccct gatggtaata gaagcacact cgagaagaaa |
| 781 | ggtgcagtta ctaaagacaa gttctcaaag gttttatggt acgcgatcac gaactatagg |
| 841 | acatgggttt tcgtgctgct atatggatac tccatgggag tagagctcac aaccgataac |
| 901 | gtcatcgctg agtacttttt cgacaggttc catcttaagc ttcataccgc cggtataatc |
| 961 | gcggcaagct ttggtatggc aaacttcttt gcccgtccta ttggtggttg ggcctcagat |
| 1021 | attgcggcta gacgcttcgg catgagaggc cgtctctgga ccctatggat catccaaacc |
| 1081 | ttaggcggtt tcttctgcct atggctaggc cgagccacca cgctcccgac cgcggttgtc |
| 1141 | ttcatgatcc tcttctctct cggcgctcaa gccgcttgtg gagctacctt tgctatcata |
| 1201 | cctttcatct cacgccgctc cttagggatc atctctggtc ttactggagc tggtggaaac |
| 1261 | ttcggctctg gtttgaccca actcgtattc ttctcgacct caacgttctc cacggaacaa |
| 1321 | gggctgacat ggatgggggt gatgattatg gcgtgtacat tacccgtcac tttagtgcac |
| 1381 | ttcccgcaat ggggaagcat gtttttgcct tccacggaag atgaagtgaa gtctacggag |
| 1441 | gagtattatt acatgaaaga gtggacagag accgagaagc gaaagggtat gcatgaaggg |
| 1501 | agtttgaagt tcgccgtgaa tagtagatcg gagcgtggac ggcgcgtggc ttctgcaccg |
| 1561 | tctcctccgc cggaacacgt ttaa |
| SEQ ID NO: 19: AtNAR2.1 translation | |
| MAIQKILFASLLICSLIQSIHGAEKVRLFKELDKGALDVTTKPS | |
| REGPGVVLDAGKDTLNITWTLSSIGSKREAEFKIIKVKLCYAPPSQVDRPWRKTHDEL | |
| FKDKTCPHKIIAKPYDKTLQSTTWTLERDIPTGTYFVRAYAVDAIGHEVAYGQSTDDA | |
| KKTNLFSVQAISGRHASLDIASICFSVFSVVALVVFFVNEKRKAKIEQSK | |
| SEQ ID NO: 20: AtNAR2.2translation | |
| MAIQKILFASLLICSLIQSIHGAEKLRLFKELDKGALDVTTKPS | |
| REGPGVVLDAGKDTLNITWTLSSIGSKREAEFKIIKVKLCYAPPSQVDRPWRKTHDEL | |
| FKDKTCPHKIIAKPYDKTLQSTTWTLERDIPTGTYFVRAYAVDAIGHEVAYGQSTDDA | |
| KKTNLFSVQAISGRHASLDIASICFSVFSVVALVVFFVNEKRKAKIEQSK | |
| SEQ ID NO: 21: AtNRT2.1translation | |
| MGDSTGEPGSSMHGVTGREQSFAFSVQSPIVHTDKTAKFDLPVD | |
| TEHKATVFKLFSFAKPHMRTFHLSWISFSTCFVSTFAAAPLVPIIRENLNLTKQDIGN | |
| AGVASVSGSIFSRLVMGAVCDLLGPRYGCAFLVMLSAPTVFSMSFVSDAAGFITVRFM | |
| IGFCLATFVSCQYWMSTMFNSQIIGLVNGTAAGWGNMGGGITQLLMPIVYEIIRRCGS | |
| TAFTAWRIAFFVPGWLHIIMGILVLNLGQDLPDGNRATLEKAGEVAKDKFGKILWYAV | |
| TNYRTWIFVLLYGYSMGVELSTDNVIAEYFFDRFHLKLHTAGLIAACFGMANFFARPA | |
| GGYASDFAAKYFGMRGRLWTLWIIQTAGGLFCVWLGRANTLVTAVVAMVLFSMGAQAA | |
| CGATFAIVPFVSRRALGIISGLTGAGGNFGSGLTQLLFFSTSHFTTEQGLTWMGVMIV | |
| ACTLPVTLVHFPQWGSMFLPPSTDPVKGTEAHYYGSEWNEQEKQKNMHQGSLRFAENA | |
| KSEGGRRVRSAATPPENTPNNV | |
| SEQ ID NO: 22: AtNRT2.2translation | |
| MGSTDEPRSSMHGVTGREQSYAFSVDGSEPTNTKKKYNLPVDAE | |
| DKATVFKLFSFAKPHMRTFHLSWISFSTCFVSTFAAAPLIPIIRENLNLTKHDIGNAG | |
| VASVSGSIFSRLVMGAVCDLLGPRYGCAFLVMLSAPTVFSMSFVSDAAGFITVRFMIG | |
| FCLATFVSCQYWMSTMFNSQIIGLVNGTAAGWGNMGGGITQLLMPIVYEIIRRCGSTA | |
| FTAWRIAFFVPGWLHIIMGILVLTLGQDLPGGNRAAMEKAGEVAKDKFGKILWYAVTN | |
| YRTWIFVLLYGYSMGVELSTDNVIAEYFFDRFHLKLHTAGIIAACFGMANFFARPAGG | |
| WASDIAAKRFGMRGRLWTLWIIQTSGGLFCVWLGRANTLVTAVVSMVLFSLGAQAACG | |
| ATFAIVPFVSRRALGIISGLTGAGGNFGSGLTQLVFFSTSRFTTEEGLTWMGVMIVAC | |
| TLPVTLIHFPQWGSMFFPPSNDSVDATEHYYVGEYSKEEQQIGMHLKSKLFADGAKTE | |
| GGSSVHKGNATNNA | |
| SEQ ID NO: 23: AtNRT2.3translation | |
| MTHNHSNEEGSIGTSLHGVTAREQVFSFSVDASSQTVQSDDPTA | |
| KFALPVDSEHRAKVFNPLSFAKPHMRAFHLGWLSFFTCFISTFAAAPLVPIIRDNLDL | |
| TKTDIGNAGVASVSGAIFSRLAMGAVCDLLGARYGTAFSLMLTAPTVFSMSFVGGPSG | |
| YLGVRFMIGFCLATFVSCQYWTSVMFNGKIIGLVNGCAGGWGDMGGGVTQLLMPMVFH | |
| VIKLAGATPFMAWRIAFFVPGFLQVVMGILVLSLGQDLPDGNLSTLQKSGQVSKDKFS | |
| KVFWFAVKNYRTWILFVLYGSSMGIELTINNVISGYFYDRFNLKLQTAGIVAASFGMA | |
| NFIARPFGGYASDVAARVFGMRGRLWTLWIFQTVGALFCIWLGRASSLPIAILAMMLF | |
| SIGTQAACGALFGVAPFVSRRSLGLISGLTGAGGNFGSGLTQLLFFSSARFSTAEGLS | |
| LMGVMAVLCTLPVAFIHFPQWGSMFLRPSTDGERSQEEYYYGSEWTENEKQQGLHEGS | |
| IKFAENSRSERGRKVALANIPTPENGTPSHV | |
| SEQ ID NO: 24: AtNRT2.4translation | |
| MADGFGEPGSSMHGVTGREQSYAFSVESPAVPSDSSAKFSLPVD | |
| TEHKAKVFKLLSFEAPHMRTFHLAWISFFTCFISTFAAAPLVPIIRDNLNLTRQDVGN | |
| AGVASVSGSIFSRLVMGAVCDLLGPRYGCAFLVMLSAPTVFSMSFVGGAGGYITVRFM | |
| IGFCLATFVSCQYWMSTMFNGQIIGLVNGTAAGWGNMGGGVTQLLMPMVYEIIRRLGS | |
| TSFTAWRMAFFVPGWMHIIMGILVLTLGQDLPDGNRSTLEKKGAVTKDKFSKVLWYAI | |
| TNYRTWVFVLLYGYSMGVELTTDNVIAEYFFDRFHLKLHTAGIIAASFGMANFFARPI | |
| GGWASDIAARRFGMRGRLWTLWIIQTLGGFFCLWLGRATTLPTAVVFMILFSLGAQAA | |
| CGATFAIIPFISRRSLGIISGLTGAGGNFGSGLTQLVFFSTSTFSTEQGLTWMGVMIM | |
| ACTLPVTLVHFPQWGSMFLPSTEDEVKSTEEYYYMKEWTETEKRKGMHEGSLKFAVNS | |
| RSERGRRVASAPSPPPEHV | |
| SEQ ID NO: 25: AtNAR2.1 promoter | |
| 481 | gatgaagaca tcggtggtat gaatcttctg attttgaatc tggtaacacc atcaccatcc |
| 541 | aaaataagaa aaaaaaaata cgaaaaatga ttaaagagag cgtctcccat cccaattact |
| 601 | aaaaaaaaat gggaaagaaa ccacccggca attgctggta gccgtaaaaa ccaaacaaga |
| 661 | aaatagatat tacttttgtc ctcccaaaga ttttgtccag ttaaccaact tttcgaagcc |
| 721 | atgtcttacg ttttcagtat atgcctctaa ggcaacttgt attttctcaa agtgtggttt |
| 781 | ccatattgtc ataactcggc ttacctagaa aaacaagtaa caatggtcac aatgtatagt |
| 841 | gcaatgtcaa aagagaatca gacataatga ataaagatgg aaacatatta acctcaagat |
| 901 | agtcgtagga aacgtcgaat ggttgggtga aatgtggggt caaaagcctg cttacatgta |
| 961 | tgtaacacct cgatggactt atcggttaag tattgaattt ttggttggag ataaatggtt |
| 1021 | aatgtcacat acacatcttt gatgcttgga atccatttct gtgatatcta agagaagaat |
| 1081 | tcaaaggtta ataagaataa agcaataatg acttgtctta taacgaaaac caaaaacttt |
| 1141 | cagaataaaa ccttttgaag agtttggcta agatcttgtt gccgcagagt cactatatat |
| 1201 | gcttgcaacc ataataaaaa aaaagaggat catttaagat tgatcactat gacaatttta |
| 1261 | tcgatgttaa ttatgaacct tcttatagag ttacctgagt tttatttagg tttagaccaa |
| 1321 | gcaacttccc atcgatctga acacaagtaa aaaaaaagta aagaaagctt ttgaatatgt |
| 1381 | acatacatca gatccaaggg acaccatgtt tcttacattt tcaagctttg agccaagttc |
| 1441 | cgcgactttc ttttctgcga tttcagctcg agtttccact tccagacttc tcttgcgttg |
| 1501 | catttccact tcttctcgta gttcaagtat ctgccaatgc agaactaaat tgaggtaaca |
| 1561 | aaacaagaag gaaatacaca cctcaaacta caaaacacca acctgtttct gaagttcata |
| 1621 | agccgttcct tctgcttcac tggattctca gtctaaataa aaaagcatca acagcaacaa |
| 1681 | aagtgtaaat aagtttcttc tatacataac aattttgaat acatgaacca aagattaatt |
| 1741 | aaaaatatca cttgaagaga tccaaccaca tcaaagatga tgcagcttga gtaattgtta |
| 1801 | actctttcca actttggaaa gagacaaaac gtcaaagtga tttccagaaa cagagaacgg |
| 1861 | aagctcaaaa gtctctcttg tgccaaatat attaaaccct aaaaaattca acttctatcc |
| 1921 | gaatttctca gaaacaaaac aaagacacat aacttcatat gaaattccag tgaacggtta |
| 1981 | cctgaggaag aaccttattt gttatcagtg taaaataaca gagaagaggt acgagtaaaa |
| 2041 | gaaacaaaag tgacttagga aacgccattg ttggagactg ctcactggaa gatagagagt |
| 2101 | cgtgagagac agtgataaag cgtatcaagt catatagagg gtcttcttat ctttttcttt |
| 2161 | aacatgtgag ggttgagtta attatgcggg ctgattatag agtttttaaa ttgaatttac |
| 2221 | gattgttttt ttcttacata taaatgcaat ctatatttgt gttcggaata acccatctaa |
| SEQ ID NO: 26: AtNAR2.2 promoter | |
| 481 | atttctgtga tatctaagag aagaattcaa aggttaataa gaataaagca ataatgactt |
| 541 | gtcttataac gaaaaccaaa aactttcaga ataaaacctt ttgaagagtt tggctaagat |
| 601 | cttgttgccg cagagtcact atatatgctt gcaaccataa taaaaaaaaa gaggatcatt |
| 661 | taagattgat cactatgaca attttatcga tgttaattat gaaccttctt atagagttac |
| 721 | ctgagtttta tttaggttta gaccaagcaa cttcccatcg atctgaacac aagtaaaaaa |
| 781 | aaagtaaaga aagcttttga atatgtacat acatcagatc caagggacac catgtttctt |
| 841 | acattttcaa gctttgagcc aagttccgcg actttctttt ctgcgatttc agctcgagtt |
| 901 | tccacttcca gacttctctt gcgttgcatt tccacttctt ctcgtagttc aagtatctgc |
| 961 | caatgcagaa ctaaattgag gtaacaaaac aagaaggaaa tacacacctc aaactacaaa |
| 1021 | acaccaacct gtttctgaag ttcataagcc gttccttctg cttcactgga ttctcagtct |
| 1081 | aaataaaaaa gcatcaacag caacaaaagt gtaaataagt ttcttctata cataacaatt |
| 1141 | ttgaatacat gaaccaaaga ttaattaaaa atatcacttg aagagatcca accacatcaa |
| 1201 | agatgatgca gcttgagtaa ttgttaactc tttccaactt tggaaagaga caaaacgtca |
| 1261 | aagtgatttc cagaaacaga gaacggaagc tcaaaagtct ctcttgtgcc aaatatatta |
| 1321 | aaccctaaaa aattcaactt ctatccgaat ttctcagaaa caaaacaaag acacataact |
| 1381 | tcatatgaaa ttccagtgaa cggttacctg aggaagaacc ttatttgtta tcagtgtaaa |
| 1441 | ataacagaga agaggtacga gtaaaagaaa caaaagtgac ttaggaaacg ccattgttgg |
| 1501 | agactgctca ctggaagata gagagtcgtg agagacagtg ataaagcgta tcaagtcata |
| 1561 | tagagggtct tcttatcttt ttctttaaca tgtgagggtt gagttaatta tgcgggctga |
| 1621 | ttatagagtt tttaaattga atttacgatt gtttttttct tacatataaa tgcaatctat |
| 1681 | atttgtgttc ggaataaccc atctaatatt actccatgta ttaaactaaa atattttgca |
| 1741 | tgttttggta gatcaacttt ttgaatgatc acagacctac aaacatcaac cctctaatta |
| 1801 | tccaatttta ccataatcca cgagctctta aaattcattt ttaatatata taattaaaat |
| 1861 | agttcaaaca gtttaaactt tgtgaccaag ttaaaatatt taaaatagtt tgactttgtg |
| 1921 | atcaacatat ttaaatataa tcaatatttt catttttaag ccggaaaatc acgtcttaca |
| 1981 | aatatatttc tgatagacac acctataatt ccaaaatttt gacttttaaa acaaacaaaa |
| 2041 | aaaaatctca ttaataccac tacatacgtt tttacaaaaa taccattaaa agatattttt |
| 2101 | tcaaatacta aaaaaaacta aaactaaact ttaaacctat aaaacactaa atcctataaa |
| 2161 | gtttatattc gagtataaac cttaaaagtt caaccctaaa tcctgaaagc aagaccctaa |
| 2221 | acccaaactc aaacttttaa attataaatc cttaaacaaa atcattttta gtctttatgg |

Hordeum Vulgare

| SEQ ID NO: 27: HvNAR2.1 AY253448.1 mRNA, complete cds | |
|---|---|
| 1 | aaccttctct caccgagcag ctgcgagctc cagcccaatt tccaagctag aatggcacgg |
| 61 | tcggagctgg tcatggtgtt gctagtggtg gtcctcgccg ccggctgctg cacgtcggcg |
| 121 | ggcgccgtgg cgtacctctc caagctgcct gttaccctcg acgtcaccgc atcccccagt |
| 181 | cccggccaag ttcttcacgc cggcgaggac gtgatcacgg tgacatgggc cttgaacacg |
| 241 | acacaggccg gcaaggacgc cgactacaag aacgtgaagg tgagcctctg ctacgcgccg |
| 301 | gtgagccaga aggagcgcga gtggcgcaag acccacgacg acctcaagaa ggacaagacc |
| 361 | tgccagttca aggtcaccca gcaggcctac cccggcgccg gcaaggtcga gtaccgcgtc |
| 421 | gccctcgaca tccccaccgc cacctactac gtgcgcgcct acgcgctgga cgcctcgggc |
| 481 | acccaggtcg cctacggcca gaccgcgccc accgcggcct tcaatgttgt cagcatcacc |
| 541 | ggcgtcacca cctctatcaa ggtcgctgct ggcgttttct ccgccttctc cgtggcctcc |
| 601 | ctcgccttct tcttcttcat tgagaaacgc aagaagaaca actaaactcg caagccggaa |
| 661 | ccgtggcatg gcaaggtgtt cgtccgtgcg cgacttcttc ccgtcatttg taacgtacgc |
| 721 | acggctgtac tgtaccgtac atgtaagaga ttgctggtat tccctttctc ggagactccc |
| 781 | gtaaaaaaaa aaaaaaaaaa aaaaa |
| SEQ ID NO: 28: HvNAR2.2 AY253449.1 mRNA, complete cds | |
| 1 | ccacgcgtcc gcagccccat ccatcaaacc ttctctgacc gagcagcagc tgcgagctcg |
| 61 | agcccgcccc aagttcgacg acggcgatgg cacggtcgga cctggtcatg gcgttgctgg |
| 121 | tggccgtcct cgccgccggc tgctgcgcgt cggccggcgc cgtggcgtac ctctccaagc |
| 181 | tgaccgtgac cctcgacgtc accgcctccc ccactcctgg ccaagttctc cacgccggcg |
| 241 | aggacgtgat cacggtgacg tgggccctga acgcgacccg gccggccggc gacgacgccg |
| 301 | cctacaagag cgtcaaggtc agcctctgct acgcgccggc gagccagaag gagcgcgagt |
| 361 | ggcgcaagac ccacgacgac ctcaagaagg acaagacctg ccagttcaag gtcacccagc |
| 421 | agccctacgc cgccggcgcc gccggcggca gggtcgagta ccgcgtcgcc ctcgacatcc |
| 481 | ccaccgccgc ctactacgtg cgcgcctacg cgctcgacgc ctccggcacg caggtggcct |
| 541 | acggccagac cgcgcccgcc accgccttcg acgtcgtcag catcacgggc gtcaccacct |
| 601 | ccatcaaggt cgccgccggc gtcttctcca ccttctccgt cgtctccctc gccttcttct |
| 661 | tcttcatcga gaagcgcaag aagaataact aaactcacaa ggtgttcgtc cgtgcgcggc |
| 721 | tgcttcttct tcttcttctt cttgtcattt gcaacgtaca cataatatta ctgtatgtaa |
| 781 | gagagtagtg ttgatgttct cttttcgggg agctcacgtg attgttgatg ttgtaccaga |
| 841 | ggagttgcaa gctggagtgt atgttagatt gttagtacgt caaaaaaaaa aaaaaaaaa |
| SEQ ID NO: 29: HvNAR2.3 AY253450.1 mRNA, complete cds | |
| 1 | atggctcggc aaggcatggt cacggcgctg ctgctgctgg tcctcgccgc cggctgctgc |
| 61 | gcatcggcgg gcgccgtggc gtacctctcc aagctgcctg tgaccctcga cgtcaccgcc |
| 121 | tcccccactc ccggccaagt tcttcacgcc ggcgaggacg tgatcacggt gacgtgggcc |
| 181 | ctgaacgcga gccagccggc cggcaaggac gccgactaca agaacgtgaa ggtgagcctc |
| 241 | tgctacgcgc cggtgagcca gaaggagcgc gagtggcgca aaacccacga cgacctcaag |
| 301 | aaggacaaga cctgccagtt caagatcacc cagcaggcct accccggcgc cggcaaggtt |
| 361 | gagtatcgcg tcgccctcga catccccacc gccacttact acgtgcgcgc ctacgcgctc |
| 421 | gacgcctcgg gcacccaggt cgcctacggc caaaccgcgc caaccgccgc cttcaacgtc |
| 481 | gtcagcatca cgggagtcac cacctccatc aaggtcgccg ccggcgtctt ctccgccttc |
| 541 | tccgtcgcct ccctcgcctt cttcttcttc attgagaaac gcaagaagaa caactaa |
| SEQ ID NO: 30: HVNRT2.1 U34198.1 MRNA, COMPLETE CDS | |
| 1 | gaattcgcgg ccgctccctt actacattgc aagccaagct caagagcagc agcaacagcc |
| 61 | accattagct gcttctagtt gttggcaaag atggaggtcg aggcgggcgc ccatggcgac |
| 121 | actgccgcga gcaagttcac gctgccggta gactccgagc acaaggccaa gtccttcagg |
| 181 | ctcttctcct tcgccaaccc gcacatgcgc accttccatc tctcgtggat ctccttcttc |
| 241 | acttgcttca tctccacctt cgccgcagcg ccccttgtcc ccatcattcg tgataacctc |
| 301 | aaccttgcca aggccgacat cggcaatgcc ggtgtggcat ccgtttctgg gtccatcttc |
| 361 | tccaggcttg ccatgggtgc catctgcgat ctcctcgggc cgcggtatgg atgtgcattc |
| 421 | ctcgtcatgc tctcggcacc gaccgttttc tgcatggccg ttatcgatga tgcctcaggg |
| 481 | tacatcgccg tccgctttct cattggcttc tcgcttgcta cgttcgtgtc atgccaatat |
| 541 | tggatgagca ccatgtttaa tagcaagatc atcggcacag tcaacggcct cgctgctgga |
| 601 | tggggcaaca tgggtggtgg cgccacgcag ctcatcatgc cgctcgtctt ccatgcaatc |
| 661 | cagaagtgtg gtgccacgcc cttcgtagcg tggcgtattg cctacttcgt gcccggaatg |
| 721 | atgcacatcg tgatgggctt gttggtactc accatggggc aagatctccc tgatgggaac |
| 781 | ctcgcaagtc tccagaagaa gggagacatg gccaaggaca agttctccaa ggtcctttgg |
| 841 | ggcgccgtta ccaactaccg aacatggatc tttgtcctcc tctatggcta ctgcatgggt |
| 901 | gtcgagctca ccaccgacaa tgtcattgcc gagtactact tcgaccactt ccacctagac |
| 961 | ctccgtgccg ccggtaccat cgctgcctgc ttcggcatgg ccaacatcgt cgcacgtcct |
| 1021 | acgggtggct acctctctga ccttggcgcc cgctatttcg gcatgcgtgc tcgcctctgg |
| 1081 | aatatctgga tcctccaaac cgctggtggc gctttctgca tctggctcgg tcgtgcatcg |
| 1141 | gccctccctg cctcggtgac cgccatggtc ctcttctcca tctgcgccca ggctgcgtgt |
| 1201 | ggtgctatct ttggtgtcgc acccttcgtc tccaggcgtt cccttggcat catttccggg |
| 1261 | ttgaccggtg ctggtggaaa cgtgggcgca gggctcacac agcttctctt cttcacgtca |
| 1321 | tcgcaatact ccactggtag gggtctcgag tacatgggca tcatgatcat ggcatgcacg |
| 1381 | ctgcccgtcg ctcttgtgca cttcccacaa tggggatcca tgttcttccc tgccagcgcc |
| 1441 | gacgccacgg aggaggagta ctacgcctcg gagtggtccg aagaggagaa agccaagggt |
| 1501 | ctccatatcg ccggccaaaa atttgctgag aattcccgct cggagcgcgg taggcgcaac |
| 1561 | gtcatccttg ccacgtccgc cacaccaccc aacaatacgc cccagcacgt atgagactgg |
| 1621 | attgtttttc atacctatgt acaagtactg aactacagtg cacgttcgta tatatatacg |
| 1681 | cctgcaacat cggctgtaat aaggcgtatg aatttacatt tgtagtgtag gcctgtgtaa |
| 1741 | tgcgtttctt acgcacgaaa tgtttggtct gtgcatgcac gcatgcgagg gtacctgtgc |
| 1801 | tctgaattta caacagcttt gaggcggccg cgaattc |
| SEQ ID NO: 31: HVNRT2.2 (HVBCH2) U34290.1 MRNA, COMPLETE CDS | |
| 1 | ttacaagctc catctgagag cagcagcaac caccattaga gacacactta gttgccagtg |
| 61 | cgactaagct agctagctcg aggaagatgg aggtggagtc gagctcgcat ggcgccggcg |
| 121 | acgaggctgc gagcaagttc tcgctgcccg tggactcgga gcacaaggcc aagtccatca |
| 181 | ggctcttctc cttcgccaac ccccacatgc gcaccttcca cctctcctgg atctccttct |
| 241 | tcacctgctt cgtctccacc ttcgctgccg cgcccctcgt ccctatcatc cgcgacaacc |
| 301 | taaacctcgc caaggccgac atcggcaacg ccggtgtggc gtccgtgtcc gggtctatct |
| 361 | tctcgaggct cgccatgggg gccatctgcg atctccttgg ccctcgatat ggatgcgcct |
| 421 | tcctcgtcat gctcgcagca cccaccgtct tctgcatgtc cctcatcgat gatgcggcgg |
| 481 | gctacatcac ggtccgcttc ctcatcggct tctccctcgc gacgtttgtg tcgtgccagt |
| 541 | attggatgag caccatgttc aacagcaaga tcatcggcac cgtcaacggc ctggcggccg |
| 601 | gctggggcaa catgggtggt ggtgccaccc agctcattat gccactcgtc ttccacgcca |
| 661 | tccagaagtg tggtgccacg cccttcgtcg catggcgcat cgcctacttc gtgccaggaa |
| 721 | tgatgcacgt ggtgatgggc ttgctcgtgc tcaccatggg acaggatctc cccgatggta |
| 781 | accttgcaag cctccagaag aagggggaga tggccaagga caagttctcc aaggttgtgt |
| 841 | ggggtgctgt tacaaactac cgtacatgga tcttcgttct tctttacgga tactgcatgg |
| 901 | gtgttgagct caccaccgac aacgtcatcg ccgagtacta cttcgaccac tttcaccttg |
| 961 | accttcgaac atccggcacc attgccgcct gttttggcat ggccaacatc gttgctcggc |
| 1021 | ctgcgggtgg ctacctctcc gacctcggtg cccgctactt cggcatgcgt gcccgcctct |
| 1081 | ggaacatctg gatcctccag accgctggtg gcgcattctg cctctggctc ggccgtgcaa |
| 1141 | aagccctccc cgaatccatc actgccatgg tcctcttctc catctgcgct caggcagcat |
| 1201 | gtggtgcagt ctttggtgtc atccccttcg tctcccgccg ctccctcggc atcatttcgg |
| 1261 | gcttgagtgg agccggtggg aactttggcg ccgggctgac acaattgctc ttcttcactt |
| 1321 | cgtcgaagta tggcaccggc agggggcttg agtacatggg tatcatgatc atggcctgca |
| 1381 | cgctccctgt ggcgcttgtg cacttcccac agtggggttc catgctcttg ccgccaaacg |
| 1441 | ccaacgccac cgaggaggag ttctatgccg ccgaatggag cgaggaggag aagaagaagg |
| 1501 | gtctccatat ccctggccaa aagtttgccg agaattcccg ctcggagcgt ggcaggcgca |
| 1561 | acgtcatcct tgccacagcc gccacacccc ccaacaacac tccccaacac gcataagact |
| 1621 | cgagcttttc tttacctgtg tacacgtaca gtgcgcgtat tatacacaca tcgatcgtgt |
| 1681 | atatacgcct ggaatccgca agcagtatgt tttttgaaaa aaaaaaagcg gccgcgaatt |
| 1741 | c |
| SEQ ID NO: 32: HVNRT2.3 (HVBCH3) AF091115.1 MRNA, COMPLETE CDS | |
| 1 | tctcagttgc cactgcagct gatcaagcaa gctagctcca aacctccaag gaggaagcag |
| 61 | agaaggagac tagctcgatc aagcaaggtc caaatggagg tggaggctgg tgcccatggc |
| 121 | gacacggcgg cgagcaagtt cacgttgccc gtggactccg agcacaaggc caagtccttc |
| 181 | aggctcttct ccttcgccaa cccacacatg cgcacctttc acctatcgtg gatatccttc |
| 241 | ttcacatgct tcgtctccac ctttgccgcg gcgcccctgg tgcccatcat ccgcgacaac |
| 301 | ctgaacctcg ccaaggccga catagggaat gccggtgtgg catctgtgtc tgggtctatc |
| 361 | ttctcgaggc ttgccatggg cgccatctgc gaccttttgg ggccgcggta tgggtgtgcc |
| 421 | ttcctcgtca tgctctcagc gccaaccgtc ttctgcatgg ccgtcatcga tgacgcctca |
| 481 | gggtacatcg ccgtacgctt cctcatcggc ttctcccttg ccacctttgt gtcgtgccaa |
| 541 | tactggatga gcaccatgtt caacagtaaa atcatcggca cggtcaatgg cctcgcggcc |
| 601 | ggctggggca acatgggcgg tggtgccaca caactcatca tgccgcttgt tttccacgcc |
| 661 | atccaaaaat gtggtgccac accatttgtg gcatggcgta ttgcctactt cgtgcccgga |
| 721 | atgatgcaca tcgtgatggg cttgctggta ctcaccatgg ggcaagatct ccctgatggg |
| 781 | aacctcgcga gcctccagaa gagaggagac atggccaagg acaagttctc caaggtcctt |
| 841 | tggggcgccg tcaccaacta ccggacatgg atctttgtcc tcctatatgg ctactgcatg |
| 901 | ggtgtcgaac tcaccactga caatgtcatt gccgagtact acttcgacca cttccaccta |
| 961 | gaccttcgcg ccgctggtac catcgccgcc tgcttcggta tggccaacat agtcgcacgt |
| 1021 | cctatgggcg gctacctctc tgaccttggc gcccgctatt tcggcatgcg tgccctttgg |
| 1081 | aacatctgga tcctccaaac cgctggtggc gctttctgca tctggctcgg tcgtgcatcg |
| 1141 | gccctccctg cctcggtgac cgccatggtc ctcttctcca tctgtgccca ggctgcctgt |
| 1201 | ggtgctatct ttggtgtcgc acccttcgtc tccaggcgtt cccttggcat catttccggg |
| 1261 | ttgaccggtg ccggtggaaa cgtgggcgca ggactcacac aacttctatt cttcacctca |
| 1321 | tcgcaatact ccactggtag gggtctcgag tacatgggca tcatgatcat ggcatgcacg |
| 1381 | ctgcccgtcg ctcttgtgca ctttccgcaa tggggatcca tgttcttccc ggccagcgct |
| 1441 | gatgccactg aggaggagta ctatgcttcc gagtggtcgg aggaggagaa gggcaagggt |
| 1501 | ctccatatcg caggccaaaa gttcgccgag aactcccgct cggagcgcgg caggcgcaac |
| 1561 | gtcatctttg ccacatccgc cacgccgccc aacaacacac cccagcaggt ataaggcatt |
| 1621 | tttttttgtt acctatgaat tttacagctc atggcgtata tatacaaaca gtatatttac |
| 1681 | gtttgcagcc ccagcgtaat aagttgtatg ggggtttatc tttttactat ggtaaaccta |
| 1741 | aggacatgta ttgtcaaatt gagtccgaaa ttaatacatg aacagtgttg atgtttgtgt |
| 1801 | atgcttgaaa aaaaaaaaaa aaaaa |
| SEQ ID NO: 33: HVNRT2.4 (HVBCH4) AF091116.1 MRNA, COMPLETE CDS | |
| 1 | caccactgca agcatattta ggcttagtta gctccaagga gcaaagctaa aaagaaccta |
| 61 | gctaggctag ctcgatccag ctagctcagt agatatggag gtggaggccg gagctcatgg |
| 121 | cgatgcggcg gcgagcaagt tcacgctgcc cgtggactcc gagcacaagg ccaagtcctt |
| 181 | caggctcttc tccttcgcca acccgcacat gcgcaccttc cacctctcgt ggatctcctt |
| 241 | cttcacctgc ttcgtctcca cctttgccgc tgctccgttg gtgcccatca tccgcgacaa |
| 301 | cctcaacctc gccaaggccg acatcggcaa tgccggtgtg gcgtccgtgt ccggctccat |
| 361 | cttctcgagg ctcgccatgg gcgccatttg tgacctgctt ggcccgcggt acggttgtgc |
| 421 | ctttctcgtc atgctatcgg cgccaaccgt cttctgcatg gccgtcatcg acgacgcgtc |
| 481 | gggatacatc gcagtccgct tcctcatcgg cttctccctc gcaaccttcg tgtcatgcca |
| 541 | gtactggatg agcacaatgt tcaacagtaa aatcatcggc acggttaatg gcctcgcagc |
| 601 | cgggtggggc aacatgggtg gcggggccac acagctcatc atgcccctcg tcttccatgc |
| 661 | catccaaaag tgtggtgcca caccctttgt ggcatggcgt atcgcctact tcgtgccggg |
| 721 | gatgatgcac atcgtgatgg gcctactcgt gctcaccatg ggacaagacc tccctgatgg |
| 781 | gaacctcgca agcctgcaga agaagggaga catggccaag gacaagttct ccaaggtcct |
| 841 | ttggggcgcc gttaccaact accggacatg gatctttgtc ctcctctatg gctactgcat |
| 901 | gggtgtcgag ctcaccactg gcaatgtcat tgccgagtac tacttcgatc acttccacct |
| 961 | aaacctccgt gccgccggta ccatcgccgc ttgcttcggc atggccaaca tcgtcgcacg |
| 1021 | tcctatgggc ggctacctct ccgaccttgg tgctcgctac ttcggtatgc gtgctcgcct |
| 1081 | ttggaacatc tggatccttc agacagctgg cggcgccttt tgcatctggc ttgggcgcgc |
| 1141 | ctcggccctc cccgcctcag tgactgccat ggtcctcttc tccatctgcg cccaggctgc |
| 1201 | gtgtggtgct atctttggtg tcgaaccctt cgtctccagg cgttcccttg gcatcatttc |
| 1261 | cgggttgacc ggtgctggtg gaaacgtggg cgcagggctc acacagcttc tcttcttcac |
| 1321 | ttcgtcgcaa tactccactg gcaggggtct tgagtacatg ggcatcatga tcatggcatg |
| 1381 | caccttaccc gtcgctctcg ttcacttccc tcagtggggc tctatgttct tggctgccag |
| 1441 | tgccgacgcc acggaggagg agtactacgc ctcagagtgg tcagaggagg agaagagcaa |
| 1501 | gggtctccat atcgcaggac aaaagtttgc tgagaactcc cgctcggaac gcggcaggcg |
| 1561 | caacgtcatc cttgccacat ccgccacacc acccaacaac acgcccctac acgtataagt |
| 1621 | ttcaaatttt gtgttacaca agaaatgtac atcttgctga gtatatatac acatcgtata |
| 1681 | ttttagtaaa aaaaaaaaaa aaaa |
| | SEQ ID NO: 34: HvNAR2.1 translation |
| | MARSELVMVLLVVVLAAGCCTSAGAVAYLSKLPVTLDVTASPSP |
| | GQVLHAGEDVITVTWALNTTQAGKDADYKNVKVSLCYAPVSQKEREWRKTHDDLKKDK |
| | TCQFKVTQQAYPGAGKVEYRVALDIPTATYYVRAYALDASGTQVAYGQTAPTAAFNVV |
| | SITGVTTSIKVAAGVFSAFSVASLAFFFFIEKRKKNN |
| | SEQ ID NO: 35: HvNAR2.2 translation |
| | MARSDLVMALLVAVLAAGCCASAGAVAYLSKLTVTLDVTASPTP |
| | GQVLHAGEDVITVTWALNATRPAGDDAAYKSVKVSLCYAPASQKEREWRKTHDDLKKD |
| | KTCQFKVTQQPYAAGAAGGRVEYRVALDIPTAAYYVRAYALDASGTQVAYGQTAPATA |
| | FDVVSITGVTTSIKVAAGVFSTFSVVSLAFFFFIEKRKKNN |
| | SEQ ID NO: 36: HvNAR2.3 translation |
| | MARQGMVTALLLLVLAAGCCASAGAVAYLSKLPVTLDVTASPTP |
| | GQVLHAGEDVITVTWALNASQPAGKDADYKNVKVSLCYAPVSQKEREWRKTHDDLKKD |
| | KTCQFKITQQAYPGAGKVEYRVALDIPTATYYVRAYALDASGTQVAYGQTAPTAAFNV |
| | VSITGVTTSIKVAAGVFSAFSVASLAFFFFIEKRKKNN |
| | SEQ ID NO: 37: HvNRT2.1 translation |
| | MEVEAGAHGDTAASKFTLPVDSEHKAKSFRLFSFANPHMRTFHL |
| | SWISFFTCFISTFAAAPLVPIIRDNLNLAKADIGNAGVASVSGSIFSRLAMGAICDLL |
| | GPRYGCAFLVMLSAPTVFCMAVIDDASGYIAVRFLIGFSLATFVSCQYWMSTMFNSKI |
| | IGTVNGLAAGWGNMGGGATQLIMPLVFHAIQKCGATPFVAWRIAYFVPGMMHIVMGLL |
| | VLTMGQDLPDGNLASLQKKGDMAKDKFSKVLWGAVTNYRTWIFVLLYGYCMGVELTTD |
| | NVIAEYYFDHFHLDLRAAGTIAACFGMANIVARPTGGYLSDLGARYFGMRARLWNIWI |
| | LQTAGGAFCIWLGRASALPASVTAMVLFSICAQAACGAIFGVAPFVSRRSLGIISGLT |
| | GAGGNVGAGLTQLLFFTSSQYSTGRGLEYMGIMIMACTLPVALVHFPQWGSMFFPASA |
| | DATEEEYYASEWSEEEKAKGLHIAGQKFAENSRSERGRRNVILATSATPPNNTPQHV |
| | SEQ ID NO: 38: HvNRT2.2 (HvBCH2) translation |
| | MEVESSSHGAGDEAASKFSLPVDSEHKAKSIRLFSFANPHMRTF |
| | HLSWISFFTCFVSTFAAAPLVPIIRDNLNLAKADIGNAGVASVSGSIFSRLAMGAICD |
| | LLGPRYGCAFLVMLAAPTVFCMSLIDDAAGYITVRFLIGFSLATFVSCQYWMSTMFNS |
| | KIIGTVNGLAAGWGNMGGGATQLIMPLVFHAIQKCGATPFVAWRIAYFVPGMMHVVMG |
| | LLVLTMGQDLPDGNLASLQKKGEMAKDKFSKVVWGAVTNYRTWIFVLLYGYCMGVELT |
| | TDNVIAEYYFDHFHLDLRTSGTIAACFGMANIVARPAGGYLSDLGARYFGMRARLWNI |
| | WILQTAGGAFCLWLGRAKALPESITAMVLFSICAQAACGAVFGVIPFVSRRSLGIISG |
| | LSGAGGNFGAGLTQLLFFTSSKYGTGRGLEYMGIMIMACTLPVALVHFPQWGSMLLPP |
| | NANATEEEFYAAEWSEEEKKKGLHIPGQKFAENSRSERGRRNVILATAATPPNNTPQHA |
| | SEQ ID NO: 39: HvNRT2.3 (HvBCH3) translation |
| | MEVEAGAHGDTAASKFTLPVDSEHKAKSFRLFSFANPHMRTFHL |
| | SWISFFTCFVSTFAAAPLVPIIRDNLNLAKADIGNAGVASVSGSIFSRLAMGAICDLL |
| | GPRYGCAFLVMLSAPTVFCMAVIDDASGYIAVRFLIGFSLATFVSCQYWMSTMFNSKI |
| | IGTVNGLAAGWGNMGGGATQLIMPLVFHAIQKCGATPFVAWRIAYFVPGMMHIVMGLL |
| | VLTMGQDLPDGNLASLQKRGDMAKDKFSKVLWGAVTNYRTWIFVLLYGYCMGVELTTD |
| | NVIAEYYFDHFHLDLRAAGTIAACFGMANIVARPMGGYLSDLGARYFGMRALWNIWIL |
| | QTAGGAFCIWLGRASALPASVTAMVLFSICAQAACGAIFGVAPFVSRRSLGIISGLTG |
| | AGGNVGAGLTQLLFFTSSQYSTGRGLEYMGIMIMACTLPVALVHFPQWGSMFFPASAD |
| | ATEEEYYASEWSEEEKGKGLHIAGQKFAENSRSERGRRNVIFATSATPPNNTPQQV |
| | SEQ ID NO: 40: HvNRT2.4 (HvBCH4) translation |
| | MEVEAGAHGDAAASKFTLPVDSEHKAKSFRLFSFANPHMRTFHL |
| | SWISFFTCFVSTFAAAPLVPIIRDNLNLAKADIGNAGVASVSGSIFSRLAMGAICDLL |
| | GPRYGCAFLVMLSAPTVFCMAVIDDASGYIAVRFLIGFSLATFVSCQYWMSTMFNSKI |
| | IGTVNGLAAGWGNMGGGATQLIMPLVFHAIQKCGATPFVAWRIAYFVPGMMHIVMGLL |
| | VLTMGQDLPDGNLASLQKKGDMAKDKFSKVLWGAVTNYRTWIFVLLYGYCMGVELTTG |
| | NVIAEYYFDHFHLNLRAAGTIAACFGMANIVARPMGGYLSDLGARYFGMRARLWNIWI |
| | LQTAGGAFCIWLGRASALPASVTAMVLFSICAQAACGAIFGVEPFVSRRSLGIISGLT |
| | GAGGNVGAGLTQLLFFTSSQYSTGRGLEYMGIMIMACTLPVALVHFPQWGSMFLAASA |
| | DATEEEYYASEWSEEEKSKGLHIAGQKFAENSRSERGRRNVILATSATPPNNTPLHV |

Zea Mays

| SEQ ID NO: 41: ZmNAR2.1 AY968678.1 mRNA, complete cds | |
|---|---|
| 1 | gctcagatcc ctcgcctcgt gtcgtgtctc cggtcgacga cgaccaacag ccagtgtggg |
| 61 | ccagacggac accgccgagc tatagcgctt ggtgatagca agggacgacc ggcggccgga |
| 121 | ccggagcacg tacgtacgta ccgcagcgat ggctcggcag caaagcgtgc acgccttgtg |
| 181 | tgtgctggcg gcacttctct tcgccgcctc cctgccgtcg ccggccgccg cgggggtgca |
| 241 | cctctcctcg ctgcccaaag cgctcgacgt caccacctcc gccaaacccg gccaagtcct |
| 301 | gcacgccggc gtggactcgc tgacggtgac gtggagcctg aacgccacgg agccggccgg |
| 361 | cgccgacgcc gggtacaagg gcgtgaaggt gaagctgtgc tacgcgccgg cgagccagaa |
| 421 | ggaccgcggg tggcgcaagt ccgaggacga catcagcaag gacaaggcgt gccagttcaa |
| 481 | ggtcaccgag caggcgtacg cggcggcggc gcccggcagc ttccagtacg ccgtcgcccg |
| 541 | cgacgtcccc tcgggctcct actacctgcg cgccttcgcc acggacgcgt cgggcgccga |
| 601 | ggtggcctac ggccagacgg cgcccaccgc cgccttcgac gtcgccggca tcaccggcat |
| 661 | ccacgcctct ctcaagatcg ccgccggcgt cttctcggcc ttctccgtcg tcgcgctcgc |
| 721 | cttcttcttc gtcatcgaga cccgcaagaa gaacaagtag aacgagttgc ggctgcgcgc |
| 781 | catacatgca tacatgtaaa tcgtcggcgg cgatgagtgg ctgtcgttgc tgattcattg |
| 841 | gtgcgcgcga ctattttggt gtatcatgta agttactttt ctgcagtgtg tgcgtcaaaa |
| 901 | ttaccaaata ataacttaag tttctctaca taaaaaaaaa aaaaaaaaaa aaaaaaaaaa |
| 961 | a |
| SEQ ID NO: 42: ZmNAR2.2 AY968679.1 mRNA, complete cds | |
| 1 | cctcgtcaca caccacaggc tgtgtagagc gcgcgcctgg catgacgatg gctcgtcctg |
| 61 | gggcggcttt gccgctgctg ctggtcgtgg tcggcgcttg ctgcgcgcgc ctggcggcgg |
| 121 | cagtgcacct ctccgcgctc ggcaggacac tcatcgtcga ggcgtcgccg aaggccggac |
| 181 | aagtcctgca cgccggcgag gacacgataa ccgtgacatg gcacctcaac gcgtcggcgt |
| 241 | ccagcgtcgg gtacaaggcg ctggaggtga ccctctgcta cgcgccggcg agccaggagg |
| 301 | accgcgggtg gcgcaaggcc aacgacgact tgagcaagga caaggcgtgc cagttcagga |
| 361 | tcgcccggca tgcatacgcc ggcggccagg ggacgctccg gtacagggtc gcccgcgacg |
| 421 | tccccaccgc gtcctaccac gtgcgcgcct acgcgctgga cgcgtccggg gcgccggtgg |
| 481 | gctacggcca gaccgcgccc gcctactact tccacgtcgc gggcgtctcg ggcgtccacg |
| 541 | cgtccctccg ggtcgccgcc gccgtgctct ccgcgttctc catcgccgcg ctcgccttct |
| 601 | ttgtcgtcgt cgagaagagg aggaaggacg agtaggccgt aagcggcaca cgcgatatat |
| 661 | accagggagg cgccaccgtg cgagctagcg cttggtgagc cgtcatgttg tgtagtgcag |
| 721 | ttgtaacaca agtattaagc taagcacaag tcgtaccatg tatccagctc cagcatgaat |
| 781 | ccaaacccag cgtcagtcac aagctggacc tgcatggtgc ctgtcaacca aaattcttct |
| 841 | tggatcatac taatactatc gtctacagca tatagaactt gtatcatact aatttacatg |
| 901 | gcaccttctg ctaaaaaaaa aaaaaaaaaa aaaaaa |
| SEQ ID NO: 43: ZmNRT2.1 AY129953.1 mRNA, complete cds | |
| 1 | acgcggggaa gcacaagcaa ccagccagct agtttccaag ggatcacctg ctctctagca |
| 61 | ctagcagcaa tggcggccgt cggcgctccg ggcagctctc tgcacggagt cacggggcgc |
| 121 | gagccggcgt tcgccttctc cacggagcac gaggaggcgg cgagcaatgg tggcaagttc |
| 181 | gacctgccgg tggactcaga gcacaaggcg aagagcgtcc gtctcttctc cgtggcgaac |
| 241 | ccacacatgc gcaccttcca cctctcctgg atctccttct tcacctgctt cgtgtccacc |
| 301 | ttcgccgccg cgccgctggt ccccatcatc cgcgacaacc tcaacctcac caaggccgac |
| 361 | atcggcaacg cgggcgtggc ctcggtgtcg ggctccatct tctcccgcct caccatgggc |
| 421 | gccgtctgcg acctgctggg cccgcgctac ggctgcgcct tcctcatcat gctgtccgcg |
| 481 | cccaccgtgt tctgcatgtc gctcatcgac gacgccgcgg gctacatcac cgtcaggttc |
| 541 | ctcatcggct tctccctcgc caccttcgtc tcctgccagt actggatgag caccatgttc |
| 601 | agcagcaaga tcatcggcac cgtcaacggg ctcgccgccg gatggggcac aatgggaagg |
| 661 | cggcgccacg cagctcatat gccgctcgtc tacgacgtca tccgcaagtg cggcgccacg |
| 721 | ccattcacgg cctggcgcct cgcctacttc gtgccgggcc tcatgcacgt cgtcatgggc |
| 781 | gtcctggtgc tcacgctggg gcaggacctc cccgacggca acctcaggtc gctgcagaag |
| 841 | aagggcaacg tcaacaagga cagcttctcc aaggtcatgt ggtacgccgt catcaactac |
| 901 | cgtacctgga tctttgtcct cctctacggc tactgcatgg gcgtcgagct caccaccgac |
| 961 | aacgtcatcg ccgagtacat gtacgaccgc ttcgacctcg acctccgcgt cgctgggacc |
| 1021 | atcgccgcct gcttcggcat ggccaacatc gtcgcacgcc ccatgggcgg catcatgtcc |
| 1081 | gacatgggcg cgcgctactg gggcatgcgc gctcgcctct ggaacatctg gatcctccag |
| 1141 | accgccggcg gcgccttctg cctctggctg gggcgcgcca gcaccctccc cgtctccgtc |
| 1201 | gtcgccatgg tgctcttctc cttctgcgcg caggcggcat gcggcgccat cttcggggtt |
| 1261 | atcccctttg tctcccgccg ctccctcggc atcatctccg gcatgacggg cgccggcggc |
| 1321 | aacttcggcg ccgggctcac gcagctgctc ttctttacct cctcgaccta ctccacgggc |
| 1381 | agggggctgg agtacatggg catcatgatc atggcgtgca cgctgcccgt ggtgttcgtg |
| 1441 | cacttccctc agtgggggtc catgttcttt ccgcccagcg ccaccgccga cgaggagggc |
| 1501 | tactacgcct ccgagtggaa cgacgacgag aagagcaagg gactccatag cgccagcctc |
| 1561 | aagttcgccg agaacagccg ctcagagcgc ggcaagcgaa acgtcatcca ggccgacgcc |
| 1621 | gccgccacgc cggagcatgt ctaagtctac tactaagatg gatcgatcga cgatcaccta |
| 1681 | tacctctttg tatgtacgaa tatgccttgt tattactgcg cgcgcgcata tacaatacac |
| 1741 | gtgtgctccg ttgacatgag ttagaaaaaa aaaaaaaaaa aaaaaaaaa |
| SEQ ID NO: 44: ZmNRT2.2 AY559405.1 mRNA, complete cds | |
| 1 | atggcggccg tcggcgctcc ggggagctct ctgcacggag tcacggggcg cgagccggcg |
| 61 | ttcgcattct ccacggagca cgaggaggcg gcgagcaatg gcggcaagtt cgacctgccg |
| 121 | gtggactcgg agcacaaggc gaagagcgtc cggctcttct ccgtggcgaa cccgcacatg |
| 181 | cgcaccttcc acctctcctg gatctccttc ttcacctgct tcgtgtccac cttcgccgcc |
| 241 | gcgccgctgg tccccatcat ccgcgacaac ctcaacctca ccaaggccga catcggcaac |
| 301 | gcgggcgtgg cctccgtgtc gggctccatc ttctcccgcc tcaccatggg cgccgtctgc |
| 361 | gacctgctgg gcccgcgcta cggctgcgcc ttcctcatca tgctgtccgc gcccaccgtg |
| 421 | ttctgcatgt cgctcatcga cgacgccgcg ggctacatca ccgtcaggtt cctcatcggc |
| 481 | ttctccctcg ccaccttcgt ctcctgccag tactggatga gcaccatgtt cagcagcaag |
| 541 | atcatcggca ccgtcaacgg gctcgccgcc ggatggggca acatgggagg cggcgccacg |
| 601 | cagctcatca tgccgctcgt ctacgacgtc atccgcaagt gcggcgccac gcccttcacg |
| 661 | gcgtggcgcc tcgcctactt cgtgccgggc ctcatgcacg tcgtcatggg cgtcctggtg |
| 721 | ctcacgctgg ggcaggacct ccccgacggc aacctcaggt cgctgcagaa gaagggcaac |
| 781 | gtcaacaagg acagcttctc caaggtcatg tggtacgccg tcatcaacta ccgcacctgg |
| 841 | atcttcgtcc tcctctacgg ctactgcatg ggcgtcgagc tcaccaccga caacgtcatc |
| 901 | gccgagtaca tgtacgaccg cttcgacctc gacctccgcg tcgccgggac catcgccgcc |
| 961 | tgcttcggca tggccaacat cgtcgcgcgc cccatgggcg gcatcatgtc cgacatgggc |
| 1021 | gcgcgctact ggggcatgcg cgctcgcctc tggaacatct ggatcctcca gaccgccggc |
| 1081 | ggcgccttct gcctctggct gggacgcgcc agcaccctcc ccgtctccgt cgtcgccatg |
| 1141 | gtgctcttct ccttctgcgc gcaggcggcc tgcggcgcca tcttcggggt catccccttc |
| 1201 | gtctcccgcc gctccctcgg catcatctcc ggcatgacgg gcgccggcgg caacttcggc |
| 1261 | gcggggctca cgcagctgct cttcttcacc tcctcaacct actccacggg cagggggcta |
| 1321 | gagtacatgg gcatcatgat catggcgtgc acgctacctg tggtgttcgt gcacttcccg |
| 1381 | cagtgggggt ccatgttctt cccgcccagc gccaccgccg acgaggaggg ctactacgcc |
| 1441 | tccgagtgga acgacgacga gaagagcaag ggactccata gcgccagcct caagtttgcc |
| 1501 | gagaacagcc gctcagagcg cggcaagcga aacgtcatcc aggccgatgc cgccgccacg |
| 1561 | ccggagcatg tctaa |
| | SEQ ID NO: 45: ZmNAR2.1 translation |
| | MARQQSVHALCVLAALLFAASLPSPAAAGVHLSSLPKALDVTTS |
| | AKPGQVLHAGVDSLTVTWSLNATEPAGADAGYKGVKVKLCYAPASQKDRGWRKSEDDI |
| | SKDKACQFKVTEQAYAAAAPGSFQYAVARDVPSGSYYLRAFATDASGAEVAYGQTAPT |
| | AAFDVAGITGIHASLKIAAGVFSAFSVVALAFFFVIETRKKNK |
| | SEQ ID NO: 46: ZmNAR2.2 translation |
| | MARPGAALPLLLVVVGACCARLAAAVHLSALGRTLIVEASPKAG |
| | QVLHAGEDTITVTWHLNASASSVGYKALEVTLCYAPASQEDRGWRKANDDLSKDKACQ |
| | FRIARHAYAGGQGTLRYRVARDVPTASYHVRAYALDASGAPVGYGQTAPAYYFHVAGV |
| | SGVHASLRVAAAVLSAFSIAALAFFVVVEKRRKDE |
| | SEQ ID NO: 47: ZmNRT2.1 translation |
| | MAAVGAPGSSLHGVTGREPAFAFSTEHEEAASNGGKFDLPVDSE |
| | HKAKSVRLFSVANPHMRTFHLSWISFFTCFVSTFAAAPLVPIIRDNLNLTKADIGNAG |
| | VASVSGSIFSRLTMGAVCDLLGPRYGCAFLIMLSAPTVFCMSLIDDAAGYITVRFLIG |
| | FSLATFVSCQYWMSTMFSSKIIGTVNGLAAGWGTMGRRRHAAHMPLVYDVIRKCGATP |
| | FTAWRLAYFVPGLMHVVMGVLVLTLGQDLPDGNLRSLQKKGNVNKDSFSKVMVVYAVIN |
| | YRTWIFVLLYGYCMGVELTTDNVIAEYMYDRFDLDLRVAGTIAACFGMANIVARPMGG |
| | IMSDMGARYWGMRARLWNIWILQTAGGAFCLWLGRASTLPVSVVAMVLFSFCAQAACG |
| | AIFGVIPFVSRRSLGIISGMTGAGGNFGAGLTQLLFFTSSTYSTGRGLEYMGIMIMAC |
| | TLPVVFVHFPQWGSMFFPPSATADEEGYYASEWNDDEKSKGLHSASLKFAENSRSERG |
| | KRNVIQADAAATPEHV |
| | SEQ ID NO: 48: ZmNRT2.2 translation |
| | MAAVGAPGSSLHGVTGREPAFAFSTEHEEAASNGGKFDLPVDSE |
| | HKAKSVRLFSVANPHMRTFHLSWISFFTCFVSTFAAAPLVPIIRDNLNLTKADIGNAG |
| | VASVSGSIFSRLTMGAVCDLLGPRYGCAFLIMLSAPTVFCMSLIDDAAGYITVRFLIG |
| | FSLATFVSCQYWMSTMFSSKIIGTVNGLAAGWGNMGGGATQLIMPLVYDVIRKCGATP |
| | FTAWRLAYFVPGLMHVVMGVLVLTLGQDLPDGNLRSLQKKGNVNKDSFSKVMWYAVIN |
| | YRTWIFVLLYGYCMGVELTTDNVIAEYMYDRFDLDLRVAGTIAACFGMANIVARPMGG |
| | IMSDMGARYWGMRARLWNIWILQTAGGAFCLWLGRASTLPVSVVAMVLFSFCAQAACG |
| | AIFGVIPFVSRRSLGIISGMTGAGGNFGAGLTQLLFFTSSTYSTGRGLEYMGIMIMAC |
| | TLPVVFVHFPQWGSMFFPPSATADEEGYYASEWNDDEKSKGLHSASLKFAENSRSERG |
| | KRNVIQADAAATPEHV |
| SEQ ID NO: 49: ZmNAR2.1 promoter | |
| 1 | gctcagatcc ctcgcctcgt gtcgtgtctc cggtcgacga cgaccaacag ccagtgtggg |
| 61 | ccagacggac accgccgagc tatagcgctt ggtgatagca agggacgacc ggcggccgga |
| 121 | ccggagcacg tacgtacgta ccgcagcgat ggctcggcag caaagcgtgc aggccttgtg |
| 181 | tgtgctggcg gcgcttctct tcgccgcctc cctgccgtcg ccggccgccg cgggggtgca |
| 241 | cctctcctcg ctgcccaaag cgctcgacgt caccacctcc gccaaacccg gccaaggtgc |
| 301 | gcgcgcgttc cggcccggct catagtcata gccaaaggat tagcactttg attacttgct |
| 361 | cggttaattc atagtcctat tcttctctat gtttgaaacc cccctttaga tttgttcatt |
| 421 | cacaatcaag gagctagctg attaaaatac acacgattgc cataaaatat atgcttctcg |
| 481 | cagtcctgca cgccggcgtg gactcgctga cggtgacgtg gagcctgaac gccacggagc |
| 541 | cggccggcgc cgacgccggg tacaagggcg tgaaggtgaa gctgtgctac gcgccggcga |
| 601 | gccagaagga ccgcgggtgg cgcaagtccg aggacgacat cagcaaggac aaggcgtgcc |
| 661 | agttcaaggt caccgagcag gcgtacgcgg cggcggcgcc cggcagcttc cagtacgccg |
| 721 | tcgcccgcga cgtcccctcg ggctcctact acctgcgcgc cttcgccacg gacgcgtcgg |
| 781 | gcgccgaggt ggcctacggc cagacggcgc ccaccgccgc cttcgacgtc gccggcatca |
| 841 | ccggcatcca cgcctctctc aagatcgccg ccggcgtctt ctcggccttc tccgtcgtcg |
| 901 | cgctcgcctt cttcttcgtc atcgagaccc gcaagaagaa caagtagaac gagttgcggc |
| 961 | tgcgcgccat acatgcatac atgtaaatcg tcggcggcga tgagtggctg tcgttgctga |
| 1021 | ttcattggtg cgcgcgacta ttttggtgta tcatgtaagt tacttttctg cagtgtgtgc |
| 1081 | gtcaaaatta ccaaataata acttaagttt ctctgctgat ccggttttcg attacgaatg |
| 1141 | gaggggctca aaatatatat agtctgctcg aaagtggatt atattgccca cttattacaa |
| 1201 | atttatttat ttctagttta ttttgcagta atcattgaaa aggccagccc agcgtctctc |
| 1261 | ttcgatttaa gtaaacaata gcataaaatc cgcctcctca tctaagcgtt taccactcta |
| 1321 | ttaagctaca tctttcgata aaatggtaga gcatgtgtca ttgacctgca agattcaatg |
| 1381 | ctctatcctc tgagctgttc gcctgttctc aagagtgagc aaacatgagg ggcatcaaag |
| 1441 | atcatttcca tatgaaaatc tcggtcaact gcgacgcaaa gaagaatcgt tggacacttc |
| 1501 | tccaaacgcg atgaaaggcc aagaaggtta tgccacactg tttccacggg aattagtcgg |
| 1561 | cacaccggtg atctatcgtt tgaagaaacg aaaagccaat gaactttgtc aacggcacag |
| 1621 | aaaaaatgac gatagcgtca caatcttcca acaaaagcat atatttccac gaaaccgaac |
| 1681 | cattttgttg acgctttttt ggagcgccaa atactcaaga agaaccggcg gcggtgctct |
| 1741 | ctggtcaggc gcggacggtc cgcggcctgg ggccggacgg tccgcgacct ggcgcgaggg |
| 1801 | ctagagtttc ctgcctgacg gccggacggt ccgcgcccta gggccggacg gtccgcgcgt |
| 1861 | gcgcaggggc ggcggaagat caccggcggc gcctggatct cgctcccggg agggaccccg |
| 1921 | tcggggagga gagatcccag gggttgtctt aggctcgggc cggccgacct agactcctct |
| 1981 | aatcggcgta gagtcgaaga gaggtgaaga atttggggat taagaggcta aactagaact |
| 2041 | actcctaatt gtactggaaa taaatgcgaa tagaagttgt attgattcga ttg |
| SEQ ID NO: 50: ZmNAR2.2 promoter | |
| 1 | atgtgatcac aagactgtca tgttttgtca tagcaccctg atgcttagta catccaatga |
| 61 | tccaagcaca atcttggcat gcctgcagat tggtagcaca tccatttgat ctcactccat |
| 121 | gtaagtgggt caacactaag gtattatatg tttgcaccca gatcattatg tgatagcatg |
| 181 | ctaccaccac catagcccac catatgacca ccaggaatac gctggggtga tcatgcctga |
| 241 | agcattggat attgatgtgg attttgatct tataatgtca tccttactaa taattatttg |
| 301 | aacgttgtgc aggtaatcaa tgctttagtg aattttattc ttgatatact tgtcataatg |
| 361 | tagcattctt gtgagcaaat cattatctgt gttaaggtca tggtcatgga cctgcagcaa |
| 421 | tatcaaatga aacaaatgtt tgaaatgata tctggtgtga catttttagt gag attgtag |
| 481 | tggtatattg gtaaagttgt attttgatca cttggagttt tcgtttactt cacccactgc |
| 541 | tgtataattt ccttgttctg aataagcatt agaacataac aattctaact agcattttac |
| 601 | acgattgaac aaacccaagc accccaatct ctacatcaat agctctgtgc tgctaatctg |
| 661 | ctagcacttc aatatttctg ttatttattt gcctaatttg gttgagtctg gcttgtgaag |
| 721 | tgtattaaca atagttaaaa attgtatatt gtgatatttg ttgatgactt ggttatattt |
| 781 | gtggtgtttg tttgaattta tctgactata agcaaaattt caatattttt tgttttaatg |
| 841 | tgctgacaca ctcaagtgga tttgtgcaca taatatccat caaaagaaga tgcttgtata |
| 901 | attttatatt tgatactgtt gccatgagta cacctctttt cagtttttag ttggagcttt |
| 961 | ttttccctac atacacttag ctcatgttta gattgccagg atcctagcac atttcatgtg |
| 1021 | aattggtgct gatttgatgc atctaaacaa gcattaaaag tgtgtattta tgatttttat |
| 1081 | tagtttttgc tagcagacct tcaggctgct gaaatttctt caatgtatag gttctgtttt |
| 1141 | ctacttttct tccacattgg tcaggcaata tgtttttgtg tcacactcaa aaaacacttc |
| 1201 | tgaattttat aaacagtgat ttctgtataa ttctgattcc aaacaatggt ggtatgtctt |
| 1261 | cttgtactct tataatgtac tgatgatatc gggttttatc ttctattatg gttgtaaggc |
| 1321 | ttgatgcctg aaactgctga ttattaaatc actttcaagt ttcaacacac ctaaatttgg |
| 1381 | gatatgggat agattacatt atgctagggt aatcgaattt tctttttttc taccctttcg |
| 1441 | ctgttggtgg ttaaatgatt cttaacatga tctgataccc acattttatt ttgggaaaat |
| 1501 | aacatttttt gttcgtctaa ttcagtttaa gtgtttttag ggcaattcgg tttcagtatc |
| 1561 | tttttcaatc ttccgtgaac agttaaaaat tgaagttatt taactaattt ggtactctag |
| 1621 | cctgtcaaaa aaaatacttg tgttgattcc ttctctgaaa atttttccat ttagtttctc |
| 1681 | gggctgtatt cccgtctgat ttgaatattc gttcaccaaa cctttcaggc cggagtctga |
| 1741 | tgaagaaaga aatttgaaag aagaaatcaa cctcctgaag gtggatctta aagaaatcga |
| 1801 | gggaaagatg agtaatggtt ctgagcagac atcggtagat gcaaaagatt tgtctgagaa |
| 1861 | gatatctatg ttggaaagcc agcttgagca gctttcaagg gagttggatg acaagattcg |
| 1921 | atttgggcaa aggccacgtt ctggtgcagg cagggttaca acacttgcac ccactagttt |
| 1981 | aggggaggaa ccacaggcta cagtggtgga cagaccacgt tctcgtggtg gtatggaacc |
| 2041 | acccccaagg caggaagaaa gatggggatt tcaaggaagc cgagaaaggg gctcgtttgg |

Triticum Aestivum

| SEQ ID NO: 51: TaNAR2.1 AY763794.1 mRNA, complete cds | |
|---|---|
| 1 | tcctctcttg ccttctccga tcgacaacag cagccaagca ctaactagcc gcgcgcaggg |
| 61 | atggctcgcc aaggtatggt cacggcgctg ttgctggtgg tcctcgccgc cggctgctgc |
| 121 | gcgtcggcgg gcgccgtggc gtacctctcc aagctgccgg tgaccctcga cgtcaccgcc |
| 181 | tcccccagtc ccggccaagt tcttcacgcc ggcgaggacg tgatcacggt gacgtgggcc |
| 241 | ctgaacgcga gccagccggc cggcaaggac gtcgactaca agaacgtgaa ggtgagcctc |
| 301 | tgctacgcgc cggtgagcca gaaggagcgc gagtggcgca agacccacga cgacctcaag |
| 361 | aaggacaaga cctgccagtt caaggtcacc cagcaggcct accccggcac cggcaaggtc |
| 421 | gagtaccgcg tcgccctcga catccccacc gccacctact acgtgcgcgc ctacgcgctc |
| 481 | gacgcctccg gcacccaggt cgcctacggc cagaccgcgc cctcctccgc cttcaacgtc |
| 541 | gtcagcataa ccggcgtcac cacctccatc aaggtcgccg ccggcgtctt ctccgccttc |
| 601 | tccgtcgcct ccctcgcatt cttcttcttc attgagaaac gcaagaagaa caactagatg |
| 661 | tggagatcgg aaccgtagca aagtttgttg gctttctctg gccggtcgtt ttctctctac |
| 721 | atgtaacagt atcagtacgg accagcacgt acgtacgtac ggtgcaaact gtacggataa |
| 781 | atgttcgggt gca |
| SEQ ID NO: 52: TaNAR2.2 AY763795.1 mRNA, complete cds | |
| 1 | ttctctgacc gagcagctgc gagctcgatc gagcccaagt tcgacggcga tggcacagtc |
| 61 | gaagctggtc atggcgttgc tggtggcggt cctcgccgcc ggctgctgcg cgtcggccgg |
| 121 | cgccgtggcg tacctctcca agcttcctgt gaccctcgac gtcatcgcat cccccagccc |
| 181 | cggccaagtt ctccatgccg gcgaggacgt gatcacagtg acgtgggccc tcaacgcgtc |
| 241 | tcggccggcc ggcgacgacg ccgcctacaa gaacgtgaag gtcagcctct gctacgcgcc |
| 301 | ggcgagccag aaggagcgcg agtggcgcaa gacccacgac gacctcaaga aagacaagac |
| 361 | ctgccagttc aaggtcgccc agcagcccta cgccggcgcc ggcggcaggg tcgagtaccg |
| 421 | cgtcgccctc gacatcccca ccgccaccta ctacgtgcgc gcctacgcgc tcgacgcctc |
| 481 | cggcacgcag gtcgcctacg gccagaccgc gcccgccgcc gccttcaacg tcgtcagcat |
| 541 | cacgggcgtc accacctcca tcaaggtcgc cgccggcgtc ttctccacct tctccgtcgt |
| 601 | ctccctcgcc ttcttcttct tcattgagaa gcgcaagaag aataactaag ctcagaaccg |
| 661 | tacgtggcaa ggtgttcgtc cgtgcgcgac ttcttcctct tgtcctttgc aacgtacgca |
| 721 | caaaaggtgt acatgtaatg taagagagtg ttggtgtttc c |
| SEQ ID NO: 53: TaNRT2 AF288688 mRNA, complete cds | |
| 1 | aagctagcac caagcctcca aggagcaaga agagaagaag ccttgctcga tcaagcaagg |
| 61 | tcgaaatgga ggtggaggcc agcgcccatg gcgacacggc ggcgagcaag ttcacgctgc |
| 121 | ccgtggactc cgagcacaag gccaagtcct tcagactctt ctccttcgcc aacccccaca |
| 181 | tgcgtacctt ccacctctcc tggatatcct tcttcacctg cttcgtctcc accttcgcgg |
| 241 | cggcaccgtt ggtgcccatc atccgtgaca acctcaacct cgctaaggcc gacataggga |
| 301 | atgccggtgt ggcatctgtg tctgggtcca tcttctccag gcttgccatg ggtgccatct |
| 361 | gcgacctttt agggccgcgg tatggctgcg ccttcctcgt catgctctca gcacccactg |
| 421 | tgttttgcat ggctgctatc gacgatgcgt caggctacat cgccgtacgc ttcctcattg |
| 481 | gcttctccct cgccaccttc gtgtcatgcc aatattggat gagcaccatg ttcaacagta |
| 541 | agatcattgg cacggtgaat ggcctcgcgg ccggctgggg caacatgggc ggtggtgcca |
| 601 | cacaactcat catgccgctt gttttccatg ccatccaaaa gtgtggtgcc acacccttcg |
| 661 | tggcatggcg tattgcctat ttcgtgccgg gaatgatgca catcgtcatg gggttgcttg |
| 721 | tgctcactat gggccaagat ctccccgacg gcaaccttgc gagtctccag aagaaggggg |
| 781 | acatggccaa ggacaaattc tcgaaggtcc tttggggtgc ggtcaccaac taccggacat |
| 841 | ggatattcgt cctcctctac ggctactgca tgggtgtcga gctcaccacc gacaacgtca |
| 901 | tcgccgagta ctactacgac cacttccacc ttgaccttcg cgccgctggc accattgccg |
| 961 | cttgcttcgg catggccaac atcgtcgcgc gtcctatggg tggctatctc tctgaccttg |
| 1021 | gtgcccgcta cttcggcatg cgtgctcggc tctggaacat ctggatcctc cagaccgctg |
| 1081 | gtggcgcttt ctgcatctgg ctcggtcgtg catcggccct tcctgcctca gtcacggcca |
| 1141 | tggtcctctt ttccatttgt gcacaagctg cttgtggtgc tgtatttggc gtcgcaccct |
| 1201 | tcgtttccag gcgttccctt ggcatcatct ccgggctgac cggcgctggt ggcaatgttg |
| 1261 | gcgcagggct aacgcaactt cttttcttca catcgtcgca atactccacc gggaggggtc |
| 1321 | tcgagtacat gggcatcatg atcatggcat gcacattacc cgtcgctctg gtgcacttcc |
| 1381 | cccaatgggg ctccatgttc ttcccggcta gcgctgatgc cacggaagag gaatactatg |
| 1441 | cttctgagtg gtcggaggag gagaagggca agggtctcca tattacaggc caaaagttcg |
| 1501 | cagagaactc ccgctcagag cgcggcaggc gcaacgtcat ccttgccaca tccgccacgc |
| 1561 | cacccaacaa cacaccccag cacgtataag gcccttattt ttatgtcacc taagaatttt |
| 1621 | actgttcatc acgtatatat acaaaccgta tatctacgtc tgcagcccca gcgtaataag |
| 1681 | ttgtatgggg atttatgttt ctactagtaa acttaaggaa acgctgcttt tgcgttcctg |
| 1741 | ctctgtacgc atgaaatgta atatcaattt gagtccgaaa ttactacaaa aaaaaa |
| | SEQ ID NO: 54: TaNAR2.1 translation |
| | MARQGMVTALLLVVLAAGCCASAGAVAYLSKLPVTLDVTASPSP |
| | GQVLHAGEDVITVTWALNASQPAGKDVDYKNVKVSLCYAPVSQKEREWRKTHDDLKKD |
| | KTCQFKVTQQAYPGTGKVEYRVALDIPTATYYVRAYALDASGTQVAYGQTAPSSAFNV |
| | VSITGVTTSIKVAAGVFSAFSVASLAFFFFIEKRKKNN |
| | SEQ ID NO: 55: TaNAR2.2 translation |
| | MAQSKLVMALLVAVLAAGCCASAGAVAYLSKLPVTLDVIASPSP |
| | GQVLHAGEDVITVTWALNASRPAGDDAAYKNVKVSLCYAPASQKEREWRKTHDDLKKD |
| | KTCQFKVAQQPYAGAGGRVEYRVALDIPTATYYVRAYALDASGTQVAYGQTAPAAAFN |
| | VVSITGVTTSIKVAAGVFSTFSVVSLAFFFFIEKRKKNN |
| | SEQ ID NO: 56: TaNRT2 translation |
| | MEVEASAHGDTAASKFTLPVDSEHKAKSFRLFSFANPHMRTFHL |
| | SWISFFTCFVSTFAAAPLVPIIRDNLNLAKADIGNAGVASVSGSIFSRLAMGAICDLL |
| | GPRYGCAFLVMLSAPTVFCMAAIDDASGYIAVRFLIGFSLATFVSCQYWMSTMFNSKI |
| | IGTVNGLAAGWGNMGGGATQLIMPLVFHAIQKCGATPFVAWRIAYFVPGMMHIVMGLL |
| | VLTMGQDLPDGNLASLQKKGDMAKDKFSKVLWGAVTNYRTWIFVLLYGYCMGVELTTD |
| | NVIAEYYYDHFHLDLRAAGTIAACFGMANIVARPMGGYLSDLGARYFGMRARLWNIWI |
| | LQTAGGAFCIWLGRASALPASVTAMVLFSICAQAACGAVFGVAPFVSRRSLGIISGLT |
| | GAGGNVGAGLTQLLFFTSSQYSTGRGLEYMGIMIMACTLPVALVHFPQWGSMFFPASA |
| | DATEEEYYASEWSEEEKGKGLHITGQKFAENSRSERGRRNVILATSATPPNNTPQHV |

Chlamydomonas Reinhardtii

SEQ ID NO: 57: CrNRT2.3 AJ223296.2 mRNA, complete cds
atggacgttttccagtat acgacactgg acaagggcgc tggttctgcg cttttccgtg cacctcgtctaacatatatc
ggtgccgcag acgtccagagcgag acgcgcaaga atgtctacag gcgtcgacta cttgggacatggcgcggtga
aggcgactga ggggccgccg gtcaacccgt caggccgcaa gtacccttacgagctcgact cggagggcaa ggccaaaagc
attcccgtgt ggcgcttcac caacccgcacatgggcgcct ttcatctgtcct ggttcgcctt cttcatttcc
ttcctcgccaccttcgcgcc ggcctcgctg ctgcccatca tccgcgacga cctgttcctg accaaggcgcagctgggcaa
cgccggtgtg gcggccgtgt gcggcgccat cgcggcacgc gtgctcatgggcgtgtttgt ggacatcgtg ggcccccgcta
cggcaccgcagccaccatgt tgatgaccgc tccggccgtg ttctgcatgg ccctggtcac cgacttcgccacgttcgccg
ccgtgcgctt cttcatcggc ctcagcctct gcatgttcgt gtgctgtcagt tctggtgcgg caccatgttc aacgtccaaa
tagtgggcac tgccaacgcc atcgccgggg gctggggcaa catgggcggc ggcgcgtgtc acttcatcat gccgctcatc
taccagggca tcaaggacgg cggcgtgccg ggataccaggcctggcg ctgggccttc ttcgtgccgg ctgtcttcta
catcgccacg gccctggccaccctggccct gggcattgac caccccagcg gcaaggacta ccgcgacctg
aaaaaggagggggcgctcaa gtccaagggc gccatgtggc cagtcatcaa gtgcggcctc ggcaactacaggtct
tggatcctgg ccctgacgta cggctactccttcggtgttg agttgacggt ggacaacatt atcgtggagt acatgtttga
ccagttcgggctgtcgttga
cgtggcgggcgcgctgggcggcatgtttggcatgatgaaccttttcagccgggccagcggcggcatgat cagcgacct
catcgccaag cccttcggaa tgcgcggtcg catctgcgctctctggatca tccagaccct ggggggcatc ttctgcgtca
tcctgggccg ggtcc acaacagcct gacctccacc atcgtcatca tgatcatcttctccatcttc tgccagcaag cctgcggcct
gcacttcggc atcacgccct tcgtgtcgcgccgcgcctac ggcgtggtct ccggcctcgt gggcgcaggc ggcaacaccg
gcgccgccatcacacaagcc atctggttcg ccggcaccgc cccctggcag ctgacc ctcaccaagt accagggtct
ggagtacatgggataccaga ccattggtct gacgctggcg ctgttcttca tctggttccc catgtggggctccatgctga
ccggaccgcg cgagggcgca acagag
gaggacta ctacatcaag gagtggagtgcggaggaagtggctgacggcctgcaccacaccagcctgcg ctttgcaatg
gagtcccgct cgcagcgcgg cacacgcacc agcacccagaccaaggtgat gtcggtcggc gacggcgccg
gcagcaacaa ggcggaggtg gtggtggtggcggcggcgca gcagggcgcg gtgccgatgg catctgtgga
ggaggggagc agcggccgcagcagcagctc ggggggccac cagcagcagg atgcgcatatacttcact gcatacgtac
tgttgttcaa aaagcgccgc gagtgggcga gcgggagagcgagcgggaga gggactga
SEQ ID NO: 58: CrNRT2.3 translation
MDVFQYTTLDKGAGSALFRAPRLTYIGAADVQTRRARMSTGVDY
LGHGAVKATEGPPVNPSGRKYPYELDSEGKAKSIPVWRFTNPHMGAFHLSWFAFFISF
LATFAPASLLPIIRDDLFLTKAQLGNAGVAAVCGAIAARVLMGVFVDIVGPRYGTAAT
MLMTAPAVFCMALVTDFATFAAVRFFIGLSLCMFVCCQFWCGTMFNVQIVGTANAIAG
GWGNMGGGACHFIMPLIYQGIKDGGVPGYQAWRWAFFVPAVFYIATALATLALGIDHP
SGKDYRDLKKEGALKSKGAMWPVIKCGLGNYRSWILALTYGYSFGVELTVDNIIVEYM
FDQFGLSLTVAGALGGMFGMMNLFSRASGGMISDLIAKPFGMRGRICALWIIQTLGGI
FCVILGRVHNSLTSTIVIMIIFSIFCQQACGLHFGITPFVSRRAYGVVSGLVGAGGNT
GAAITQAIWFAGTAPWQLTLTKYQGLEYMGYQTIGLTLALFFIWFPMWGSMLTGPREG
ATEEDYYIKEWSAEEVADGLHHTSLRFAMESRSQRGTRTSTQTKVMSVGDGAGSNKAE
VVVVAAAQQGAVPMASVEEGSSGRSSSSGGHQQQDAHILHCIRTVVQKAPRVGERESE
RERD

Glycine Max

| SEQ ID NO: 59: GmNRT2 AF047718.1 mRNA, complete cds | |
|---|---|
| 1 | tcacactttc ttccttaatt ttctagctct tgctacgtac ttgaattcaa ttagttatta |
| 61 | atggctgaga ttgagggttc tcccggaagc tccatgcatg gagtaacagg aagagaacaa |
| 121 | acatttgtag cctcagttgc ttctccaatt gtccctacag acaccacagc caaatttgct |
| 181 | ctcccagtgg attcagaaca caaggccaag gttttcaaac tcttctccct ggccaatccc |
| 241 | cacatgagaa ccttccacct ttcttggatc tccttcttca cctgcttcgt ctcgacattc |
| 301 | gcagcagcac ctcttgtgcc catcatccgc gacaacctta acctcaccaa aagcgacatt |
| 361 | ggaaacgccg gggttgcttc tgtctccgga agcatcttct caaggctcgc aatgggtgca |
| 421 | gtctgtgaca tgttgggtcc acgctatggc tgcgccttcc tcatcatgct ttcggcccct |
| 481 | acggtgttct gcatgtcctt tgtgaaagat gctgcggggt acatagcggt tcggttcttg |
| 541 | attgggttct cgttggcgac gtttgtgtcg tgccagtact ggatgagcac gatgttcaac |
| 601 | agtaagatta tagggcttgc gaatgggact gctgcggggt gggggaacat gggtggtgga |
| 661 | gccactcagc tcataatgcc tttggtgtat gagcttatca gaagagctgg ggctactccc |
| 721 | ttcactgctt ggaggattgc cttctttgtt ccgggtttca tgcatgtcat catggggatt |
| 781 | cttgtcctca ctctaggcca ggacttgcct gatggaaacc tcggggcctt gcggaagaag |
| 841 | ggtgatgtag ctaaagacaa gttttccaag gtgctatggt atgccataac aaattacagg |
| 901 | acatggattt ttgctctcct ctatgggtac tccatgggag ttgaattaac aactgacaat |
| 961 | gtcattgctg agtatttcta tgacagattt aatctcaagc tacacactgc tggaatcatt |
| 1021 | gctgcttcat ttggaatggc aaacttagtt gctcgacctt ttggtggata tgcttcagat |
| 1081 | gttgcagcca ggctgtttgg catgagggga agactctgga ccctttggat cctccaaacc |
| 1141 | ttaggagggg ttttctgtat ttggcttggc cgtgccaatt ctcttcctat tgctgtattg |
| 1201 | gccatgatcc tgttctctat aggagctcaa gctgcatgtg gtgcaacttt tggcatcatt |
| 1261 | cctttcatct caagaaggtc tttggggatc atatcaggtc taactggtgc aggtggaaac |
| 1321 | tttgggtctg gcctcaccca attggtcttc ttttcaacct ccaaattctc tactgccaca |
| 1381 | ggtctctcct tgatgggtgt aatgatagtg gcttgcactc taccagtgag tgttgttcac |
| 1441 | ttcccacagt ggggtagcat gtttctacca ccctcaaaag atgtcagcaa atccactgaa |
| 1501 | gaattctatt acacctctga atggaatgag gaagagaagc agaagggttt gcaccagcaa |
| 1561 | agtctcaaat ttgctgagaa tagccgatct gagagaggaa agcgagtggc ttcagcacca |
| 1621 | acacctccaa atgcaactcc cactcatgtc tagccatagc acttcaatca aagaagatca |
| 1681 | tgaaacataa ttactgagca gtattgggaa tgaagaacca tgagttgaag aattttctaa |
| 1741 | taagaaatct tgtaacatgt agacatagaa tgttctggtt ctggtttgcg tgtggtgtaa |
| 1801 | gagttgtcta cttgtggtaa gtcataagta tcataatcag tatgtcaatg cagatcttga |
| 1861 | tgctgagtat caatagtatc aaaaaaaaaa |

SEQ ID NO: 60: GmNRT2 translation
MAEIEGSPGSSMHGVTGREQTFVASVASPIVPTDTTAKFALPVD
SEHKAKVFKLFSLANPHMRTFHLSWISFFTCFVSTFAAAPLVPIIRDNLNLTKSDIGN
AGVASVSGSIFSRLAMGAVCDMLGPRYGCAFLIMLSAPTVFCMSFVKDAAGYIAVRFL
IGFSLATFVSCQYWMSTMFNSKIIGLANGTAAGWGNMGGGATQLIMPLVYELIRRAGA
TPFTAWRIAFFVPGFMHVIMGILVLTLGQDLPDGNLGALRKKGDVAKDKFSKVLWYAI
TNYRTWIFALLYGYSMGVELTTDNVIAEYFYDRFNLKLHTAGIIAASFGMANLVARPF
GGYASDVAARLFGMRGRLWTLWILQTLGGVFCIWLGRANSLPIAVLAMILFSIGAQAA
CGATFGIIPFISRRSLGIISGLTGAGGNFGSGLTQLVFFSTSKFSTATGLSLMGVMIV
ACTLPVSVVHFPQWGSMFLPPSKDVSKSTEEFYYTSEWNEEEKQKGLHQQSLKFAENS
RSERGKRVASAPTPPNATPTHV

1. A method for increasing growth, yield, biomass, agricultural nitrogen use efficiency (ANUE), N recovery efficiency (NRE), stress tolerance and/or total N content of a plant and/or mitigating the effects of stress on a plant, the method comprising altering the expression profile of a NRT2 nucleic acid in a plant, wherein preferably the NRT2 nucleic acid comprises a sequence encoding a NRT2.1, NRT2.2 and/or NRT2.3a polypeptide as defined in SEQ ID NOs: 2, 4 and 6 respectively, or a functional homologue or variant thereof.

2. The method of claim 1, wherein the method comprises introducing and expressing into a plant a nucleic acid construct comprising a NRT 2.1, NRT 2.2 and/or NRT2.3a nucleic acid sequence operably linked to a nitrate-inducible promoter, wherein preferably the nitrate-inducible promoter is a NAR2.1 promoter comprising a sequence as defined in SEQ ID No: 7 or a functional homologue or variant thereof; or
introducing a mutation into the plant genome, wherein said mutation is the insertion of at least one or more additional copy of
a NRT2.1, NRT 2.2 and/or NRT2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or
a NRT 2.1, NRT 2.2 or NRT 2.3a gene sequence operably linked to a NAR2.1 promoter sequence;
wherein such mutation is introduced using targeted genome editing.

3. (canceled)

4. (canceled)

5. (canceled)

6. (canceled)

7. The method of claim 1, wherein the expression profile of a NRT2 nucleic acid is altered compared to a control plant, wherein preferably, altering the expression profile comprises altering the relative expression ratios of NRT2 to NAR in a plant, preferably, wherein said ratio is reduced compared to the ratio in a control plant, and more preferably, wherein the NRT2.1:NAR2.1, NRT2.2:NAR 2.1 or NRT2.3a:NAR2.1 ratio is below at least 7:1, preferably below 6:1, more preferably below 5:1, and even more preferably 4.7:1 in plant culms compared with a ratio of at least below 10:1, preferably below 9:1, more preferably below 8:1 and even more preferably 7.2:1 in control plants, and wherein the ratio is lower than that in control plants.

8. (canceled)

9. (canceled)

10. The method of claim 1, wherein the plant is selected from rice, maize, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar, forage or turf grass.

11. The method of claim 1, wherein the stress tolerance is tolerance to abiotic stress, preferably wherein the abiotic stress is drought, cold and/or high salt conditions, or the stress is abiotic stress, preferably wherein the abiotic stress is cold and/or high salt conditions.

12. (canceled)

13. A plant obtained or obtainable by the method as defined in claim 1.

14. A nucleic acid construct comprising a nucleic acid sequence encoding any one of SEQ ID Nos: 2, 4 or 6, a functional variant or homolog thereof, operably linked to a regulatory sequence, wherein said regulatory sequence is a nitrate-inducible promoter, and wherein preferably the nitrate-inducible promoter is a NAR2.1 promoter comprising a sequence as defined in SEQ ID No: 7 or a functional homologue or variant thereof.

15. A vector or a host cell comprising a nucleic acid construct of claim 14.

16. (canceled)

17. The host cell of claim 15, wherein the cell is a bacterial or plant cell.

18. A transgenic plant expressing the nucleic acid construct of claim 14.

19. The transgenic plant of claim 18, wherein the plant is selected from rice, maize, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar, forage or turf grass.

20. (canceled)

21. (canceled)

22. (canceled)

23. (canceled)

24. (canceled)

25. (canceled)

26. (canceled)

27. A method of producing a mutant plant that has increased growth, biomass, yield, agricultural nitrogen use efficiency (ANUE), N recovery efficiency (NRE), improved stress tolerance and/or total N content of a plant or of mitigating the effects of stress on a plant, the method comprising introducing a mutation into the plant genome, wherein said mutation is introduced by mutagenesis or targeted genome editing, and wherein said mutation introduces at least one or more additional copy of
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or
a NRT 2.1, NRT 2.2 or NRT 2.3a gene sequence operably linked to a NAR2.1 promoter sequence.

28. The method of claim 27, wherein said NRT2.1 gene sequence comprises SEQ ID NO: 1 or a functional homologue or variant thereof said NRT 2.2 sequence comprises SEQ ID NO: 3 or a functional homologue or variant thereof and said NRT2.3a sequence comprises SEQ ID NO: 5 or a functional homologue or variant thereof and wherein preferably said sequence encodes a NRT2.1 protein as defined in SEQ ID NO: 2 or a functional homologue or variant thereof, a NRT2.2 protein as defined in SEQ ID NO: 4 or a functional variant or homologue thereof and a NRT 2.3a protein as defined in SEQ ID NO: 6 or a functional homologue or variant thereof.

29. The method of claim 27, wherein the NAR2.1 promoter sequence is SEQ ID NO: 7 or a functional homologue or variant thereof

30. The method of claim 27, wherein the mutation is introduced using ZFNs, TALENs or CRISPR/Cas9.

31. (canceled)

32. The genetically altered plant of claim 45, wherein said plant carries a mutation in its genome and wherein said mutation introduces one or more additional copy of a
a NRT2.1, 2.2 or 2.3a gene sequence such that at least one sequence is operably linked to an endogenous nitrate-inducible promoter, preferably an endogenous NAR2.1 promoter sequence;
a NAR 2.1 promoter sequence, such that said promoter sequence is operably linked to at least one endogenous NRT2.1, 2.2 or 2.3a gene sequence and/or
a NRT 2.1, NRT 2.2 or NRT 2.3a gene sequence operably linked to a NAR2.1 promoter sequence;
into the plant genome.

33. The genetically altered plant of claim 32, wherein said mutation is introduced using mutagenesis or targeted genome editing, wherein preferably the mutation is introduced using ZFNs, TALENs or CRISPR/Cas9.

34. (canceled)

35. The genetically altered plant of claim 32, wherein said NRT2.1 gene sequence comprises SEQ ID NO: 1 or a functional homologue or variant thereof, said NRT 2.2 sequence comprises SEQ ID NO: 3 or a functional homologue or variant thereof and said NRT2.3a sequence comprises SEQ ID NO: 5 or a functional homologue or variant thereof and wherein preferably said sequence encodes a NRT2.1 protein as defined in SEQ ID NO: 2 or a functional homologue or variant thereof, a NRT2.2 protein as defined in SEQ ID NO: 4 or a functional variant or homologue thereof and a NRT 2.3a protein as defined in SEQ ID NO: 6 or a functional homologue or variant thereof.

36. The genetically altered plant of claim 32, wherein said NRT 2.1a promoter sequence comprises a sequence as defined in SEQ ID NO: 7 or a functional homologue or variant thereof.

37. (canceled)

38. (canceled)

39. (canceled)

40. (canceled)

41. (canceled)

42. (canceled)

43. (canceled)

44. (canceled)

45. A genetically altered plant characterised by a lower expression ratio of NRT2.1:NAR2.1, NRT2.2: NAR 2.1 and/or NRT2.3a:NAR2.1 ratio compared to said ratio in a control plant.

46. (canceled)

47. (canceled)

48. (canceled)

49. (canceled)

50. (canceled)

51. (canceled)

52. (canceled)


SEQUENCE LISTING (60 sequences)

SEQ ID NO: 1 - DNA, 1928 bp, Oryza sativa
aattgcatccgagctcacctagcttctctcttgcaaccagcgattcgatcgattccatctccaagaagcagcggctagcagcagctagtagttgccatggactcgtcgacggtgggcgctccggggagctcgctgcacggcgtgacggggcgcgagccggcgttcgcgttctcgacggaggtgggcggcgaggacgcggcggcggcgagcaagttcgacttgccggtggactcggagcacaaggcgaagacgatcaggttgctgtcgttcgcgaacccgcatatgaggacgttccacctatcatggatctccttcttctcctgcttcgtctccaccttcgccgccgcccctctcgtccccatcatccgcgacaacctcaacctcaccaaggccgacatcggcaacgccggcgtcgcctccgtctccggctccatcttctccaggctcgccatgggcgccatctgcgacatgctcggcccgcgctacggctgcgccttcctcatcatgctcgccgcgcccaccgtcttctgcatgtcgctcatcgactccgccgcggggtacatcgccgtgcgcttcctcatcggcttctccctcgccaccttcgtgtcatgccagtactggatgagcaccatgttcaacagcaagatcatcggcctcgtcaacggcctcgccgccgggtggggaaacatgggcggcggcgcgacgcagctcatcatgccgctcgtctacgacgtgatccgcaagtgcggcgcgacgccgttcacggcgtggaggctggcctacttcgtgccggggacgctgcacgtggtgatgggcgtgctggtgctgacgctggggcaggacctccccgacggcaacctgcgcagcctgcagaagaagggtgacgtcaacagggacagcttctccagggtgctctggtacgccgtcaccaactaccgcacctggatcttcgtcctcctctacggctactccatgggcgtcgagctcaccaccgacaacgtcatcgccgagtacttctacgatcgcttcgacctcgacctccgcgtcgccggcatcatcgccgcatccttcggcatggccaacatcgtcgcgcgccccaccggcggcctcctctcggacctcggcgcgcgctacttcggcatgcgcgcccgcctctggaacatttggatcctccagaccgccggcggcgcgttctgcctcctgctcggccgcgcatccaccctccccacctccgtcgtctgcatggtcctcttctccttctgcgcgcaggccgcctgcggcgccatcttcggcgtcatccccttcgtctcccgccgctcgctcggcatcatctccggcatgaccggcgccggcggcaacttcggcgccgggctcacgcagctgctcttcttcacgtcgtcgaggtactccacgggcacggggctggagtacatgggcatcatgatcatggcgtgcacgctgccggtggtgctcgtccatttcccgcagtggggctccatgttcctcccgcccaacgccggcgccgaggaggagcactactacggctccgagtggagcgaacaggagaagagcaagggcctccacggtgcaagtctcaagttcgccgagaactcccgctccgagcgtggccgccgcaacgtcatcaacgccgccgccgccgccgccacgccgcccaacaactcgccggagcacgcctaaggcgtaaacaattctgcgaccgagaccagcaatacgctggagttcgactcgatgataacacgccggagcacgtccttgttgcaaacggtgatgaaattaagagtgaaatatttccttaggaattgaatcctttgaaattaattcctttggaatttctccgatacaaacggaggtataataagggagaggcatttatacctatgtacatgttacacttttttgaaaaaaaa

SEQ ID NO: 2 - protein, 533 aa, Oryza sativa
MDSSTVGAPGSSLHGVTGREPAFAFSTEVGGEDAAAASKFDLPVDSEHKAKTIRLLSFANPHMRTFHLSWISFFSCFVSTFAAAPLVPIIRDNLNLTKADIGNAGVASVSGSIFSRLAMGAICDMLGPRYGCAFLIMLAAPTVFCMSLIDSAAGYIAVRFLIGFSLATFVSCQYWMSTMFNSKIIGLVNGLAAGWGNMGGGATQLIMPLVYDVIRKCGATPFTAWRLAYFVPGTLHVVMGVLVLTLGQDLPDGNLRSLQKKGDVNRDSFSRVLWYAVTNYRTWIFVLLYGYSMGVELTTDNVIAEYFYDRFDLDLRVAGIIAASFGMANIVARPTGGLLSDLGARYFGMRARLWNIWILQTAGGAFCLLLGRASTLPTSVVCMVLFSFCAQAACGAIFGVIPFVSRRSLGIISGMTGAGGNFGAGLTQLLFFTSSRYSTGTGLEYMGIMIMACTLPVVLVHFPQWGSMFLPPNAGAEEEHYYGSEWSEQEKSKGLHGASLKFAENSRSERGRRNVINAAAAAATPPNNSPEHA

SEQ ID NO: 3 - DNA, 1791 bp, Oryza sativa
aaagcaacagcagcagaagtgcacttttgcattctatttccaatcaatccaagaggctagagctagcggccaagatcatcgtcaccggcggcatggactcgtcgacggtgggcgctccggggagctcgctgcacggcgtgacggggcgcgagccggcgttcgcgttctcgacggaggtgggcggcgaggacgcggcggcggcgagcaagttcgacttgccggtggactcggagcacaaggcgaagacgatcaggttgctgtcgttcgcgaacccgcatatgaggacgttccacctatcatggatctccttcttctcctgcttcgtctccaccttcgccgccgcccctctcgtccccatcatccgcgacaacctcaacctcaccaaggccgacatcggcaacgccggcgtcgcctccgtctccggctccatcttctccaggctcgccatgggcgccatctgcgacatgctcggcccgcgctacggctgcgccttcctcatcatgctcgccgcgcccaccgtcttctgcatgtcgctcatcgactccgccgcggggtacatcgccgtgcgcttcctcatcggcttctccctcgccaccttcgtgtcatgccagtactggatgagcaccatgttcaacagcaagatcatcggcctcgtcaacggcctcgccgccgggtggggaaacatgggcggcggcgcgacgcagctcatcatgccgctcgtctacgacgtgatccgcaagtgcggcgcgacgccgttcacggcgtggaggctggcctacttcgtgccggggacgctgcacgtggtgatgggcgtgctggtgctgacgctggggcaggacctccccgacggcaacctgcgcagcctgcagaagaagggtgacgtcaacagggacagcttctccagggtgctctggtacgccgtcaccaactaccgcacctggatcttcgtcctcctctacggctactccatgggcgtcgagctcaccaccgacaacgtcatcgccgagtacttctacgatcgcttcgacctcgacctccgcgtcgccggcatcatcgccgcatccttcggcatggccaacatcgtcgcgcgccccaccggcggcctcctctcggacctcggcgcgcgctacttcggcatgcgcgcccgcctctggaacatttggatcctccagaccgccggcggcgcgttctgcctcctgctcggccgcgcatccaccctccccacctccgtcgtctgcatggtcctcttctccttctgcgcgcaggccgcctgcggcgccatcttcggcgtcatccccttcgtctcccgccgctcgctcggcatcatctccggcatgaccggcgccggcggcaacttcggcgccgggctcacgcagctgctcttcttcacgtcgtcgaggtactccacgggcacggggctggagtacatgggcatcatgatcatggcgtgcacgctgccggtggtgctcgtccatttcccgcagtggggctccatgttcctcccgcccaacgccggcgccgaggaggagcactactacggctccgagtggagcgaacaggagaagagcaagggcctccacggtgcaagtctcaagttcgccgagaactcccgctccgagcgtggccgccgcaacgtcatcaacgccgccgccgccgccgccacgccgcccaacaactcgccggagcacgcctaattaagaggccaagttaattaattatgcatgcatgtataaactgttgaacgttttgttaccgttttctttatactatctagagtatgtcgtcatggag

SEQ ID NO: 4 - protein, 533 aa, Oryza sativa; identical to SEQ ID NO: 2

SEQ ID NO: 5 - DNA, 1893 bp, Oryza sativa
agtcactagctaagctgctagccttgctaccacgtgttggagatggaggctaagccggtggcgatggaggtggagggggtcgaggcggcggggggcaagccgcggttcaggatgccggtggactccgacctcaaggcgacggagttctggctcttctccttcgcgaggccacacatggcctccttccacatggcgtggttctccttcttctgctgcttcgtgtccacgttcgccgcgccgccgctgctgccgctcatccgcgacaccctcgggctcacggccacggacatcggcaacgccgggatcgcgtccgtgtcgggcgccgtgttcgcgcgtctggccatgggcacggcgtgcgacctggtcgggcccaggctggcctccgcgtctctgatcctcctcaccacaccggcggtgtactgctcctccatcatccagtccccgtcggggtacctcctcgtgcgcttcttcacgggcatctcgctggcgtcgttcgtgtcggcgcagttctggatgagctccatgttctcggcccccaaagtggggctggccaacggcgtggccggcggctggggcaacctcggcggcggcgccgtccagctgctcatgccgctcgtgtacgaggccatccacaagatcggtagcacgccgttcacggcgtggcgcatcgccttcttcatcccgggcctgatgcagacgttctcggccatcgccgtgctggcgttcgggcaggacatgcccggcggcaactacgggaagctccacaagactggcgacatgcacaaggacagcttcggcaacgtgctgcgccacgccctcaccaactaccgcggctggatcctggcgctcacctacggctacagcttcggcgtcgagctcaccatcgacaacgtcgtgcaccagtacttctacgaccgcttcgacgtcaacctccagaccgccgggctcatcgccgccagcttcgggatggccaacatcatctcccgccccggcggcgggctactctccgactggctctccagccggtacggcatgcgcggcaggctgtgggggctgtggactgtgcagaccatcggcggcgtcctctgcgtggtgctcggaatcgtcgacttctccttcgccgcgtccgtcgccgtgatggtgctcttctccttcttcgtccaggccgcgtgcgggctcaccttcggcatcgtgccgttcgtgtcgcggaggtcgctggggctcatctccgggatgaccggcggcgggggcaacgtgggcgccgtgctgacgcagtacatcttcttccacggcacaaagtacaagacggagaccgggatcaagtacatggggctcatgatcatcgcgtgcacgctgcccgtcatgctcatctacttcccgcagtggggcggcatgctcgtaggcccgaggaagggggccacggcggaggagtactacagccgggagtggtcggatcacgagcgcgagaagggtttcaacgcggccagcgtgcggttcgcggagaacagcgtgcgcgagggcgggaggtcgtcggcgaatggcggacagcccaggcacaccgtccccgtcgacgcgtcgccggccggggtgtgaagaatgccacggacaataaggtcgcggttgtagtacaactgtacaaattgatggtacgtgtcgtttgaccgcgcgcgcgcacagtgtgggtcgtggcctcgtgggcttagtggagtacagtgaggggtgtacgtgtgtcgtggcgcgcgcggtcacctcggtggccttgggattgggggggcactatacgctagtactccagatatatacgggtttgatttacttctgtggatcggcgcttgttggtggtttgctccctgtggtttttgtgatggtaatcatactcatactcaaacagtc

SEQ ID NO: 6 - protein, 516 aa, Oryza sativa
MEAKPVAMEVEGVEAAGGKPRFRMPVDSDLKATEFWLFSFARPHMASFHMAWFSFFCCFVSTFAAPPLLPLIRDTLGLTATDIGNAGIASVSGAVFARLAMGTACDLVGPRLASASLILLTTPAVYCSSIIQSPSGYLLVRFFTGISLASFVSAQFWMSSMFSAPKVGLANGVAGGWGNLGGGAVQLLMPLVYEAIHKIGSTPFTAWRIAFFIPGLMQTFSAIAVLAFGQDMPGGNYGKLHKTGDMHKDSFGNVLRHALTNYRGWILALTYGYSFGVELTIDNVVHQYFYDRFDVNLQTAGLIAASFGMANIISRPGGGLLSDWLSSRYGMRGRLWGLWTVQTIGGVLCVVLGIVDFSFAASVAVMVLFSFFVQAACGLTFGIVPFVSRRSLGLISGMTGGGGNVGAVLTQYIFFHGTKYKTETGIKYMGLMIIACTLPVMLIYFPQWGGMLVGPRKGATAEEYYSREWSDHEREKGFNAASVRFAENSVREGGRSSANGGQPRHTVPVDASPAGV

SEQ ID NO: 7 - DNA, 1701 bp, Oryza sativa
gattccccacctctcccacctcactcctacctcactcctagtcctctgccgaaagtacttcctccgtttcacaatgtaagtcattctaatatttttcacattcatattgatgtttgaatctagattgatatatatgtttagattcgttagcatcgatatgaatatgggaaatgctagaatgacttatattgtgaaacagagtgagtatcatgtaaaagttagaaggaaaaaaatagagctgtttgtgatgatatgggtgtggttgtgttgtgtgagccgatgtccattgtactgtactcattttaaatgtacgtaccgttaacttatatagttatatgcgtttgatcatttgtcaaaatttagtgaaactttaaaatttattatacttaaagtatatttaatgataaatttaaaataaaataaactttcagctgttatgttcaaaatcaacatcgtcagatattttaaattaaaggtagtacttttaaaaaaaggatttttgcggtgtgtcgtggcgaaactgctaccaagtttcaatgatcatatgccatttcataggataattactctcatcgtggtaagtaagaatcgattgcctattttcggcaggctgttgtttcaaagcatcgatctgcttggacaacttgagcaaagctagctagaactgggtcgatataattgcagcactaggcaatcaagagacggagctggccaccagctagctgagctgagctgatatgatcaacacagtgcagacttggtcgtgttcgagttcgatcgacggatggctgtcctgctcttgcgctcatgcatgtcatctcttcggaagtaggagtacagcagtacttgaggaatattattagagagtaagttgaactgttttcaatagttcagggtgtaaactaagctgaggaattgttaggaggttaaatgctgtggcaaaatagtttggaggagcgaaatgatttttttttcatatgaaaaacatctaaatttattttttgccaaaacactagtatatcatcaaattttcatccattaagaacgccttctcaatattaataattccaatgtgatatcttaatgctcaatgaacctaaaatagtttggatgagtgaaatggactctttttgagtttttttccatatgaaaacatctaaatttatttttttttgccaaaacactggtatatcatcaaattttcctccattaagaacgccttctcaacgttaataactccaatgttattatcttaatgccaaatgaacctaccatgaacgtcatgctcacaatttaattaacaacaaccgaggcactcaagatcattcgcggttgccgcttctcaccggttgcctgaacccttgggacccctccaaaagcttaattacccccaaaaccgcatgatctctctcttctcttctcttctcacacgtcgtcaaagcctctgactttggatatccccgaccccactaaacttaatcaacttgatcattacaacaattaagttgcctcttgaatccaacgaagtagctggtcaactctccgagctcgtagcctcgctctcccgcctataaattcaccgatcgatcgatcgatcgatctcagcatcagcagcagcagcagattcatttcttggtcttcgtctccgtctccgtccttgggttgatatccagaatcagtcggtttggtttgtcagcaatg

SEQ ID NO: 8 - DNA, 693 bp, Oryza sativa
attcatttcttggtcttcgtctccgtctccgtccttgggttgatatccagaatcagtcggtttggtttgtcagcaatggcgaggctagccggcgttgctgctctctcgttggtgctcgtcttgctcggcgccggcgtgccccggccggcggccgccgccgcggcgaagacgcaggtgttcctctccaagctgcccaaagcgctcgtcgtcggcgtctcgcccaagcacggtgaagtcgtgcacgccggcgagaacacggtgacggtgacgtggtcgctgaacacgtcggagccggcgggcgccgacgcggcgttcaagagcgtgaaggtgaagctgtgctacgcgccggcgagccggacggaccgcgggtggcgcaaggcctccgacgacctgcacaaggacaaggcgtgccagttcaaggtcaccgtgcagccgtacgccgccggcgccggcaggttcgactacgtggtggcgcgcgacatcccgacggcgtcctacttcgtgcgcgcctacgcggtggacgcgtccggcacggaggtggcctacgggcagagctcgccggacgccgccttcgacgtcgccgggatcaccggcatccacgcctccctcaaggtcgccgccggcgtcttctccaccttctccatcgccgcgctcgccttcttcttcgtcgtcgagaagcgcaagaaggacaag

SEQ ID NO: 9 - protein, 206 aa, Oryza sativa
MARLAGVAALSLVLVLLGAGVPRPAAAAAAKTQVFLSKLPKALVVGVSPKHGEVVHAGENTVTVTWSLNTSEPAGADAAFKSVKVKLCYAPASRTDRGWRKASDDLHKDKACQFKVTVQPYAAGAGRFDYVVARDIPTASYFVRAYAVDASGTEVAYGQSSPDAAFDVAGITGIHASLKVAAGVFSTFSIAALAFFFVVEKRKKDK

SEQ ID NO: 10 - DNA, 794 bp, Oryza sativa
acctctagcagagaacgatcatggctcggtttggggcggtaattcaccgcgtgtttctaccgctgttgctgctccttgtagttctcggtgcttgccatgtcacgccggcggcggcggcggcgggggcgcgcctctccgcgctcgcgaaggcgctcgtcgtcgaggcgtcgccccgtgccggccaagtcctgcacgccggcgaggacgccatcaccgtgacatggtcgctgaacgcgacggcggcggcggcggcggccggggcggatgccggctacaaggcggtgaaggtgaccctgtgctacgcgccggcgagccaggtgggccgcgggtggcgcaaggcccacgacgacctgagcaaggacaaggcgtgtcagttcaagatcgcccagcagccgtacgacggcgccggcaagttcgagtacacggtggcacgcgacgtcccgacggcgtcgtactacgtgcgcgcctacgcgctcgacgcgtcgggggcgcgggtggcctatggcgagacggcgccctcggccagcttcgccgtcgcgggcatcaccggcgtcaccgcgtccatcgaggtcgccgccggcgtgctctccgcgttctccgtcgccgcgctcgccgtcttcctcgtcctcgagaacaagaagaagaacaagtgattgtggtttgcttgtgttagtagtctgtacaattgtaatgtgtgactgtgtacgacagcacggagtgtgtctacagcgcccagcacacacacctccgtatgttcaacctacaaaaccaacggaataatagatggatctttg

SEQ ID NO: 11 - protein, 210 aa, Oryza sativa
MARFGAVIHRVFLPLLLLLVVLGACHVTPAAAAAGARLSALAKALVVEASPRAGQVLHAGEDAITVTWSLNATAAAAAAGADAGYKAVKVTLCYAPASQVGRGWRKAHDDLSKDKACQFKIAQQPYDGAGKFEYTVARDVPTASYYVRAYALDASGARVAYGETAPSASFAVAGITGVTASIEVAAGVLSAFSVAALAVFLVLENKKKNK

SEQ ID NO: 12 - DNA, 1825 bp, Oryza sativa
ttgggctttttcttctgttgtgcttgtgcttgtgttggtccggaggttttatgggctacttgggcaaaattgcattgccaacaggcgaactgccatagtaaaaagagaaaattcataaaatgtcatcgataaatatcccaattcaaacgaattcgctatccatctgtcgacagattttcgtttaaagatttgtcgatttttgggccatatgatttgcgtggagatttgtgctaactaatacgatggataaaagagacaagtctgtgcgtagcgggggatcgattccaagagcactagttttccctgttaaacgtacaccacagagcaattaagccaagttggttcaagacaaaataaagtttatattgaagttacagtgttagaaaactaaaattacaatgtaattatactatagttacatgtatgtaaatatggtgcaattatagtgtaacaataatataattaaaattacagtgcaattatactataattacatatgtaactatggtgtaattataatgtaacaacaatgtaattaaaattacagtgcaattataccataattacatatgtaactatggtgtaattatagtgtaacaacaatgtaattaaaattacagtgtaactataccataattacatttgtaactatggtgtaattatagtgtaacaacaatgtaactttagtagatggattggtaatatctttgtatgcactaaaaaatatctagtagatatcacactatacatgagcagatgagctctcaaggattaataattaatatggataaataaccaactaattttgattaagaaacgagagtgtgaattaacgtcggaagcccatccatactatcgattagtagatgcgggacctcagcagtgctgtacacgtgtcatccatctatcgtttatgggtttaatccaaaggtacaaattttgacaaatttatgatgaaaaatctgtcgacagatgtgtagactctccccaattcaaaaaatatcatcgataaatctaatctctttagaaatgtcatcgtacaagtgcttttgttctagagttaccatcgttgttaagttttcggttgcatccatctgttaagtgctataaaaagatcattttaccctataaattattaaaggttgattaaaaattttgctcttttcgttagtttacactcaattagtttgctctttacactaaaatattgaaaattgactgaaagagtaaaagttttaatcatttaataatttgtaggggtaaaatggttttttatagcactcaacgaatggctacaaacgaaaatctaacgacgatgatatttttagaataaaagcgcttgtacaataatatttttaggaagctgtgttcgttaatggtatttcttggattggagggctcgtgatttttctccggcaaacagtgtaccgtgttctgaccaccaccaacagactgtctgactactaccgttaacgtggtactgctactagtctactactactactgtgcagtgtactacagataccacacagctgcattagcctgcattcgccgcttcacatcgccatggccctcaaaaggtcgaccgaggtgccccatcgagccgacgagacagccaaacgtacgtgctccgacagtcagacccgcgtgaaccatcaagctccgactttcggaaccacccatcgctaccctcctcgaccccacaaagttgaactcccccgatctccctccctctccacgagtcaacttactcgacgctaccacgcctatatataagctaccgctccgctcaaatggcctccacctctagcagagaacgatcatg

SEQ ID NO: 13 - DNA, 853 bp, Arabidopsis thaliana
gatacaattacaatatgtagagtatcttataggtgacgtaaccatgaaatatagaattctttggaatctgaaactgaattattcagttgataaatgataaacaaatactcatatctcatcctttggcatggcgatccagaagatcctctttgcttcacttctcatatgctcactgatccaatccatccacggggcggaaaaagtaagactcttcaaagagctggacaaaggtgcacttgatgtcaccactaaacccagccgagaaggaccaggtgttgttttggatgccggcaaggatacgttgaacattacatggacgctaagctcgattgggtctaaaagagaggctgaatttaagatcatcaaagttaagctatgctacgctccacctagccaagttgaccgaccatggcgcaaaacccatgacgagctcttcaaagacaagacctgcccacacaagatcatagccaagccttatgacaaaacacttcaatcaactacttggactcttgagcgtgacatccccaccggaacctacttcgttcgtgcctacgcggttgatgccattggccatgaagttgcctatggacagagcaccgacgatgccaagaaaaccaatctcttcagcgttcaggctatcagtggccgccacgcgtccctagatattgcctccatctgtttcagtgtcttctccgtcgtggctcttgtcgtcttctttgtcaatgagaagaggaaggccaagatagagcaaagcaaatgagtcgtttactttgcgtatttgtgacgttgaacccaaaaaaagttgactttgaactttcttgtttaccaattccttttgtcttgttgcacactt

SEQ ID NO: 14 - DNA, 859 bp, Arabidopsis thaliana
taaaagtcagcaaaacacaaggcatattcctcttctcttcctcagccttatttttctgatattcagtttcaaggatatatccatggcgatccagaagatcctctttgcttcacttctcatatgctcactgatccaatccatccacggggcggaaaaactaagactcttcaaagagctggacaaaggtgcacttgatgtcaccactaaacccagccgagaaggaccaggtgttgttttggatgccggcaaggatacgttgaacattacatggacgctaagctcgattgggtctaaaagagaggctgaatttaagatcatcaaagttaagctatgctacgctccacctagccaagttgaccgaccatggcgcaaaacccatgacgagctcttcaaagacaagacctgcccacacaagatcatagccaagccttatgacaaaacacttcaatcaactacttggactcttgagcgtgacatccccaccggaacctacttcgttcgtgcctacgcggttgatgccattggccatgaagttgcctatggacagagcaccgacgatgccaagaaaaccaatctcttcagcgttcaggctatcagtggccgccacgcgtccctagatattgcctccatctgtttcagtgtcttctccgtcgtggctcttgtcgtcttctttgtcaatgagaagaggaaggccaagatagagcaaagcaaatgagtcgtttactttgcgtatttgtgacgttgaacccaaaaaaagttgactttgaactttcttgtttaccaattccttttgtcttgttgcacacttcttctttcttatatgcttttatttatgtgtttgtacaattaagccattgat

SEQ ID NO: 15 - DNA, 1900 bp, Arabidopsis thaliana
atcgatcaaataaacttgaatcaaatctcaaacttgcaaagaaacttgaaatattttataacaatgggtgattctactggtgagccggggagctccatgcatggagtcaccggtagagaacaaagctttgctttctcggtgcaatcaccaattgtgcataccgacaagacggccaagttcgaccttccggtggacacagagcataaggcaacggttttcaagctcttctccttcgccaaacctcacatgagaacgttccatctctcgtggatctctttctccacatgttttgtctcgactttcgcagctgcaccacttgtccctatcatccgggagaatctcaacctcaccaaacaagacattggaaacgccggagttgcctctgtctctgggagtatcttctctaggctcgtgatgggagccgtgtgtgatcttttgggtccccgttacggttgtgccttccttgtgatgttgtctgccccaacggtgttctccatgagcttcgtgagtgacgcagcaggcttcataacggtgaggttcatgattggtttttgcctggcgacgtttgtgtcttgtcaatactggatgagcactatgttcaacagtcagatcattggtctggtgaatgggacagcagccggatggggaaacatgggtggcggcataacgcagttgctcatgcccattgtgtatgaaatcattaggcgctgcggttccacagccttcacggcctggaggatcgccttctttgtacccggttggttgcacatcatcatgggaatcttggtgctcaatctaggtcaagatctgccagatggaaatcgagctaccttggagaaagcgggagaagttgccaaagacaaattcggaaagattctgtggtatgccgttacaaactacaggacttggatcttcgttcttctctacggatactccatgggagttgagttgagcactgataatgttatcgccgagtacttctttgacaggtttcacttgaagctccacacagcagggctcatagcagcatgtttcggaatggccaatttctttgctcgtccagcaggaggctacgcatctgactttgcagccaagtacttcgggatgagagggaggttgtggacgttgtggatcatacagacggctggtggcctcttctgtgtgtggctcggccgcgccaacacccttgtaactgccgttgtggctatggtgctcttctctatgggggcacaagctgcttgcggagccacctttgcaattgtgccctttgtctcccggcgagctctaggcatcatctcgggtttaaccggggctggagggaactttggatcagggctcacacaactcctcttcttctcgacctcacacttcacaactgaacaagggctaacgtggatgggagtgatgatagtcgcttgcacgttacctgtgaccttagttcactttcctcaatggggaagcatgttcttgcctccttccacagatccagtgaaaggtacagaggctcattattatggttctgagtggaatgagcaggagaagcagaagaacatgcatcaaggaagcctccggtttgccgagaacgccaagtcagagggtggacgccgcgtccgctctgctgctacgccgcctgagaacacacccaacaatgtttgatcatacattccacccacggtggaatggtgaaggatgatcgcatataagaatatgtcacacagtgaaaaaaaaaaatgcaaatgttatcaatgcttgcataacattactatctatctttcatttactaaacaaaccttttgctttttgccttgaaatctttttattatatatcaaaatatatctctatgtcttgagatttgattattttgcatatatcattaatgatttgataatattggaactg

SEQ ID NO: 16 - DNA, 1709 bp, Arabidopsis thaliana
aaacttgaattttctcaaaggaacttgatacgtttaaaatacatgggttctactgatgagcccagaagttccatgcatggagttaccggtagagaacagagctatgctttctcggtagatggtagtgagccgaccaacacaaagaaaaagtacaatctgccggtggacgcggaggataaggcaacggttttcaagctcttctccttcgccaaacctcacatgagaacgttccacctctcgtggatctctttctccacatgttttgtttcgacgttcgcagctgcaccacttatcccgatcatcagggagaatcttaacctcaccaaacatgacattggaaacgctggagttgcctccgtctcggggagtatcttctctaggctcgtgatgggagccgtgtgtgatcttttgggtcctcgttacggttgtgccttccttgtgatgttgtctgccccaacggtgttctccatgagcttcgtgagtgacgcagcaggcttcataacggtgaggttcatgattggtttttgcctggcgacgtttgtgtcttgtcaatactggatgagcactatgttcaacagtcagatcatcggtctggtgaacgggacagcagccggatggggaaacatgggtggcggcataacgcagttgctcatgcccattgtgtatgaaatcattaggcgctgcggatcaacagcgttcacggcctggaggatcgccttctttgtccccggttggttgcacatcatcatgggaatcttggtgctcacgctaggtcaagatctgccaggtggaaacagagctgccatggagaaagcgggagaagttgccaaagacaaattcggaaagattctatggtacgccgttacaaattacaggacttggattttcgttcttctgtatggatattccatgggagttgagttaagcacagacaatgttatcgccgagtacttctttgacaggtttcacttgaagcttcacacagcggggattatagcagcatgtttcggaatggccaatttctttgctcgtccagcaggaggctgggcatctgacattgcagccaagcgcttcggaatgcgagggaggttgtggactttgtggatcattcagacgtccggtggtctcttttgtgtgtggctcggacgtgccaacaccctcgtcactgccgttgtatctatggtcctcttctctttaggagcacaagccgcttgcggagccacctttgctatcgtgccctttgtctcccggcgagctctaggcattatctcgggtttaaccggggctggagggaactttgggtcaggactcacacagctcgtctttttctcgacttcgcgcttcacaactgaagaagggctaacgtggatgggagtgatgatagttgcttgcacgttgcctgttaccttaatccactttcctcagtggggaagcatgttcttccctccttccaacgattcggtcgacgctacggagcactattatgttggcgaatatagtaaggaggagcagcagattggcatgcatttaaaaagcaaactgtttgctgatggagccaagaccgagggaggcagcagcgtccacaaagggaacgcaaccaacaatgcttgatcatgtgtcattgatatcaagaaattaataatttcacttatgtgaaatggacataaactgttggaaaataaagaaccatttctttcatcatttgcttt

SEQ ID NO: 17 - DNA, 1620 bp, Arabidopsis thaliana
atgactcacaaccattctaatgaagaaggctccattggaacctccttgcatggagttacagcaagagaacaagtcttctctttctccgtcgatgcttcgtctcaaacagtccaatcagacgatccaacagctaaattcgcccttccggttgattccgaacatcgagccaaagtgttcaacccactctcttttgctaaacctcacatgagagccttccacttaggatggctctcattcttcacatgcttcatctccaccttcgcggcagcaccattagtccccatcatccgcgacaacctcgacctcactaaaaccgacattggaaacgccggagtcgcatccgtctctggtgccattttctcaaggttagccatgggagcggtttgtgatctcctcggtgcacgatatgggactgccttctccctcatgctaaccgccccaaccgtcttctcaatgtcgtttgtgggtggccctagcggatacttaggcgtccggttcatgatcggattctgtctcgccacgtttgtatcatgccagtattggaccagcgttatgttcaacggtaagatcataggactagtgaacggctgtgcaggcgggtggggtgatatgggcggtggagtgactcaactcctaatgccgatggtcttccacgtcatcaaacttgccggagccactccgttcatggcctggcggatagctttcttcgttcccggatttcttcaagttgttatgggcattctcgtcctcagtctcggccaagatctccctgacggtaacctaagtacccttcagaagagtggtcaagtctctaaagacaaattctccaaggttttctggtttgctgtgaagaactacagaacatggattttattcgttctttatggatcttccatgggaattgaattaactatcaacaacgttatctccggatatttttacgacaggtttaaccttaagcttcaaacagctggtatagtagcagccagctttggaatggctaacttcatcgcccgtcccttcggtggttacgcttctgatgtagcggctcgggtttttggcatgagaggccggttatggaccttatggatctttcaaaccgtaggagctcttttctgtatctggctaggtcgagctagttcacttcccatagcaatcctagcaatgatgctcttctcaatcggtacacaagcagcttgcggagccctcttcggagttgcaccttttgtctcgcgccgctctctagggctcatatcgggactaaccggcgcaggaggaaacttcgggtccggtttgactcaactgcttttcttctcatcagcgaggtttagtacagctgagggactctcattgatgggcgttatggcggttttgtgcacactcccagttgcgtttatacattttccgcaatggggaagcatgtttttaagaccgtcgaccgatggagaaagatcacaggaggaatattattacggttctgagtggacggagaatgagaaacaacaaggattgcacgaaggaagcatcaaatttgcagagaatagtaggtcagagagaggccggaaagtagctttggctaacattccaacgccggagaacggaactccaagtcatgtttga

SEQ ID NO: 18 - DNA, 1674 bp, Arabidopsis thaliana
atggccgatggttttggtgaaccgggaagctcaatgcatggagtcaccggcagagaacaaagctatgcattctctgtcgagtctccggcagttccttccgactcatcagcaaaattttctctccccgtggacaccgaacacaaagccaaagtcttcaaactcttatcctttgaagctccacatatgagaactttccatcttgcttggatctcattcttcacttgcttcatttccactttcgctgctgctcctcttgtccccatcattagagataaccttaatctcacaagacaagatgtcggaaatgctggtgttgcttctgtctctggcagtatcttctctaggcttgttatgggagcagtttgtgatctccttgggccacgttatggctgtgctttcctcgtcatgctctctgctccaaccgtcttctccatgtctttcgttggtggtgccggagggtacataacggtgaggttcatgatcgggttctgcctggcgactttcgtgtcatgccagtattggatgagcacaatgttcaatggtcagatcataggtctagtgaacgggacagcggcagggtgggggaacatgggcggtggggtcactcagttgctcatgccaatggtctatgagatcatccgacggttagggtccacgtccttcaccgcatggaggatggctttcttcgtccccgggtggatgcacatcatcatggggatcttggtcttgactctagggcaagacctccctgatggtaatagaagcacactcgagaagaaaggtgcagttactaaagacaagttctcaaaggttttatggtacgcgatcacgaactataggacatgggttttcgtgctgctatatggatactccatgggagtagagctcacaaccgataacgtcatcgctgagtactttttcgacaggttccatcttaagcttcataccgccggtataatcgcggcaagctttggtatggcaaacttctttgcccgtcctattggtggttgggcctcagatattgcggctagacgcttcggcatgagaggccgtctctggaccctatggatcatccaaaccttaggcggtttcttctgcctatggctaggccgagccaccacgctcccgaccgcggttgtcttcatgatcctcttctctctcggcgctcaagccgcttgtggagctacctttgctatcatacctttcatctcacgccgctccttagggatcatctctggtcttactggagctggtggaaacttcggctctggtttgacccaactcgtattcttctcgacctcaacgttctccacggaacaagggctgacatggatgggggtgatgattatggcgtgtacattacccgtcactttagtgcacttcccgcaatggggaagcatgtttttgccttccacggaagatgaagtgaagtctacggaggagtattattacatgaaagagtggacagagaccgagaagcgaaagggtatgcatgaagggagtttgaagttcgccgtgaatagtagatcggagcgtggacggcgcgtggcttctgcaccgtctcctccgccggaacacgtttaattcccgcaatggggaagcatgtttttgccttccacggaagatgaagtgaagtctacggaggagtattattacatgaaagagtggacagag

SEQ ID NO: 19 - protein, 210 aa, Arabidopsis thaliana
MAIQKILFASLLICSLIQSIHGAEKVRLFKELDKGALDVTTKPSREGPGVVLDAGKDTLNITWTLSSIGSKREAEFKIIKVKLCYAPPSQVDRPWRKTHDELFKDKTCPHKIIAKPYDKTLQSTTWTLERDIPTGTYFVRAYAVDAIGHEVAYGQSTDDAKKTNLFSVQAISGRHASLDIASICFSVFSVVALVVFFVNEKRKAKIEQSK

SEQ ID NO: 20 - protein, 210 aa, Arabidopsis thaliana
MAIQKILFASLLICSLIQSIHGAEKLRLFKELDKGALDVTTKPSREGPGVVLDAGKDTLNITWTLSSIGSKREAEFKIIKVKLCYAPPSQVDRPWRKTHDELFKDKTCPHKIIAKPYDKTLQSTTWTLERDIPTGTYFVRAYAVDAIGHEVAYGQSTDDAKKTNLFSVQAISGRHASLDIASICFSVFSVVALVVFFVNEKRKAKIEQSK

SEQ ID NO: 21 - protein, 530 aa, Arabidopsis thaliana
MGDSTGEPGSSMHGVTGREQSFAFSVQSPIVHTDKTAKFDLPVDTEHKATVFKLFSFAKPHMRTFHLSWISFSTCFVSTFAAAPLVPIIRENLNLTKQDIGNAGVASVSGSIFSRLVMGAVCDLLGPRYGCAFLVMLSAPTVFSMSFVSDAAGFITVRFMIGFCLATFVSCQYWMSTMFNSQIIGLVNGTAAGWGNMGGGITQLLMPIVYEIIRRCGSTAFTAWRIAFFVPGWLHIIMGILVLNLGQDLPDGNRATLEKAGEVAKDKFGKILWYAVTNYRTWIFVLLYGYSMGVELSTDNVIAEYFFDRFHLKLHTAGLIAACFGMANFFARPAGGYASDFAAKYFGMRGRLWTLWIIQTAGGLFCVWLGRANTLVTAVVAMVLFSMGAQAACGATFAIVPFVSRRALGIISGLTGAGGNFGSGLTQLLFFSTSHFTTEQGLTWMGVMIVACTLPVTLVHFPQWGSMFLPPSTDPVKGTEAHYYGSEWNEQEKQKNMHQGSLRFAENAKSEGGRRVRSAATPPENTPNNV

SEQ ID NO: 22 - protein, 522 aa, Arabidopsis thaliana
MGSTDEPRSSMHGVTGREQSYAFSVDGSEPTNTKKKYNLPVDAEDKATVFKLFSFAKPHMRTFHLSWISFSTCFVSTFAAAPLIPIIRENLNLTKHDIGNAGVASVSGSIFSRLVMGAVCDLLGPRYGCAFLVMLSAPTVFSMSFVSDAAGFITVRFMIGFCLATFVSCQYWMSTMFNSQIIGLVNGTAAGWGNMGGGITQLLMPIVYEIIRRCGSTAFTAWRIAFFVPGWLHIIMGILVLTLGQDLPGGNRAAMEKAGEVAKDKFGKILWYAVTNYRTWIFVLLYGYSMGVELSTDNVIAEYFFDRFHLKLHTAGIIAACFGMANFFARPAGGWASDIAAKRFGMRGRLWTLWIIQTSGGLFCVWLGRANTLVTAVVSMVLFSLGAQAACGATFAIVPFVSRRALGIISGLTGAGGNFGSGLTQLVFFSTSRFTTEEGLTWMGVMIVACTLPVTLIHFPQWGSMFFPPSNDSVDATEHYYVGEYSKEEQQIGMHLKSKLFADGAKTEGGSSVHKGNATNNA

SEQ ID NO: 23 - protein, 539 aa, Arabidopsis thaliana
MTHNHSNEEGSIGTSLHGVTAREQVFSFSVDASSQTVQSDDPTAKFALPVDSEHRAKVFNPLSFAKPHMRAFHLGWLSFFTCFISTFAAAPLVPIIRDNLDLTKTDIGNAGVASVSGAIFSRLAMGAVCDLLGARYGTAFSLMLTAPTVFSMSFVGGPSGYLGVRFMIGFCLATFVSCQYWTSVMFNGKIIGLVNGCAGGWGDMGGGVTQLLMPMVFHVIKLAGATPFMAWRIAFFVPGFLQVVMGILVLSLGQDLPDGNLSTLQKSGQVSKDKFSKVFWFAVKNYRTWILFVLYGSSMGIELTINNVISGYFYDRFNLKLQTAGIVAASFGMANFIARPFGGYASDVAARVFGMRGRLWTLWIFQTVGALFCIWLGRASSLPIAILAMMLFSIGTQAACGALFGVAPFVSRRSLGLISGLTGAGGNFGSGLTQLLFFSSARFSTAEGLSLMGVMAVLCTLPVAFIHFPQWGSMFLRPSTDGERSQEEYYYGSEWTENEKQQGLHEGSIKFAENSRSERGRKVALANIPTPENGTPSHV

SEQ ID NO: 24 - protein, 527 aa, Arabidopsis thaliana [sequence omitted]

SEQ ID NO: 25 - DNA, 1800 bp, Arabidopsis thaliana
gatgaagacatcggtggtatgaatcttctgattttgaatctggtaacaccatcaccatccaaaataagaaaaaaaaaatacgaaaaatgattaaagagagcgtctcccatcccaattactaaaaaaaaatgggaaagaaaccacccggcaattgctggtagccgtaaaaaccaaacaagaaaatagatattacttttgtcctcccaaagattttgtccagttaaccaacttttcgaagccatgtcttacgttttcagtatatgcctctaaggcaacttgtattttctcaaagtgtggtttccatattgtcataactcggcttacctagaaaaacaagtaacaatggtcacaatgtatagtgcaatgtcaaaagagaatcagacataatgaataaagatggaaacatattaacctcaagatagtcgtaggaaacgtcgaatggttgggtgaaatgtggggtcaaaagcctgcttacatgtatgtaacacctcgatggacttatcggttaagtattgaatttttggttggagataaatggttaatgtcacatacacatctttgatgcttggaatccatttctgtgatatctaagagaagaattcaaaggttaataagaataaagcaataatgacttgtcttataacgaaaaccaaaaactttcagaataaaaccttttgaagagtttggctaagatcttgttgccgcagagtcactatatatgcttgcaaccataataaaaaaaaagaggatcatttaagattgatcactatgacaattttatcgatgttaattatgaaccttcttatagagttacctgagttttatttaggtttagaccaagcaacttcccatcgatctgaacacaagtaaaaaaaaagtaaagaaagcttttgaatatgtacatacatcagatccaagggacaccatgtttcttacattttcaagctttgagccaagttccgcgactttcttttctgcgatttcagctcgagtttccacttccagacttctcttgcgttgcatttccacttcttctcgtagttcaagtatctgccaatgcagaactaaattgaggtaacaaaacaagaaggaaatacacacctcaaactacaaaacaccaacctgtttctgaagttcataagccgttccttctgcttcactggattctcagtctaaataaaaaagcatcaacagcaacaaaagtgtaaataagtttcttctatacataacaattttgaatacatgaaccaaagattaattaaaaatatcacttgaagagatccaaccacatcaaagatgatgcagcttgagtaattgttaactctttccaactttggaaagagacaaaacgtcaaagtgatttccagaaacagagaacggaagctcaaaagtctctcttgtgccaaatatattaaaccctaaaaaattcaacttctatccgaatttctcagaaacaaaacaaagacacataacttcatatgaaattccagtgaacggttacctgaggaagaaccttatttgttatcagtgtaaaataacagagaagaggtacgagtaaaagaaacaaaagtgacttaggaaacgccattgttggagactgctcactggaagatagagagtcgtgagagacagtgataaagcgtatcaagtcatatagagggtcttcttatctttttctttaacatgtgagggttgagttaattatgcgggctgattatagagtttttaaattgaatttacgattgtttttttcttacatataaatgcaatctatatttgtgttcggaataacccatctaa

SEQ ID NO: 26 - DNA, 1800 bp, Arabidopsis thaliana
atttctgtgatatctaagagaagaattcaaaggttaataagaataaagcaataatgacttgtcttataacgaaaaccaaaaactttcagaataaaaccttttgaagagtttggctaagatcttgttgccgcagagtcactatatatgcttgcaaccataataaaaaaaaagaggatcatttaagattgatcactatgacaattttatcgatgttaattatgaaccttcttatagagttacctgagttttatttaggtttagaccaagcaacttcccatcgatctgaacacaagtaaaaaaaaagtaaagaaagcttttgaatatgtacatacatcagatccaagggacaccatgtttcttacattttcaagctttgagccaagttccgcgactttcttttctgcgatttcagctcgagtttccacttccagacttctcttgcgttgcatttccacttcttctcgtagttcaagtatctgccaatgcagaactaaattgaggtaacaaaacaagaaggaaatacacacctcaaactacaaaacaccaacctgtttctgaagttcataagccgttccttctgcttcactggattctcagtctaaataaaaaagcatcaacagcaacaaaagtgtaaataagtttcttctatacataacaattttgaatacatgaaccaaagattaattaaaaatatcacttgaagagatccaaccacatcaaagatgatgcagcttgagtaattgttaactctttccaactttggaaagagacaaaacgtcaaagtgatttccagaaacagagaacggaagctcaaaagtctctcttgtgccaaatatattaaaccctaaaaaattcaacttctatccgaatttctcagaaacaaaacaaagacacataacttcatatgaaattccagtgaacggttacctgaggaagaaccttatttgttatcagtgtaaaataacagagaagaggtacgagtaaaagaaacaaaagtgacttaggaaacgccattgttggagactgctcactggaagatagagagtcgtgagagacagtgataaagcgtatcaagtcatatagagggtcttcttatctttttctttaacatgtgagggttgagttaattatgcgggctgattatagagtttttaaattgaatttacgattgtttttttcttacatataaatgcaatctatatttgtgttcggaataacccatctaatattactccatgtattaaactaaaatattttgcatgttttggtagatcaactttttgaatgatcacagacctacaaacatcaaccctctaattatccaattttaccataatccacgagctcttaaaattcatttttaatatatataattaaaatagttcaaacagtttaaactttgtgaccaagttaaaatatttaaaatagtttgactttgtgatcaacatatttaaatataatcaatattttcatttttaagccggaaaatcacgtcttacaaatatatttctgatagacacacctataattccaaaattttgacttttaaaacaaacaaaaaaaaatctcattaataccactacatacgtttttacaaaaataccattaaaagatattttttcaaatactaaaaaaaactaaaactaaactttaaacctataaaacactaaatcctataaagtttatattcgagtataaaccttaaaagttcaaccctaaatcctgaaagcaagaccctaaacccaaactcaaacttttaaattataaatccttaaacaaaatcatttttagtctttatgg

SEQ ID NO: 27 - DNA, 805 bp, Hordeum vulgare
aaccttctctcaccgagcagctgcgagctccagcccaatttccaagctagaatggcacggtcggagctggtcatggtgttgctagtggtggtcctcgccgccggctgctgcacgtcggcgggcgccgtggcgtacctctccaagctgcctgttaccctcgacgtcaccgcatcccccagtcccggccaagttcttcacgccggcgaggacgtgatcacggtgacatgggccttgaacacgacacaggccggcaaggacgccgactacaagaacgtgaaggtgagcctctgctacgcgccggtgagccagaaggagcgcgagtggcgcaagacccacgacgacctcaagaaggacaagacctgccagttcaaggtcacccagcaggcctaccccggcgccggcaaggtcgagtaccgcgtcgccctcgacatccccaccgccacctactacgtgcgcgcctacgcgctggacgcctcgggcacccaggtcgcctacggccagaccgcgcccaccgcggccttcaatgttgtcagcatcaccggcgtcaccacctctatcaaggtcgctgctggcgttttctccgccttctccgtggcctccctcgccttcttcttcttcattgagaaacgcaagaagaacaactaaactcgcaagccggaaccgtggcatggcaaggtgttcgtccgtgcgcgacttcttcccgtcatttgtaacgtacgcacggctgtactgtaccgtacatgtaagagattgctggtattccctttctcggagactcccgtaaaaaaaaaaaaaaaaaaaaaaa

SEQ ID NO: 28 - DNA, 899 bp, Hordeum vulgare
ccacgcgtccgcagccccatccatcaaaccttctctgaccgagcagcagctgcgagctcgagcccgccccaagttcgacgacggcgatggcacggtcggacctggtcatggcgttgctggtggccgtcctcgccgccggctgctgcgcgtcggccggcgccgtggcgtacctctccaagctgaccgtgaccctcgacgtcaccgcctcccccactcctggccaagttctccacgccggcgaggacgtgatcacggtgacgtgggccctgaacgcgacccggccggccggcgacgacgccgcctacaagagcgtcaaggtcagcctctgctacgcgccggcgagccagaaggagcgcgagtggcgcaagacccacgacgacctcaagaaggacaagacctgccagttcaaggtcacccagcagccctacgccgccggcgccgccggcggcagggtcgagtaccgcgtcgccctcgacatccccaccgccgcctactacgtgcgcgcctacgcgctcgacgcctccggcacgcaggtggcctacggccagaccgcgcccgccaccgccttcgacgtcgtcagcatcacgggcgtcaccacctccatcaaggtcgccgccggcgtcttctccaccttctccgtcgtctccctcgccttcttcttcttcatcgagaagcgcaagaagaataactaaactcacaaggtgttcgtccgtgcgcggctgcttcttcttcttcttcttcttgtcatttgcaacgtacacataatattactgtatgtaagagagtagtgttgatgttctcttttcggggagctcacgtgattgttgatgttgtaccagaggagttgcaagctggagtgtatgttagattgttagtacgtcaaaaaaaaaaaaaaaaaa

SEQ ID NO: 29 - DNA, 597 bp, Hordeum vulgare
atggctcggcaaggcatggtcacggcgctgctgctgctggtcctcgccgccggctgctgcgcatcggcgggcgccgtggcgtacctctccaagctgcctgtgaccctcgacgtcaccgcctcccccactcccggccaagttcttcacgccggcgaggacgtgatcacggtgacgtgggccctgaacgcgagccagccggccggcaaggacgccgactacaagaacgtgaaggtgagcctctgctacgcgccggtgagccagaaggagcgcgagtggcgcaaaacccacgacgacctcaagaaggacaagacctgccagttcaagatcacccagcaggcctaccccggcgccggcaaggttgagtatcgcgtcgccctcgacatccccaccgccacttactacgtgcgcgcctacgcgctcgacgcctcgggcacccaggtcgcctacggccaaaccgcgccaaccgccgccttcaacgtcgtcagcatcacgggagtcaccacctccatcaaggtcgccgccggcgtcttctccgccttctccgtcgcctccctcgccttcttcttcttcattgagaaacgcaagaagaacaactaa

SEQ ID NO: 30 - DNA, 1837 bp, Hordeum vulgare
gaattcgcggccgctcccttactacattgcaagccaagctcaagagcagcagcaacagccaccattagctgcttctagttgttggcaaagatggaggtcgaggcgggcgcccatggcgacactgccgcgagcaagttcacgctgccggtagactccgagcacaaggccaagtccttcaggctcttctccttcgccaacccgcacatgcgcaccttccatctctcgtggatctccttcttcacttgcttcatctccaccttcgccgcagcgccccttgtccccatcattcgtgataacctcaaccttgccaaggccgacatcggcaatgccggtgtggcatccgtttctgggtccatcttctccaggcttgccatgggtgccatctgcgatctcctcgggccgcggtatggatgtgcattcctcgtcatgctctcggcaccgaccgttttctgcatggccgttatcgatgatgcctcagggtacatcgccgtccgctttctcattggcttctcgcttgctacgttcgtgtcatgccaatattggatgagcaccatgtttaatagcaagatcatcggcacagtcaacggcctcgctgctggatggggcaacatgggtggtggcgccacgcagctcatcatgccgctcgtcttccatgcaatccagaagtgtggtgccacgcccttcgtagcgtggcgtattgcctacttcgtgcccggaatgatgcacatcgtgatgggcttgttggtactcaccatggggcaagatctccctgatgggaacctcgcaagtctccagaagaagggagacatggccaaggacaagttctccaaggtcctttggggcgccgttaccaactaccgaacatggatctttgtcctcctctatggctactgcatgggtgtcgagctcaccaccgacaatgtcattgccgagtactacttcgaccacttccacctagacctccgtgccgccggtaccatcgctgcctgcttcggcatggccaacatcgtcgcacgtcctacgggtggctacctctctgaccttggcgcccgctatttcggcatgcgtgctcgcctctggaatatctggatcctccaaaccgctggtggcgctttctgcatctggctcggtcgtgcatcggccctccctgcctcggtgaccgccatggtcctcttctccatctgcgcccaggctgcgtgtggtgctatctttggtgtcgcacccttcgtctccaggcgttcccttggcatcatttccgggttgaccggtgctggtggaaacgtgggcgcagggctcacacagcttctcttcttcacgtcatcgcaatactccactggtaggggtctcgagtacatgggcatcatgatcatggcatgcacgctgcccgtcgctcttgtgcacttcccacaatggggatccatgttcttccctgccagcgccgacgccacggaggaggagtactacgcctcggagtggtccgaagaggagaaagccaagggtctccatatcgccggccaaaaatttgctgagaattcccgctcggagcgcggtaggcgcaacgtcatccttgccacgtccgccacaccacccaacaatacgccccagcacgtatgagactggattgtttttcatacctatgtacaagtactgaactacagtgcacgttcgtatatatatacgcctgcaacatcggctgtaataaggcgtatgaatttacatttgtagtgtaggcctgtgtaatgcgtttcttacgcacgaaatgtttggtctgtgcatgcacgcatgcgagggtacctgtgctctgaatttacaacagctttgaggcggccgcgaattc

SEQ ID NO: 31 - DNA, 1741 bp, Hordeum vulgare
ttacaagctccatctgagagcagcagcaaccaccattagagacacacttagttgccagtgcgactaagctagctagctcgaggaagatggaggtggagtcgagctcgcatggcgccggcgacgaggctgcgagcaagttctcgctgcccgtggactcggagcacaaggccaagtccatcaggctcttctccttcgccaacccccacatgcgcaccttccacctctcctggatctccttcttcacctgcttcgtctccaccttcgctgccgcgcccctcgtccctatcatccgcgacaacctaaacctcgccaaggccgacatcggcaacgccggtgtggcgtccgtgtccgggtctatcttctcgaggctcgccatgggggccatctgcgatctccttggccctcgatatggatgcgccttcctcgtcatgctcgcagcacccaccgtcttctgcatgtccctcatcgatgatgcggcgggctacatcacggtccgcttcctcatcggcttctccctcgcgacgtttgtgtcgtgccagtattggatgagcaccatgttcaacagcaagatcatcggcaccgtcaacggcctggcggccggctggggcaacatgggtggtggtgccacccagctcattatgccactcgtcttccacgccatccagaagtgtggtgccacgcccttcgtcgcatggcgcatcgcctacttcgtgccaggaatgatgcacgtggtgatgggcttgctcgtgctcaccatgggacaggatctccccgatggtaaccttgcaagcctccagaagaagggggagatggccaaggacaagttctccaaggttgtgtggggtgctgttacaaactaccgtacatggatcttcgttcttctttacggatactgcatgggtgttgagctcaccaccgacaacgtcatcgccgagtactacttcgaccactttcaccttgaccttcgaacatccggcaccattgccgcctgttttggcatggccaacatcgttgctcggcctgcgggtggctacctctccgacctcggtgcccgctacttcggcatgcgtgcccgcctctggaacatctggatcctccagaccgctggtggcgcattctgcctctggctcggccgtgcaaaagccctccccgaatccatcactgccatggtcctcttctccatctgcgctcaggcagcatgtggtgcagtctttggtgtcatccccttcgtctcccgccgctccctcggcatcatttcgggcttgagtggagccggtgggaactttggcgccgggctgacacaattgctcttcttcacttcgtcgaagtatggcaccggcagggggcttgagtacatgggtatcatgatcatggcctgcacgctccctgtggcgcttgtgcacttcccacagtggggttccatgctcttgccgccaaacgccaacgccaccgaggaggagttctatgccgccgaatggagcgaggaggagaagaagaagggtctccatatccctggccaaaagtttgccgagaattcccgctcggagcgtggcaggcgcaacgtcatccttgccacagccgccacaccccccaacaacactccccaacacgcataagactcgagcttttctttacctgtgtacacgtacagtgcgcgtattatacacacatcgatcgtgtatatacgcctggaatccgcaagcagtatgttttttgaaaaaaaaaaagcggccgcgaattc

SEQ ID NO: 32 - DNA, 1825 bp, Hordeum vulgare
tctcagttgccactgcagctgatcaagcaagctagctccaaacctccaaggaggaagcagagaaggagactagctcgatcaagcaaggtccaaatggaggtggaggctggtgcccatggcgacacggcggcgagcaagttcacgttgcccgtggactccgagcacaaggccaagtccttcaggctcttctccttcgccaacccacacatgcgcacctttcacctatcgtggatatccttcttcacatgcttcgtctccacctttgccgcggcgcccctggtgcccatcatccgcgacaacctgaacctcgccaaggccgacatagggaatgccggtgtggcatctgtgtctgggtctatcttctcgaggcttgccatgggcgccatctgcgaccttttggggccgcggtatgggtgtgccttcctcgtcatgctctcagcgccaaccgtcttctgcatggccgtcatcgatgacgcctcagggtacatcgccgtacgcttcctcatcggcttctcccttgccacctttgtgtcgtgccaatactggatgagcaccatgttcaacagtaaaatcatcggcacggtcaatggcctcgcggccggctggggcaacatgggcggtggtgccacacaactcatcatgccgcttgttttccacgccatccaaaaatgtggtgccacaccatttgtggcatggcgtattgcctacttcgtgcccggaatgatgcacatcgtgatgggcttgctggtactcaccatggggcaagatctccctgatgggaacctcgcgagcctccagaagagaggagacatggccaaggacaagttctccaaggtcctttggggcgccgtcaccaactaccggacatggatctttgtcctcctatatggctactgcatgggtgtcgaactcaccactgacaatgtcattgccgagtactacttcgaccacttccacctagaccttcgcgccgctggtaccatcgccgcctgcttcggtatggccaacatagtcgcacgtcctatgggcggctacctctctgaccttggcgcccgctatttcggcatgcgtgccctttggaacatctggatcctccaaaccgctggtggcgctttctgcatctggctcggtcgtgcatcggccctccctgcctcggtgaccgccatggtcctcttctccatctgtgcccaggctgcctgtggtgctatctttggtgtcgcacccttcgtctccaggcgttcccttggcatcatttccgggttgaccggtgccggtggaaacgtgggcgcaggactcacacaacttctattcttcacctcatcgcaatactccactggtaggggtctcgagtacatgggcatcatgatcatggcatgcacgctgcccgtcgctcttgtgcactttccgcaatggggatccatgttcttcccggccagcgctgatgccactgaggaggagtactatgcttccgagtggtcggaggaggagaagggcaagggtctccatatcgcaggccaaaagttcgccgagaactcccgctcggagcgcggcaggcgcaacgtcatctttgccacatccgccacgccgcccaacaacacaccccagcaggtataaggcatttttttttgttacctatgaattttacagctcatggcgtatatatacaaacagtatatttacgtttgcagccccagcgtaataagttgtatgggggtttatctttttactatggtaaacctaaggacatgtattgtcaaattgagtccgaaattaatacatgaacagtgttgatgtttgtgtatgcttgaaaaaaaaaaaaaaaaaa

SEQ ID NO: 33 - DNA, 1704 bp, Hordeum vulgare
caccactgcaagcatatttaggcttagttagctccaaggagcaaagctaaaaagaacctagctaggctagctcgatccagctagctcagtagatatggaggtggaggccggagctcatggcgatgcggcggcgagcaagttcacgctgcccgtggactccgagcacaaggccaagtccttcaggctcttctccttcgccaacccgcacatgcgcaccttccacctctcgtggatctccttcttcacctgcttcgtctccacctttgccgctgctccgttggtgcccatcatccgcgacaacctcaacctcgccaaggccgacatcggcaatgccggtgtggcgtccgtgtccggctccatcttctcgaggctcgccatgggcgccatttgtgacctgcttggcccgcggtacggttgtgcctttctcgtcatgctatcggcgccaaccgtcttctgcatggccgtcatcgacgacgcgtcgggatacatcgcagtccgcttcctcatcggcttctccctcgcaaccttcgtgtcatgccagtactggatgagcacaatgttcaacagtaaaatcatcggcacggttaatggcctcgcagccgggtggggcaacatgggtggcggggccacacagctcatcatgcccctcgtcttccatgccatccaaaagtgtggtgccacaccctttgtggcatggcgtatcgcctacttcgtgccggggatgatgcacatcgtgatgggcctactcgtgctcaccatgggacaagacctccctgatgggaacctcgcaagcctgcagaagaagggagacatggccaaggacaagttctccaaggtcctttggggcgccgttaccaactaccggacatggatctttgtcctcctctatggctactgcatgggtgtcgagctcaccactggcaatgtcattgccgagtactacttcgatcacttccacctaaacctccgtgccgccggtaccatcgccgcttgcttcggcatggccaacatcgtcgcacgtcctatgggcggctacctctccgaccttggtgctcgctacttcggtatgcgtgctcgcctttggaacatctggatccttcagacagctggcggcgccttttgcatctggcttgggcgcgcctcggccctccccgcctcagtgactgccatggtcctcttctccatctgcgcccaggctgcgtgtggtgctatctttggtgtcgaacccttcgtctccaggcgttcccttggcatcatttccgggttgaccggtgctggtggaaacgtgggcgcagggctcacacagcttctcttcttcacttcgtcgcaatactccactggcaggggtcttgagtacatgggcatcatgatcatggcatgcaccttacccgtcgctctcgttcacttccctcagtggggctctatgttcttggctgccagtgccgacgccacggaggaggagtactacgcctcagagtggtcagaggaggagaagagcaagggtctccatatcgcaggacaaaagtttgctgagaactcccgctcggaacgcggcaggcgcaacgtcatccttgccacatccgccacaccacccaacaacacgcccctacacgtataagtttcaaattttgtgttacacaagaaatgtacatcttgctgagtatatatacacatcgtatattttagtaaaaaaaaaaaaaaaaa

SEQ ID NO: 34 - protein, 197 aa, Hordeum vulgare
MARSELVMVLLVVVLAAGCCTSAGAVAYLSKLPVTLDVTASPSPGQVLHAGEDVITVTWALNTTQAGKDADYKNVKVSLCYAPVSQKEREWRKTHDDLKKDKTCQFKVTQQAYPGAGKVEYRVALDIPTATYYVRAYALDASGTQVAYGQTAPTAAFNVVSITGVTTSIKVAAGVFSAFSVASLAFFFFIEKRKKNN

SEQ ID NO: 35 - protein, 201 aa, Hordeum vulgare
MARSDLVMALLVAVLAAGCCASAGAVAYLSKLTVTLDVTASPTPGQVLHAGEDVITVTWALNATRPAGDDAAYKSVKVSLCYAPASQKEREWRKTHDDLKKDKTCQFKVTQQPYAAGAAGGRVEYRVALDIPTAAYYVRAYALDASGTQVAYGQTAPATAFDVVSITGVTTSIKVAAGVFSTFSVVSLAFFFFIEKRKKNN

SEQ ID NO: 36 - protein, 198 aa, Hordeum vulgare
MARQGMVTALLLLVLAAGCCASAGAVAYLSKLPVTLDVTASPTPGQVLHAGEDVITVTWALNASQPAGKDADYKNVKVSLCYAPVSQKEREWRKTHDDLKKDKTCQFKITQQAYPGAGKVEYRVALDIPTATYYVRAYALDASGTQVAYGQTAPTAAFNVVSITGVTTSIKVAAGVFSAFSVASLAFFFFIEKRKKNN

SEQ ID NO: 37 - protein, 507 aa, Hordeum vulgare
MEVEAGAHGDTAASKFTLPVDSEHKAKSFRLFSFANPHMRTFHLSWISFFTCFISTFAAAPLVPIIRDNLNLAKADIGNAGVASVSGSIFSRLAMGAICDLLGPRYGCAFLVMLSAPTVFCMAVIDDASGYIAVRFLIGFSLATFVSCQYWMSTMFNSKIIGTVNGLAAGWGNMGGGATQLIMPLVFHAIQKCGATPFVAWRIAYFVPGMMHIVMGLLVLTMGQDLPDGNLASLQKKGDMAKDKFSKVLWGAVTNYRTWIFVLLYGYCMGVELTTDNVIAEYYFDHFHLDLRAAGTIAACFGMANIVARPTGGYLSDLGARYFGMRARLWNIWILQTAGGAFCIWLGRASALPASVTAMVLFSICAQAACGAIFGVAPFVSRRSLGIISGLTGAGGNVGAGLTQLLFFTSSQYSTGRGLEYMGIMIMACTLPVALVHFPQWGSMFFPASADATEEEYYASEWSEEEKAKGLHIAGQKFAENSRSERGRRNVILATSATPPNNTPQHV

SEQ ID NO: 38 - protein, 509 aa, Hordeum vulgare
MEVESSSHGAGDEAASKFSLPVDSEHKAKSIRLFSFANPHMRTFHLSWISFFTCFVSTFAAAPLVPIIRDNLNLAKADIGNAGVASVSGSIFSRLAMGAICDLLGPRYGCAFLVMLAAPTVFCMSLIDDAAGYITVRFLIGFSLATFVSCQYWMSTMFNSKIIGTVNGLAAGWGNMGGGATQLIMPLVFHAIQKCGATPFVAWRIAYFVPGMMHVVMGLLVLTMGQDLPDGNLASLQKKGEMAKDKFSKVVWGAVTNYRTWIFVLLYGYCMGVELTTDNVIAEYYFDHFHLDLRTSGTIAACFGMANIVARPAGGYLSDLGARYFGMRARLWNIWILQTAGGAFCLWLGRAKALPESITAMVLFSICAQAACGAVFGVIPFVSRRSLGIISGLSGAGGNFGAGLTQLLFFTSSKYGTGRGLEYMGIMIMACTLPVALVHFPQWGSMLLPPNANATEEEFYAAEWSEEEKKKGLHIPGQKFAENSRSERGRRNVILATAATPPNNTPQHA

SEQ ID NO: 39 - protein, 506 aa, Hordeum vulgare
MEVEAGAHGDTAASKFTLPVDSEHKAKSFRLFSFANPHMRTFHLSWISFFTCFVSTFAAAPLVPIIRDNLNLAKADIGNAGVASVSGSIFSRLAMGAICDLLGPRYGCAFLVMLSAPTVFCMAVIDDASGYIAVRFLIGFSLATFVSCQYWMSTMFNSKIIGTVNGLAAGWGNMGGGATQLIMPLVFHAIQKCGATPFVAWRIAYFVPGMMHIVMGLLVLTMGQDLPDGNLASLQKRGDMAKDKFSKVLWGAVTNYRTWIFVLLYGYCMGVELTTDNVIAEYYFDHFHLDLRAAGTIAACFGMANIVARPMGGYLSDLGARYFGMRALWNIWILQTAGGAFCIWLGRASALPASVTAMVLFSICAQAACGAIFGVAPFVSRRSLGIISGLTGAGGNVGAGLTQLLFFTSSQYSTGRGLEYMGIMIMACTLPVALVHFPQWGSMFFPASADATEEEYYASEWSEEEKGKGLHIAGQKFAENSRSERGRRNVIFATSATPPNNTPQQV

SEQ ID NO: 40 - protein, 507 aa, Hordeum vulgare
MEVEAGAHGDAAASKFTLPVDSEHKAKSFRLFSFANPHMRTFHLSWISFFTCFVSTFAAAPLVPIIRDNLNLAKADIGNAGVASVSGSIFSRLAMGAICDLLGPRYGCAFLVMLSAPTVFCMAVIDDASGYIAVRFLIGFSLATFVSCQYWMSTMFNSKIIGTVNGLAAGWGNMGGGATQLIMPLVFHAIQKCGATPFVAWRIAYFVPGMMHIVMGLLVLTMGQDLPDGNLASLQKKGDMAKDKFSKVLWGAVTNYRTWIFVLLYGYCMGVELTTGNVIAEYYFDHFHLNLRAAGTIAACFGMANIVARPMGGYLSDLGARYFGMRARLWNIWILQTAGGAFCIWLGRASALPASVTAMVLFSICAQAACGAIFGVEPFVSRRSLGIISGLTGAGGNVGAGLTQLLFFTSSQYSTGRGLEYMGIMIMACTLPVALVHFPQWGSMFLAASADATEEEYYASEWSEEEKSKGLHIAGQKFAENSRSERGRRNVILATSATPPNNTPLHV

SEQ ID NO: 41 - DNA, 961 bp, Zea mays
gctcagatccctcgcctcgtgtcgtgtctccggtcgacgacgaccaacagccagtgtgggccagacggacaccgccgagctatagcgcttggtgatagcaagggacgaccggcggccggaccggagcacgtacgtacgtaccgcagcgatggctcggcagcaaagcgtgcacgccttgtgtgtgctggcggcacttctcttcgccgcctccctgccgtcgccggccgccgcgggggtgcacctctcctcgctgcccaaagcgctcgacgtcaccacctccgccaaacccggccaagtcctgcacgccggcgtggactcgctgacggtgacgtggagcctgaacgccacggagccggccggcgccgacgccgggtacaagggcgtgaaggtgaagctgtgctacgcgccggcgagccagaaggaccgcgggtggcgcaagtccgaggacgacatcagcaaggacaaggcgtgccagttcaaggtcaccgagcaggcgtacgcggcggcggcgcccggcagcttccagtacgccgtcgcccgcgacgtcccctcgggctcctactacctgcgcgccttcgccacggacgcgtcgggcgccgaggtggcctacggccagacggcgcccaccgccgccttcgacgtcgccggcatcaccggcatccacgcctctctcaagatcgccgccggcgtcttctcggccttctccgtcgtcgcgctcgccttcttcttcgtcatcgagacccgcaagaagaacaagtagaacgagttgcggctgcgcgccatacatgcatacatgtaaatcgtcggcggcgatgagtggctgtcgttgctgattcattggtgcgcgcgactattttggtgtatcatgtaagttacttttctgcagtgtgtgcgtcaaaattaccaaataataacttaagtttctctacataaaaaaaaaaaaaaaaaaaaaaaaaaaaaa

SEQ ID NO: 42 - DNA, 936 bp, Zea mays
cctcgtcacacaccacaggctgtgtagagcgcgcgcctggcatgacgatggctcgtcctggggcggctttgccgctgctgctggtcgtggtcggcgcttgctgcgcgcgcctggcggcggcagtgcacctctccgcgctcggcaggacactcatcgtcgaggcgtcgccgaaggccggacaagtcctgcacgccggcgaggacacgataaccgtgacatggcacctcaacgcgtcggcgtccagcgtcgggtacaaggcgctggaggtgaccctctgctacgcgccggcgagccaggaggaccgcgggtggcgcaaggccaacgacgacttgagcaaggacaaggcgtgccagttcaggatcgcccggcatgcatacgccggcggccaggggacgctccggtacagggtcgcccgcgacgtccccaccgcgtcctaccacgtgcgcgcctacgcgctggacgcgtccggggcgccggtgggctacggccagaccgcgcccgcctactacttccacgtcgcgggcgtctcgggcgtccacgcgtccctccgggtcgccgccgccgtgctctccgcgttctccatcgccgcgctcgccttctttgtcgtcgtcgagaagaggaggaaggacgagtaggccgtaagcggcacacgcgatatataccagggaggcgccaccgtgcgagctagcgcttggtgagccgtcatgttgtgtagtgcagttgtaacacaagtattaagctaagcacaagtcgtaccatgtatccagctccagcatgaatccaaacccagcgtcagtcacaagctggacctgcatggtgcctgtcaaccaaaattcttcttggatcatactaatactatcgtctacagcatatagaacttgtatcatactaatttacatggcaccttctgctaaaaaaaaaaaaaaaaaaaaaaaa

SEQ ID NO: 43 - DNA, 1789 bp, Zea mays
acgcggggaagcacaagcaaccagccagctagtttccaagggatcacctgctctctagcactagcagcaatggcggccgtcggcgctccgggcagctctctgcacggagtcacggggcgcgagccggcgttcgccttctccacggagcacgaggaggcggcgagcaatggtggcaagttcgacctgccggtggactcagagcacaaggcgaagagcgtccgtctcttctccgtggcgaacccacacatgcgcaccttccacctctcctggatctccttcttcacctgcttcgtgtccaccttcgccgccgcgccgctggtccccatcatccgcgacaacctcaacctcaccaaggccgacatcggcaacgcgggcgtggcctcggtgtcgggctccatcttctcccgcctcaccatgggcgccgtctgcgacctgctgggcccgcgctacggctgcgccttcctcatcatgctgtccgcgcccaccgtgttctgcatgtcgctcatcgacgacgccgcgggctacatcaccgtcaggttcctcatcggcttctccctcgccaccttcgtctcctgccagtactggatgagcaccatgttcagcagcaagatcatcggcaccgtcaacgggctcgccgccggatggggcacaatgggaaggcggcgccacgcagctcatatgccgctcgtctacgacgtcatccgcaagtgcggcgccacgccattcacggcctggcgcctcgcctacttcgtgccgggcctcatgcacgtcgtcatgggcgtcctggtgctcacgctggggcaggacctccccgacggcaacctcaggtcgctgcagaagaagggcaacgtcaacaaggacagcttctccaaggtcatgtggtacgccgtcatcaactaccgtacctggatctttgtcctcctctacggctactgcatgggcgtcgagctcaccaccgacaacgtcatcgccgagtacatgtacgaccgcttcgacctcgacctccgcgtcgctgggaccatcgccgcctgcttcggcatggccaacatcgtcgcacgccccatgggcggcatcatgtccgacatgggcgcgcgctactggggcatgcgcgctcgcctctggaacatctggatcctccagaccgccggcggcgccttctgcctctggctggggcgcgccagcaccctccccgtctccgtcgtcgccatggtgctcttctccttctgcgcgcaggcggcatgcggcgccatcttcggggttatcccctttgtctcccgccgctccctcggcatcatctccggcatgacgggcgccggcggcaacttcggcgccgggctcacgcagctgctcttctttacctcctcgacctactccacgggcagggggctggagtacatgggcatcatgatcatggcgtgcacgctgcccgtggtgttcgtgcacttccctcagtgggggtccatgttctttccgcccagcgccaccgccgacgaggagggctactacgcctccgagtggaacgacgacgagaagagcaagggactccatagcgccagcctcaagttcgccgagaacagccgctcagagcgcggcaagcgaaacgtcatccaggccgacgccgccgccacgccggagcatgtctaagtctactactaagatggatcgatcgacgatcacctatacctctttgtatgtacgaatatgccttgttattactgcgcgcgcgcatatacaatacacgtgtgctccgttgacatgagttagaaaaaaaaaaaaaaaaaaaaaaaaa

SEQ ID NO: 44 - DNA, 1575 bp, Zea mays
atggcggccgtcggcgctccggggagctctctgcacggagtcacggggcgcgagccggcgttcgcattctccacggagcacgaggaggcggcgagcaatggcggcaagttcgacctgccggtggactcggagcacaaggcgaagagcgtccggctcttctccgtggcgaacccgcacatgcgcaccttccacctctcctggatctccttcttcacctgcttcgtgtccaccttcgccgccgcgccgctggtccccatcatccgcgacaacctcaacctcaccaaggccgacatcggcaacgcgggcgtggcctccgtgtcgggctccatcttctcccgcctcaccatgggcgccgtctgcgacctgctgggcccgcgctacggctgcgccttcctcatcatgctgtccgcgcccaccgtgttctgcatgtcgctcatcgacgacgccgcgggctacatcaccgtcaggttcctcatcggcttctccctcgccaccttcgtctcctgccagtactggatgagcaccatgttcagcagcaagatcatcggcaccgtcaacgggctcgccgccggatggggcaacatgggaggcggcgccacgcagctcatcatgccgctcgtctacgacgtcatccgcaagtgcggcgccacgcccttcacggcgtggcgcctcgcctacttcgtgccgggcctcatgcacgtcgtcatgggcgtcctggtgctcacgctggggcaggacctccccgacggcaacctcaggtcgctgcagaagaagggcaacgtcaacaaggacagcttctccaaggtcatgtggtacgccgtcatcaactaccgcacctggatcttcgtcctcctctacggctactgcatgggcgtcgagctcaccaccgacaacgtcatcgccgagtacatgtacgaccgcttcgacctcgacctccgcgtcgccgggaccatcgccgcctgcttcggcatggccaacatcgtcgcgcgccccatgggcggcatcatgtccgacatgggcgcgcgctactggggcatgcgcgctcgcctctggaacatctggatcctccagaccgccggcggcgccttctgcctctggctgggacgcgccagcaccctccccgtctccgtcgtcgccatggtgctcttctccttctgcgcgcaggcggcctgcggcgccatcttcggggtcatccccttcgtctcccgccgctccctcggcatcatctccggcatgacgggcgccggcggcaacttcggcgcggggctcacgcagctgctcttcttcacctcctcaacctactccacgggcagggggctagagtacatgggcatcatgatcatggcgtgcacgctacctgtggtgttcgtgcacttcccgcagtgggggtccatgttcttcccgcccagcgccaccgccgacgaggagggctactacgcctccgagtggaacgacgacgagaagagcaagggactccatagcgccagcctcaagtttgccgagaacagccgctcagagcgcggcaagcgaaacgtcatccaggccgatgccgccgccacgccggagcatgtctaa

SEQ ID NO: 45 - protein, 203 aa, Zea mays
MARQQSVHALCVLAALLFAASLPSPAAAGVHLSSLPKALDVTTSAKPGQVLHAGVDSLTVTWSLNATEPAGADAGYKGVKVKLCYAPASQKDRGWRKSEDDISKDKACQFKVTEQAYAAAAPGSFQYAVARDVPSGSYYLRAFATDASGAEVAYGQTAPTAAFDVAGITGIHASLKIAAGVFSAFSVVALAFFFVIETRKKNK

SEQ ID NO: 46 - protein, 195 aa, Zea mays
MARPGAALPLLLVVVGACCARLAAAVHLSALGRTLIVEASPKAGQVLHAGEDTITVTWHLNASASSVGYKALEVTLCYAPASQEDRGWRKANDDLSKDKACQFRIARHAYAGGQGTLRYRVARDVPTASYHVRAYALDASGAPVGYGQTAPAYYFHVAGVSGVHASLRVAAAVLSAFSIAALAFFVVVEKRRKDE

SEQ ID NO: 47 - protein, 524 aa, Zea mays
MAAVGAPGSSLHGVTGREPAFAFSTEHEEAASNGGKFDLPVDSEHKAKSVRLFSVANPHMRTFHLSWISFFTCFVSTFAAAPLVPIIRDNLNLTKADIGNAGVASVSGSIFSRLTMGAVCDLLGPRYGCAFLIMLSAPTVFCMSLIDDAAGYITVRFLIGFSLATFVSCQYWMSTMFSSKIIGTVNGLAAGWGTMGRRRHAAHMPLVYDVIRKCGATPFTAWRLAYFVPGLMHVVMGVLVLTLGQDLPDGNLRSLQKKGNVNKDSFSKVMWYAVINYRTWIFVLLYGYCMGVELTTDNVIAEYMYDRFDLDLRVAGTIAACFGMANIVARPMGGIMSDMGARYWGMRARLWNIWILQTAGGAFCLWLGRASTLPVSVVAMVLFSFCAQAACGAIFGVIPFVSRRSLGIISGMTGAGGNFGAGLTQLLFFTSSTYSTGRGLEYMGIMIMACTLPVVFVHFPQWGSMFFPPSATADEEGYYASEWNDDEKSKGLHSASLKFAENSRSERGKRNVIQADAAATPEHV

SEQ ID NO: 48 - protein, 524 aa, Zea mays
MAAVGAPGSSLHGVTGREPAFAFSTEHEEAASNGGKFDLPVDSEHKAKSVRLFSVANPHMRTFHLSWISFFTCFVSTFAAAPLVPIIRDNLNLTKADIGNAGVASVSGSIFSRLTMGAVCDLLGPRYGCAFLIMLSAPTVFCMSLIDDAAGYITVRFLIGFSLATFVSCQYWMSTMFSSKIIGTVNGLAAGWGNMGGGATQLIMPLVYDVIRKCGATPFTAWRLAYFVPGLMHVVMGVLVLTLGQDLPDGNLRSLQKKGNVNKDSFSKVMWYAVINYRTWIFVLLYGYCMGVELTTDNVIAEYMYDRFDLDLRVAGTIAACFGMANIVARPMGGIMSDMGARYWGMRARLWNIWILQTAGGAFCLWLGRASTLPVSVVAMVLFSFCAQAACGAIFGVIPFVSRRSLGIISGMTGAGGNFGAGLTQLLFFTSSTYSTGRGLEYMGIMIMACTLPVVFVHFPQWGSMFFPPSATADEEGYYASEWNDDEKSKGLHSASLKFAENSRSERGKRNVIQADAAATPEHV

SEQ ID NO: 49 - DNA, 2093 bp, Zea mays
gctcagatccctcgcctcgtgtcgtgtctccggtcgacgacgaccaacagccagtgtgggccagacggacaccgccgagctatagcgcttggtgatagcaagggacgaccggcggccggaccggagcacgtacgtacgtaccgcagcgatggctcggcagcaaagcgtgcaggccttgtgtgtgctggcggcgcttctcttcgccgcctccctgccgtcgccggccgccgcgggggtgcacctctcctcgctgcccaaagcgctcgacgtcaccacctccgccaaacccggccaaggtgcgcgcgcgttccggcccggctcatagtcatagccaaaggattagcactttgattacttgctcggttaattcatagtcctattcttctctatgtttgaaacccccctttagatttgttcattcacaatcaaggagctagctgattaaaatacacacgattgccataaaatatatgcttctcgcagtcctgcacgccggcgtggactcgctgacggtgacgtggagcctgaacgccacggagccggccggcgccgacgccgggtacaagggcgtgaaggtgaagctgtgctacgcgccggcgagccagaaggaccgcgggtggcgcaagtccgaggacgacatcagcaaggacaaggcgtgccagttcaaggtcaccgagcaggcgtacgcggcggcggcgcccggcagcttccagtacgccgtcgcccgcgacgtcccctcgggctcctactacctgcgcgccttcgccacggacgcgtcgggcgccgaggtggcctacggccagacggcgcccaccgccgccttcgacgtcgccggcatcaccggcatccacgcctctctcaagatcgccgccggcgtcttctcggccttctccgtcgtcgcgctcgccttcttcttcgtcatcgagacccgcaagaagaacaagtagaacgagttgcggctgcgcgccatacatgcatacatgtaaatcgtcggcggcgatgagtggctgtcgttgctgattcattggtgcgcgcgactattttggtgtatcatgtaagttacttttctgcagtgtgtgcgtcaaaattaccaaataataacttaagtttctctgctgatccggttttcgattacgaatggaggggctcaaaatatatatagtctgctcgaaagtggattatattgcccacttattacaaatttatttatttctagtttattttgcagtaatcattgaaaaggccagcccagcgtctctcttcgatttaagtaaacaatagcataaaatccgcctcctcatctaagcgtttaccactctattaagctacatctttcgataaaatggtagagcatgtgtcattgacctgcaagattcaatgctctatcctctgagctgttcgcctgttctcaagagtgagcaaacatgaggggcatcaaagatcatttccatatgaaaatctcggtcaactgcgacgcaaagaagaatcgttggacacttctccaaacgcgatgaaaggccaagaaggttatgccacactgtttccacgggaattagtcggcacaccggtgatctatcgtttgaagaaacgaaaagccaatgaactttgtcaacggcacagaaaaaatgacgatagcgtcacaatcttccaacaaaagcatatatttccacgaaaccgaaccattttgttgacgcttttttggagcgccaaatactcaagaagaaccggcggcggtgctctctggtcaggcgcggacggtccgcggcctggggccggacggtccgcgacctggcgcgagggctagagtttcctgcctgacggccggacggtccgcgccctagggccggacggtccgcgcgtgcgcaggggcggcggaagatcaccggcggcgcctggatctcgctcccgggagggaccccgtcggggaggagagatcccaggggttgtcttaggctcgggccggccgacctagactcctctaatcggcgtagagtcgaagagaggtgaagaatttggggattaagaggctaaactagaactactcctaattgtactggaaataaatgcgaatagaagttgtattgattcgattg

SEQ ID NO: 50 - DNA, 2100 bp, Zea mays
atgtgatcacaagactgtcatgttttgtcatagcaccctgatgcttagtacatccaatgatccaagcacaatcttggcatgcctgcagattggtagcacatccatttgatctcactccatgtaagtgggtcaacactaaggtattatatgtttgcacccagatcattatgtgatagcatgctaccaccaccatagcccaccatatgaccaccaggaatacgctggggtgatcatgcctgaagcattggatattgatgtggattttgatcttataatgtcatccttactaataattatttgaacgttgtgcaggtaatcaatgctttagtgaattttattcttgatatacttgtcataatgtagcattcttgtgagcaaatcattatctgtgttaaggtcatggtcatggacctgcagcaatatcaaatgaaacaaatgtttgaaatgatatctggtgtgacatttttagtgagattgtagtggtatattggtaaagttgtattttgatcacttggagttttcgtttacttcacccactgctgtataatttccttgttctgaataagcattagaacataacaattctaactagcattttacacgattgaacaaacccaagcaccccaatctctacatcaatagctctgtgctgctaatctgctagcacttcaatatttctgttatttatttgcctaatttggttgagtctggcttgtgaagtgtattaacaatagttaaaaattgtatattgtgatatttgttgatgacttggttatatttgtggtgtttgtttgaatttatctgactataagcaaaatttcaatattttttgttttaatgtgctgacacactcaagtggatttgtgcacataatatccatcaaaagaagatgcttgtataattttatatttgatactgttgccatgagtacacctcttttcagtttttagttggagctttttttccctacatacacttagctcatgtttagattgccaggatcctagcacatttcatgtgaattggtgctgatttgatgcatctaaacaagcattaaaagtgtgtatttatgatttttattagtttttgctagcagaccttcaggctgctgaaatttcttcaatgtataggttctgttttctacttttcttccacattggtcaggcaatatgtttttgtgtcacactcaaaaaacacttctgaattttataaacagtgatttctgtataattctgattccaaacaatggtggtatgtcttcttgtactcttataatgtactgatgatatcgggttttatcttctattatggttgtaaggcttgatgcctgaaactgctgattattaaatcactttcaagtttcaacacacctaaatttgggatatgggatagattacattatgctagggtaatcgaattttctttttttctaccctttcgctgttggtggttaaatgattcttaacatgatctgatacccacattttattttgggaaaataacattttttgttcgtctaattcagtttaagtgtttttagggcaattcggtttcagtatctttttcaatcttccgtgaacagttaaaaattgaagttatttaactaatttggtactctagcctgtcaaaaaaaatacttgtgttgattccttctctgaaaatttttccatttagtttctcgggctgtattcccgtctgatttgaatattcgttcaccaaacctttcaggccggagtctgatgaagaaagaaatttgaaagaagaaatcaacctcctgaaggtggatcttaaagaaatcgagggaaagatgagtaatggttctgagcagacatcggtagatgcaaaagatttgtctgagaagatatctatgttggaaagccagcttgagcagctttcaagggagttggatgacaagattcgatttgggcaaaggccacgttctggtgcaggcagggttacaacacttgcacccactagtttaggggaggaaccacaggctacagtggtggacagaccacgttctcgtggtggtatggaaccacccccaaggcaggaagaaagatggggatttcaaggaagccgagaaaggggctcgtttgg

SEQ ID NO: 51 - DNA, 793 bp, Triticum aestivum
tcctctcttgccttctccgatcgacaacagcagccaagcactaactagccgcgcgcagggatggctcgccaaggtatggtcacggcgctgttgctggtggtcctcgccgccggctgctgcgcgtcggcgggcgccgtggcgtacctctccaagctgccggtgaccctcgacgtcaccgcctcccccagtcccggccaagttcttcacgccggcgaggacgtgatcacggtgacgtgggccctgaacgcgagccagccggccggcaaggacgtcgactacaagaacgtgaaggtgagcctctgctacgcgccggtgagccagaaggagcgcgagtggcgcaagacccacgacgacctcaagaaggacaagacctgccagttcaaggtcacccagcaggcctaccccggcaccggcaaggtcgagtaccgcgtcgccctcgacatccccaccgccacctactacgtgcgcgcctacgcgctcgacgcctccggcacccaggtcgcctacggccagaccgcgccctcctccgccttcaacgtcgtcagcataaccggcgtcaccacctccatcaaggtcgccgccggcgtcttctccgccttctccgtcgcctccctcgcattcttcttcttcattgagaaacgcaagaagaacaactagatgtggagatcggaaccgtagcaaagtttgttggctttctctggccggtcgttttctctctacatgtaacagtatcagtacggaccagcacgtacgtacgtacggtgcaaactgtacggataaatgttcgggtgca

SEQ ID NO: 52 - DNA, 761 bp, Triticum aestivum
ttctctgaccgagcagctgcgagctcgatcgagcccaagttcgacggcgatggcacagtcgaagctggtcatggcgttgctggtggcggtcctcgccgccggctgctgcgcgtcggccggcgccgtggcgtacctctccaagcttcctgtgaccctcgacgtcatcgcatcccccagccccggccaagttctccatgccggcgaggacgtgatcacagtgacgtgggccctcaacgcgtctcggccggccggcgacgacgccgcctacaagaacgtgaaggtcagcctctgctacgcgccggcgagccagaaggagcgcgagtggcgcaagacccacgacgacctcaagaaagacaagacctgccagttcaaggtcgcccagcagccctacgccggcgccggcggcagggtcgagtaccgcgtcgccctcgacatccccaccgccacctactacgtgcgcgcctacgcgctcgacgcctccggcacgcaggtcgcctacggccagaccgcgcccgccgccgccttcaacgtcgtcagcatcacgggcgtcaccacctccatcaaggtcgccgccggcgtcttctccaccttctccgtcgtctccctcgccttcttcttcttcattgagaagcgcaagaagaataactaagctcagaaccgtacgtggcaaggtgttcgtccgtgcgcgacttcttcctcttgtcctttgcaacgtacgcacaaaaggtgtacatgtaatgtaagagagtgttggtgtttcc

SEQ ID NO: 53 - DNA, 1796 bp, Triticum aestivum
aagctagcaccaagcctccaaggagcaagaagagaagaagccttgctcgatcaagcaaggtcgaaatggaggtggaggccagcgcccatggcgacacggcggcgagcaagttcacgctgcccgtggactccgagcacaaggccaagtccttcagactcttctccttcgccaacccccacatgcgtaccttccacctctcctggatatccttcttcacctgcttcgtctccaccttcgcggcggcaccgttggtgcccatcatccgtgacaacctcaacctcgctaaggccgacatagggaatgccggtgtggcatctgtgtctgggtccatcttctccaggcttgccatgggtgccatctgcgaccttttagggccgcggtatggctgcgccttcctcgtcatgctctcagcacccactgtgttttgcatggctgctatcgacgatgcgtcaggctacatcgccgtacgcttcctcattggcttctccctcgccaccttcgtgtcatgccaatattggatgagcaccatgttcaacagtaagatcattggcacggtgaatggcctcgcggccggctggggcaacatgggcggtggtgccacacaactcatcatgccgcttgttttccatgccatccaaaagtgtggtgccacacccttcgtggcatggcgtattgcctatttcgtgccgggaatgatgcacatcgtcatggggttgcttgtgctcactatgggccaagatctccccgacggcaaccttgcgagtctccagaagaagggggacatggccaaggacaaattctcgaaggtcctttggggtgcggtcaccaactaccggacatggatattcgtcctcctctacggctactgcatgggtgtcgagctcaccaccgacaacgtcatcgccgagtactactacgaccacttccaccttgaccttcgcgccgctggcaccattgccgcttgcttcggcatggccaacatcgtcgcgcgtcctatgggtggctatctctctgaccttggtgcccgctacttcggcatgcgtgctcggctctggaacatctggatcctccagaccgctggtggcgctttctgcatctggctcggtcgtgcatcggcccttcctgcctcagtcacggccatggtcctcttttccatttgtgcacaagctgcttgtggtgctgtatttggcgtcgcacccttcgtttccaggcgttcccttggcatcatctccgggctgaccggcgctggtggcaatgttggcgcagggctaacgcaacttcttttcttcacatcgtcgcaatactccaccgggaggggtctcgagtacatgggcatcatgatcatggcatgcacattacccgtcgctctggtgcacttcccccaatggggctccatgttcttcccggctagcgctgatgccacggaagaggaatactatgcttctgagtggtcggaggaggagaagggcaagggtctccatattacaggccaaaagttcgcagagaactcccgctcagagcgcggcaggcgcaacgtcatccttgccacatccgccacgccacccaacaacacaccccagcacgtataaggcccttatttttatgtcacctaagaattttactgttcatcacgtatatatacaaaccgtatatctacgtctgcagccccagcgtaataagttgtatggggatttatgtttctactagtaaacttaaggaaacgctgcttttgcgttcctgctctgtacgcatgaaatgtaatatcaatttgagtccgaaattactacaaaaaaaaa

SEQ ID NO: 54 - protein, 198 aa, Triticum aestivum
MARQGMVTALLLVVLAAGCCASAGAVAYLSKLPVTLDVTASPSPGQVLHAGEDVITVTWALNASQPAGKDVDYKNVKVSLCYAPVSQKEREWRKTHDDLKKDKTCQFKVTQQAYPGTGKVEYRVALDIPTATYYVRAYALDASGTQVAYGQTAPSSAFNVVSITGVTTSIKVAAGVFSAFSVASLAFFFFIEKRKKNN

SEQ ID NO: 55 - protein, 199 aa, Triticum aestivum
MAQSKLVMALLVAVLAAGCCASAGAVAYLSKLPVTLDVIASPSPGQVLHAGEDVITVTWALNASRPAGDDAAYKNVKVSLCYAPASQKEREWRKTHDDLKKDKTCQFKVAQQPYAGAGGRVEYRVALDIPTATYYVRAYALDASGTQVAYGQTAPAAAFNVVSITGVTTSIKVAAGVFSTFSVVSLAFFFFIEKRKKNN

SEQ ID NO: 56 - protein, 507 aa, Triticum aestivum
MEVEASAHGDTAASKFTLPVDSEHKAKSFRLFSFANPHMRTFHLSWISFFTCFVSTFAAAPLVPIIRDNLNLAKADIGNAGVASVSGSIFSRLAMGAICDLLGPRYGCAFLVMLSAPTVFCMAAIDDASGYIAVRFLIGFSLATFVSCQYWMSTMFNSKIIGTVNGLAAGWGNMGGGATQLIMPLVFHAIQKCGATPFVAWRIAYFVPGMMHIVMGLLVLTMGQDLPDGNLASLQKKGDMAKDKFSKVLWGAVTNYRTWIFVLLYGYCMGVELTTDNVIAEYYYDHFHLDLRAAGTIAACFGMANIVARPMGGYLSDLGARYFGMRARLWNIWILQTAGGAFCIWLGRASALPASVTAMVLFSICAQAACGAVFGVAPFVSRRSLGIISGLTGAGGNVGAGLTQLLFFTSSQYSTGRGLEYMGIMIMACTLPVALVHFPQWGSMFFPASADATEEEYYASEWSEEEKGKGLHITGQKFAENSRSERGRRNVILATSATPPNNTPQHV

SEQ ID NO: 57 - DNA, 1887 bp, Chlamydomonas reinhardtii
atggacgttttccagtatacgacactggacaagggcgctggttctgcgcttttccgtgcacctcgtctaacatatatcggtgccgcagacgtccagagcgagacgcgcaagaatgtctacaggcgtcgactacttgggacatggcgcggtgaaggcgactgaggggccgccggtcaacccgtcaggccgcaagtacccttacgagctcgactcggagggcaaggccaaaagcattcccgtgtggcgcttcaccaacccgcacatgggcgcctttcatctgtcctggttcgccttcttcatttccttcctcgccaccttcgcgccggcctcgctgctgcccatcatccgcgacgacctgttcctgaccaaggcgcagctgggcaacgccggtgtggcggccgtgtgcggcgccatcgcggcacgcgtgctcatgggcgtgtttgtggacatcgtgggcccccgctacggcaccgcagccaccatgttgatgaccgctccggccgtgttctgcatggccctggtcaccgacttcgccacgttcgccgccgtgcgcttcttcatcggcctcagcctctgcatgttcgtgtgctgtcagttctggtgcggcaccatgttcaacgtccaaatagtgggcactgccaacgccatcgccgggggctggggcaacatgggcggcggcgcgtgtcacttcatcatgccgctcatctaccagggcatcaaggacggcggcgtgccgggataccaggcctggcgctgggccttcttcgtgccggctgtcttctacatcgccacggccctggccaccctggccctgggcattgaccaccccagcggcaaggactaccgcgacctgaaaaaggagggggcgctcaagtccaagggcgccatgtggccagtcatcaagtgcggcctcggcaactacaggtcttggatcctggccctgacgtacggctactccttcggtgttgagttgacggtggacaacattatcgtggagtacatgtttgaccagttcgggctgtcgttgacgtggcgggcgcgctgggcggcatgtttggcatgatgaaccttttcagccgggccagcggcggcatgatcagcgacctcatcgccaagcccttcggaatgcgcggtcgcatctgcgctctctggatcatccagaccctggggggcatcttctgcgtcatcctgggccgggtccacaacagcctgacctccaccatcgtcatcatgatcatcttctccatcttctgccagcaagcctgcggcctgcacttcggcatcacgcccttcgtgtcgcgccgcgcctacggcgtggtctccggcctcgtgggcgcaggcggcaacaccggcgccgccatcacacaagccatctggttcgccggcaccgccccctggcagctgaccctcaccaagtaccagggtctggagtacatgggataccagaccattggtctgacgctggcgctgttcttcatctggttccccatgtggggctccatgctgaccggaccgcgcgagggcgcaacagaggaggactactacatcaaggagtggagtgcggaggaagtggctgacggcctgcaccacaccagcctgcgctttgcaatggagtcccgctcgcagcgcggcacacgcaccagcacccagaccaaggtgatgtcggtcggcgacggcgccggcagcaacaaggcggaggtggtggtggtggcggcggcgcagcagggcgcggtgccgatggcatctgtggaggaggggagcagcggccgcagcagcagctcggggggccaccagcagcaggatgcgcatatacttcactgcatacgtactgttgttcaaaaagcgccgcgagtgggcgagcgggagagcgagcgggagagggactga

SEQ ID NO: 58 - protein, 628 aa, Chlamydomonas reinhardtii
MDVFQYTTLDKGAGSALFRAPRLTYIGAADVQTRRARMSTGVDYLGHGAVKATEGPPVNPSGRKYPYELDSEGKAKSIPVWRFTNPHMGAFHLSWFAFFISFLATFAPASLLPIIRDDLFLTKAQLGNAGVAAVCGAIAARVLMGVFVDIVGPRYGTAATMLMTAPAVFCMALVTDFATFAAVRFFIGLSLCMFVCCQFWCGTMFNVQIVGTANAIAGGWGNMGGGACHFIMPLIYQGIKDGGVPGYQAWRWAFFVPAVFYIATALATLALGIDHPSGKDYRDLKKEGALKSKGAMWPVIKCGLGNYRSWILALTYGYSFGVELTVDNIIVEYMFDQFGLSLTVAGALGGMFGMMNLFSRASGGMISDLIAKPFGMRGRICALWIIQTLGGIFCVILGRVHNSLTSTIVIMIIFSIFCQQACGLHFGITPFVSRRAYGVVSGLVGAGGNTGAAITQAIWFAGTAPWQLTLTKYQGLEYMGYQTIGLTLALFFIWFPMWGSMLTGPREGATEEDYYIKEWSAEEVADGLHHTSLRFAMESRSQRGTRTSTQTKVMSVGDGAGSNKAEVVVVAAAQQGAVPMASVEEGSSGRSSSSGGHQQQDAHILHCIRTVVQKAPRVGERESERERD

SEQ ID NO: 59 - DNA, 1890 bp, Glycine max
tcacactttcttccttaattttctagctcttgctacgtacttgaattcaattagttattaatggctgagattgagggttctcccggaagctccatgcatggagtaacaggaagagaacaaacatttgtagcctcagttgcttctccaattgtccctacagacaccacagccaaatttgctctcccagtggattcagaacacaaggccaaggttttcaaactcttctccctggccaatccccacatgagaaccttccacctttcttggatctccttcttcacctgcttcgtctcgacattcgcagcagcacctcttgtgcccatcatccgcgacaaccttaacctcaccaaaagcgacattggaaacgccggggttgcttctgtctccggaagcatcttctcaaggctcgcaatgggtgcagtctgtgacatgttgggtccacgctatggctgcgccttcctcatcatgctttcggcccctacggtgttctgcatgtcctttgtgaaagatgctgcggggtacatagcggttcggttcttgattgggttctcgttggcgacgtttgtgtcgtgccagtactggatgagcacgatgttcaacagtaagattatagggcttgcgaatgggactgctgcggggtgggggaacatgggtggtggagccactcagctcataatgcctttggtgtatgagcttatcagaagagctggggctactcccttcactgcttggaggattgccttctttgttccgggtttcatgcatgtcatcatggggattcttgtcctcactctaggccaggacttgcctgatggaaacctcggggccttgcggaagaagggtgatgtagctaaagacaagttttccaaggtgctatggtatgccataacaaattacaggacatggatttttgctctcctctatgggtactccatgggagttgaattaacaactgacaatgtcattgctgagtatttctatgacagatttaatctcaagctacacactgctggaatcattgctgcttcatttggaatggcaaacttagttgctcgaccttttggtggatatgcttcagatgttgcagccaggctgtttggcatgaggggaagactctggaccctttggatcctccaaaccttaggaggggttttctgtatttggcttggccgtgccaattctcttcctattgctgtattggccatgatcctgttctctataggagctcaagctgcatgtggtgcaacttttggcatcattcctttcatctcaagaaggtctttggggatcatatcaggtctaactggtgcaggtggaaactttgggtctggcctcacccaattggtcttcttttcaacctccaaattctctactgccacaggtctctccttgatgggtgtaatgatagtggcttgcactctaccagtgagtgttgttcacttcccacagtggggtagcatgtttctaccaccctcaaaagatgtcagcaaatccactgaagaattctattacacctctgaatggaatgaggaagagaagcagaagggtttgcaccagcaaagtctcaaatttgctgagaatagccgatctgagagaggaaagcgagtggcttcagcaccaacacctccaaatgcaactcccactcatgtctagccatagcacttcaatcaaagaagatcatgaaacataattactgagcagtattgggaatgaagaaccatgagttgaagaattttctaataagaaatcttgtaacatgtagacatagaatgttctggttctggtttgcgtgtggtgtaagagttgtctacttgtggtaagtcataagtatcataatcagtatgtcaatgcagatcttgatgctgagtatcaatagtatcaaaaaaaaaa

SEQ ID NO: 60 - protein, 530 aa, Glycine max
MAEIEGSPGSSMHGVTGREQTFVASVASPIVPTDTTAKFALPVDSEHKAKVFKLFSLANPHMRTFHLSWISFFTCFVSTFAAAPLVPIIRDNLNLTKSDIGNAGVASVSGSIFSRLAMGAVCDMLGPRYGCAFLIMLSAPTVFCMSFVKDAAGYIAVRFLIGFSLATFVSCQYWMSTMFNSKIIGLANGTAAGWGNMGGGATQLIMPLVYELIRRAGATPFTAWRIAFFVPGFMHVIMGILVLTLGQDLPDGNLGALRKKGDVAKDKFSKVLWYAITNYRTWIFALLYGYSMGVELTTDNVIAEYFYDRFNLKLHTAGIIAASFGMANLVARPFGGYASDVAARLFGMRGRLWTLWILQTLGGVFCIWLGRANSLPIAVLAMILFSIGAQAACGATFGIIPFISRRSLGIISGLTGAGGNFGSGLTQLVFFSTSKFSTATGLSLMGVMIVACTLPVSVVHFPQWGSMFLPPSKDVSKSTEEFYYTSEWNEEEKQKGLHQQSLKFAENSRSERGKRVASAPTPPNATPTHV